(19) United States
(12) Patent Application Publication
(10) Pub. No.: US 2014/0030269 A1
(43) Pub. Date: Jan. 30, 2014

(54) Staphylococcus aureus Specific Human Recombinant Polyclonal Antibodies and Uses Thereof

(71) Applicant: Excelimmune, Inc., Woburn, MA (US)

(72) Inventor: Vincent W. Coljee, Watertown, MA (US)

(73) Assignee: Excelimmune, Inc., Woburn, MA (US)

(21) Appl. No.: 13/951,645

(22) Filed: Jul. 26, 2013

Related U.S. Application Data
(63) Continuation of application No. 13/820,238.
(60) Provisional application No. 61/379,580, filed on Sep. 2, 2010.
(60) Provisional application No. 61/380,934, filed on Sep. 8, 2010.

(51) Int. Cl. C07K 16/12

(52) U.S. Cl. CPC C07K 16/1271
USPC 424/142.1; 530/389.5; 530/388.15

(57) ABSTRACT

Human antibodies that bind to one or more strains Staphylococcus aureus strains, including methicillin resistant strains, are disclosed. Also disclosed are human recombinant polyclonal antibody compositions, and therapeutic methods for using the antibodies.

### CROSS-REFERENCE TO RELATED APPLICATIONS

This application is a continuation of prior application Ser. No. 13/820,238, filed Mar. 1, 2013, which is a national phase entry of International Application No. PCT/US2011/050422, filed Sep. 2, 2011, which claims the benefit of and priority to U.S. Provisional Application No. 61/379,580, filed Sep. 2, 2010 and U.S. Provisional Application No. 61/380,934, filed Sep. 8, 2010; the entire contents of all of which are incorporated herein by reference.

### SEQUENCE LISTING

In accordance with 37 C.F.R. §1.52(e)(5), the present specification makes reference to a Sequence Listing (entitled “Sequence Listing”, created on Jul. 25, 2013 as a .txt file, and is 333 kilobytes). The entire contents of the Sequence Listing are incorporated herein by reference.

### FIELD OF THE INVENTION

The field of the invention is molecular biology, immunology and infectious disease. More particularly, the field is anti-Staphylococcus aureus antibodies and therapeutic human recombinant polyclonal antibodies.

### BACKGROUND

Antibiotic-resistant strains of Staphylococcus aureus (S. aureus) are on the rise and are a major health concern, both in the hospital setting and, increasingly, in the community at large. Methicillin-resistant S. aureus (MRSA) is currently the leading cause of death due to infectious disease in the United States, surpassing deaths from HIV/AIDS and tuberculosis combined. The death rate from invasive MRSA infection is now 20% (Heron, M. P. et al., 2008. 56: p. 1-52). Resistant forms of S. aureus have emerged within a few years of the introduction of each new class of antibiotics (Kirby, W. M., SCIENCE (1944) 99:452-453; Boucher, H. W. et al., CLIN INFECT DIS (2009) 48:1-12). For example, in the last decade, the first cases of vancomycin-resistant S. aureus (VRSA), and vancomycin-intermediate resistant S. aureus (VISA) have been reported. Given the current status in research and development of new antibiotics, resistance in bacteria is increasing faster than our ability to develop novel antibiotics to treat resistant strains (Boucher, H. W. and G. Sakoulas, CLIN INFECT DIS (2007) 45:601-8; Walsh, C., NAT REV MICROBIOL (2003) 1:65-70). Alternative treatments are therefore needed to replace those that are increasingly ineffective.

Recent research has shown that humans infected with S. aureus have a diverse humoral immune reaction to the organism (Verkaik, N. J. et al., EUR J CLIN MICROBIOL INFECT DIS (2010) 29:509-18; Verkaik, N. J. et al., CLIN INFECT DIS (2010) 50:61-8). Likewise, studies on the S. aureus exoproteome indicate that few strains express the same spectrum of antigens, and even antigens expressed in common between strains may be expressed at widely varying levels and in dissimilar temporal expression patterns (von Eiff, C. et al., DIAGN MICROBIOL INFECT DIS (2004) 49:157-62; Bania, J. et al., INT J FOOD MICROBIOL (2006) 108: 36-41; Ziebandt, A. K. et al., PROTEOMICS (2010) 10:1634-44).

One peptide that appears to be conserved among S. aureus strains and plays a role in the hemolytic activity of S. aureus is delta-hemolysin (δ-HL; also known as delta-toxin). Recent studies have shown that the hemolytic activity of delta-toxin may be modulated by interaction with lipid raft regions of the cell membrane (Pokorny, A. and P. F. Almeida, BIOCHEMISTRY (2005) 44(27): 9538-44). In addition to delta toxin, there have been six more small cytolytic peptides identified. These peptides were identified by genome analysis (Wang, R. et al., NATURE MEDICINE (2007) 13: 1510-1514). The peptides are represented in all strains tested by Wang et al. (2007) and had higher levels of expression in community acquired (CA) MRSA strains. The expression of these peptides are theorized to be one means by which CA-MRSA strains have a greater virulence compared to hospital acquired (HA) strains. Deletion of any of the six cytolytic peptides decreases virulence of MRSA strains in various in vitro and in vivo models (Wang, R. et al., NATURE MEDICINE (2007) 13: 1510-1514).

Thus, there is a need for improved therapeutic agents that can be used to treat and/or prevent S. aureus infections.

### SUMMARY

The invention is based, in part, upon the discovery of human polyclonal antibody compositions that bind one or more strains of S. aureus. In one aspect, the disclosed polyclonal antibody compositions comprise at least three different human antibodies that individually bind one or more S. aureus strains. In certain embodiments, the disclosed polyclonal antibody compositions bind to at least three different strains of S. aureus. Exemplary polyclonal antibodies described herein contain specific S. aureus binding sites based on the CDRs of individual antibodies of the polyclonal antibody composition. The individual antibodies of the polyclonal antibody composition may bind proteins that are known or likely to be involved in S. aureus virulence (e.g., delta-toxin) or, alternatively, they may bind previously uncharacterized S. aureus target proteins. The disclosed human polyclonal antibody compositions can neutralize the activity of S. aureus toxins, cell surface antigens and/or immunomodulating antigens. In an exemplary embodiment, the disclosed human polyclonal antibody compositions are broad-spectrum therapeutic antibodies with neutralizing activity against multiple S. aureus proteins that are present on one or more S. aureus strains. In certain embodiments, the disclosed recombinant polyclonal antibody compositions mimic the natural human immune response. It is contemplated herein that the disclosed recombinant polyclonal antibody compositions provide protection against a range of virulence factors. Such polyclonal antibodies can be used as therapeutic agents to treat S. aureus infections including antibiotic-resistant S. aureus strains.

Also disclosed herein are isolated, individual human antibodies, or antigen binding fragments thereof, that specifically bind one or more strains of S. aureus, including antibiotic resistant strains (e.g., MRSA). In one embodiment, an isolated human antibody as described herein binds (e.g., specifically binds) to S. aureus delta-toxin (e.g., antibody 5.6.H9 and antibody 5.55.D2). In another embodiment, an isolated human antibody described herein binds (e.g., specifically binds) to the toxin phenol soluble modulin beta-1 (e.g., antibody 22.18A.E9).

These and other aspects and advantages of the invention will become apparent upon consideration of the following figures, detailed description, and claims. As used herein, “including” means without limitation, and examples cited are non-limiting.

### DESCRIPTION OF THE DRAWINGS

The invention can be more completely understood with reference to the following drawings.

FIG. 1 (prior art) is a schematic representation of a typical naturally-occurring antibody. Naturally occurring antibodies are multimeric proteins that contain four polypeptide chains. Two of the polypeptide chains are called heavy chains (H chains), and two of the polypeptide chains are called light chains (L chains). The immunoglobulin heavy and light chains are connected by an interchain disulfide bond. The immunoglobulin heavy chains are connected by interchain disulfide bonds. A light chain consists of one variable region (V_{L}) and one constant region (C_{L}). The heavy chain consists of one variable region (V_{H}) and at least three constant regions (CH₁, CH₂ and CH₃). The variable regions determine the specificity of the antibody. Each variable region comprises three hypervariable regions also known as complementarity determining regions (CDRs) flanked by four relatively conserved framework regions (FRs). The three CDRs, referred to as CDR₁, CDR₂, and CDR₃, contribute to the antibody binding specificity.

FIG. 2 is a series of panels showing data from a representative plate (plate 5.6) of isolated fully human antibodies. FIG. 2A is a representative FACS analysis showing the population of CD38+/CD19+ plasma cells targeted for cloning; FIG. 2B shows the amplified variable heavy and light antibody genes from individual plasma cells in a 96-well plate; FIG. 2C shows representative data from an antibody expression ELISA showing that the majority of amplified antibody cognate pairs are cloned and are able to express antibody; FIGS. 2D and E show data from screening ELISA assays in which a well H9 was scored as a positive against S. aureus protein in FIG. 2D and wells H2 and A12 were scored as positives in FIG. 2E. The hits were confirmed with multiple replicates in the ELISA shown in FIG. 2F.

FIG. 3 is a graph showing that an anti-S. aureus recombinant polyclonal antibody mixture of five antibodies can protect mice from a challenge with live S. aureus (community acquired-MRSA strain, USA300) at 3.5×10⁸ CFU or 5×10⁸ CFU (⋄, 5 antibody composition at 3.5×10⁸ CFU; □, 5 antibody composition at 5×10⁸ CFU; ▴, PBS at 3.5×10⁸ CFU (negative control); and x, vancomycin at 2×10⁹ CFU (positive control).

FIG. 4 is a graph showing that anti-S. aureus recombinant polyclonal antibody mixtures of five antibodies at a dose of 1 mg/kg at 72 hours shift the LD₅₀ curve (e.g., increase resistance to S. aureus infection) in a live S. aureus bacterial challenge (community acquired-MRSA strain, USA300) in mice (♦, PBS LD₅₀=2.75×10⁸ CFU; ◯, five antibody composition LD₅₀=5.0×10⁸ CFU.

FIG. 5 is a graph showing that an anti-S. aureus recombinant polyclonal antibody mixture of seven antibodies can protect mice from a challenge with live S. aureus (methicillin-sensitive S. aureus strain, Wood-46) at 2×10⁸ CFU or 4×10⁸ CFU (0, 7 antibody composition at 2×10⁸ CFU; ▪, 7 antibody composition at 4×10⁸ CFU; ◯, PBS at 2×10⁸ CFU (negative control); ▴, PBS at 4×10⁸ CFU (negative control); and x, vancomycin at 4×10⁸ CFU (positive control).

FIG. 6 is a graph showing that anti-S. aureus recombinant polyclonal antibody mixtures of seven antibodies at a dose of 1 mg/kg at 72 hours shift the LD₅₀ curve (e.g., increase resistance to S. aureus infection) in a live S. aureus bacterial challenge (methicillin-sensitive S. aureus strain, Wood-46) in mice (♦, PBS LD₅₀=1.8×10⁸ CFU; −◯, seven antibody composition LD₅₀=3.75×10⁸ CFU.

FIG. 7 is a graph showing that anti-S. aureus recombinant polyclonal antibody mixtures of five, 10 and 19 antibodies can protect mice from a challenge with live S. aureus (USA300) at 1×10⁹ CFU (□, 5 antibody composition #2 at 1×10⁹ CFU; x, 5 antibody composition #3 at 1×10⁹ CFU; , 10 antibody composition at 1×10⁹ CFU; 0, 19 antibody composition at 1×10⁹ CFU; and ▴, PBS at 1×10⁹ CFU (negative control).

FIG. 8 is a graph showing the number of anti-S. aureus antibodies that react with various strains of S. aureus.

FIG. 9 is a graph showing that anti-S. aureus recombinant polyclonal antibody mixtures with the capacity of binding to the cell surface of the Wood-46 S. aureus strain can enhance the ability of white blood cells to opsonize the bacteria.

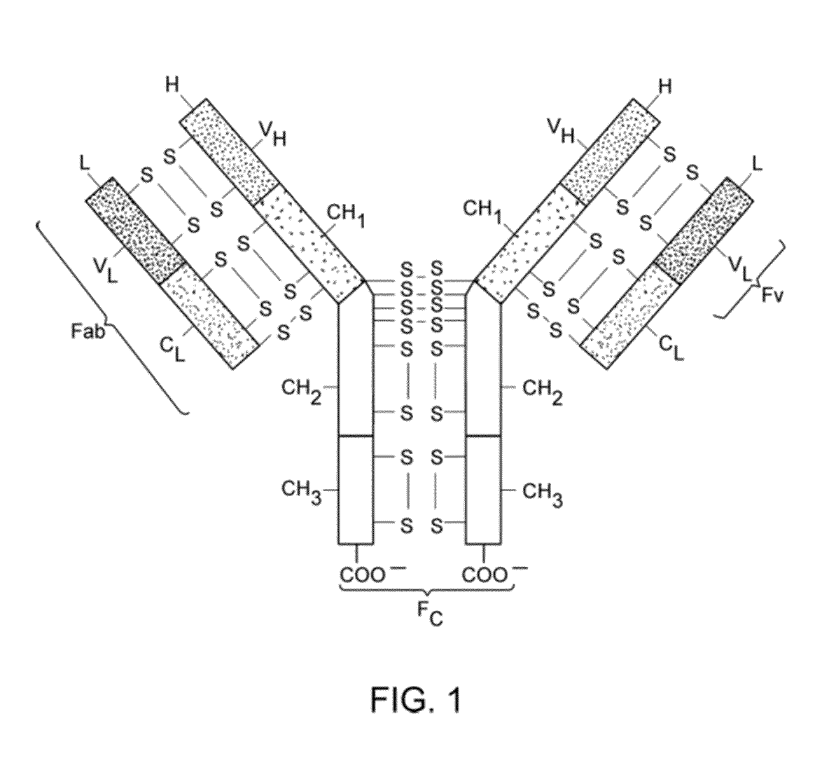

### DETAILED DESCRIPTION

The human recombinant polyclonal anti-S. aureus antibody compositions disclosed herein are based, in part, on the antigen binding sites of certain human antibodies that have been selected on the basis of binding and neutralizing activity of one or more S. aureus strains. The terms “anti-S. aureus polyclonal antibody” and “anti-S. aureus recombinant polyclonal antibody” describe a composition of recombinantly produced diverse antibody molecules, where the individual members of the polyclonal composition are capable of binding to at least one epitope on S. aureus or an S. aureus secreted protein (e.g., a toxin or immunomodulator) or a cell surface antigen and where the polyclonal composition as a whole is capable of neutralizing S. aureus. In an exemplary embodiment, an anti-S. aureus polyclonal antibody neutralizes S. aureus and/or one or more antibiotic-resistant S. aureus strains. It is contemplated that the disclosed anti-S. aureus polyclonal antibodies are essentially free from immunoglobulin molecules that do not bind to S. aureus or variant strains thereof (e.g., at least 70%, 75%, 80%, 85%, 90%, 95%, 98%, 99%, or 100% of the antibodies contained in the polyclonal composition bind to one or more strains of S. aureus).

As described herein, the diversity of antibodies included in an anti-S. aureus recombinant polyclonal antibody composition provide a surprising benefit over monoclonal and biclonal (e.g., a mixture of two monoclonal antibodies) antibodies because lower dosages of the polyclonal antibody composition may be administered to prevent or treat S. aureus infection. For example, it is contemplated herein that the synergistic action of the individual component antibodies in the polyclonal composition allow the polyclonal composition to be effective at lower doses than is possible with conventional monoclonal antibody therapy. In addition, because a polyclonal antibody composition binds to multiple different proteins, the composition as a whole can use lower amounts of each individual antibody to prevent or treat S. aureus infections. Further, unlike monoclonal or biclonal antibody compositions, polyclonal antibody compositions do not present the same concerns regarding the generation of drug resistance to a single or small number of agents (e.g., development of resistant S. aureus strains due to the monovalent nature of a monoclonal antibody's mode of action). It is also contemplated herein that the use of human antibodies in the polyclonal antibody composition are less likely to evoke an immune response compared to monoclonal antibodies, even fully human monoclonal antibodies, due to the complex nature of the polyclonal composition.

In view of the neutralizing activity of the disclosed polyclonal antibodies, they are useful for modulating the growth and/or colonization of one or more S. aureus strains including antibiotic-resistant S. aureus strains in a host cell; reducing or killing one or more strains of S. aureus including antibiotic-resistant S. aureus strains; and/or treating or preventing a S. aureus infection including infection with an antibiotic-resistant strain in a mammal. An anti-S. aureus polyclonal antibody may bind to S. aureus antigens in a multivalent manner, which may result in synergistic neutralization, improved phagocytosis of infected cells by macrophages, improved antibody-dependent cellular cytotoxicity (ADCC) against infected cells, and/or increased complement activity. It is contemplated herein that S. aureus is a multifaceted pathogen that may be neutralized using a multifaceted antibody approach that targets various antigens thereby enhancing the capacity of the immune system (e.g., opsonization) to eliminate these bacteria.

In one embodiment, the diversity of the recombinant polyclonal antibody is located in the variable regions (e.g., V_{H} and V_{L} regions) of the individual antibodies in the polyclonal antibody composition, in particular, in the CDR₁, CDR₂, and CDR₃ regions of the immunoglobulin heavy and/or light chains. For example, the individual antibodies of the polyclonal antibody composition contain (a) an immunoglobulin heavy chain variable region comprising the structure CDR_{H1}-CDR_{H2}-CDR_{H3} and (b) an immunoglobulin light chain variable region comprising the structure CDR_{L1}-CDR_{L2}-CDR_{L3}, wherein the heavy chain variable region and the light chain variable region together define a single binding site for binding to one or more S. aureus strains by binding to an antigen of S. aureus or an S. aureus antigenic epitope. An individual antibody of the polyclonal composition may also bind an S. aureus secreted protein or an antigenic epitope on the secreted protein. In some embodiments, each individual antibody of the polyclonal composition binds to an epitope, which is not bound by any other member of the polyclonal composition. In other embodiments, one or more S. aureus antigens or antigenic epitopes may be bound by more than one individual antibody of the polyclonal composition.

An individual antibody molecule of the recombinant polyclonal antibody composition may be characterized by its variable region sequences (i.e., V_{H} and V_{L} region), or by its CDR₁, CDR₂, and CDR₃ regions of immunoglobulin heavy and light chains. When the individual antibodies of the polyclonal composition are defined by their heavy and light chain CDR regions, it is contemplated that the CDR regions are interposed between human immunoglobulin framework regions (FRs).

It is understood that each of the antibodies discussed above can be an intact tetrameric antibody. Alternatively, the antibody can be an antigen-binding fragment of an antibody. Antibody fragments include Fab, Fab′, F(ab′)₂, and Fv fragments, and single chain antibodies (e.g., scFv).

An antibody, or antigen binding fragment thereof, may also be conjugated to an effector agent such as a small molecule toxin, a drug, or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

Human antibodies are selected based on binding to one or more S. aureus strains. Individual antibodies of the polyclonal composition may bind a protein associated with S. aureus virulence, such as, but not limited to coagulase, leukocidin (Luk), panton-valentine leukocidin (PVL), aureolysin, staphylokinase (SAK), beta-hemolysin (β-HL), delta-hemolysin (δ-HL), gamma-hemolysin (γ-HL), alpha-toxin (α-toxin), staphylococcal complement inhibitor (SCIN), enterotoxins, and adhesions (e.g., clumping factor A (ClfA), clumping factor B (ClfB), fibronectin binding protein (FnbpA), and fibronectin binding protein B (FnbpB). Additional S. aureus target proteins for binding by a S. aureus antibody include SdrD (a cell surface protein containing serine-aspartate (SD) repeats with organization and sequence similarity to fibrinogen-binding clumping factors ClfA and ClfB), IsaA, Aux1 (a transmembrane protein phosphatase), and LP309 (a lipoprotein). The disclosed antibodies may also bind to uncharacterized S. aureus proteins.

In certain embodiments, an anti-S. aureus polyclonal antibody binds to at least two, at least three, at least four, at least five, at least six, at least seven, at least eight, at least nine, at least ten, at least 15, at least 20, at least 25 or more S. aureus proteins. In an exemplary embodiment, an anti-S. aureus polyclonal antibody binds to at least three S. aureus proteins. In certain embodiments, the three proteins may include a cell surface antigen, a toxin and/or an immunomodulator produced by S. aureus. In one embodiment, an anti-S. aureus polyclonal antibody binds at least a cell surface antigen on one or more strains of S. aureus and a toxin produced by one or more strains of S. aureus. In another embodiment, an anti-S. aureus polyclonal antibody binds at least a cell surface antigen on one or more strains of S. aureus and an immunomodulator produced by one or more strains of S. aureus. In yet another embodiment, an anti-S. aureus polyclonal antibody binds at least a toxin produced by one or more strains of S. aureus and an immunomodulator produced by one or more strains of S. aureus. In certain embodiments, the at least 2, 3, 4, 5, 10, 15, 20, 25 or more proteins are on more than one strain of S. aureus. It is contemplated herein that the broad spectrum nature of the disclosed anti-S. aureus antibody compositions may be attributed to the inclusion of individuals antibodies that bind a protein present on more than one strain of S. aureus (e.g., conserved proteins). In addition, the broad spectrum efficacy of the disclosed polyclonal antibodies may be attributed to the inclusion of certain antibodies that bind antigens associated with only one or two S. aureus strains.

An anti-S. aureus polyclonal antibody composition may also be composed of individual antibodies raised by the immune response of a donor (e.g., a human), which has been vaccinated or infected with S. aureus. Further, if antibodies to a particular antigen are known to be relevant and/or effective in the protection, neutralization and/or elimination of S. aureus infection, such antibodies may be raised by immunization of a donor with that particular antigen.

It is contemplated herein that the disclosed recombinant polyclonal antibodies are not naturally occurring antibodies isolated from human blood. Exemplary recombinant polyclonal antibodies described herein are a mixture of antibodies, wherein each individual antibody may be expressed from a cell or cell line transfected with an expression vector comprising the coding sequence of the antibody, which is not naturally associated with the cell.

A human recombinant polyclonal anti-S. aureus antibody disclosed herein may comprise at least three, at least 4, at least 5, at least 7, at least 10, at least 15, at least 20, at least 25 or more antibodies. In exemplary embodiments, the recombinant polyclonal antibodies disclosed herein may comprise about 3 to about 30 antibodies, about 3 to about 25 antibodies, about 3 to about 20 antibodies, about 3 to about 15 antibodies, about 3 to about 10 antibodies, about 3 to about 5 antibodies, about 5 to about 30 antibodies, about 5 to about 25 antibodies, about 5 to about 20 antibodies, about 5 to about 15 antibodies, about 5 to about 10 antibodies, about 8 to about 30 antibodies, about 8 to about 25 antibodies, about 8 to about 20 antibodies, about 8 to about 15 antibodies, about 8 to about 10 antibodies, about 10 to about 30 antibodies, about 10 to about 25 antibodies, about 10 to about 20 antibodies, about 10 to about 15 antibodies, about 15 to about 25 antibodies, about 15 to about 20 antibodies, about 20 to about 25 antibodies, and about 25 to about 30 antibodies.

A polyclonal anti-S. aureus antibody may comprise immunoglobulin heavy and light chain variable regions or, heavy and light chain CDR regions, from two, three, four, five, ten or more of the following antibodies as disclosed herein: 1.62.B9, 5.11.H10, 5.6.H2, 5.6.H9, 5.17.F8, 5.19.F12, 5.23.C9, 5.23.C12, 5.27.A11, 8.51.G11, 9.51.H9, 18.43.D8, 22.22.E7, 8.51.G10, 5.24A.A7, 5.24A.F3, 5.8B.H4, 26.51.E1, 22.21.A7, 22.18A.E9, 5.52.H10, 5.15.C1, 5.54.E6, 5.55.D2, 22.14.A1, 26.53.B4, 5.63.E2, 5.64.G4, 43.52.A11, 43.52.E12 and 43.62.E2, wherein each of the disclosed antibodies comprise the immunoglobulin heavy and light chain CDR1, CDR2, CDR3 sequences set forth in Table 3. For example, antibody 5.6.H9 comprises the CDR1, CDR2, and CDR3 sequences of an immunogloblulin heavy chain amino acid sequence of SEQ ID NO: 38 and the CDR1, CDR2, and CDR3 of an immunoglobulin light chain amino acid sequence of SEQ ID NO: 40. Also, as disclosed in Table 3, for example, antibody 5.6.H9 comprises an immunoglobulin heavy chain comprising a CDR_{H1} comprising the amino acid sequence of SEQ ID NO: 41, a CDR_{H2} comprising amino acid sequence of SEQ ID NO: 42 and a CDR_{H3} comprising an amino acid sequence of SEQ ID NO: 43; and an immunoglobulin light chain comprising a CDR_{L1} comprising the amino acid sequence of SEQ ID NO: 44, a CDR_{L2} comprising the amino acid sequence of DAS and a CDR_{L3} comprising an amino acid sequence of SEQ ID NO: 45.

Exemplary recombinant polyclonal antibody compositions including two antibodies disclosed herein are shown in Table 1.

TABLE 1

| | 5.6.H9 | 5.11.H10 | 5.27.A11 | 5.17.F8 | 18.43.D8 | 5.19.F12 | 8.51.G11 | 8.51.G10 | 22.22.E7 | 5.23.C9 | 5.23.C12 |
|---|---|---|---|---|---|---|---|---|---|---|---|
| 5.6.H9 | | X | X | X | X | X | X | X | X | X | X |
| 5.11.H10 | X | | X | X | X | X | X | X | X | X | X |
| 5.27.A11 | X | X | | X | X | X | X | X | X | X | X |
| 5.17.F8 | X | X | X | | X | X | X | X | X | X | X |
| 18.43.D8 | X | X | X | X | | X | X | X | X | X | X |
| 5.19.F12 | X | X | X | X | X | | X | X | X | X | X |
| 8.51.G11 | X | X | X | X | X | X | | X | X | X | X |
| 8.51.G10 | X | X | X | X | X | X | X | | X | X | X |
| 22.22.E7 | X | X | X | X | X | X | X | X | | X | X |
| 5.23.C9 | X | X | X | X | X | X | X | X | X | | X |
| 5.23.C12 | X | X | X | X | X | X | X | X | X | X | |
| 9.51.H9 | X | X | X | X | X | X | X | X | X | X | X |
| 5.24A.F3 | X | X | X | X | X | X | X | X | X | X | X |
| 5.24A.A7 | X | X | X | X | X | X | X | X | X | X | X |
| 5.8B.H4 | X | X | X | X | X | X | X | X | X | X | X |
| 5.52.H10 | X | X | X | X | X | X | X | X | X | X | X |
| 22.18A.E9 | X | X | X | X | X | X | X | X | X | X | X |
| 22.21.A7 | X | X | X | X | X | X | X | X | X | X | X |
| 5.15.C1 | X | X | X | X | X | X | X | X | X | X | X |
| 5.54.E6 | X | X | X | X | X | X | X | X | X | X | X |
| 5.55.D2 | X | X | X | X | X | X | X | X | X | X | X |
| 26.51.E1 | X | X | X | X | X | X | X | X | X | X | X |
| 22.14.A1 | X | X | X | X | X | X | X | X | X | X | X |
| 26.53.B4 | X | X | X | X | X | X | X | X | X | X | X |
| 5.63.E2 | X | X | X | X | X | X | X | X | X | X | X |
| 5.64.G4 | X | X | X | X | X | X | X | X | X | X | X |
| 43.52.A11 | X | X | X | X | X | X | X | X | X | X | X |
| 43.52.E12 | X | X | X | X | X | X | X | X | X | X | X |
| 43.62.E2 | X | X | X | X | X | X | X | X | X | X | X |

| | 9.51.H9 | 5.24A.F3 | 5.24A.A7 | 5.8B.H4 | 5.52.H10 | 22.18A.E9 | 22.21.A7 | 5.15.C1 | 5.54.E6 | 5.55.D2 |
|---|---|---|---|---|---|---|---|---|---|---|
| 5.6.H9 | X | X | X | X | X | X | X | X | X | X |
| 5.11.H10 | X | X | X | X | X | X | X | X | X | X |
| 5.27.A11 | X | X | X | X | X | X | X | X | X | X |
| 5.17.F8 | X | X | X | X | X | X | X | X | X | X |
| 18.43.D8 | X | X | X | X | X | X | X | X | X | X |
| 5.19.F12 | X | X | X | X | X | X | X | X | X | X |
| 8.51.G11 | X | X | X | X | X | X | X | X | X | X |
| 8.51.G10 | X | X | X | X | X | X | X | X | X | X |
| 22.22.E7 | X | X | X | X | X | X | X | X | X | X |
| 5.23.C9 | X | X | X | X | X | X | X | X | X | X |
| 5.23.C12 | X | X | X | X | X | X | X | X | X | X |
| 9.51.H9 | | X | X | X | X | X | X | X | X | X |
| 5.24A.F3 | X | | X | X | X | X | X | X | X | X |
| 5.24A.A7 | X | X | | X | X | X | X | X | X | X |
| 5.8B.H4 | X | X | X | | X | X | X | X | X | X |
| 5.52.H10 | X | X | X | X | | X | X | X | X | X |
| 22.18A.E9 | X | X | X | X | X | | X | X | X | X |
| 22.21.A7 | X | X | X | X | X | X | | X | X | X |
| 5.15.C1 | X | X | X | X | X | X | X | | X | X |
| 5.54.E6 | X | X | X | X | X | X | X | X | | X |
| 5.55.D2 | X | X | X | X | X | X | X | X | X | |
| 26.51.E1 | X | X | X | X | X | X | X | X | X | X |
| 22.14.A1 | X | X | X | X | X | X | X | X | X | X |
| 26.53.B4 | X | X | X | X | X | X | X | X | X | X |
| 5.63.E2 | X | X | X | X | X | X | X | X | X | X |
| 5.64.G4 | X | X | X | X | X | X | X | X | X | X |
| 43.52.A11 | X | X | X | X | X | X | X | X | X | X |
| 43.52.E12 | X | X | X | X | X | X | X | X | X | X |
| 43.62.E2 | X | X | X | X | X | X | X | X | X | X |

| | 26.51.E1 | 22.14.A1 | 26.53.B4 | 5.63.E2 | 5.64.G4 | 43.52.A11 | 43.52.E12 | 43.62.E2 |
|---|---|---|---|---|---|---|---|---|
| 5.6.H9 | X | X | X | X | X | X | X | X |
| 5.11.H10 | X | X | X | X | X | X | X | X |
| 5.27.A11 | X | X | X | X | X | X | X | X |
| 5.17.F8 | X | X | X | X | X | X | X | X |
| 18.43.D8 | X | X | X | X | X | X | X | X |
| 5.19.F12 | X | X | X | X | X | X | X | X |
| 8.51.G11 | X | X | X | X | X | X | X | X |
| 8.51.G10 | X | X | X | X | X | X | X | X |
| 22.22.E7 | X | X | X | X | X | X | X | X |
| 5.23.C9 | X | X | X | X | X | X | X | X |
| 5.23.C12 | X | X | X | X | X | X | X | X |
| 9.51.H9 | X | X | X | X | X | X | X | X |
| 5.24A.F3 | X | X | X | X | X | X | X | X |
| 5.24A.A7 | X | X | X | X | X | X | X | X |
| 5.8B.H4 | X | X | X | X | X | X | X | X |
| 5.52.H10 | X | X | X | X | X | X | X | X |
| 22.18A.E9 | X | X | X | X | X | X | X | X |
| 22.21.A7 | X | X | X | X | X | X | X | X |
| 5.15.C1 | X | X | X | X | X | X | X | X |
| 5.54.E6 | X | X | X | X | X | X | X | X |
| 5.55.D2 | X | X | X | X | X | X | X | X |
| 26.51.E1 | | X | X | X | X | X | X | X |
| 22.14.A1 | X | | X | X | X | X | X | X |
| 26.53.B4 | X | X | | X | X | X | X | X |
| 5.63.E2 | X | X | X | | X | X | X | X |
| 5.64.G4 | X | X | X | X | | X | X | X |
| 43.52.A11 | X | X | X | X | X | | X | X |
| 43.52.E12 | X | X | X | X | X | X | | X |
| 43.62.E2 | X | X | X | X | X | X | X | |

Exemplary recombinant polyclonal antibody compositions including three antibodies disclosed herein include: 5.6.H9, 22.18A.E9 and 5.55.D2; 5.6.H9, 22.18A.E9 and 9.51.H9; 5.6 H9, 22.18A.E9 and 5.11.H10; 5.6 H9, 22.18A.E9 and 5.23.C12; 5.6.H9, 22.18A.E9 and 5.52.H10; 5.6.H9, 22.18A.E9 and 18.43.D8; 5.6 H9, 22.18A.E9 and 8.51 G10; 5.6.H9, 22.18A.E9 and 8.51.G11.

It is also contemplated herein that the disclosed recombinant human polyclonal antibody compositions may include one or more antibodies that compete with one of the disclosed antibodies for binding to one or more S. aureus strains, for example, under the conditions described in Example 4. Exemplary S. aureus strains for determining whether an antibody competes with binding to a disclosed antibody include ATCC Strain BAA-1717 (also known as USA300), ATCC Strain 10832 (also known as Wood-46), NRS071 (also known as Sanger 252), NRS100 (also known as COL), NRS382 (also known as strain 626), NRS384 (also known as LAC), NRS123 (also known as MW2), NRS001 (also known as Mu50), NRS072 (also known as Sanger 476), NRS102 (also known as Reynolds), NRS111 (also known as FR1913), NRS144 (also known as RN4220) and USA300. Other exemplary S. aureus stains include USA100, USA200, USA400 and USA500 types.

It is contemplated herein that the disclosed polyclonal antibody compositions bind at least 3 different S. aureus strains. For example, in exemplary embodiments, the disclosed polyclonal antibody compositions bind at least 4, at least 5, at least 6, at least 7, at least 8, at least 9, at least 10, at least 11, or at least 12 different S. aureus strains, e.g., the strains identified in Table 4.

In one embodiment, an antibody provided herein competes with 5.6.H9 comprising CDR1, CDR2 and CDR3 of heavy chain amino acid sequence SEQ ID NO: 38 and CDR1, CDR2, CDR3 of light chain amino acid sequence SEQ ID NO: 40 for binding to one or more S. aureus strains. Exemplary strains for a competitive binding assay with 5.6.H9, for example, under the conditions set forth in Example 4, are set forth in Table 4.

In another embodiment, an antibody provided herein competes with 22.18A.E9 comprising CDR1, CDR2 and CDR3 of heavy chain amino acid sequence SEQ ID NO: 173 and CDR1, CDR2, CDR3 of light chain amino acid sequence SEQ ID NO: 175 for binding to one or more S. aureus. Exemplary strains for a competitive binding assay with 22.18A.E9, for example, under the conditions set forth in Example 4, are set forth in Table 4.

In another embodiment, an antibody provided herein competes with 5.11.H10 comprising CDR1, CDR2 and CDR3 of heavy chain amino acid sequence SEQ ID NO: 11 and CDR1, CDR2, CDR3 of light chain amino acid sequence SEQ ID NO: 13 for binding to one or more S. aureus strains. Exemplary strains for a competitive binding assay with 5.11.H10, for example, under the conditions set forth in Example 4, are set forth in Table 4.

In another embodiment, an antibody provided herein competes with 5.27.A11 comprising CDR1, CDR2 and CDR3 of heavy chain amino acid sequence SEQ ID NO: 20 and CDR1, CDR2, CDR3 of light chain amino acid sequence SEQ ID NO: 22 for binding to one or more S. aureus strains. Exemplary strains for a competitive binding assay with 5.27.A11, for example, under the conditions set forth in Example 4, are set forth in Table 4.

In another embodiment, an antibody provided herein competes with 5.17.F8 comprising CDR1, CDR2 and CDR3 of heavy chain amino acid sequence SEQ ID NO: 47 and CDR1, CDR2, CDR3 of light chain amino acid sequence SEQ ID NO: 49 for binding to one or more S. aureus strains. Exemplary strains for a competitive binding assay with 5.17.F8, for example, under the conditions set forth in Example 4, are set forth in Table 4.

In another embodiment, an antibody provided herein competes with 5.19.F12 comprising CDR1, CDR2 and CDR3 of heavy chain amino acid sequence SEQ ID NO: 56 and CDR1, CDR2, CDR3 of light chain amino acid sequence SEQ ID NO: 58 for binding to one or more S. aureus strains. Exemplary strains for a competitive binding assay with 5.19.F12, for example, under the conditions set forth in Example 4, are set forth in Table 4.

In another embodiment, an antibody provided herein competes with 5.23.C9 comprising CDR1, CDR2 and CDR3 of heavy chain amino acid sequence SEQ ID NO: 65 and CDR1, CDR2, CDR3 of light chain amino acid sequence SEQ ID NO: 67 for binding to one or more S. aureus strains. Exemplary strains for a competitive binding assay with 5.23.C9, for example, under the conditions set forth in Example 4, are set forth in Table 4.

In another embodiment, an antibody provided herein competes with 5.23.C12 comprising CDR1, CDR2 and CDR3 of heavy chain amino acid sequence SEQ ID NO: 74 and CDR1, CDR2, CDR3 of light chain amino acid sequence SEQ ID NO: 76 for binding to one or more S. aureus strains. Exemplary strains for a competitive binding assay with 5.23.C12, for example, under the conditions set forth in Example 4, are set forth in Table 4.

In another embodiment, an antibody provided herein competes with 8.51.G11 comprising CDR1, CDR2 and CDR3 of heavy chain amino acid sequence SEQ ID NO: 83 and CDR1, CDR2, CDR3 of light chain amino acid sequence SEQ ID NO: 85 for binding to one or more S. aureus strains. Exemplary strains for a competitive binding assay with 8.51.G11, for example, under the conditions set forth in Example 4, are set forth in Table 4.

In another embodiment, an antibody provided herein competes with 9.51.H9 comprising CDR1, CDR2 and CDR3 of heavy chain amino acid sequence SEQ ID NO: 92 and CDR1, CDR2, CDR3 of light chain amino acid sequence SEQ ID NO: 94 for binding to one or more S. aureus strains. Exemplary strains for a competitive binding assay with 9.51.H9, for example, under the conditions set forth in Example 4, are set forth in Table 4.

In another embodiment, an antibody provided herein competes with 18.43.D8 comprising CDR1, CDR2 and CDR3 of heavy chain amino acid sequence SEQ ID NO: 101 and CDR1, CDR2, CDR3 of light chain amino acid sequence SEQ ID NO: 103 for binding to one or more S. aureus strains. Exemplary strains for a competitive binding assay with 18.43.D8, for example, under the conditions set forth in Example 4, are set forth in Table 4.

In another embodiment, an antibody provided herein competes with 22.22.E7 comprising CDR1, CDR2 and CDR3 of heavy chain amino acid sequence SEQ ID NO: 110 and CDR1, CDR2, CDR3 of light chain amino acid sequence SEQ ID NO: 112 for binding to one or more S. aureus strains. Exemplary strains for a competitive binding assay with 22.22.E7, for example, under the conditions set forth in Example 4, are set forth in Table 4.

In another embodiment, an antibody provided herein competes with 8.51.G10 comprising CDR1, CDR2 and CDR3 of heavy chain amino acid sequence SEQ ID NO: 149 and CDR1, CDR2, CDR3 of light chain amino acid sequence SEQ ID NO: 151 for binding to one or more S. aureus strains. Exemplary strains for a competitive binding assay with 8.51.G10, for example, under the conditions set forth in Example 4, are set forth in Table 4.

In another embodiment, an antibody provided herein competes with 5.24A.A7 comprising CDR1, CDR2 and CDR3 of heavy chain amino acid sequence SEQ ID NO: 153 and CDR1, CDR2, CDR3 of light chain amino acid sequence SEQ ID NO: 155 for binding to one or more S. aureus strains. Exemplary strains for a competitive binding assay with 5.24A.A7, for example, under the conditions set forth in Example 4, are set forth in Table 4.

In another embodiment, an antibody provided herein competes with 5.24A.F3 comprising CDR1, CDR2 and CDR3 of heavy chain amino acid sequence SEQ ID NO: 157 and CDR1, CDR2, CDR3 of light chain amino acid sequence SEQ ID NO: 159 for binding to one or more S. aureus strains. Exemplary strains for a competitive binding assay with 5.24A.F3, for example, under the conditions set forth in Example 4, are set forth in Table 4.

In another embodiment, an antibody provided herein competes with 5.8B.H4 comprising CDR1, CDR2 and CDR3 of heavy chain amino acid sequence SEQ ID NO: 161 and CDR1, CDR2, CDR3 of light chain amino acid sequence SEQ ID NO: 163 for binding to one or more S. aureus strains. Exemplary strains for a competitive binding assay with 5.8B.H4, for example, under the conditions set forth in Example 4, are set forth in Table 4.

In another embodiment, an antibody provided herein competes with 26.51.E1 comprising CDR1, CDR2 and CDR3 of heavy chain amino acid sequence SEQ ID NO: 165 and CDR1, CDR2, CDR3 of light chain amino acid sequence SEQ ID NO: 167 for binding to one or more S. aureus strains. Exemplary strains for a competitive binding assay with 26.51.E1, for example, under the conditions set forth in Example 4, are set forth in Table 4.

In another embodiment, an antibody provided herein competes with 22.21.A7 comprising CDR1, CDR2 and CDR3 of heavy chain amino acid sequence SEQ ID NO: 169 and CDR1, CDR2, CDR3 of light chain amino acid sequence SEQ ID NO: 171 for binding to one or more S. aureus strains. Exemplary strains for a competitive binding assay with 22.21.A7, for example, under the conditions set forth in Example 4, are set forth in Table 4.

In another embodiment, an antibody provided herein competes with 5.52.H1 comprising CDR1, CDR2 and CDR3 of heavy chain amino acid sequence SEQ ID NO: 177 and CDR1, CDR2, CDR3 of light chain amino acid sequence SEQ ID NO: 179 for binding to one or more S. aureus strains. Exemplary strains for a competitive binding assay with 5.52.H1, for example, under the conditions set forth in Example 4 are set forth in Table 4.

In another embodiment, an antibody provided herein competes with 5.15.C1 comprising CDR1, CDR2 and CDR3 of heavy chain amino acid sequence SEQ ID NO: 181 and CDR1, CDR2, CDR3 of light chain amino acid sequence SEQ ID NO: 183 for binding to one or more S. aureus strains. Exemplary strains for a competitive binding assay with 5.15.C1, for example, under the conditions set forth in Example 4, are set forth in Table 4.

In another embodiment, an antibody provided herein competes with 5.54.E6 comprising CDR1, CDR2 and CDR3 of heavy chain amino acid sequence SEQ ID NO: 185 and CDR1, CDR2, CDR3 of light chain amino acid sequence SEQ ID NO: 187 for binding to one or more S. aureus strains. Exemplary strains for a competitive binding assay with 5.54.E6, for example, under the conditions set forth in Example 4, are set forth in Table 4.

In another embodiment, an antibody provided herein competes with 5.55.D2 comprising CDR1, CDR2 and CDR3 of heavy chain amino acid sequence SEQ ID NO: 189 and CDR1, CDR2, CDR3 of light chain amino acid sequence SEQ ID NO: 191 for binding to one or more S. aureus strains. Exemplary strains for a competitive binding assay with 5.55.D2, for example, under the conditions set forth in Example 4, are set forth in Table 4.

In another embodiment, an antibody provided herein competes with 22.14.A1 comprising CDR1, CDR2 and CDR3 of heavy chain amino acid sequence SEQ ID NO: 193 and CDR1, CDR2, CDR3 of light chain amino acid sequence SEQ ID NO: 195 for binding to one or more S. aureus strains. Exemplary strains for a competitive binding assay with 22.14.A1, for example, under the conditions set forth in Example 4 are set forth in Table 4.

In another embodiment, an antibody provided herein competes with 26.53.B4 comprising CDR1, CDR2 and CDR3 of heavy chain amino acid sequence SEQ ID NO: 197 and CDR1, CDR2, CDR3 of light chain amino acid sequence SEQ ID NO: 199 for binding to one or more S. aureus strains. Exemplary strains for a competitive binding assay with 26.53.B4, for example, under the conditions set forth in Example 4, are set forth in Table 4.

In another embodiment, an antibody provided herein competes with 5.63.E2 comprising CDR1, CDR2 and CDR3 of heavy chain amino acid sequence SEQ ID NO: 201 and CDR1, CDR2, CDR3 of light chain amino acid sequence SEQ ID NO: 203 for binding to one or more S. aureus strains. Exemplary strains for a competitive binding assay with 5.63.E2, for example, under the conditions set forth in Example 4, are set forth in Table 4.

In another embodiment, an antibody provided herein competes with 5.64.G4 comprising CDR1, CDR2 and CDR3 of heavy chain amino acid sequence SEQ ID NO: 205 and CDR1, CDR2, CDR3 of light chain amino acid sequence SEQ ID NO: 207 for binding to one or more S. aureus strains. Exemplary strains for a competitive binding assay with 5.64.G4, for example, under the conditions set forth in Example 4, are set forth in Table 4.

In another embodiment, an antibody provided herein competes with 43.52.A11 comprising CDR1, CDR2 and CDR3 of heavy chain amino acid sequence SEQ ID NO: 209 and CDR1, CDR2, CDR3 of light chain amino acid sequence SEQ ID NO: 211 for binding to one or more S. aureus strains. Exemplary strains for a competitive binding assay with 43.52.A11, for example, under the conditions set forth in Example 4, are set forth in Table 4.

In another embodiment, an antibody provided herein competes with 43.52.E12 comprising CDR1, CDR2 and CDR3 of heavy chain amino acid sequence SEQ ID NO: 213 and CDR1, CDR2, CDR3 of light chain amino acid sequence SEQ ID NO: 215 for binding to one or more S. aureus strains. Exemplary strains for a competitive binding assay with 43.52.E12, for example, under the conditions set forth in Example 4, are set forth in Table 4.

In another embodiment, an antibody provided herein competes with 43.62.E2 comprising CDR1, CDR2 and CDR3 of heavy chain amino acid sequence SEQ ID NO: 217 and CDR1, CDR2, CDR3 of light chain amino acid sequence SEQ ID NO: 219 for binding to one or more S. aureus strains. Exemplary strains for a competitive binding assay with 43.62.E2, for example, under the conditions set forth in Example 4, are set forth in Table 4.

In another aspect, isolated human antibodies are also disclosed (e.g., isolated, individual antibodies or antigen binding fragments thereof). Exemplary human antibodies include an isolated human antibodies that bind S. aureus delta-toxin. One exemplary antibody that binds S. aureus delta-toxin comprises CDR1, CDR2 and CDR3 of heavy chain amino acid sequence SEQ ID NO: 38 and CDR1, CDR2, CDR3 of light chain amino acid sequence SEQ ID NO: 40 of 5.6.H9. Another exemplary antibody that bind S. aureus delta-toxin comprises CDR1, CDR2 and CDR3 of heavy chain amino acid sequence SEQ ID NO: 189 and CDR1, CDR2, CDR3 of light chain amino acid sequence SEQ ID NO: 191 of 5.55.D2.

Also provided herein is an isolated human antibody that binds S. aureus phenol soluble modulin beta-1. The antibody comprises CDR1, CDR2 and CDR3 of heavy chain amino acid sequence SEQ ID NO: 173 and CDR1, CDR2, CDR3 of light chain amino acid sequence SEQ ID NO: 175 of 22.18A.E9.

Additional exemplary isolated human antibodies disclosed herein include: a human antibody that binds one or more S. aureus strains comprising CDR1, CDR2 and CDR3 of heavy chain amino acid sequence SEQ ID NO: 92 and CDR1, CDR2, CDR3 of light chain amino acid sequence SEQ ID NO: 94 of 9.51.H9;

a human antibody that binds one or more S. aureus strains comprising CDR1, CDR2 and CDR3 of heavy chain amino acid sequence SEQ ID NO: 11 and CDR1, CDR2, CDR3 of light chain amino acid sequence SEQ ID NO: 13 of 5.11.H10;

a human antibody that binds one or more S. aureus strains comprising CDR1, CDR2 and CDR3 of heavy chain amino acid sequence SEQ ID NO: 74 and CDR1, CDR2, CDR3 of light chain amino acid sequence SEQ ID NO: 76 of 5.23.C12;

a human antibody that binds one or more S. aureus strains comprising CDR1, CDR2 and CDR3 of heavy chain amino acid sequence SEQ ID NO: 177 and CDR1, CDR2, CDR3 of light chain amino acid sequence SEQ ID NO: 179 of 5.52.H10;

a human antibody that binds one or more S. aureus strains comprising CDR1, CDR2 and CDR3 of heavy chain amino acid sequence SEQ ID NO: 101 and CDR1, CDR2, CDR3 of light chain amino acid sequence SEQ ID NO: 103 of 18.43.D8;

a human antibody that binds one or more S. aureus strains comprising CDR1, CDR2 and CDR3 of heavy chain amino acid sequence SEQ ID NO: 149 and CDR1, CDR2, CDR3 of light chain amino acid sequence SEQ ID NO: 151 of 8.51.G10; and

a human antibody that binds one or more S. aureus strains comprising CDR1, CDR2 and CDR3 of heavy chain amino acid sequence SEQ ID NO: 83 and CDR1, CDR2, CDR3 of light chain amino acid sequence SEQ ID NO: 85 of 8.51.G11.

### Production of Antibodies

Methods for producing antibodies of the invention are known in the art. In certain embodiments, an anti-S. aureus polyclonal antibody composition may be produced from a single manufacturing cell line or a mixture of cell lines producing individual monoclonal antibodies. In other embodiments, DNA molecules encoding light chain variable regions and heavy chain variable regions can be chemically synthesized using the sequence information provided herein. Synthetic DNA molecules can be ligated to other appropriate nucleotide sequences, including, e.g., constant region coding sequences, and expression control sequences, to produce conventional gene expression constructs encoding the desired antibodies. Production of defined gene constructs is within routine skill in the art. Alternatively, the sequences provided herein can be cloned out of hybridomas or B-cells by conventional hybridization techniques or polymerase chain reaction (PCR) techniques, using synthetic nucleic acid probes whose sequences are based on sequence information provided herein, or prior art sequence information regarding genes encoding the heavy and light chains of human antibodies in hybridoma cells.

As disclosed herein, individual anti-S. aureus antibodies may be characterized by their variable region (V_{H} and V_{L} sequences) or by their heavy and light chain CDR sequences. Each antibody will have a pair of sequences if defined by its variable region sequences (i.e., V_{H} and V_{L} cognate pairs) or a set of sequences if defined by its CDR sequences (i.e., three heavy chain CDRs and three light chain CDRs). V_{H} and V_{L} pairs can be expressed as full-length antibodies, Fab fragments or other antibody fragments that have binding specificity to a S. aureus associated antigen. Specific V_{H} and V_{L} pairs are identified in Table 2 in Example 1. Specific heavy and light chain CDR sets are identified in Table 3 in Example 1.

Nucleic acids encoding desired antibodies (e.g., V_{H} and V_{L} pairs) can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukayotic host cells, e.g., CHO cells, it is first inserted into an expression vector containing a suitable eukaryotic promoter, a secretion signal, IgG enhancers, and various introns. This expression vector optionally contains sequences encoding all or part of a constant region, enabling an entire, or a part of, a heavy or light chain to be expressed. The gene construct can be introduced into eukaryotic host cells using convention techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of, a light chain or light chain variable region).

The expression vector may also include constant regions for the heavy and/or light chain. It is contemplated that the choice of the constant region may vary for the individual antibodies included in the polyclonal composition. For example, it may be desirous to have IgG1 constant regions for certain antibodies and IgG2 constant regions for other antibodies depending on the desired effector function to clear or destroy antigen (e.g., ADCC, phagocytosis, increased complement activity (e.g., via the classic and/or alternative complement pathways), binding to mass cells and/or basinophiles). Heavy chain constant regions may be selected from the isotypes IgG1, IgG2, IgG3, IgG4, IgA1, IgA2, IgM, IgD, and IgE. Light chain constant regions may be either kappa or lambda.

A polypeptide comprising an immunoglobulin heavy chain variable region or light chain variable region can be produced by growing a host cell transfected with an expression vector encoding such variable region, under conditions that permit expression of the polypeptide. Following expression, the polypeptide can be harvested and purified using techniques well known in the art, e.g., affinity tags such as glutathione-S-transferase (GST) and histidine tags.

A human antibody that binds to one or more S. aureus strains, or an antigen-binding fragment of the antibody, can be produced by growing a host cell transfected with: (a) an expression vector that encodes a complete or partial immunoglobulin heavy chain, and a separate expression vector that encodes a complete or partial immunoglobulin light chain; or (b) a single expression vector that encodes both chains, under conditions that permit expression of both chains. The intact antibody (or antigen-binding fragment) can be harvested and purified using techniques well known in the art, e.g., Protein A, Protein G, affinity tags such as glutathione-S-transferase (GST) and histidine tags. It is within ordinary skill in the art to express the heavy chain and the light chain from a single expression vector or from two separate expression vectors.

### Use of Antibodies

Anti-S. aureus polyclonal antibodies as described herein can be used to treat one or more strains of S. aureus and/or prevent infection of one or more strains of S. aureus, including antibiotic resistant strains of S. aureus, e.g., penicillin-resistant strains, methicillin-resistant strains (MRSA) (e.g., community acquired MRSA (CA-MRSA), hospital-acquired MRSA (HA-MRSA)), vancomycin-resistant strains (VRSA), and vancomycin-intermediate resistant strains (VISA). The disclosed antibodies may also be used to treat methicillin-sensitive strains (e.g., MSSA). S. aureus infected host cells (e.g., mammalian host cells, e.g., human host cells) are exposed to a therapeutically effective amount of the antibody so as to inhibit infectivity of S. aureus by inhibiting growth and/or colonization, and/or by inducing phagocytosis, and/or killing the S. aureus. In some embodiments, the antibodies inhibit infectivity of S. aureus by at least 30%, 40%, 50%, 60%, 70%, 80%, 90%, 95%, 98%, 99%, or 100%.

In certain embodiments, individual human anti-S. aureus antibodies may be used to treat one or more strains of S. aureus and/or prevent infection of one or more strains of S. aureus.

As used herein, “treat”, “treating” and “treatment” mean the treatment of a disease in a mammal, e.g., in a human. This includes: (a) inhibiting the disease or infection, i.e., arresting its development or progression; (b) relieving the disease or infection, i.e., causing regression of the disease state or infection; and/or (c) curing the disease or infection.

Exemplary diseases that can be treated or prevented using the disclosed antibodies include, but are not limited to, invasive or toxigenic diseases associated with pathogenic S. aureus strains. Invasive diseases include pneumonia (e.g., S. aureus pneumonia), meningitis, Bacteremia, osteomyelitis, sepsis (e.g., septic arthritis, septic thrombophlebitis), and endocarditis (e.g., acute bacterial endocarditis). Toxic diseases include, but are not limited to, Staphylococcal food poisoning, scalded skin syndrome, and toxic shock syndrome (TSS). Additional examples of diseases and disorders that may be treated include skin abscesses, cellulitis, upper respiratory tract infections (e.g., otis media, bacterial trachetis, acute epiglottitis, thyroiditis), lower respiratory tract infections (e.g., empyema, lung abscess), heart, gastrointestinal (e.g., secretory diarrhea, splenic abscess, retroperitoneal abscess), CNS (e.g., cerebral abscess), eye (e.g., blepharitis, conjunctivitis, keratitis, endophthalmitis, preseptal and orbital cellulitis, darcryocystitis), kidney, urinary, skin (e.g., impetigo, folloculitis, cutaneous abscesses, cellulitis, wound infection, bacterial myositis), and bone and joint infections. It is contemplated herein that certain disorders associated with polymicrobial infections including a S. aureus infection may be treated or prevented using the disclosed antibodies. Exemplary disorders associated with polymicrobial infections (including S. aureus infections) include cystic fibrosis (e.g., infections with S. aureus and Pseudomonas), upper and lower respiratory tract infections, pneumonia, septicemia and skin infections.

Generally, a therapeutically effective amount of active component is in the range of 0.1 mg/kg to 100 mg/kg, e.g., 1 mg/kg to 100 mg/kg, 1 mg/kg to 10 mg/kg. In an exemplary embodiment, a human recombinant polyclonal antibody may be administered at 1 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount and the disease being treated. Exemplary dosing frequencies are once per day, once every 2 days, once every three days, once every four days, once every five days, once every six days, once per week, once every two weeks, once every month, once every six months, and once a year. In some embodiments of the invention, dosing is once every two weeks. A preferred route of administration is parenteral, e.g., intravenous or subcutaneous. Formulation of antibody-based drugs is within ordinary skill in the art. In some embodiments of the invention, the antibody is lyophilized and reconstituted in buffered saline at the time of administration.

For therapeutic use, one or more disclosed antibodies, or an antigen binding fragments thereof, can be combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing one or more of the disclosed antibodies can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. An exemplary route of administration for monoclonal antibodies is IV infusion. Useful formulations can be prepared by methods well known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### EXAMPLES

The following Examples are merely illustrative and are not intended to limit the scope or content of the invention in any way.

### Example 1

### Sequence Analysis of Anti-S. aureus Antibodies

This example describes the sequence analysis of the anti-S. aureus antibodies disclosed herein.

Individual anti-S. aureus antibodies were isolated from human donor individuals (e.g., healthy individuals) who had been exposed to S. aureus including the S. aureus strains methicillin sensitive S. aureus (MSSA), hospital-acquired-MRSA, and community-acquired-MRSA. The individual antibodies (e.g., V_{H} and V_{L} regions) were sequenced by Sanger dideoxy-sequencing and were analyzed using IMGT/V-Quest software (Montpellier, France) to identify and confirm variable region sequences.

The nucleic acid sequences encoding and the protein sequences defining heavy and light chain variable regions of the anti-S. aureus antibodies are shown below (amino terminal signal peptide sequences are not shown). CDR sequences (IMGT definition) are indicated by bold font and underlining in the nucleic acid and amino acid sequences.

Nucleic Acid Sequence Encoding the 1.62.B9 Heavy Chain Variable Region (SEQ ID NO: 1)

GAGGTGCAGCTGGTGGAGTCTGGGGGAGGCTTGGTAAAGCCTGGGGGGTC
CCTTAGACTCTCCTGTGCAGCCTCTGGATTCACTTTCAGTAACGCCTGGA
TGACCTGGGTCCGCCAGGCTCCAGGGAAGGGGCTGGAGTGGGTTGGCCGT
ATTAAGAGCAAAACTGATGGTGGGACAACAGACTACGCTGCACCCGTGAA
AGGCAGATTCACCATCTCAAGAGATGATTCAATAAACACACTGTATCTGC
AAATGAACAGCCTGAAAACCGAGGACACAGCCGTGTATTATTGTAGGACG
GGGGGCTACTGGGGCCAGGGAACCCTGGTCACCGTCTCGAGTG

Protein Sequence Defining the 1.62.B9 Heavy Chain Variable Region (SEQ ID NO: 2)

EVQLVESGGGLVKPGGSLRLSCAASGFTFSNAWMTWVRQAPGKGLEWVGR
IKSKTDGGTTDYAAPVKGRFTISRDDSINTLYLQMNSLKTEDTAVYYCRT
GGYWGQGTLVTVSS

Nucleic Acid Sequence Encoding the 1.62.B9 Lambda Chain Variable Region (SEQ ID NO: 3)

CAGGCTGTGGTGACTCAGGAGCCCTCACTGACTGTGTCCCCAGGAGGGAC
AGTCACTCTCACCTGTGGCTCCAGCACTGGAGCTGTCACCAGTGGTCATT
ATCCCTACTGGTTCCAGCAGAAGCCTGGCCAAGCCCCCAGGACACTGATT
TATGATACAAGCAAGAAACATTCCTGGACACCTGCCCGGTTCTCAGGCTC
CCTCCTTGGGGGCAAAGCTGCCCTGACCCTTTCGGGTGCGCAGCCTGAGG
ATGAGGCTGAGTATTACTGCTTGGTCTCCGATAGTGGTGCTCGGATCTTC
GGACCTGGGACCAAGGTCACCGTCCTAG

Protein Sequence Defining the 1.62.B9 Lambda Chain Variable Region (SEQ ID NO: 4)

QAVVTQEPSLTVSPGGTVTLTCGSSTGAVTSGHYPYWFQQKPGQAPRTLI
YDTSKKHSWTPARFSGSLLGGKAALTLSGAQPEDEAEYYCLVSDSGARIF
GPGTKVTVL

Nucleic Acid Sequence Encoding the 5.11.H10 Heavy Chain Variable Region (SEQ ID NO: 10)

CAGGTTCAGCTGGTGCAGTCTGGACCTGAAATGAAGAAGCCTGGGGCCTC
AGTGAAGGTCTCCTGTCAGGCCTCTGGTTACACCTTTACCATTAATGGAA
TTAGTTGGGTGCGACAGGCCCCTGGACAACGGCCTGAGTGGATGGGGTGG
ATCAGCACTAGTAATGGTCAGACAAACTTTGCACAGAATTTCCAGGGCAG
ACTCACCTTGACCACAGACACATCCACGACCACGGCCTTCATGGAGTTGA
TGAGCCTCACATCTGCCGACACGGCCGTATACTACTGTGCGAGAAATCCA
TACGGCAATTCGCGCAGATTCTTTGACTACTGGGGCCAGGGCACCCTGGT
CACCGTCTCGAGTG

Protein Sequence Defining the 5.11.H10 Heavy Chain Variable Region (SEQ ID NO: 11)

QVQLVQSGPEMKKPGASVKVSCQASGYTFTINGISWVRQAPGQRPEWMGW
ISTSNGQTNFAQNFQGRLTLTTDTSTTTAFMELMSLTSADTAVYYCARNP
YGNSRRFFDYWGQGTLVTVSS

Nucleic Acid Sequence Encoding the 5.11.H10 Kappa Chain Variable Region (SEQ ID NO: 12)

GACATCCAGATGACCCAGTCTCCATCCTCCCTGTCTGCCTCTGTTGGCGA
CAGAGTCATCATCACTTGCCGGACAAGTCAGACCATCACCAATTACATAA
ATTGGTATCAGCAAAAACCAGGGAAAGCCCCGCAGCTCCTGATCTATGGT
GTATCCAGTTTGCAAAGTGGGGTCCCATCAAGGTTCAGTGGCGGTGGATC
TGGGACAGAATTCACTCTCACCATCAGCAGTCTGCAACCTGAAGACTTTG
CAACTTATTACTGTCAACAGAGTTATAGGGCCCCTCAAACATTCGGCCAA
GGGACCAAGCTGGAAATCAAAC

Protein Sequence Defining the 5.11.H10 Kappa Chain Variable Region (SEQ ID NO: 13)

DIQMTQSPSSLSASVGDRVIITCRTSQTITNYINWYQQKPGKAPQLLIYG
VSSLQSGVPSRFSGGGSGTEFTLTISSLQPEDFATYYCQQSYRAPQTFGQ
GTKLEIK

Nucleic Acid Sequence Encoding the 5.27.A11 Heavy Chain Variable Region (SEQ ID NO: 19)

GAGGTGCAGCTGGTGGAGTCTGGGGGAGGCTTGGTGCAGCCGGGGGGGTC
TCTGAGACTCTCTTGTGCAGCCTCTGGTTTCGCCTTCAGCAGCCACGAGA
TGCACTGGGTTCGTCAAGGGATAGGACACAGTTTGGAGTGGGTCTCAGCT
ATTGGCCTTGCTGGTGACACATATTATTCAGACTCCGTGAAGGGCCGCTT
CACCATCTCCAGAGAAAACGCCAAGAACACTTTGTATCTTGAAATGAACA
ATTTGAGAGTCGGCGACACGGCTGTCTATTACTGTGTCAAAGAAGGGGCT
CATTGGGAATTTGACTCCTGGGGCCAGGGAACCCTGGTCACCGTCTCGA
GTG

Protein Sequence Defining the 5.27.A11 Heavy Chain Variable Region (SEQ ID NO: 20)

EVQLVESGGGLVQPGGSLRLSCAASGFAFSSHEMHWVRQGIGHSLEWVSA
IGLAGDTYYSDSVKGRFTISRENAKNTLYLEMNNLRVGDTAVYYCVKEGA
HWEFDSWGQGTLVTVSS

Nucleic Acid Sequence Encoding the 5.27.A11 Lambda Chain Variable Region (SEQ ID NO: 21)

CAGTCTGTGCTGACGCAGCCGCCCTCAGTGTCTGGGGCCCCTGGACAGAG
GGTCATCATCTCCTGCACTGGGAGCAGCTCCAACCTCGGGGCAAGTCTTG
ATGTACACTGGTACCAGCGCCTTCCAGGGACAGGCCCCCGACTCCTCATC
TATGCTAACAACAATCGGCCCTCAGGGGTCCCTGACCGATTCTCTGGCTC
CAAGTCTGGCACCTCAGCCTCCCTGGCCATCACCGGGCTCCAGGCTGAAG
ATGAGGGTGATTATTACTGCCAGTCCTATGACGGCAGCCTGAGGGTGTTC
GGCGGAGGGACCAGGCTGACCGTCCTAG

Protein Sequence Defining the 5.27.A11 Lambda Chain Variable Region (SEQ ID NO: 22)

QSVLTQPPSVSGAPGQRVIISCTGSSSNLGASLDVHWYQRLPGTGPRLLI
YANNNRPSGVPDRFSGSKSGTSASLAITGLQAEDEGDYYCQSYDGSLRVF
GGGTRLTVL

Nucleic Acid Sequence Encoding the 5.6.H2 Heavy Chain Variable Region (SEQ ID NO: 28)

CAGGTGCAGCTGGTGGAGTCTGGGGGAGGCTTGTTCAAGCCTGGAGGGTC
CCTGAGACTGTCCTGTGCAGCCTCTGGATTCACCTTCAGTGACTACTACA
TGAGCTGGATCCGGCAGGCTCCAGGGAAGGGGCTGGAGTGGATTTCATAC
ATTAGTAGTAGTGGTAGGACCATAAAGTACGCAGACTCTGTGAAGGGCCG
ATTCACCATCTCCAGGGACAACGCCAAGAACTCACTTTATCTGCAAATGA
ACAGCCTGAGAGCCGAGGACACGGCCGTGTATTATTGTGCGAGAGATGGA
GGATATTGTGGAAGTGCAAACTGCCTTCATGACGCTTTTGATATCTGGGG
CCAAGGGACAATGGTCACCGTCTCGAGTG

Protein Sequence Defining the 5.6.H2 Heavy Chain Variable Region (SEQ ID NO: 29)

QVQLVESGGGLFKPGGSLRLSCAASGFTFSDYYMSWIRQAPGKGLEWISY
ISSSGRTIKYADSVKGRFTISRDNAKNSLYLQMNSLRAEDTAVYYCARDG
GYCGSANCLHDAFDIWGQGTMVTVSS

Nucleic Acid Sequence Encoding the 5.6.H2 Kappa Chain Variable Region (SEQ ID NO: 30)

GACATCCAGATGACCCAGTCTCCTTCCACCCTGTCTGCATCTGTAGGAGA
CAGAGTCACCATCACTTGCCGGGCCAGTCAGAGTATTAGTAGCTGGTTGG
CCTGGTATCAGCAGAAACCAGGGAAAGCCCCTAAGCTCCTGATCTATAAG
GCGTCTAGTTTAGAAAGTGGGGTCCCATCAAGGTTCAGCGGCAGTGGATC
TGGGACAGAATTCACTCTCACCATCAGCAGCCTGCAGCCTGATGATTTTG
CAACTTATTACTGCCAACAGTATAATAGTTATTCGACGTTCGGCCAAGGG
ACCAAGGTGGAAATCAAAC

Protein Sequence Defining the 5.6.H2 Kappa Chain Variable Region (SEQ ID NO: 31)

DIQMTQSPSTLSASVGDRVTITCRASQSISSWLAWYQQKPGKAPKLLIYK
ASSLESGVPSRFSGSGSGTEFTLTISSLQPDDFATYYCQQYNSYSTFGQG
TKVEIK

Nucleic Acid Sequence Encoding the 5.6.H9 Heavy Chain Variable Region (SEQ ID NO: 37)

CAGGTCACCTTGAAGGAGTCTGGTCCCACGCTGGTGAAACCCACACAGAC
CCTCACGCTGACGTGCACCTTCTCTGGGTTTTCGCTCAGCACTAGTGGAG
AGGGTGTGGCCTGGATCCGTCAGGCCCCAGGAAAGGCCCTGGAGTGGCTT
GCACTCATTTATTGGGATGATGATAAGCGCTACAACCCATCTCTGAAGAG
CAGGCTCACCATCACCAAGGACAACTCCAGAAACCAAGTGGTCCTTACAA
TGACCAACATGGACCCTGTGGACACAGCCACATATTACTGTGTACACAGG
GGGATGAATAGGGTTTTTGGGGTAGTTTATAATTCGGGCTGGTTCGACCC
CTGGGGCCAGGGAACCCTGGTCACCGTCTCGAGTG

Protein Sequence Defining the 5.6.H9 Heavy Chain Variable Region (SEQ ID NO: 38)

QVTLKESGPTLVKPTQTLTLTCTFSGFSLSTSGEGVAWIRQAPGKALEWL
ALIYWDDDKRYNPSLKSRLTITKDNSRNQVVLTMTNMDPVDTATYYCVHR
GMNRVFGVVYNSGWFDPWGQGTLVTVSS

Nucleic Acid Sequence Encoding the 5.6.H9 Kappa Chain Variable Region (SEQ ID NO: 39)

GAAATTGTGTTGACGCAGTCTCCAGGCACCCTGTCTTTGTCTCCAGGGGA
AAGAGCCACCCTCTCCTGTAGGGCCAGTCAGAGCGTTAGAAGCAGCCAGC
TAGCCTGGTACCAGCAGAAGCCTGGCCAGGCTCCCAGCCTCCTCATGTAT
GACGCATCTAAGAGGGCCACTGGCATCCCAGACAGGTTCAGTGGCAGTGG
GTCTGGGACAGACTTCACTCTCACCATCAGCAGACTGGAGCCTGAAGATT
TTGTAGTGTATTTCTGTCAGCAGTATGATACATCACCGTGGACGTTCGGC
CAAGGGACCAAGGTGGAAATCAAAC

Protein Sequence Defining the 5.6.H9 Kappa Chain Variable Region (SEQ ID NO: 40)

EIVLTQSPGTLSLSPGERATLSCRASQSVRSSQLAWYQQKPGQAPSLLMY
DASKRATGIPDRFSGSGSGTDFTLTISRLEPEDFVVYFCQQYDTSPWTFG
QGTKVEIK

Nucleic Acid Sequence Encoding the 5.17.F8 Heavy Chain Variable Region (SEQ ID NO: 46)

CAGGTGCAGCTACAGCAGTGGGGCGCAGGACTGTTGAAGCCTTCGGAGAC
CCTGTCCCTCAGTTGCGCTGTCTCCGGTACGTCCTTCAGTGATTACTACT
GGGCCTGGATCCGTCAGCCCCCAGGGAAGGGGCTGGAGTGGATTGGGGAA
ATCAATCAGAGTGGACACACTAACGACAACCCGTCCTTCAAGAGTCGAGT
CACCATGTTAGTAGACAAGTCCAAGAACCAGTTCTCCCTGAGGATGACTT
CTGTGACCGCCGCGGACACGGCTGTATATTACTGTGCGAGATTTCCTTGG
GTGGGACCCGAGGGTTTTGACCACTGGGGCCAGGGAACCCTGGTCACCGT
CTCGAGTG

Protein Sequence Defining the 5.17.F8 Heavy Chain Variable Region (SEQ ID NO: 47)

QVQLQQWGAGLLKPSETLSLSCAVSGTSFSDYYWAWIRQPPGKGLEWIGE
INQSGHTNDNPSFKSRVTMLVDKSKNQFSLRMTSVTAADTAVYYCARFPW
VGPEGFDHWGQGTLVTVSS

Nucleic Acid Sequence Encoding the 5.17.F8 Kappa Chain Variable Region (SEQ ID NO: 48)

GAAATTGTGCTGACTCAGTCTCCAGACTTTCAGTCTGGGACTCCAAAGGA
GATGGTCACCATCACCTGCCGGGCCAGTCAGACCATTGGTAACAGCTTAC
ACTGGTACCAACAGAAACCAGGTCAGTCTCCAAAGCTCCTCATCAAATAT
GGTTCCCAGTCCATGTCAGGGGTCCCCTCGCGGTTCAGTGGCAGTGGATT
TGGGACAGATTTCACCCTGACCATCAACAGCCTGGAAGCTGAAGATGCTG
CAGTGTATTACTGTCTTCAGAGTGGTGGTTTTCCGTGGACGTTCGGCCAA
GGGACCAAGGTGGAGATCAGACGAACTGTGGCTGCACCATCTGTCTTCAT
CTTCCCGCCATCTGATGAGCAGTTGAAATCTG

Protein Sequence Defining the 5.17.F8 Kappa Chain Variable Region (SEQ ID NO: 49)

EIVLTQSPDFQSGTPKEMVTITCRASQTIGNSLHWYQQKPGQSPKLLIKY
GSQSMSGVPSRFSGSGFGTDFTLTINSLEAEDAAVYYCLQSGGFPWTFGQ
GTKVEIR

Nucleic Acid Sequence Encoding the 5.19.F12 Heavy Chain Variable Region (SEQ ID NO: 55)

CAGGTCCAGCTGGTACAGTCTGGGGCTGAGATGAAGAAGCCTGGGGCCTC
AGTGAAGGTCTCCTGCAAGGGTTCCGGATCCAGTCTCCCTTATCTTTCGA
TACAATGGGTGCGACAGGCCCCTGGAAAAGGCCTTGAGTGGATGGGAGGC
TTTGATCCTGAAGACGGTGAAACAATCTACAACGAGAAATTCGAGGGCAG
AATCACCATGGCCGAGGACACATCGACAGACACAGCCTACATGGAACTCA
GCAGCCTCACATCTGACGACACGGCCGTCTATTATTGCGCAACAAGCCTC
ATCCGTAACGGTCTTTATGAAGCGTTCGAACACTGGGGCCAGGGAACCCT
GGTCACCGTCTCGAGTG

Protein Sequence Defining the 5.19.F12 Heavy Chain Variable Region (SEQ ID NO: 56)

QVQLVQSGAEMKKPGASVKVSCKGSGSSLPYLSIQWVRQAPGKGLEWMGG
FDPEDGETIYNEKFEGRITMAEDTSTDTAYMELSSLTSDDTAVYYCATSL
IRNGLYEAFEHWGQGTLVTVSS

Nucleic Acid Sequence Encoding the 5.19.F12 Kappa Chain Variable Region (SEQ ID NO: 57)

GACATCCAGATGACCCAGTCTCCATCCTCCCTGTCTGCATCTGTAGGAGA
CAGAGTCACCATCACTTGCCAGGCGAGTGATGACATTAAAAAGAATTTAA
ATTGGTATCAACAAAAAACAGGGACAGCCCCTAAGCTCCTGGTCTACGAC
GCATCCAATTTGGAGACAGGGGTCCCATCGAGGTTCCGTGGACGTGCATC
TGGGACACAATATACTTTCACCATCACGAATCTGCAGCCTCAAGATTCTG
GAATATATTACTGTCAACAATATGACAATGTCCCGCTCAGTTTTGGCGGA
GGGACCAAGGTGGAGGTCAGAC

Protein Sequence Defining the 5.19.F12 Kappa Chain Variable Region (SEQ ID NO: 58)

DIQMTQSPSSLSASVGDRVTITCQASDDIKKNLNWYQQKTGTAPKLLVYD
ASNLETGVPSRFRGRASGTQYTFTITNLQPQDSGIYYCQQYDNVPLSFGG
GTKVEVR

Nucleic Acid Sequence Encoding the 5.23.C9 Heavy Chain Variable Region (SEQ ID NO: 64)

CAGGTGCAGCTGGTGCAGTCTGGGGCTGAGGTGAAGATGCCTGAGTCCTC
GGTGAGGGTCTCCTGCAGGATTTCTGGAGGCAACTTCAAAAATTATATTT
TTAACTGGGTGCGACAGGCCCCCGGACAAGGGCTTGAGTGGGTTGGAAGG
ATCAACCCAAAAGTTGGAAATGAAAATCACATACAGAGATATGGGAACAG
ACTCACCCTCACCGCGGACGATTCCAGGAGAACAGTTTACATGGAGTTGA
GCAGCCTGACAACAGACGACACGGCCGTATATTATTGTGCGACATTACGT
GTTATTGGTAACCGGAGACTACGACCGGTGGGCGGAATTTTTGATATCTG
GGGCCAAGGGACAATGGTCACCGTCTCGAGTG

Protein Sequence Defining the 5.23.C9 Heavy Chain Variable Region (SEQ ID NO: 65)

QVQLVQSGAEVKMPESSVRVSCRISGGNFKNYIFNWVRQAPGQGLEWVGR
INPKVGNENHIQRYGNRLTLTADDSRRTVYMELSSLTTDDTAVYYCATLR
VIGNRRLRPVGGIFDIWGQGTMVTVSS

Nucleic Acid Sequence Encoding the 5.23.C9 Kappa Chain Variable Region (SEQ ID NO: 66)

GACATCCAGATGACCCAGTCTCCATCCTCCCTGTCTGCGTCTGTGGGAGA
CAGAGTCACCATCACTTGCCAGGCGAGTCAGGACGTGAGTCACTATGTAA
ATTGGTATCAACAAAAACCAGGGAAAGCCCCAAAAGCCCTGGTCTACGAG
GCATCCAATTTGGAAACAGGGGTCCCATCAAGATTCAGTGGAAGTGGATC
TGGGACACATTTCACCTTCACCATCAACAGCCTGCAGCCTGAAGATATCG
CGACTTATTTCTGTCAACATGATGCAGAGCTGCCTCCCTCTTTCGGAGGA
GGGACCAAGGTGGAGATCAAAC

Protein Sequence Defining the 5.23.C9 Kappa Chain Variable Region (SEQ ID NO: 67)

DIQMTQSPSSLSASVGDRVTITCQASQDVSHYVNWYQQKPGKAPKALVYE
ASNLETGVPSRFSGSGSGTHFTFTINSLQPEDIATYFCQHDAELPPSFGG
GTKVEIK

Nucleic Acid Sequence Encoding the 5.23.C12 Heavy Chain Variable Region (SEQ ID NO: 73)

CAGGTTCAGCTGGTGCAGTCTGGAGCTGAAGTGAAGAAGCCTGGGGCCT
CAGTGAAGGTCTCCTGCAAGTCTTCTGGTCACTCCTTTACCGCCTATG
GCATCAGCTGGGTGCGACAGGCCCCTGGACAAGGGCTTGAGTGGATGG
GGTGGATCAGCACTAACAATGGCAACACAAACTATAGAGAGAAGTTC
CAGGACAGACTCTCCTTGACCACGGACACATCCACGAGCACAGTCTACA
TGGAACTGAGGAGCCTGACATCTGACGACACGGCCGTGTATTACTGTG
CGAGAAATCCTTATGGCAATTCGCGGAGATTCTTAGACTACTGGGGCC
AGGGAACCCTGGTCACCGTCTCGAGTG

Protein Sequence Defining the 5.23.C12 Heavy Chain Variable Region (SEQ ID NO: 74)

QVQLVQSGAEVKKPGASVKVSCKSSGHSFTAYGISWVRQAPGQGLEW
MGWISTNNGNTNYREKFQDRLSLTTDTSTSTVYMELRSLTSDDTAVY
YCARNPYGNSRRFLDYWGQGTLVTVSS

Nucleic Acid Sequence Encoding the 5.23.C12 Kappa Chain Variable Region (SEQ ID NO: 75)

GACATCCAGATGACCCAGTCTCCATCCTCCCTGTCTGCATCTGTCGGAG
ACAGAGTCATCATCACTTGCCGGGCAAGTCAGAACATTAGCCCCTAT
TTAAATTGGTATCAGCAGAAGCCAGGGAGAGCCCCTGAGCTCCTGATCT
ATGGTACGACCACTGTACAAACTGGGGTCCCGTCACGGTTCAGTGGC
ATTGGATCTGGGACAGATTTCACTCTCACCATCAGCAGTCTGCAACCTG
AAGATTTTGCAACTTATTACTGTCAACAGAGCTACAGGGCCCCTCAAA
CGTTCGGCCAAGGGACCAAGGTGGAAGTCAAGC

Protein Sequence Defining the 5.23.C12 Kappa Chain Variable Region (SEQ ID NO: 76)

DIQMTQSPSSLSASVGDRVIITCRASQNISPYLNWYQQKPGRAPELL
IYGTTTVQTGVPSRFSGIGSGTDFTLTISSLQPEDFATYYCQQSYR
APQTFGQGTKVEVK

Nucleic Acid Sequence Encoding the 8.51.G11 Heavy Chain Variable Region (SEQ ID NO: 82)

CAGGTCACCTTGAAGGAGTCTGGTCCTCCGCTGGTGAATCCCACAGAGA
CCCTCACGGTGACCTGCAATTTCCCTGGCTTCTCATTCACCTCTAGTG
GAGAGGGTGTGGGCTGGATCCGTCAGCCCCCAGGAAAGGCCCTGGAAT
GGCTTGCTCACATTTATTGGGATGGCGATAAGCGGTTCAACACATCT
CTGAAAAACAGACTCAACATCACCAAGGACACCTCTAAGAATCAGGTGG
TCCTCACTTTGACCAACGTGGCCCCTGTGGACACAGCCACGTATTTCT
GTGTCCACCTGACACGCCCATCCTTTTGGAGTCCCTATTACCTTTTTGA
CCTCTGGGGCCAGGGAACCCTGGTCACCGTCTCGAGTG

Protein Sequence Defining the 8.51.G11 Heavy Chain Variable Region (SEQ ID NO: 83)

QVTLKESGPPLVNPTETLTVTCNFPGFSFTSSGEGVGWIRQPPGKALE
WLAHIYWDGDKRFNTSLKNRLNITKDTSKNQVVLTLTNVAPVDTATYF
CVHLTRPSFWSPYYLFDLWGQGTLVTVSS

Nucleic Acid Sequence Encoding the 8.51.G11 Kappa Chain Variable Region (SEQ ID NO: 84)

GACATCCAGTTGACCCAGTCTCCGCCCTCCCTGTCTGCATCTGTCGGAG
ACAGTGTCACCATAACTTGCCGGGCAAGTCAATATATTAGTACGTAT
TTAAATTGGTATCAGCAGAAACCGGGGAAAGCCCCCAGACTCTTGATC
TATGGTGCATCCAATTTGCACAGTGGGGTCTCATCAAGGTTCAGTG
GCAGTAGATCTGGGACAGATTTCACTCTCACCATAACCAGTCTACAGC
CTGAAGATTTTGCGACTTACTACTGCCAACAGAGTTCCACTTTGTGG
TCGTTCGGCCAAGGGACCAAGGTGGAGATGCACC

Protein Sequence Defining the 8.51.G11 Kappa Chain Variable Region (SEQ ID NO: 85)

DIQLTQSPPSLSASVGDSVTITCRASQYISTYLNWYQQKPGKAPRLL
IYGASNLHSGVSSRFSGSRSGTDFTLTITSLQPEDFATYYCQQSST
LWSFGQGTKVEMH

Nucleic Acid Sequence Encoding the 9.51.H9 Heavy Chain Variable Region (SEQ ID NO: 91)

CAGGTTCAGCTGGTGCAGTCTGGAGGTGAAATGAGGAAACCTGGGGCCT
CATTGAAGGTCTCCTGCAAGGCCTCTAATTACACCTTCACCAGTTTTG
GTCTGACCTGGCTGCGACAGGCCCCTGGGCAAGGCCCTGAGTGGCTGG
GCTGGATCAGTCCTTATAATGGAAACACCAACTATGCACAGAACTTC
CGCGACAGACTCACCATGACCACAGACACGTCCACGAGCACAGCCTACA
TGGAGCTGAAGAGCCTGACATCTGACGACACGGCCGTCTATTACTGTG
CCAGAACTAACTGGGGGAATTGGTACCTCGATCTCTGGGGCCCTGG
CACCCTGGTCACTGTCTCGAGTG

Protein Sequence Defining the 9.51.H9 Heavy Chain Variable Region (SEQ ID NO: 92)

QVQLVQSGGEMRKPGASLKVSCKASNYTFTSFGLTWLRQAPGQGPEW
LGWISPYNGNTNYAQNFRDRLTMTTDTSTSTAYMELKSLTSDDTAVY
YCARTNWGNWYLDLWGPGTLVTVSS

Nucleic Acid Sequence Encoding the 9.51.H9 Kappa Chain Variable Region (SEQ ID NO: 93)

GACATCGTGATGACCCAGTCTCCAGACTCCCTGGCTGTGGCTCTGGGCG
AGCGGGCCACCATCAGCTGCAAGTCCAGCCAGACTATCTTGTTCGGCT
CCAACAACAAGAACTACTTACAGTGGTTTCAGCAGAAACCCGGACAGC
CTCCTAAGTTACTCATTTACTGGGCATCTACCCGGGAATCCGGGGTC
CCTGACCGATTCAGTGGCGACGGGTCTGGGACAGACTTCACTCTCTCCA
TCGACAACCTACAGGCTGAAGATGTGGCACTTTATTATTGCCAGCAAT
ATTTTAGTAATCCGTATACTTTCGGCCAGGGGACCAAATTGGAGATCA
AAC

Protein Sequence Defining the 9.51.H9 Kappa Chain Variable Region (SEQ ID NO: 94)

DIVMTQSPDSLAVALGERATISCKSSQTILFGSNNKNYLQWFQQKPG
QPPKLLIYWASTRESGVPDRFSGDGSGTDFTLSIDNLQAEDVALYY
CQQYFSNPYTFGQGTKLEIK

Nucleic Acid Sequence Encoding the 18.43.D8 Heavy Chain Variable Region (SEQ ID NO: 100)

CAGGTCCAGCTTGTGCAGTCTGGGGCTGAGGTGAAGAAGCCTGGGGCCT
CAGTGAAAATTGCCTGCAAGACCTCTGGATACATCTTCGTTGACTATG
CTGTACAATGGGTGCGCCAGGCCCCCGGACAGAGGCTTGAGTGGATGG
GGTGGCTCCAGTCAGCCAGTGGCAAGACGAGCTCGTCACATAGTCTC
CAAGACAGAGTCTCCATTACCCGGGACACATCCGCGAACACAGCCTACT
TGGAACTGAGGAGTCTGACTTCTGAAGATACGGCTGTCTACTACTGTG
CGAGAGGGAGTATAAGTCATTACGCCCCCTTTGACTACTGGGGCCAGG
GAACCCTGGTCACCGTCTCGAGTG

Protein Sequence Defining the 18.43.D8 Heavy Chain Variable Region (SEQ ID NO: 101)

QVQLVQSGAEVKKPGASVKIACKTSGYIFVDYAVQWVRQAPGQRLEW
MGWLQSASGKTSSSHSLQDRVSITRDTSANTAYLELRSLTSEDTAVY
YCARGSISHYAPFDYWGQGTLVTVSS

Nucleic Acid Sequence Encoding the 18.43.D8 Kappa Chain Variable Region (SEQ ID NO: 102)

GACATCGTGATGACCCAGTCTCCAGACTCCCTGGCTGTGTCTCTGGGCG
AGAGGGCCACCATCGACTGCAAGTCCAGTCAGAATATTTTATATACTT
CCAACAATAAGAGTTTCTTAGCTTGGTACCAACAGAAACCAGGACAGC
CTCCTAAATTGCTCATTTACTGGGCGTCTACCCGAGAGTTCGGGGTC
CCTGCGCGATTCAGTGGCAGCGGGTCTGGGACAGATTTCACTCTCACCA
TCAACAACCTGCAGGGTGAAGATGTGGCCATTTATTACTGTCAACAGT
ATTTTTCTACTCCATACACTTTTGGCCAGGGGACCAAGCTGGAGATCA
AAC

Protein Sequence Defining the 18.43.D8 Kappa Chain Variable Region (SEQ ID NO: 103)

DIVMTQSPDSLAVSLGERATIDCKSSQNILYTSNNKSFLAWYQQKPGQP
PKLLIYWASTREFGVPARFSGSGSGTDFTLTINNLQGEDVAIYYCQQYF
STPYTFGQGTKLEIK

Nucleic Acid Sequence Encoding the 22.22.E7 Heavy Chain Variable Region (SEQ ID NO: 109)

CAGGTGCAGCTGCAGGAGTCGGGCCCAGGACTGGTGAAGCCCTCGGAGA
CCCTGTCCCTCACGTGCACTGTCTCTGGTGCCTACATCACTAATGACTAC
TGGAGCTGGATCCGGCAGCCCGCCGGGAAGGGACTGGCGTGGATTGGGCGT
CTCTATACCAGTGGGAGCACCAACTACAACCCCTCCCTCAAGAGTCGAG
TCACCATGTCAGTAGACACGTCCAAAAACCAGTTCTCCCTGAGGCTCAC
CTCTGTGACCGCCGCGGACACGGCCGTTTATTATTGTGCGAGAGATGAG
TGTAGTGGTGGTGGGTGCTTCCATGAGAACTGGGGCCAGGGAACCCTGG
TCACCGTCTCGAGTG

Protein Sequence Defining the 22.22.E7 Heavy Chain Variable Region (SEQ ID NO: 110)

QVQLQESGPGLVKPSETLSLTCTVSGAYITNDYWSWIRQPAGKGLAWIG
RLYTSGSTNYNPSLKSRVTMSVDTSKNQFSLRLTSVTAADTAVYYCARDE
CSGGGCFHENWGQGTLVTVSS

Nucleic Acid Sequence Encoding the 22.22.E7 Kappa Chain Variable Region (SEQ ID NO: 111)

GAAATTGTGTTGACGCAGTCTCCAGGCACCCTGTCTTTGTCTCCAGGGG
AAAGAGCCACCCTCTCCTGCAGGGCCAGCCAGAGTATTAGCAGCAGCTA
CTTGGCCTGGTACCAGCAGAAGCGTGGCCAGGCTCCCAGGCTCCTCATC
TATGGTGCATCCAGTAGGGCCACTGGCATCCCAGACAGGTTCAGTGGCA
GTGGGACTGGGACAGACTTCACTCTCACCATCAGCAGACTGGAGCCTGA
CGATTTTGCACTTTATTACTGTCAGCAGTATGATAGGTCACCGCTCACT
TTCGGCGGAGGGACCAAGGTGGAGATCAAAC

Protein Sequence Defining the 22.22.E7 Kappa Chain Variable Region (SEQ ID NO: 112)

EIVLTQSPGTLSLSPGERATLSCRASQSISSSYLAWYQQKRGQAPRLLI
YGASSRATGIPDRFSGSGTGTDFTLTISRLEPDDFALYYCQQYDRSPLT
FGGGTKVEIK

Nucleic Acid Sequence Encoding the 8.51.G10 Heavy Chain Variable Region (SEQ ID NO: 148)

CAGATCACCTTGAAGGAGTCTGGTCCTACGCTGGTGAACCCCACACAGA
CCCTCACGCTGACCTGCACCTTTTCTGGGTTCTCATTCACCTCTAGTGG
AGAGGCTGTGGGCTGGATCCGTCAGCCCCCAGGAAAGGCCCTGGAATGG
CTTGCTCACATTTATTGGGATGATGATCCGCGTTATAGTACATCTCTGA
AGAGCAGACTCACCATCACCAAGGACACCTCCAAAAATCAGGTGGTCCT
CTCTATGACCGACATTGACCCTGCGGACACAGGCACATATTACTGTGCA
CACCTGAGACGCCCATCCCTTTGGAGTCCTTATTATCTTTTTGACTGCT
GGGGCCAGGGAACCCTGGTCACCGTCTCGAGTG

Protein Sequence Defining the 8.51.G10 Heavy Chain Variable Region (SEQ ID NO: 149)

QITLKESGPTLVNPTQTLTLTCTFSGFSFTSSGEAVGWIRQPPGKALEW
LAHIYWDDDPRYSTSLKSRLTITKDTSKNQVVLSMTDIDPADTGTYYCA
HLRRPSLWSPYYLFDCWGQGTLVTVSS

Nucleic Acid Sequence Encoding the 8.51.G10 Kappa Chain Variable Region (SEQ ID NO: 150)

GACATCCAGATGACCCAGTCTCCATCCTCCCTGTCTGCATCTGTTGGAG
ACAGAGTCACCATCACTTGCCGGGCAAGTCAGAGTATTAGTAGCTATTT
AAATTGGTATCAACTGAAACCAGGACAAGCCCCTAAGCTCCTGATCTAT
GGTGCATCCAGTTTGGAAAATGGGGTCCCTTCACGGTTCAGTGGCAGTG
GCTCTGGGCCAGATTTCACTCTCACCATTAGCAGTCTGCAACCTGAAGA
TTTTGCAACTTACTACTGTCAACAGAGTTACAAAATGTGGTCATTCGGC
CAAGGGACCAAGGTGGAAATCCAAC

Protein Sequence Defining the 8.51.G10 Kappa Chain Variable Region (SEQ ID NO: 151)

DIQMTQSPSSLSASVGDRVTITCRASQSISSYLNWYQLKPGQAPKLLIY
GASSLENGVPSRFSGSGSGPDFTLTISSLQPEDFATYYCQQSYKMWSF
GQGTKVEIQ

Nucleic Acid Sequence Encoding the 5.24A.A7 Heavy Chain Variable Region (SEQ ID NO: 152)

CAGGTTCAGCTGGTGCAGTCTGGAGTTGAGGTGAAGAAGCCTGGGGCCT
CAGTGAAGGTCTCCTGCAAGGCTTCTGGCTACACCTTTACCAACTTTGG
CTTCAGTTGGGTGCGGCAGGCCCCAGGACAAGGGCTTGAGTGGCTGGGA
TGGATCAACACTCATAACGGTAACACAAACTATGCACCGAGATTCCAAG
ACAGAGTCACCCTGACCACAGACACATCCACGACCACAGCCTACATGGA
GCTGAGGAGCCTGAGATCTGAGGACACGGCCGTGTATTATTGTGCGAGA
GATCTTGGGGGAAGTAGTTCCGCCTACTGGGGACAGGGAACCCTGGTCA
CCGTCTCGAGTG

Protein Sequence Defining the 5.24A.A7 Heavy Chain Variable Region (SEQ ID NO: 153)

QVQLVQSGVEVKKPGASVKVSCKASGYTFTNFGFSWVRQAPGQGLEWLG
WINTHNGNTNYAPRFQDRVTLTTDTSTTTAYMELRSLRSEDTAVYYCA
RDLGGSSSAYWGQGTLVTVSS

Nucleic Acid Sequence Encoding the 5.24A.A7 Kappa Chain Variable Region (SEQ ID NO: 154)

GAAATTGTGTTGACGCAGTCTCCAGGCACCCTGTCTTTGTCTCCAGGGG
AGAGAGCCACCCTCTCTTGCAGGGCCAGTCACAGTGGTACCAACAACTA
CATCGCCTGGTACCAGCAGAAACCTGGCCAGCCTCCCAGGCTCCTCATC
TATGGTGCATCCAGCAGGGCCCCTGGCATCTCAGAGAGGTTCAGCGGCA
GTGGGTCTGGGACAGACTTCACTCTCATCATCAGCAGACTGGAGCCTGA
AGACTTTGGAGTATATTACTGTGAGCAGTATGGGAACTCACCTCTGACG
TTCGGCCAAGGGACCAAGGTGGAAATCAAAC

Protein Sequence Defining the 5.24A.A7 Kappa Chain Variable Region (SEQ ID NO: 155)

EIVLTQSPGTLSLSPGERATLSCRASHSGTNNYIAWYQQKPGQPPRLLI
YGASSRAPGISERFSGSGSGTDFTLIISRLEPEDFGVYYCEQYGNSPLT
FGQGTKVEIK

Nucleic Acid Sequence Encoding the 5.24A.F3 Heavy Chain Variable Region (SEQ ID NO: 156)

CAGGTGCAGCTGCAGGAGTCGGGCCCAGGACTGGTGGAGTCTTCGGACG
TCCTGTCCCTCACCTGCACTGTCTCTGGTGGCTCCATCAGTAATGACTA
TTGGGCCTGGATCCGGCAGACCCCAGGGAAGGGACTGGAGTGGATTGGG
TCTGTCTATTACAGTGGCAGAACCAGTTTCAGCCCCTCCCTCAAGAGTC
GAGTCACTATGTCAGTGGACACGACCAAGAACCAATTCTTCCTGAACCT
GGGCTCTCTGACCGTTGCGGACACGGCCGTGTATTACTGTGCGAGAACT
ACAAGGTTGTCCTTTGACTGGTTAGTACACTACTATGGAATGGACGTCT
GGGGCCAGGGGACCACGGTCACCGTCTCGAGTG

Protein Sequence Defining the 5.24A.F3 Heavy Chain Variable Region (SEQ ID NO: 157)

QVQLQESGPGLVESSDVLSLTCTVSGGSISNDYWAWIRQTPGKGLEWIG
SVYYSGRTSFSPSLKSRVTMSVDTTKNQFFLNLGSLTVADTAVYYCART
TRLSFDWLVHYYGMDVWGQGTTVTVSS

Nucleic Acid Sequence Encoding the 5.24A.F3 Kappa Chain Variable Region (SEQ ID NO: 158)

GACATCCAGATGACCCAGTCTCCATCGTCCCTGACTGCATCTGTAGGAGA
CAGAGTCACCATTATTTGTCGGGCAAGTCAGCCCATTGGCAGCTATTTGA
ATTGGTATCAGCAGAGTCCAGGGAAAGCCCCTAAACTCCTCATCTATGCT
GCATCCACTTTGCAGAGTGGGGTCCCATCAAGGTTCAGTGGCAGTGGATC
TGGGACAGATTTCACTCTCAACATCAGTGGTCTGCAACCTGAAGATTTTG
CAACTTACTACTGTCAACAGAGTTACAATAACCCTTTGTTCACCTTCGGC
CCTGGGACCAAAGTGGATATCAAACGAACTGTGGCTGCACCATCTGTCTT
CATCTTCCCGCCATCTGATGAGCAGTTGAAATCTG

Protein Sequence Defining the 5.24A.F3 Kappa Chain Variable Region (SEQ ID NO: 159)

DIQMTQSPSSLTASVGDRVTIICRASQPIGSYLNWYQQSPGKAPKLLIYA
ASTLQSGVPSRFSGSGSGTDFTLNISGLQPEDFATYYCQQSYNNPLFTFG
PGTKVDIK

Nucleic Acid Sequence Encoding the 5.8B.H4 Heavy Chain Variable Region (SEQ ID NO: 160)

GAGGTGCAGCTGGTGGAGTCTGGGGGAAGTCTGGTACGGCCGGGGGGGTC
CCTGCGACTCTCCTGTGCAGCCTCTGGATTCATCTTTGATGATTATGGCA
TGAGTTGGGTCCGCCAAGTTCCAGGGGAGGGCCTGGAGTGGGTCGCCGGC
ATTACTTGGGATGGCGTGAACACAGATTATGCAGACTCCGTGAAGGGCCG
CTTCACCATCTCCAGAGACAACGCCAAGAACTCCCTGTTTCTCCTAATGA
ACAGTCTCAGAGCCGAGGACACGGCCCTGTATCACTGTGCGAGAGCCAGA
TACTACCAAGGCACTGGCCTCCGGTCCTACCTCGACGAGTGGGGCCAGGG
AACCCTGGTCACCGTCTCGAGTG

Protein Sequence Defining the 5.8B.H4 Heavy Chain Variable Region (SEQ ID NO: 161)

EVQLVESGGSLVRPGGSLRLSCAASGFIFDDYGMSWVRQVPGEGLEWVAG
ITWDGVNTDYADSVKGRFTISRDNAKNSLFLLMNSLRAEDTALYHCARAR
YYQGTGLRSYLDEWGQGTLVTVSS

Nucleic Acid Sequence Encoding the 5.8B.H4 Kappa Chain Variable Region (SEQ ID NO: 162)

GACATCCAGATGACCCAGTCTCCATCCTCCGTGTCTGCATCTGTAGGAGA
AAGAGTCACTATCACTTGTCGGGCGACTCAGGGTATTGACAACTGGTTGG
CCTGGTATCAGCATAAACCAGGGAAAGCCCCGAAGCTCCTGATTTATGCT
GCATCCACTTTGCAAAGTGGGGTCCCGTCCAGGTTCAGCGGCAGTGGATC
TGGGACAGATTTCACTCTCACCATCAGCAGCGTGCAGCCTGAAGATGTTG
CAACTTACTTTTGTCAACAGGCTAAGAATTTCCCTCGGGGAGGATTCACT
TTCGGCCCTGGGACCAAAGTCGCTCTCAAACGAACTGTGGCTGCACCATC
TGTCTTCATCTTCCCGCCATCTGATGAGCAGTT

Protein Sequence Defining the 5.8B.H4 Kappa Chain Variable Region (SEQ ID NO: 163)

DIQMTQSPSSVSASVGERVTITCRATQGIDNWLAWYQHKPGKAPKLLIYA
ASTLQSGVPSRFSGSGSGTDFTLTISSVQPEDVATYFCQQAKNFPRGGFT
FGPGTKVALK

Nucleic Acid Sequence Encoding the 26.51.E1 Heavy Chain Variable Region (SEQ ID NO: 164)

CAGGTGCAGCTGGTGGAGTCTGGGGGAGGCGTGGTCCAGCCTGGGAGGTC
CCTGAGACTCTCCTGTGCAGCCTCTGGATTCACCTTCAGTAAATTTGCCA
TGCACTGGGTCCGCCAGGCTCCAGGCAAGGGGCTGGAGTGGGTGGCAGCT
ATAGCGTCTGATGGATATAATAAATACTATGCAGACTCCGTGAAGGGCCA
ATTCACCGTCTCCAGAGACACTTCCAGGAACACGCTGTATCTCCAAATGG
ACAGCCTGAGACCTGATGACACGGCTATTTATTACTGTGCGTACGGTCAC
TTGTCTTTTTGGTTCGACCCCTGGGGCCAGGGAACCCTGGTCACCGTCTC
GAGTG

Protein Sequence Defining the 26.51.E1 Heavy Chain Variable Region (SEQ ID NO: 165)

QVQLVESGGGVVQPGRSLRLSCAASGFTFSKFAMHWVRQAPGKGLEWVAA
IASDGYNKYYADSVKGQFTVSRDTSRNTLYLQMDSLRPDDTAIYYCAYGH
LSFWFDPWGQGTLVTVSS

Nucleic Acid Sequence Encoding the 26.51.E1 Kappa Chain Variable Region (SEQ ID NO: 166)

GACATCCAGATGACCCAGTCTCCATCCTCCCTGTCCGCATCTGTAGGAGA
CAGAGTCACCATCACTTGCCGGGCAAGTCAGGACATTGGAAATGGCTTAG
GCTGGTATCAACAGATACCAGGGAAAGCCCCTAAGCGCCTGATCTATGCT
GCATCCTCTTTGCAAAGTGGGGTCCCATCAAGGTTCAGCGGCAGTGGTTC
TGGGACAGAGTTCACTCTCACAATCCACAGCCTGCAGCCTGAAGATTTTG
CAACTTATTACTGTCTACAGCATAATTTTTACCCTCGCACTTTTGGCCAG
GGGACCAAGCTGGAGATCAAGCGAACTGTGGCTGCACCATCTGTCTTCAT
CTTCCCGCCATCTGATGAGCAGTTGAAATCTG

Protein Sequence Defining the 26.51.E1 Kappa Chain Variable Region (SEQ ID NO: 167)

DIQMTQSPSSLSASVGDRVTITCRASQDIGNGLGWYQQIPGKAPKRLIYA
ASSLQSGVPSRFSGSGSGTEFTLTIHSLQPEDFATYYCLQHNFYPRTFGQ
GTKLEIK

Nucleic Acid Sequence Encoding the 22.21.A7 Heavy Chain Variable Region (SEQ ID NO: 168)

CAGGTGCAGCTGGTGCAGTCTGGGGCTGAGGTGAAGAAGCCTGGGGCCTC
AGTGAAGGTCTCCTGCAAGGCTTCTGGATACACCTTCACCGACTACTATA
TACACTGGGTGCGACAGGCCCCTGGACAAGGGCTTGAGTGGATGGGATGG
ATCAACCCTTATAGTGGTGGCACAAACTATGCACCGAAGTTTCAGGGCAG
GTTCACCATGGCCAGGGACACGTCCATCACCACAACCTACTTGGACCTGG
GCAGGCTGACGTCTAGCGACACGGCCATTTATTTCTGTGCGAGAGATTTC
TTCAGAGCAGCAGCTGGCACGGCCGGAGACTACTGGGGCCAGGGAACCCT
GGTCACCGTCTCGAGTG

Protein Sequence Defining the 22.21.A7 Heavy Chain Variable Region (SEQ ID NO: 169)

QVQLVQSGAEVKKPGASVKVSCKASGYTFTDYYIHWVRQAPGQGLEWMGW
INPYSGGTNYAPKFQGRFTMARDTSITTTYLDLGRLTSSDTAIYFCARDF
FRAAAGTAGDYWGQGTLVTVSS

Nucleic Acid Sequence Encoding the 22.21.A7 Kappa Chain Variable Region (SEQ ID NO: 170)

GACATCGTGATGACCCAGTCTCCAGAAGACTCCCTGGCTGTGTCTCTGGG
CGAGAGGGCCACCATCAACTGCTCCAGCCAGAGTGCTTTATACAGCCCCA
GCAGTAAGACCTACTTAGCTTGGTACCAGCAGAGACCGGGACACCCTCCT
AAGCTTCTCATTTACTGGGCATCTACCCGGGTATCCGGGGTCCCTGACCG
ATTCAGTGGCAGCGGGTCTGGGACAGATTTCACTCTCACCATCGGCAGCC
TGCAGGCTGAAGATGTGGCAGTTTATTACTGTCAGTACTATTATGGTACT
GTCACTTTCGGCGGAGGGACCAAGGTGGAGATCAAACGAACTGTGGCTGC
ACCATCTGTCTTCATCTTCCCGCCATCTGATGAGCAGTTGAAATCTG

Protein Sequence Defining the 22.21.A7 Kappa Chain Variable Region (SEQ ID NO: 171)

ADIVMTQSPDSLAVSLGERATINCSSQSALYSPSSKTYLAWYQQRPGHPP
KLLIYWASTRVSGVPDRFSGSGSGTDFTLTIGSLQAEDVAVYYCQYYYGT
VTFGGGTKVEIK

Nucleic Acid Sequence Encoding the 22.18A.E9 Heavy Chain Variable Region (SEQ ID NO: 172)

CAGGTGCAGCTGCAGGAGTCGGGCCCAAGACTGGTGAAGCCTTCGGAGAC
CCTGTCCCTCACCTGCACTGTCTCTGGTGGGTCCATCAATAGTTATTATT
GGAGTTGGATCCGGCAGCCCGTCGGGAAGGGACTGGAGTGGATTGGGCGT
ATGTATCCCGGTGGTACCTCCAGTTACAATTCATCCCTCAAGAGTCGAGT
CATCATGTCGGTAGACACGTCGAAGAACCAGTTTTCTCTGAACCTCACTT
CTGTGACCGCCGCAGACACGGCCGTATATTTCTGTGCGAGAGATCCAACA
TATCACCATGGCACTAGTGGTTATGTGGGGACTTTTGATCTCTGGGGCCA
AGGGACAATGGTCACCGTCTCGAGTG

Protein Sequence Defining the 22.18A.E9 Heavy Chain Variable Region (SEQ ID NO: 173)

QVQLQESGPRLVKPSETLSLTCTVSGGSINSYYWSWIRQPVGKGLEWIGR
MYPGGTSSYNSSLKSRVIMSVDTSKNQFSLNLTSVTAADTAVYFCARDPT
YHHGTSGYVGTFDLWGQGTMVTVSS

Nucleic Acid Sequence Encoding the 22.18A.E9 Kappa Chain Variable Region (SEQ ID NO: 174)

GAAATAGTGATGACGCAGTCTCCAGCCACCCTGTCTGCGTCTCCAGGGG
AAAGAGTCACCCTCTCCTGCAGGGCCAGTCCGAGTGTTCGCAACAATTT
AGCCTGGTACCAGCAGAAACCTGGCCAGCCTCCTAGGCTCCTCATCTAT
GGTGCATCCACCAAGGTCATGGGTACTCCAACCAGGTTCAGTGGCAGTG
GGTCTGGGACAGAGTTCACTCTCACCATCAGCAGCCTGCAGTCTGAAGA
TTTTGCAGTTTATTACTGTCAGCAGTATAATAGCTGGCCTCCGTACACT
TTTAGCCAGGGGACCAAACTGGAGATCAAACGAACTGTGGCTGCACCAT
CTGTCTTCATCTTCCCGCCATCTGATGAGCAGTTGAAATCTG

Protein Sequence Defining the 22.18A.E9 Kappa Chain Variable Region (SEQ ID NO: 175)

DIVMTQSPATLSASPGERVTLSCRASPSVRNNLAWYQQKPGQPPRLLIY
GASTKVMGTPTRFSGSGSGTEFTLTISSLQSEDFAVYYCQQYNSWPPYT
FSQGTKLEIK

Nucleic Acid Sequence Encoding the 5.52.H10 Heavy Chain Variable Region (SEQ ID NO: 176)

GAGGTGCAGCTGGTGGAGTCGGGGGGAGGCCTGGTCAGGCCTGGGGAGT
CCCTGAGACTCTCCTGTGCAGGCTCTGGATTCACCTTCAGCATGTTTAC
CCTGAACTGGGTCCGCCAGGCTCCAGGGAAGGGACTGGAGTGGGTCGCA
TCTATTAGTGGTAGTAGCAGTTTAGTGTTTCATGAAGACTCAGTGAAGG
GCCGATTCACCATCTCCAGAGACAACGCCAAGAACTCAGTATATCTGCA
AATGAACAGCCTGAGAGCCGAAGACACGGCTATTTATTACTGTGTGAGA
GGAGATAGTCGCGACTATTGGGGCCAGGGAACCCTGGTCACCGTCTCGA
GTG

Protein Sequence Defining the 5.52.H10 Heavy Chain Variable Region (SEQ ID NO: 177)

EVQLVESGGGLVRPGESLRLSCAGSGFTFSMFTLNWVRQAPGKGLEWVA
SISGSSSLVFHEDSVKGRFTISRDNAKNSVYLQMNSLRAEDTAIYYCVR
GDSRDYWGQGTLVTVSS

Nucleic Acid Sequence Encoding the 5.52.H10 Kappa Chain Variable Region (SEQ ID NO: 178)

CAGTCTGTGTTGACGCAGCCGCCCTCAGTGTCTGCGGCCCCAGGACAGA
AGGTCACCATCTCCTGCTCTGGAAGGACCTCCAATATTGGAAATAACCA
TGTATCCTGGTACCAGCAATTCCCAGGAACAGCCCCCAAAGTCCTCATT
TATCGCAATGATAGGCGACCCTCAGGGATTCCTGATCGATTCTCTGGCT
CCAAGTCTGACACGTCAGCCACCCTGGGCATCACCGGGCTCCAGACTGG
GGACGAGGCCGATTATTACTGCGGAACATGGGACAACAGCCTGAGTGGT
TGGGTGTTCGGCGGAGGGACCAAGCTGACCGTCCTAG

Protein Sequence Defining the 5.52.H10 Kappa Chain Variable Region (SEQ ID NO: 179)

QSVLTQPPSVSAAPGQKVTISCSGRTSNIGNNHVSWYQQFPGTAPKVLI
YRNDRRPSGIPDRFSGSKSDTSATLGITGLQTGDEADYYCGTWDNSLSG
WVFGGGTKLTVL

Nucleic Acid Sequence Encoding the 5.15.C1 Heavy Chain Variable Region (SEQ ID NO: 180)

CAGGTGCAGCTGGTGCAGTCTGGGGCTGAGGTGAAGAGGCCTGGGGCCT
CAGTGAAGGTCTCCTGTGAGACTTCTGGATATAGCTTCACCACTCATGA
TTTGAACTGGGTGCGACAGGCCGCTGGACAGGGGCTTGAGTGGATGGGA
TGGATGAACCCTAACACTGGTGACACATCCTACGCACAGAGGTTCCAGG
GCAGAGTCACCATGATCAGGGACGTCTCCATAAGTACAGCCTACTTGGA
ATTGACAGGCCTGACATTTGATGACACGGCCGTCTACTTCTGTGCGAGG
TCGAATTCTGCACCTGTTTCAACTCTTTTACCCGCTGATGCTTTTGATG
TCTGGGGCCAAGGGACAATGGTCACCGTCTCGAGTG

Protein Sequence Defining the 5.15.C1 Heavy Chain Variable Region (SEQ ID NO: 181)

QVQLVQSGAEVKRPGASVKVSCETSGYSFTTDLNWVRQAAGQGLEWMGW
MNPNTGDTSYAQRFQGRVTMIRDVSISTAYLELTGLTFDDTAVYFCARS
NSAPVSTLLPADAFDVWGQGTMVTVSS

Nucleic Acid Sequence Encoding the 5.15.C1 Kappa Chain Variable Region (SEQ ID NO: 182)

GACATCCAGATGACCCAGTCTCCATCCTCCCTGTCTGCATCTATAGGAG
ACACAGTCACCATCACTTGCCGGGCAAGTCAGATCATTAACAGCTATTT
AAATTGGTATCAGCAAAAACCAGGGGAAGCCCCTAAACTCCTGATCTAT
GCTGTATCCAATTTGCACCGTGGGGTCCCCTCAAGGTTCAGTGGCAGTG
GGTCTGCGGCAGATTTCACTCTCACTATCAGTGGTCTGCAACCTGAAGA
TTTTGCAACTTACTAC

[symbol]

[symbol]

GGCCAAGGGACACGACTGGACATTAAAC

Protein Sequence Defining the 5.15.C1 Kappa Chain Variable Region (SEQ ID NO: 183)

DIQMTQSPSSLSASIGDTVTITCRASQIINSYLNWYQQKPGEAPKLLIY
AVSNLHRGVPSRFSGSGSAADFTLTISGLQPEDFATYYCQQTYTTVAIT
FGQGTRLDIK

Nucleic Acid Sequence Encoding the 5.54.E6 Heavy Chain Variable Region (SEQ ID NO: 184)

CAGGTGCAGCTGGTGCAGTCTGGGGCGGAGGTGAAGAAGCCTGGGTCCT
CGGTGAACGTCTCCTGTAAGGCCTCCGCAGGGACCCTCAGCGGTTATAC
CATCAACTGGTTGCGCCAGGCCCCTGGACGAGGGTTTGAGTGGCTGGGA
AGGATCATCCCTCACCTTGGAATAGGAAACAACGCACATAACTGGGAGG
CCAGAGTCACCCTTACCGCCGACAAGTCCACGGACACTACCTACCTGGA
GTTGCGCGACCTGAGATCTGACGACGTGGCCATTTATTACTGCGCGGTG
GAGTTTTACCGGCTGGCAACGGTCACAACCCCAACCCTTGACTTCTGGG
GCCAGGGAACCCTGGTCACCGTCTCGAGTG

Protein Sequence Defining the 5.54.E6 Heavy Chain Variable Region (SEQ ID NO: 185)

QVQLVQSGAEVKKPGSSVNVSCKASAGTLSGYTINWLRQAPGRGFEWLG
RIIPLGIGNNANWEARVTLTADKSTDTTYLELRDLRSDDVAIYYCAVEF
YRLATVTTPTLDFWGQGTLVTVSS

Nucleic Acid Sequence Encoding the 5.54.E6 Lambda Chain Variable Region (SEQ ID NO: 186)

TCCTATGTGCTGACTCAGCCACCCTCGGTGTCCGTGGCCCCAGGAACGA
CGGCCAGGATTACCTGTGAGGGGGACCACATTGGAGCTAAAAGTGTGCA
CTGGTACCAGAGGAAGCCAGGCCAGGCCCCTGTGTTGGTCGTCTATATG
AATAGTGACCGGCCCTCAGGGATCCCTGAGCGATTGTCTGGCTCCAACT
CTGGGAACACGGCCACCCTGAGTATCAGCAGGGTCGAGGCCGAGGATGA
GGCCGACTATTATTGTCAGGTTTGGGATAGTTTTCGCGATCATCAGGTA
TTCGGCGGAGGGACCAAGTTGACCGTCCTGA

Protein Sequence Defining the 5.54.E6 Lambda Chain Variable Region (SEQ ID NO: 187)

SYVLTQPPSVSVAPGTTARITCEGDHIGAKSVHWYQRKPGQAPVLVVYM
NSDRPSGIPERLSGSNSGNTATLSISRVEAEDEADYYCQVWDSFRDHQV
FGGGTKLTVL

Nucleic Acid Sequence Encoding the 5.55.D2 Heavy Chain Variable Region (SEQ ID NO: 188)

CAGGTCACCTTGAAGGAGTCTGGTCCTGCTATAGTGAAACCCACACAG
ACCCTCACGCTGACCTGCACCTTCTCTGGGTTCTCACTCCGAACTAG
TGGAGTGGGTGTGGGCTGGATCCGTCAGCCCCCAGGAAAGGCCCTT
GAGTGGCTTGCACTCATTTACTGGGATGATGATAAGCGCTACAACC
CATCTCTGAAGACCAGACTCACCATCACCAAGGACTCCTCCAGAAACC
AGGTGTTCCTCACAATGACCAACATGGACCCTGTGGACACAGCCACAT
ACTTCTGTGCACACCGACAACATATTACAAACAGTTTTGGATTGGTT
ATTGCGAATGATGCTTTTGACATTTGGGGCCAAGGGACAATGGTCA
CCGTCTCGAGTG

Protein Sequence Defining the 5.55.D2 Heavy Chain Variable Region (SEQ ID NO: 189)

QVTLKESGPAIVKPTQTLTLTCTFSGFSLRTSGVGVGWIRQPPGKA
LEWLALIYWDDDKRYNPSLKTRLTITKDSSRNQVFLTMTNMDPVDT
ATYFCARQITNSFGLVIANDAFDIWGQGTMVTVSS

Nucleic Acid Sequence Encoding the 5.55.D2 Lambda Chain Variable Region (SEQ ID NO: 190)

CAGTCTGCCCTGACTCAGCCTCCGTCCGCGTCCGGGTCTCCTGGACAG
TCAGTCACCATCTCCTGCACTGGAACCAGCAGTGACGTTGGTCGTTA
TAATTATGTCGCCTGGTATCAACAACACCCAGGCAGAGCCCCCAGA
CTCATAATTTATGGGGTCACTAAGCGGCCCTCAGGGGTCCCTGATC
GCTTCTCTGGCTCCAAGTCTGGCAACACGGCCTCCCTGACCGTCTCTG
GGCTCCAGGCTGAAGATGAGGCTGATTATTATTGCAGTTCATATGC
AGGCAGCAACTTTTTGGAGGTGTTCGGCGGAGGGACCAAGCTGACC
GTCCTAG

Protein Sequence Defining the 5.55.D2 Lambda Chain Variable Region (SEQ ID NO: 191)

QSALTQPPSASGSPGQSVTISCTGTSSDVGRYNYVAWYQQHPGRAP
RLIIYGVTKRPSGVPDRFSGSKSGNTASLTVSGLQAEDEADYYCS
SYAGSNFLEVFGGGTKLTVL

Nucleic Acid Sequence Encoding the 22.14.A1 Heavy Chain Variable Region (SEQ ID NO: 192)

CAGGTGCAGCTGGTGCAGTCTGGGGCTGAGGTGAAGAAGCCTGGGTCC
TCGGTGAAGGTCTCCTGCAAGGCTTCTGGAGGCACCTTCAGCAGCTA
TGCAATCAACTGGGTGCGACAGGCCCCTGGACAAGGTCTTGAGTGG
ATGGGAGGGATCATCCCTATGTTTGATACAGCAATCTACGCACAGA
TGTTCCAGGGCAGAGTCACCATCACCGCGGACGAATCCACGAGCACAG
CCTACATGGAGCTGAGCAGCCTGAGATCTGACGACACGGCCGTATATT
ACTGTGCGAGAGATCGAAACGATTATTATGACAGTAGTGGTTATTCA
GGCGCCTTTGACCAGTGGGGCCAGGGAACCCTGGTCACCGTCTCGA
GTG

Protein Sequence Defining the 22.14.A1 Heavy Chain Variable Region (SEQ ID NO: 193)

QVQLVQSGAEVKKPGSSVKVSCKASGGTFSSYAINWVRQAPGQGLE
WMGGIIPMFDTAIYAQMFQGRVTITAPESTSTAYMELSSLRSDDTA
VYYCARDRNDYYDSSGYSGAFDQWGQGTLVTVSS

Nucleic Acid Sequence Encoding the 22.14.A1 Kappa Chain Variable Region (SEQ ID NO: 194)

GAAATTGTGTTGACGCAGTCTCCAGGCGCCCTGTCTTTGTCTCCAGG
GGAAAGAGCCACCCTCTCCTGCAGGGCCAGTCAGAGTGTTAGCAGC
AGCTACTTAGCCTGGTACCAGCAGAAACCTGGCCAGGCTCCCAGGC
TCCTCATCTATGGTGCATCCAGCAGGGCCACTGGCATCCCAGACAG
GTTCAGTGGCAGTGGGTCTGGGACAGACTTCACTCTCACAATCAGCAG
ACTGGAGCCTGAGGATTTTGCAGTGTATTACTGTCAGCAGTATGGT
AGCTCACCTCGCACTTTTGGCCAGGGGACCAAGCTGGAGATCAAAC

Protein Sequence Defining the 22.14.A1 Kappa Chain Variable Region (SEQ ID NO: 195)

EIVLTQSPGALSLSPGERATLSCRASQSVSSSYLAWYQQKPGQAPR
LLIYGASSRATGIPDRFSGSGSGTDFTLTISRLEPEDFAVYY
CQQYGSSPRTFGQGTKLEIK

Nucleic Acid Sequence Encoding the 26.53.B4 Heavy Chain Variable Region (SEQ ID NO: 196)

GAGGTGCAGCTGGTGGAGTCCGGGGGAGGCTTGGTCCAGCCTGGGGGG
TCCCTGAAACTCTCCTGTGCAGCCTCTGGGTTCAGCTTCAGTGGCT
CTGCTGTGGAATGGGTCCGCCAGGCTTCCGGGAGAGGGCTGGAGTG
GATTGGCCATATAAGAACCAAGACCAAAAATTACGCGACAGCGTAT
GCTGCGTCGGTGCAAGGCAGGTTCACCATCTTCAGAGATGATTCAAGG
AACACGGCGTATCTGCAGATGAACAGTCTTAAAACCGAGGACACGGCCG
TGTATTATTGTTCCAGACACATCGAATTTAGCAGTTCGTCCGGGCCC
CAGATGGGCCAGGGAACCCTGGTCACCGTCTCGAGTG

Protein Sequence Defining the 26.53.B4 Heavy Chain Variable Region (SEQ ID NO: 197)

EVQLVESGGGLVQPGGSLKLSCAASGFSFSGSAVEWVRQASGRGLE
WIGIRTKTKNYATAYAASVQGRFTIFRDDSRNTAYLQMNSLKTEDT
AVYYCSRIEFSSSSGPQMGQGTLVTVSS

Nucleic Acid Sequence Encoding the 26.53.B4 Kappa Chain Variable Region (SEQ ID NO: 198)

GACATCCAGATGACCCAGTCTCCTTCCACCCTGTCTGCATCTGTAGGC
GACACAGTCACCATCACTTGCCAGGCCAGTCAGAATATTATTACTTG
GTTGGCCTGGTATCAGCAGAAACCAGGGAAAGCCCCTAACATCCTC
ATCTATAAGGCGTCTGGTTTAGAGAGTGGGGTCCCACCGAGGTTCA
GCGGCAGTGGGTCGGGGACAGAATTCACTCTCACCATCAGCAGCCTGC
AGCCTGATGATGTTGCAACTTATTATTGCCTACAATATAAAACTGA
GCCGTGGACGTTCGGCCTAGGGACCAAGGTGGAGATCAGAC

Protein Sequence Defining the 26.53.B4 Kappa Chain Variable Region (SEQ ID NO: 199)

DIQMTQSPSTLSASVGDTVTITCQASQNIITWLAWYQQKPGKAPNI
LIYKASGLESGVPPRFSGSGSGTEFTLTISSLQPDDVATYYCLQ
YKTEPWTFGLGTKVEIR

Nucleic Acid Sequence Encoding the 5.63.E2 Heavy Chain Variable Region (SEQ ID NO: 200)

GAGGTGCAGCTGGTGGAGTCTGGGGGAGGCTTGGCCCAGCCGGGGGAG
TCCCTGAGACTCTCCTGTGCAGCCTCCGGATTCACCTTTAGTGCCT
ATTGGATGAGCTGGGTCCGCCAGACTCCAGGGAAGGGGCTGGAGTG
GGTGGCCAACATTAACCAGGGTGGAGATAAGACAGACTATTTGGAC
TCTGTGAAGGGCCGATTCACCGTCTCCAGAGACAATGCCAAGAACTTA
GTGTTTCTGCAAATGGCCAGCCTGAGAGCCGACGACACGGCCGTGTAT
TACTGTGGGAGAGGTACAAACCAAGACTACTGGGGCCAGGGAACCC
TGGTCACCGTCTCGAGTG

Protein Sequence Defining the 5.63.E2 Heavy Chain Variable Region (SEQ ID NO: 201)

EVQLVESGGGLAQPGESLRLSCAASGFTFSAYWMSWVRQTPGKGLE
WVANINQGGDKTDYLDSVKGRFTVSRDNAKNLVFLQMASLRADDTA
VYYCGRGTNQDYWGQGTLVTVSS

Nucleic Acid Sequence Encoding the 5.63.E2 Lambda Chain Variable Region (SEQ ID NO: 202)

CAGTCTGTGCTGACTCAGCCACCCTCAGCGTCTGGGACCCCCGGGCAG
AGGGTCACCATCTCTTGTTCTGGAAGCAGCTCCAATATCGGAAGTA
ATAATGTATACTGGTACCAACAGGTCCCAGGAACGGCCCCCAAATT
TCTCATCTATCCGAATAGTGAGCGGCCCTCAGGGGTCCCTGACCGA
TTCTCTGCCTCCAAGTCTGGCACCTCAGCCTCCCTGGCCATCAGTGGA
CTCCGGTCCGAGGATGAGGCTACTTATTACTGTGCAGCATGGGATG
ACAGCCTAAGAACTTATGTCTTCGCATCTGGGACCAAGGTCAC

Protein Sequence Defining the 5.63.E2 Lambda Chain Variable Region (SEQ ID NO: 203)

QSVLTQPPSASGTPGQRVTISCSGSSSNIGSNNVYWYQQVPGTAPKFL
IYPNSERPSGVPDRFSASKSGTSASLAISGLRSEDEATYYCAAWDDSL
RTYVFASGTKVTVL

Nucleic Acid Sequence Encoding the 5.64.G4 Heavy Chain Variable Region (SEQ ID NO: 204)

CAGGTGCAGCTGGTGGAGTCTGGGGGAGGCGTGGTCCAGCCTGGGAGG
TCCCTGAGACTCTCCTGTGCAGCCTCCGGATTCACCCTCAGGAGTTAT
GGCATGCACTGGGTCCGCCAGGCTCCAGGCAAGGGCCTGGAGTGGGTG
GCAGTTATATCATATGATGGAAGTAAGACATATTATAGAGACTCCGTG
AAGGGCCGATTCACCATCTCCAGAGACAATTCTAAGAACACACTGTAT
TTGCAAATGAACAGCCTGCGAGTTGAGGACACGGCTGTATATTATTGT
GCGAAAGAGGATAATTTGGGATCTGATGTTTTTGATAGATGGGGCCAA
GGGACAATGGTCACCGTCTCGAGTG

Protein Sequence Defining the 5.64.G4 Heavy Chain Variable Region (SEQ ID NO: 205)

QVQLVESGGGVVQPGRSLRLSCAASGFTLRSYGMHWVRQAPGKGLEWV
AVISYDGSKTYYRDSVKGRFTISRDNSKNTLYLQMNSLRVEDTAVYYC
AKEDNLGSDVFDRWGQGTMVTVSS

Nucleic Acid Sequence Encoding the 5.64.G4 Kappa Chain Variable Region (SEQ ID NO: 206)

GACATCGTGATGACCCAGTCTCCTTCCACCCTGTCTGCATCTGTAGGA
GACAGAGTTACCATCACTTGTCGGGCCAGTCAGAGTGCCAGTCAGTGG
TTGGCCTGGTATCAGCAAAAACCAGGGAAAGCCCCTAATCTCCTAATC
TATAAGGCGTCTAATTTACATAGTGGGGTCCCATCAAGGTTCAGCGGC
AGTGGATCTGGGACCGAATTCACTCTCACCATCAGCAGCCTACAGCCT
GATGATTTTGCAACTTATTACTGCCAACAATACAACATTTATCCGTGG
ACGTTCGGCCAAGGGACCAAGGTGGAAGTGAGAC

Protein Sequence Defining the 5.64.G4 Chain Variable Region (SEQ ID NO: 207)

DIVMTQSPSTLSASVGDRVTITCRASQSASQWLAWYQQKPGKAPNLLIY
KASNLHSGVPSRFSGSGSGTEFTLTISSLQPDDFATYYCQQYNIYPWTF
GQGTKVEVR

Nucleic Acid Sequence Encoding the 43.52.A11 Heavy Chain Variable Region (SEQ ID NO: 208)

CAGGTCCAGCTGGTGCAGTCTGGGGCTGAGGTGAAGGAGCCAGGGTCC
TCGGTGAGGGTCTCCTGCAAGGCTTCTGGACGCATCTTCCGGAATTAT
GCTGTCAGCTGGGTGCGACAGGCCCCTGGACAAGGGCTTGAGTGGGTG
GGCCGGTTCATCCCTTCGGAAGGAATAACAAACTACATTCCGACATTC
CGGGACAGACTCACCTTTACTGCGGACAAGTCCACGAACACAGCCTTC
ATTGACCTGACTAACCTGAAATATGAGGACACGGCCATATATTACTGT
GTGGGTGGCAGGCCAGGCGCTTTCGTCCCCTTTGACTCCTGGGGCCAG
GGAACCCTGGTCACCGTCTCGAGTG

Protein Sequence Defining the 43.52.A11 Heavy Chain Variable Region (SEQ ID NO: 209)

QVQLVQSGAEVKEPGSSVRVSCKASGRIFRNYAVSWVRQAPGQGLEWV
GRFIPSEGITNYIPTFRDRLTFTADKSTNTAFIDLTNLKYEDTAIYYC
VGGRPGAFVPFDSWGQGTLVTVSS

Nucleic Acid Sequence Encoding the 43.52.A11 Kappa Chain Variable Region (SEQ ID NO: 210)

GAAATAGTGATGACGCAGTCTCCTGCCACCCTGTCTGTGTCTCCAGGG
GAAAGAGCCACCCTCTCCTGCAGGGCCAGTCAGAGTGTCAGGGGCAAC
TTAGCCTGGTACCAGCAAAAACCTGGCCAGGCTCCCAGGCTTCTCATG
TATGATGCTTCTACCAGGGCCACTAGTTTCCCAGCCCGGTTCAGTGGC
AGTGGGTCTGGGACAGAGTTCATCTCACCATCAGCAGTGTGCAGTCTG
AAGATTTTGCACTTTATTACTGTCAGCAGTATAATGACTGGCCCCGGA
CTTTTGGCCAGGGGACCAAGGTGGAGATCAAAC

Protein Sequence Defining the 43.52.A11 Chain Variable Region (SEQ ID NO: 211)

EIVMTQSPATLSVSPGERATLSCRASQSVRGNLAWYQQKPGQAPRLLM
YDASTRATSFPARFSGSGSGTEFTLTISSVQSEDFALYYCQQYNDWPR
TFGQGTKVEIK

Nucleic Acid Sequence Encoding the 43.52.E12 Heavy Chain Variable Region (SEQ ID NO: 212)

CAGGTGCAGCTGGTGCAGTCTGGGGCTGAGGTGAAGAAGCCTGGGTCC
TCGGTGAGGGTCTCCTGCAGGGTTTCTGGAGGCAGTTTCTCCAGTTAT
GACGTACACTGGGTGCGACAGGCCCCTGGACAAGGGCTTGAGTGGGTG
GGAAGGAGCGTCTCACGTGCTGACAGCTCAAGTCACACACGGGAGTTC
GGGGACAGAGTCACGATTATCGCGGACAAATCCACGAGTACAGTTTAT
TTGACACTGACCAGTCTGACCTCTGACGACACGGCCATCTATTACTGT
GCGGCAGGGGATTGTAGTGATGGCGGGTGCTTCCGCACACCCCTGCAC
ATCTGGGGCCAGGGCACCCTGGTCACCGTCTCGAGTG

Protein Sequence Defining the 43.52.E12 Heavy Chain Variable Region (SEQ ID NO: 213)

QVQLVQSGAEVKKPGSSVRVSCRVSGGSFSSYDVHWVRQAPGQGLEWV
GRSVSRADSSSHTREFGDRVTIIADKSTSTVYLTLTSLTSDDTAIYYC
AAGDCSDGGCFRTPLHIWGQGTLVTVSS

Nucleic Acid Sequence Encoding the 43.52.E12 Kappa Chain Variable Region (SEQ ID NO: 214)

GAAATAGTGATGACGCAGTCTCCAGCCACTCTGTCTCTGTCTCCAGGG
GAAAGAGCCACCCTCTCCTGCAGGGCCAGTCAGAGTCTGAGAACCAAC
TTAGCCTGGTACCAGCAGCGACCTGGCCAGGCTCCCAGGCTCCTCATC
TCTGGGGCATCCACGAGGGCCACTGGTATCCCAGCCAGGTTCAGTGGC
AGTGGGTCTGGGACAGAATTCACTCTCACCATTAGGAGCCTGCAGCCT
GAAGATTTTGCAGTTTATTACTGTCAACAGTATCATGACTGGCCTCGG
ACGTTCGGCCAAGGGACCAAGGTGGATTTCAAAC

Protein Sequence Defining the 43.52.E12 Kappa Chain Variable Region (SEQ ID NO: 215)

EIVMTQSPATLSLSPGERATLSCRASQSLRTNLAWYQQRPGQAPRLLI
SGASTRATGIPARFSGSGSGTEFTLTIRSLQPEDFAVYYCQQYHDWPR
TFGQGTKVDFK

Nucleic Acid Sequence Encoding the 43.62.E2 Heavy Chain Variable Region (SEQ ID NO: 216)

GAGGTGCAGCTGGTGCAGTCTGGGGCAGAAGTGAAAAAGGCCGGGGAG
TCTCTGAGGATCTCCTGTAAGGGTTCTGGATACAACTTTAATAGCTAT
TGGATTGGCTGGGTGCGCCAGATGCCCGGGAAAGGCCTGGAGTGGATG
GCAATCATCTATCCTGCTGACTCTGACAGGAGATATGGCCCGTCCTTC
CAAGGCCAGGTCACCATCTCAGTCGACAAGTCTATTAACACCGCGTAC
TTGCAGTGGAGCAGCCTGAAGGCCTCGGACACCGCCATGTATTATTGT
GCCAGACTCCTCAGAGTTAGTACTGGTTGGGAAGATGCTTTTGATCTG
TGGGGCCAAGGGACAATGGTCACCGTCTCGAGTG

Protein Sequence Defining the 43.62.E2 Heavy Chain Variable Region (SEQ ID NO: 217)

EVQLVQSGAEVKKAGESLRISCKGSGYNFNSYWIGWVRQMPGKGLEWM
AIIYPADSDRRYGPSFQGQVTISVDKSINTAYLQWSSLKASDTAMYYC
ARLLRVSTGWEDAFDLWGQGTMVTVSS

Nucleic Acid Sequence Encoding the 43.62.E2 Kappa Chain Variable Region (SEQ ID NO: 218)

GACATCGTGATGACCCAGTCTCCAGACTCCCTGGTTGTGTCTCTGGG
CGAGAGGGCCACCATCAAGTGCCAGTCCAGCCAGAGTGTTTTGTACA
GCTCCACCAATAAGAACTATTTAGCCTGGTACCAGCAGAAATCGGGA
CAGCCACCTAAGCTACTCATTTACTGGGCCTCCACCCGGGAATCCGG
GGTCCCTGACCGATTCATTGGTAGCGGCTCTAGGACAGATTTCACTC
TCACCATCAGCGACCTGCAGCCTGAGGATGCGGCAGTTTATTACTGT
CAGCAGTATTATAGTGATTATCTCACTTTCGGCGGAGGGACCAAGGT
GGAGATCAAAC

Protein Sequence Defining the 43.62.E2 Kappa Chain Variable Region (SEQ ID NO: 219)

DIVMTQSPDSLVVSLGERATIKCQSSQSVLYSSTNKNYLAWYQQKSG
QPPKLLIYWASTRESGVPDRFIGSGSRTDFTLTISDLQPEDAAVYYCQQ
YYSDYLTFGGGTKVEIK

Table 2 is a concordance table showing the SEQ ID NO. of the heavy chain and light chain variable region amino acid sequences as a fully human cognate pairs for each anti-S. aureus antibody described herein.

TABLE 2

| | Heavy Chain | Light Chain |
|---|---|---|
| | Variable Region | Variable Region |
| Antibody | (SEQ ID NO:) | (SEQ ID NO:) |
| 1.62.B9 | 2 | 4 |
| 5.11.H10 | 11 | 13 |
| 5.27.A11 | 20 | 22 |
| 5.6.H2 | 29 | 31 |
| 5.6.H9 | 38 | 40 |
| 5.17.F8 | 47 | 49 |
| 5.19.F12 | 56 | 58 |
| 5.23.C9 | 65 | 67 |
| 5.23.C12 | 74 | 76 |
| 8.51.G11 | 83 | 85 |
| 9.51.H9 | 92 | 94 |
| 18.43.D8 | 101 | 103 |
| 22.22.E7 | 110 | 112 |
| 8.51.G10 | 149 | 151 |
| 5.24A.A7 | 153 | 155 |
| 5.24A.F3 | 157 | 159 |
| 5.8B.H4 | 161 | 163 |
| 26.51.E1 | 165 | 167 |
| 22.21.A7 | 169 | 171 |
| 22.18A.E9 | 173 | 175 |
| 5.52.H10 | 177 | 179 |
| 5.15.C1 | 181 | 183 |
| 5.54.E6 | 185 | 187 |
| 5.55.D2 | 189 | 191 |
| 22.14.A1 | 193 | 195 |
| 26.53.B4 | 197 | 199 |
| 5.63.E2 | 201 | 203 |
| 5.64.G4 | 205 | 207 |
| 43.52.A11 | 209 | 211 |
| 43.52.E12 | 213 | 215 |
| 43.62.E2 | 217 | 219 |

Table 3 summarizes the heavy chain and light chain CDR sequences (IMGT definition) of the disclosed S. aureus antibodies.

TABLE 3

| | Heavy Chain | | | Light Chain | | |
|---|---|---|---|---|---|---|
| Antibody | CDR1 | CDR2 | CDR3 | CDR1 | CDR2 | CDR3 |
| 1.62.B9 | GFTFSNAW | IKSKTDGGTT | CRTGGYW | TGAVTSGHY | DTS | CLVSDSGARIF |
| | (SEQ ID NO: 5) | (SEQ ID NO: 6) | (SEQ ID NO: 7) | (SEQ ID NO: 8) | | (SEQ ID NO: 9) |
| 5.11.H10 | GYTFTING | ISTSNGQT | CARNPYGNSRRFFDYW | QTITNY | GVS | CQQSYRAPQTF |
| | (SEQ ID NO: 14) | (SEQ ID NO: 15) | (SEQ ID NO: 16) | (SEQ ID NO: 17) | | (SEQ ID NO: 18) |
| 5.27.A11 | GFAFSSHE | IGLAGDT | CVKEGAHWEFDSW | SSNLGASLD | ANN | CQSYDGSLRVF |
| | (SEQ ID NO: 23) | (SEQ ID NO: 24) | (SEQ ID NO: 25) | (SEQ ID NO: 26) | | (SEQ ID NO: 27) |
| 5.6.H2 | GFTFSDYY | ISYISSSGRTI | CARDGGYCGSANCLHDAFDIW | QSISSW | KAS | CQQYNSYSTF |
| | (SEQ ID NO: 32) | (SEQ ID NO: 33) | (SEQ ID NO: 34) | (SEQ ID NO: 35) | | (SEQ ID NO: 36) |
| 5.6.H9 | GFSLSTSGEG | IYWDDDK | CVHRGMNRVFGVVYNSGWFDPW | QSVRSSQ | DAS | CQQYDTSPWTF |
| | (SEQ ID NO: 41) | (SEQ ID NO: 42) | (SEQ ID NO: 43) | (SEQ ID NO: 44) | | (SEQ ID NO: 45) |
| 5.17.F8 | GTSFSDYY | INQSGHT | CARFPWVGPEGFDHW | QTIGNS | YGS | CLQSGGFPWTF |
| | (SEQ ID NO: 50) | (SEQ ID NO: 51) | (SEQ ID NO: 52) | (SEQ ID NO: 53) | | (SEQ ID NO: 54) |
| 5.19.F12 | GSSLPYLS | FDPEDGET | CATSLIRNGLYEAFEHW | DDIKKN | DAS | CQQYDNVPLSF |
| | (SEQ ID NO: 59) | (SEQ ID NO: 60) | (SEQ ID NO: 61) | (SEQ ID NO: 62) | | (SEQ ID NO: 63) |
| 5.23.C9 | GGNFKNYI | INPKVGNE | CATLRVIGNRRLRPVGGIFDIW | QDVSHY | EAS | CQHDAELPPSF |
| | (SEQ ID NO: 68) | (SEQ ID NO: 69) | (SEQ ID NO: 70) | (SEQ ID NO: 71) | | (SEQ ID NO: 72) |
| 5.23.C12 | GHSFTAYG | ISTNNGNT | CARNPYGNSRRFLDYW | QNISPY | GTT | CQQSYRAPQTF |
| | (SEQ ID NO: 77) | (SEQ ID NO: 78) | (SEQ ID NO: 79) | (SEQ ID NO: 80) | | (SEQ ID NO: 81) |
| 8.51.G11 | GFSFTSSGEG | IYWDGDK | CVHLTRPSFWSPYYLFDLW | QYISTYL | GAS | CQQSSTLWSF |
| | (SEQ ID NO: 86) | (SEQ ID NO: 87) | (SEQ ID NO: 88) | (SEQ ID NO: 89) | | (SEQ ID NO: 90) |
| 9.51.H9 | NYTFTSFG | ISPYNGNT | CARTNWGNWYLDLW | QTILFGSNNKNY | WAS | CQQYFSNPYTF |
| | (SEQ ID NO: 95) | (SEQ ID NO: 96) | (SEQ ID NO: 97) | (SEQ ID NO: 98) | | (SEQ ID NO: 99) |
| 18.43.D8 | GYIFVDYA | LQSASGKT | CARGSISHYAPFDYW | QNILYTSNNKSF | WAS | CQQYFSTPYTF |
| | (SEQ ID NO: 104) | (SEQ ID NO: 105) | (SEQ ID NO: 106) | (SEQ ID NO: 107) | | (SEQ ID NO: 108) |
| 22.22.E7 | GAYITNDY | LYTSGST | CARDECSGGGCFHENW | QSISSSY | GAS | CQQYDRSPLTF |
| | (SEQ ID NO: 113) | (SEQ ID NO: 114) | (SEQ ID NO: 115) | (SEQ ID NO: 116) | | (SEQ ID NO: 117) |
| 8.51.G10 | GFSFTSSGEA | IYWDDDP | CAHLRRPSLWSPYYLFDCW | QSISS | GAS | CQQSYKMWSF |
| | (SEQ ID NO: 220) | (SEQ ID NO: 221) | (SEQ ID NO: 222) | (SEQ ID NO: 223) | | (SEQ ID NO: 224) |
| 5.24A.A7 | GYTFTNFG | INTHNGNT | CARDFFRAAAGTAGDYW | HSGTNNY | GAS | CQQSYKMWSF |
| | (SEQ ID NO: 225) | (SEQ ID NO: 226) | (SEQ ID NO: 227) | (SEQ ID NO: 228) | | (SEQ ID NO: 229) |
| 5.24A.F3 | GGSISNDY | VYYSGRT | CARTTRLSFDWLVHYYGMDVW | QPIGSY | AAS | CQQSYNNPLFTF |
| | (SEQ ID NO: 230) | (SEQ ID NO: 231) | (SEQ ID NO: 232) | (SEQ ID NO: 233) | | (SEQ ID NO: 234) |
| 5.8B.H4 | GFIFDDYG | ITWDGVNT | CARARYYQGTGLRSYLDEW | QGIDNW | AAS | CQQAKNFPRGGFTF |
| | (SEQ ID NO: 235) | (SEQ ID NO: 236) | (SEQ ID NO: 237) | (SEQ ID NO: 238) | | (SEQ ID NO: 239) |
| 26.51.E1 | GFTFSKFA | IASDGYNK | CAYGHLSFWFDPW | QDIGNG | AAS | CLQHNFYPRTF |
| | (SEQ ID NO: 240) | (SEQ ID NO: 241) | (SEQ ID NO: 242) | (SEQ ID NO: 243) | | (SEQ ID NO: 244) |
| 22.21.A7 | GYTFTDYY | INPYSGGT | CARDFFRAAAGTAGDYW | QSALYSPSSKTY | WAS | CQYYYGTVTF(SEQ |
| | (SEQ ID NO: 245) | (SEQ ID NO: 246) | (SEQ ID NO: 247) | (SEQ ID NO: 248) | | ID NO: 249) |
| 22.18A.E9 | GGSINSYY | MYPGGTS | CARDPTYHHGTSGYVGTFDLW | PSVRNN | GAS | CQQYNSWPPYTF |
| | (SEQ ID NO: 250) | (SEQ ID NO: 251) | (SEQ ID NO: 252) | (SEQ ID NO: 253) | | (SEQ ID NO: 254) |
| 5.52.H10 | GFTFSMFT | ISGSSSLV | CVRGDSRDYW | TSNIGNNH | RND | CGTWDNSLSGWVF |
| | (SEQ ID NO: 255) | (SEQ ID NO: 256) | (SEQ ID NO: 257) | (SEQ ID NO: 258) | | (SEQ ID NO: 259) |
| 5.15.C1 | GYSFTTD | MNPNTGDT | CARSNSAPVSTLLPADAFDVW | QIINSY | AVS | CQQTYTTVAITF |
| | (SEQ ID NO: 260) | (SEQ ID NO: 261) | (SEQ ID NO: 262) | (SEQ ID NO: 263) | | (SEQ ID NO: 264) |
| 5.54.E6 | AGTLSGYT | IIPLGIG | CAVEFYRLATVTTPTLDFW | HIGAKS | MNS | CQVWDSFRDHQVF |
| | (SEQ ID NO: 265) | (SEQ ID NO: 266) | (SEQ ID NO: 267) | (SEQ ID NO: 268) | | (SEQ ID NO: 269) |
| 5.55.D2 | GFSLRTSGVG | IYWDDDK | CARQITNSFGLVIANDAFDIW | SSDVGRYNY | GVT | CSSYAGSNFLEVF |
| | (SEQ ID NO: 270) | (SEQ ID NO: 271) | (SEQ ID NO: 272) | (SEQ ID NO: 273) | | (SEQ ID NO: 274) |
| 22.14.A1 | GGTFSSYA | IIPMFDTA | CARDRNDYYDSSGYSGAFDQW | QSVSSSY | GAS | CQQYGSSPRTF |
| | (SEQ ID NO: 275) | (SEQ ID NO: 276) | (SEQ ID NO: 277) | (SEQ ID NO: 278) | | (SEQ ID NO: 279) |
| 26.53.B4 | GFSFSGSA | IRTKTKNYAT | CSRIEFSSSSGPQM | QNIITW | KAS | CLQYKTEPWTF |
| | (SEQ ID NO: 280) | (SEQ ID NO: 281) | (SEQ ID NO: 282) | (SEQ ID NO: 283) | | (SEQ ID NO: 284) |
| 5.63.E2 | GFTFSAYW | INQGGDKT | CGRGTNQDYW | SSNIGSNN | PNS | CAAWDDSLRTYVF |
| | (SEQ ID NO: 285) | (SEQ ID NO: 286) | (SEQ ID NO: 287) | (SEQ ID NO: 288) | | (SEQ ID NO: 289) |
| 5.64.G4. | GFTLRSYG | ISYDGSKT | CAKEDNLGSDVFDRW | QSASQW | KAS | CQQYNIYPWTF |
| | (SEQ ID NO: 290) | (SEQ ID NO: 291) | (SEQ ID NO: 292) | (SEQ ID NO: 293) | | (SEQ ID NO: 294) |
| 43.52.A11 | GRIFRNYA | FIPSEGIT | CVGGRPGAFVPFDSW | QSVRGN | DAS | CQQYNDWPRTF |
| | (SEQ ID NO: 295) | (SEQ ID NO: 296) | (SEQ ID NO: 297) | (SEQ ID NO: 298) | | (SEQ ID NO: 299) |
| 43.52.E12 | GGSFSSYD | SVSRADSS | CAAGDCSDGGCFRTPLHIW | QSLRTN | GAS | CQQYHDWPRTF |
| | (SEQ ID NO: 300) | (SEQ ID NO: 301) | (SEQ ID NO: 302) | (SEQ ID NO: 303) | | (SEQ ID NO: 304) |
| 43.62.E2 | GYNFNSYW | IYPADSDR | CARLLRVSTGWEDAFDLW | QSVLYSSTNKNY | WAS | CQQYYSDYLTF |
| | (SEQ ID NO: 305) | (SEQ ID NO: 306) | (SEQ ID NO: 307) | (SEQ ID NO: 308) | | (SEQ ID NO: 309) |

To create complete heavy and/or light chain antibody sequences, each variable sequence above can be combined with a constant region. Human constant regions for heavy chain, kappa chain, and lambda chain are known in the art. For example, a complete heavy chain comprises a heavy variable sequence followed by a human heavy chain constant sequence such as IgG1, IgG2, IgG3, IgG4, IgA1, IgA2, IgM, IgD, or IgE. A complete kappa chain comprises a kappa variable sequence followed by the human kappa light chain constant sequence, and a complete lambda chain comprises a lambda variable sequence followed by the human lambda light chain constant sequence. Exemplary human heavy chain, kappa chain, and lambda chains are shown below.

Nucleic Acid Sequence Encoding the genomic Human IgG1 Heavy Chain Constant Region (SEQ ID NO: 118)

CCTCCACCAAGGGCCCATCGGTCTTCCCCCTGGCACCCTCCTCCAAG
AGCACCTCTGGGGGCACAGCGGCCCTGGGCTGCCTGGTCAAGGACTA
CTTCCCCGAACCGGTGACGGTGTCGTGGAACTCAGGCGCCCTGACCA
GCGGCGTGCACACCTTCCCGGCTGTCCTACAGTCCTCAGGACTCTAC
TCCCTCAGCAGCGTGGTGACCGTGCCCTCCAGCAGCTTGGGCACCCA
GACCTACATCTGCAACGTGAATCACAAGCCCAGCAACACCAAGGTGG
ACAAGAAAGTTGGTGAGAGGCCAGCACAGGGAGGGAGGGTGTCTGCT
GGAAGCAGGCTCAGCGCTCCTGCCTGGACGCATCCCGGCTATGCAGC
CCCAGTCCAGGGCAGCAAGGCAGGCCCCGTCTGCCTCTTCACCCGGA
GCCTCTGCCCGCCCCACTCATGCTCAGGGAGAGGGTCTTCTGGCTTT
TTCCCAGGCTCTGGGCAGGCACAGGCTAGGTGCCCCTAACCCAGGCC
CTGCACACAAAGGGGCAGGTGCTGGGCTCAGACCTGCCAAGAGCCAT
ATCCGGGAGGACCCTGCCCCTGACCTAAGCCCACCCCAAAGGCCAAA
CTCTCCACTCCCTCAGCTCGGACACCTTCTCTCCTCCCAGATTCCAG
TAACTCCCAATCTTCTCTCTGCAGAGCCCAAATCTTGTGACAAAACT
CACACATGCCCACCGTGCCCAGGTAAGCCAGCCCAGGCCTCGCCCTC
CAGCTCAAGGCGGGACAGGTGCCCTAGAGTAGCCTGCATCCAGGGAC
AGGCCCCAGCCGGGTGCTGACACGTCCACCTCCATCTCTTCCTCAGC
ACCTGAACTCCTGGGGGGACCGTCAGTCTTCCTCTTCCCCCCAAAAC
CCAAGGACACCCTCATGATCTCCCGGACCCCTGAGGTCACATGCGTG
GTGGTGGACGTGAGCCACGAAGACCCTGAGGTCAAGTTCAACTGGTA
CGTGGACGGCGTGGAGGTGCATAATGCCAAGACAAAGCCGCGGGAGG
AGCAGTACAACAGCACGTACCGGGTGGTCAGCGTCCTCACCGTCCTG
CACCAGGACTGGCTGAATGGCAAGGAGTACAAGTGCAAGGTCTCCAA
CAAAGCCCTCCCAGCCCCCATCGAGAAAACCATCTCCAAAGCCAAAG
GTGGGACCCGTGGGGTGCGAGGGCCACATGGACAGAGGCCGGCTCGG
CCCACCCTCTGCCCTGAGAGTGACCGCTGTACCAACCTCTGTCCTAC
AGGGCAGCCCCGAGAACCACAGGTGTACACCCTGCCCCCATCCCGGG
ATGAGCTGACCAAGAACCAGGTCAGCCTGACCTGCCTGGTCAAAGGC
TTCTATCCCAGCGACATCGCCGTGGAGTGGGAGAGCAATGGGCAGCC
GGAGAACAACTACAAGACCACGCCTCCCGTGCTGGACTCCGACGGCT
CCTTCTTCCTCTACAGCAAGCTCACCGTGGACAAGAGCAGGTGGCAG
CAGGGGAACGTCTTCTCATGCTCCGTGATGCATGAGGCTCTGCACAA
CCACTACACGCAGAAGAGCCTCTCCCTGTCTCCGGGTAAA

Nucleic Acid Sequence Encoding the Human IgG1 Heavy Chain Constant Region cDNA (SEQ ID NO: 119)

CCTCCACCAAGGGCCCATCGGTCTTCCCCCTGGCACCCTCCTCCAAG
AGCACCTCTGGGGGCACAGCGGCCCTGGGCTGCCTGGTCAAGGACTA
CTTCCCCGAACCGGTGACGGTGTCGTGGAACTCAGGCGCCCTGACCA
GCGGCGTGCACACCTTCCCGGCTGTCCTACAGTCCTCAGGACTCTAC
TCCCTCAGCAGCGTGGTGACCGTGCCCTCCAGCAGCTTGGGCACCCA
GACCTACATCTGCAACGTGAATCACAAGCCCAGCAACACCAAGGTGG
ACAAGAAAGTTGAGCCCAAATCTTGTGACAAAACTCACACATGCCCA
CCGTGCCCAGCACCTGAACTCCTGGGGGGACCGTCAGTCTTCCTCTT
CCCCCCAAAACCCAAGGACACCCTCATGATCTCCCGGACCCCTGAGG
TCACATGCGTGGTGGTGGACGTGAGCCACGAAGACCCTGAGGTCAAG
TTCAACTGGTACGTGGACGGCGTGGAGGTGCATAATGCCAAGACAAA
GCCGCGGGAGGAGCAGTACAACAGCACGTACCGTGTGGTCAGCGTCC
TCACCGTCCTGCACCAGGACTGGCTGAATGGCAAGGAGTACAAGTGC
AAGGTCTCCAACAAAGCCCTCCCAGCCCCCATCGAGAAAACCATCTC
CAAAGCCAAAGGGCAGCCCCGAGAACCACAGGTGTACACCCTGCCCC
CATCCCGGGATGAGCTGACCAAGAACCAGGTCAGCCTGACCTGCCTG
GTCAAAGGCTTCTATCCCAGCGACATCGCCGTGGAGTGGGAGAGCAA
TGGGCAGCCGGAGAACAACTACAAGACCACGCCTCCCGTGCTGGACT
CCGACGGCTCCTTCTTCCTCTACAGCAAGCTCACCGTGGACAAGAGC
AGGTGGCAGCAGGGGAACGTCTTCTCATGCTCCGTGATGCATGAGGC
TCTGCACAACCACTACACGCAGAAGAGCCTCTCCCTGTCTCCGGGTA
AA

Protein Sequence Defining the Human IgG1 Heavy Chain Constant Region (SEQ ID NO: 120)

STKGPSVFPLAPSSKSTSGGTAALGCLVKDYFPEPVTVSWNSGALTS
GVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVL
TVLHQDWLNGKEYKCKVSNKALPAPIEKTISKAKGQPREPQVYTLPP
SRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK

Nucleic Acid Sequence Encoding the genomic Human IgG2 Heavy Chain Constant Region (SEQ ID NO: 121)

CCTCCACCAAGGGCCCATCGGTCTTCCCCCTGGCGCCCTGCTCCAGG
AGCACCTCCGAGAGCACAGCCGCCCTGGGCTGCCTGGTCAAGGACTA
CTTCCCCGAACCGGTGACGGTGTCGTGGAACTCAGGCGCTCTGACCA
GCGGCGTGCACACCTTCCCAGCTGTCCTACAGTCCTCAGGACTCTAC
TCCCTCAGCAGCGTGGTGACCGTGCCCTCCAGCAACTTCGGCACCCA
GACCTACACCTGCAACGTAGATCACAAGCCCAGCAACACCAAGGTGG
ACAAGACAGTTGGTGAGAGGCCAGCTCAGGGAGGGAGGGTGTCTGCT
GGAAGCCAGGCTCAGCCCTCCTGCCTGGACGCACCCCGGCTGTGCAG
CCCCAGCCCAGGGCAGCAAGGCAGGCCCCATCTGTCTCCTCACCCGG
AGGCCTCTGCCCGCCCCACTCATGCTCAGGGAGAGGGTCTTCTGGCT
TTTTCCACCAGGCTCCAGGCAGGCACAGGCTGGGTGCCCCTACCCCA
GGCCCTTCACACACAGGGGCAGGTGCTTGGCTCAGACCTGCCAAAAG
CCATATCCGGGAGGACCCTGCCCCTGACCTAAGCCGACCCCAAAGGC
CAAACTGTCCACTCCCTCAGCTCGGACACCTTCTCTCCTCCCAGATC
CGAGTAACTCCCAATCTTCTCTCTGCAGAGCGCAAATGTTGTGTCGA
GTGCCCACCGTGCCCAGGTAAGCCAGCCCAGGCCTCGCCCTCCAGCT
CAAGGCGGGACAGGTGCCCTAGAGTAGCCTGCATCCAGGGACAGGCC
CCAGCTGGGTGCTGACACGTCCACCTCCATCTCTTCCTCAGCACCAC
CTGTGGCAGGACCGTCAGTCTTCCTCTTCCCCCCAAAACCCAAGGAC
ACCCTCATGATCTCCCGGACCCCTGAGGTCACGTGCGTGGTGGTGGA
CGTGAGCCACGAAGACCCCGAGGTCCAGTTCAACTGGTACGTGGACG
GCGTGGAGGTGCATAATGCCAAGACAAAGCCACGGGAGGAGCAGTTC
AACAGCACGTTCCGTGTGGTCAGCGTCCTCACCGTTGTGCACCAGGA
CTGGCTGAACGGCAAGGAGTACAAGTGCAAGGTCTCCAACAAAGGCC
TCCCAGCCCCCATCGAGAAAACCATCTCCAAAACCAAAGGTGGGACC
CGCGGGGTATGAGGGCCACATGGACAGAGGCCGGCTCGGCCCACCCT
CTGCCCTGGGAGTGACCGCTGTGCCAACCTCTGTCCCTACAGGGCAG
CCCCGAGAACCACAGGTGTACACCCTGCCCCCATCCCGGGAGGAGAT
GACCAAGAACCAGGTCAGCCTGACCTGCCTGGTCAAAGGCTTCTACC
CCAGCGACATCGCCGTGGAGTGGGAGAGCAATGGGCAGCCGGAGAAC
AACTACAAGACCACACCTCCCATGCTGGACTCCGACGGCTCCTTCTT
CCTCTACAGCAAGCTCACCGTGGACAAGAGCAGGTGGCAGCAGGGGA
ACGTCTTCTCATGCTCCGTGATGCATGAGGCTCTGCACAACCACTAC
ACGCAGAAGAGCCTCTCCCTGTCTCCGGGTAAA

Nucleic Acid Sequence Encoding the Human IgG2 Heavy Chain Constant Region cDNA (SEQ ID NO: 122)

CCTCCACCAAGGGCCCATCGGTCTTCCCCCTGGCGCCCTGCTCCAGG
AGCACCTCCGAGAGCACAGCCGCCCTGGGCTGCCTGGTCAAGGACTA
CTTCCCCGAACCGGTGACGGTGTCGTGGAACTCAGGCGCTCTGACCA
GCGGCGTGCACACCTTCCCAGCTGTCCTACAGTCCTCAGGACTCTAC
TCCCTCAGCAGCGTGGTGACCGTGCCCTCCAGCAACTTCGGCACCCA
GACCTACACCTGCAACGTAGATCACAAGCCCAGCAACACCAAGGTGG
ACAAGACAGTTGAGCGCAAATGTTGTGTCGAGTGCCCACCGTGCCCA
GCACCACCTGTGGCAGGACCGTCAGTCTTCCTCTTCCCCCCAAAACC
CAAGGACACCCTCATGATCTCCCGGACCCCTGAGGTCACGTGCGTGG
TGGTGGACGTGAGCCACGAAGACCCCGAGGTCCAGTTCAACTGGTAC
GTGGACGGCGTGGAGGTGCATAATGCCAAGACAAAGCCACGGGAGGA
GCAGTTCAACAGCACGTTCCGTGTGGTCAGCGTCCTCACCGTTGTGC
ACCAGGACTGGCTGAACGGCAAGGAGTACAAGTGCAAGGTCTCCAAC
AAAGGCCTCCCAGCCCCCATCGAGAAAACCATCTCCAAAACCAAAGG
GCAGCCCCGAGAACCACAGGTGTACACCCTGCCCCCATCCCGGGAGG
AGATGACCAAGAACCAGGTCAGCCTGACCTGCCTGGTCAAAGGCTTC
TACCCCAGCGACATCGCCGTGGAGTGGGAGAGCAATGGGCAGCCGGA
GAACAACTACAAGACCACACCTCCCATGCTGGACTCCGACGGCTCCT
TCTTCCTCTACAGCAAGCTCACCGTGGACAAGAGCAGGTGGCAGCAG
GGGAACGTCTTCTCATGCTCCGTGATGCATGAGGCTCTGCACAACCA
CTACACGCAGAAGAGCCTCTCCCTGTCTCCGGGTAAA

Protein Sequence Defining the Human IgG2 Heavy Chain Constant Region (SEQ ID NO: 123)

STKGPSVFPLAPCSRSTSESTAALGCLVKDYFPEPVTVSWNSGALTS
GVHTFPAVLQSSGLYSLSSVVTVPSSNFGTQTYTCNVDHKPSNTKVD
KTVERKCCVECPPCPAPPVAGPSVFLFPPKPKDTLMISRTPEVTCVV
VDVSHEDPEVQFNWYVDGVEVHNAKTKPREEQFNSTFRVVSVLTVVH
QDWLNGKEYKCKVSNKGLPAPIEKTISKTKGQPREPQVYTLPPSREE
MTKNQVSLTCLVKGFYPSDIAVEWESNGQPENNYKTTPPMLDSDGSF
FLYSKLTVDKSRWQQGNVFSCSVMHEALHNHYTQKSLSLSPGK

Nucleic Acid Sequence Encoding the Human Kappa Light Chain Constant Region (SEQ ID NO: 124)

GAACTGTGGCTGCACCATCTGTCTTCATCTTCCCGCCATCTGATGAG
CAGTTGAAATCTGGAACTGCCTCTGTTGTGTGCCTGCTGAATAACTT
CTATCCCAGAGAGGCCAAAGTACAGTGGAAGGTGGATAACGCCCTCC
AATCGGGTAACTCCCAGGAGAGTGTCACAGAGCAGGACAGCAAGGAC
AGCACCTACAGCCTCAGCAGCACCCTGACGCTGAGCAAAGCAGACTA
CGAGAAACACAAAGTCTACGCCTGCGAAGTCACCCATCAGGGCCTGA
GCTCGCCCGTCACAAAGAGCTTCAACAGGGGAGAGTGT

Protein Sequence Defining the Human Kappa Light Chain Constant Region (SEQ ID NO: 125)

RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

Nucleic Acid Sequence Encoding the Human Lambda Light Chain Constant Region (SEQ ID NO: 126)

GTCAGCCCAAGGCCAACCCCACTGTCACTCTGTTCCCGCCCTCCTCTGA
GGAGCTCCAAGCCAACAAGGCCACACTAGTGTGTCTGATCAGTGACTTC
TACCCGGGAGCTGTGACAGTGGCCTGGAAGGCAGATGGCAGCCCCGTCA
AGGCGGGAGTGGAGACCACCAAACCCTCCAAACAGAGCAACAACAAGTA
CGCGGCCAGCAGCTACCTGAGCCTGACGCCCGAGCAGTGGAAGTCCCAC
AGAAGCTACAGCTGCCAGGTCACGCATGAAGGGAGCACCGTGGAGAAGA
CAGTGGCCCCTACAGAATGTTCA

Protein Sequence Defining the Human Lambda Light Chain Constant Region (SEQ ID NO: 127)

GQPKANPTVTLFPPSSEELQANKATLVCLISDFYPGAVTVAWKADGSPV
KAGVETTKPSKQSNNKYAASSYLSLTPEQWKSHRSYSCQVTHEGSTVEK
TVAPTECS

It is appreciated, however, that the variable region sequences described herein can be ligated to each of a number of other constant region sequences known to those skilled in the art to produce active full length immunoglobulin heavy and light chains.

The following sequences represent the actual or contemplated full length immunoglobulin heavy and light chain sequences (i.e., containing both the variable and constant regions sequences) for the anti-S. aureus antibodies described herein. The immunoglobulin heavy chain and light chain variable regions are shown as uppercase letters and the human IgG1, IgG2, kappa and lambda constant regions are shown in bold uppercase letters in the full length heavy and light chain sequences. CDR sequences are underlined.

Nucleic Acid Sequence Encoding the Full Length Heavy Chain Sequence of 1.62.B9 (SEQ ID NO: 128)

GAGGTGCAGCTGGTGGAGTCTGGGGGAGGCTTGGTAAAGCCTGGGGG
GTCCCTTAGACTCTCCTGTGCAGCCTCTGGATTCACTTTCAGTAACG
CCTGGATGACCTGGGTCCGCCAGGCTCCAGGGAAGGGGCTGGAGTGG
GTTGGCCGTATTAAGAGCAAAACTGATGGTGGGACAACAGACTACGC
TGCACCCGTGAAAGGCAGATTCACCATCTCAAGAGATGATTCAATAA
ACACACTGTATCTGCAAATGAACAGCCTGAAAACCGAGGACACAGCC
GTGTATTATTGTAGGACGGGGGGCTACTGGGGCCAGGGAACCCTGGT
CACCGTCTCGAGTGCCTCCACCAAGGGCCCATCGGTCTTCCCCCTGG
CGCCCTGCTCCAGGAGCACCTCCGAGAGCACAGCCGCCCTGGGCTGC
CTGGTCAAGGACTACTTCCCCGAACCGGTGACGGTGTCGTGGAACTC
AGGCGCTCTGACCAGCGGCGTGCACACCTTCCCAGCTGTCCTACAGT
CCTCAGGACTCTACTCCCTCAGCAGCGTGGTGACCGTGCCCTCCAGC
AACTTCGGCACCCAGACCTACACCTGCAACGTAGATCACAAGCCCAG
CAACACCAAGGTGGACAAGACAGTTGAGCGCAAATGTTGTGTCGAGT
GCCCACCGTGCCCAGCACCACCTGTGGCAGGACCGTCAGTCTTCCTC
TTCCCCCCAAAACCCAAGGACACCCTCATGATCTCCCGGACCCCTGA
GGTCACGTGCGTGGTGGTGGACGTGAGCCACGAAGACCCCGAGGTCC
AGTTCAACTGGTACGTGGACGGCGTGGAGGTGCATAATGCCAAGACA
AAGCCACGGGAGGAGCAGTTCAACAGCACGTTCCGTGTGGTCAGCGT
CCTCACCGTTGTGCACCAGGACTGGCTGAACGGCAAGGAGTACAAGT
GCAAGGTCTCCAACAAAGGCCTCCCAGCCCCCATCGAGAAAACCATC
TCCAAAACCAAAGGGCAGCCCCGAGAACCACAGGTGTACACCCTGCC
CCCATCCCGGGAGGAGATGACCAAGAACCAGGTCAGCCTGACCTGCC
TGGTCAAAGGCTTCTACCCCAGCGACATCGCCGTGGAGTGGGAGAGC
AATGGGCAGCCGGAGAACAACTACAAGACCACACCTCCCATGCTGGA
CTCCGACGGCTCCTTCTTCCTCTACAGCAAGCTCACCGTGGACAAGA
GCAGGTGGCAGCAGGGGAACGTCTTCTCATGCTCCGTGATGCATGAG
GCTCTGCACAACCACTACACGCAGAAGAGCCTCTCCCTGTCTCCGGG
TAAATGA

Protein Sequence Defining the Full Length Heavy Chain Sequence of 1.62.B9 (SEQ ID NO: 129)

EVQLVESGGGLVKPGGSLRLSCAASGFTFSNAWMTWVRQAPGKGLEWVG
RIKSKTDGGTTDYAAPVKGRFTISRDDSINTLYLQMNSLKTEDTAVYYC
RTGGYWGQGTLVTVSSASTKGPSVFPLAPCSRSTSESTAALGCLVKDYF
PEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLM
ISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYT
LPPSREEMTKNQVSLTCLVKGFYPSDIAVEWESNGQPENNYKTTPPMLD
SDGSFFLYSKLTVDKSRWQQGNVFSCSVMHEALHNHYTQKSLSLS
PGK

Nucleic Acid Sequence Encoding the Full Length Lambda Light Chain Sequence of 1.62.B9 (SEQ ID NO: 130)

CAGGCTGTGGTGACTCAGGAGCCCTCACTGACTGTGTCCCCAGGAGGGAC
AGTCACTCTCACCTGTGGCTCCAGCACTGGAGCTGTCACCAGTGGTCATT
ATCCCTACTGGTTCCAGCAGAAGCCTGGCCAAGCCCCCAGGACACTGATT
TATGATACAAGCAAGAAACATTCCTGGACACCTGCCCGGTTCTCAGGCTC
CCTCCTTGGGGGCAAAGCTGCCCTGACCCTTTCGGGTGCGCAGCCTGAGG
ATGAGGCTGAGTATTACTGCTTGGTCTCCGATAGTGGTGCTCGGATCTTC
GGACCTGGGACCAAGGTCACCGTCCTAGGTCAGCCCAAGGCCAACCCCAC
TGTCACTCTGTTCCCGCCCTCCTCTGAGGAGCTCCAAGCCAACAAGGCCA
CACTAGTGTGTCTGATCAGTGACTTCTACCCGGGAGCTGTGACAGTGGCC
TGGAAGGCAGATGGCAGCCCCGTCAAGGCGGGAGTGGAGACCACCAAACC
CTCCAAACAGAGCAACAACAAGTACGCGGCCAGCAGCTACCTGAGCCTGA
CGCCCGAGCAGTGGAAGTCCCACAGAAGCTACAGCTGCCAGGTCACGCAT
GAAGGGAGCACCGTGGAGAAGACAGTGGCCCCTACAGAATGTTCA

Protein Sequence Defining the Full Length Lambda Light Chain Sequence of 1.62.B9 (SEQ ID NO: 131)

QAVVTQEPSLTVSPGGTVTLTCGSSTGAVTSGHYPYWFQQKPGQAPRTLI
YDTSKKHSWTPARFSGSLLGGKAALTLSGAQPEDEAEYYCLVSDSGARIF
GPGTKVTVLGQPKANPTVTLFPPSSEELQANKATLVCLISDFYPGAVTVA
WKADGSPVKAGVETTKPSKQSNNKYAASSYLSLTPEQWKSHRSYSCQVTH
EGSTVEKTVAPTECS

Nucleic Acid Sequence Encoding the Full Length Heavy Chain Sequence of 5.11.H10 (SEQ ID NO: 132)

CAGGTTCAGCTGGTGCAGTCTGGACCTGAAATGAAGAAGCCTGGGGCCTC
AGTGAAGGTCTCCTGTCAGGCCTCTGGTTACACCTTTACCATTAATGGAA
TTAGTTGGGTGCGACAGGCCCCTGGACAACGGCCTGAGTGGATGGGGTGG
ATCAGCACTAGTAATGGTCAGACAAACTTTGCACAGAATTTCCAGGGCAG
ACTCACCTTGACCACAGACACATCCACGACCACGGCCTTCATGGAGTTGA
TGAGCCTCACATCTGCCGACACGGCCGTATACTACTGTGCGAGAAATCCA
TACGGCAATTCGCGCAGATTCTTTGACTACTGGGGCCAGGGAACCCTGGT
CACCGTCTCGAGTGCCTCCACCAAGGGCCCATCGGTCTTCCCCCTGGCGC
CCTGCTCCAGGAGCACCTCCGAGAGCACAGCCGCCCTGGGCTGCCTGGTC
AAGGACTACTTCCCCGAACCGGTGACGGTGTCGTGGAACTCAGGCGCTCT
GACCAGCGGCGTGCACACCTTCCCAGCTGTCCTACAGTCCTCAGGACTCT
ACTCCCTCAGCAGCGTGGTGACCGTGCCCTCCAGCAACTTCGGCACCCAG
ACCTACACCTGCAACGTAGATCACAAGCCCAGCAACACCAAGGTGGACAA
GACAGTTGAGCGCAAATGTTGTGTCGAGTGCCCACCGTGCCCAGCACCAC
CTGTGGCAGGACCGTCAGTCTTCCTCTTCCCCCCAAAACCCAAGGACACC
CTCATGATCTCCCGGACCCCTGAGGTCACGTGCGTGGTGGTGGACGTGAG
CCACGAAGACCCCGAGGTCCAGTTCAACTGGTACGTGGACGGCGTGGAGG
TGCATAATGCCAAGACAAAGCCACGGGAGGAGCAGTTCAACAGCACGTTC
CGTGTGGTCAGCGTCCTCACCGTTGTGCACCAGGACTGGCTGAACGGCAA
GGAGTACAAGTGCAAGGTCTCCAACAAAGGCCTCCCAGCCCCCATCGAGA
AAACCATCTCCAAAACCAAAGGGCAGCCCCGAGAACCACAGGTGTACACC
CTGCCCCCATCCCGGGAGGAGATGACCAAGAACCAGGTCAGCCTGACCTG
CCTGGTCAAAGGCTTCTACCCCAGCGACATCGCCGTGGAGTGGGAGAGCA
ATGGGCAGCCGGAGAACAACTACAAGACCACACCTCCCATGCTGGACTCC
GACGGCTCCTTCTTCCTCTACAGCAAGCTCACCGTGGACAAGAGCAGGTG
GCAGCAGGGGAACGTCTTCTCATGCTCCGTGATGCATGAGGCTCTGCACA
ACCACTACACGCAGAAGAGCCTCTCCCTGTCTCCGGGTAAATGA

Protein Sequence Defining the Full Length Heavy Chain Sequence of 5.11.H10 (SEQ ID NO: 133)

QVQLVQSGPEMKKPGASVKVSCQASGYTFTINGISWVRQAPGQRPEWMGW
ISTSNGQTNFAQNFQGRLTLTTDTSTTTAFMELMSLTSADTAVYYCARNP
YGNSRRFFDYWGQGTLVTVSSASTKGPSVFPLAPCSRSTSESTAALGCLV
KDYFPEPVTVSWNSGALTSGVHTFPAVLQSSGLYSLSSVVTVPSSNFGTQ
TYTCNVDHKPSNTKVDKTVERKCCVECPPCPAPPVAGPSVFLFPPKPKDT
LMISRTPEVTCVVVDVSHEDPEVQFNWYVDGVEVHNAKTKPREEQFNSTF
RVVSVLTVVHQDWLNGKEYKCKVSNKGLPAPIEKTISKTKGQPREPQVYT
LPPSREEMTKNQVSLTCLVKGFYPSDIAVEWESNGQPENNYKTTPPMLDS
DGSFFLYSKLTVDKSRWQQGNVFSCSVMHEALHNHYTQKSLSLSPGK

Nucleic Acid Sequence Encoding the Full Length Kappa Light Chain Sequence of 5.11.H10 (SEQ ID NO: 134)

GACATCCAGATGACCCAGTCTCCATCCTCCCTGTCTGCCTCTGTTGGCGA
CAGAGTCATCATCACTTGCCGGACAAGTCAGACCATCACCAATTACATAA
ATTGGTATCAGCAAAAACCAGGGAAAGCCCCGCAGCTCCTGATCTATGGT
GTATCCAGTTTGCAAAGTGGGGTCCCATCAAGGTTCAGTGGCGGTGGATC
TGGGACAGAATTCACTCTCACCATCAGCAGTCTGCAACCTGAAGACTTTG
CAACTTATTACTGTCAACAGAGTTATAGGGCCCCTCAAACATTCGGCCAA
GGGACCAAGCTGGAAATCAAACGAACTGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGT

Protein Sequence Defining the Full Length Kappa Light Chain Sequence of 5.11.H10 (SEQ ID NO: 135)

DIQMTQSPSSLSASVGDRVIITCRTSQTITNYINWYQQKPGKAPQLLIYG
VSSLQSGVPSRFSGGGSGTEFTLTISSLQPEDFATYYCQQSYRAPQTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Nucleic Acid Sequence Encoding the Full Length Heavy Chain Sequence of 5.27.A11 (SEQ ID NO: 136)

GAGGTGCAGCTGGTGGAGTCTGGGGGAGGCTTGGTGCAGCCGGGGGGGTC
TCTGAGACTCTCTTGTGCAGCCTCTGGTTTCGCCTTCAGCAGCCACGAGA
TGCACTGGGTTCGTCAAGGGATAGGACACAGTTTGGAGTGGGTCTCAGCT
ATTGGCCTTGCTGGTGACACATATTATTCAGACTCCGTGAAGGGCCGCTT
CACCATCTCCAGAGAAAACGCCAAGAACACTTTGTATCTTGAAATGAACA
ATTTGAGAGTCGGCGACACGGCTGTCTATTACTGTGTCAAAGAAGGGGCT
CATTGGGAATTTGACTCCTGGGGCCAGGGAACCCTGGTCACCGTCTCGAG
TGCCTCCACCAAGGGCCCATCGGTCTTCCCCCTGGCGCCCTGCTCCAGGA
GCACCTCCGAGAGCACAGCCGCCCTGGGCTGCCTGGTCAAGGACTACTTC
CCCGAACCGGTGACGGTGTCGTGGAACTCAGGCGCTCTGACCAGCGGCGT
GCACACCTTCCCAGCTGTCCTACAGTCCTCAGGACTCTACTCCCTCAGCA
GCGTGGTGACCGTGCCCTCCAGCAACTTCGGCACCCAGACCTACACCTGC
AACGTAGATCACAAGCCCAGCAACACCAAGGTGGACAAGACAGTTGAGCG
CAAATGTTGTGTCGAGTGCCCACCGTGCCCAGCACCACCTGTGGCAGGAC
CGTCAGTCTTCCTCTTCCCCCCAAAACCCAAGGACACCCTCATGATCTCC
CGGACCCCTGAGGTCACGTGCGTGGTGGTGGACGTGAGCCACGAAGACCC
CGAGGTCCAGTTCAACTGGTACGTGGACGGCGTGGAGGTGCATAATGCCA
AGACAAAGCCACGGGAGGAGCAGTTCAACAGCACGTTCCGTGTGGTCAGC
GTCCTCACCGTTGTGCACCAGGACTGGCTGAACGGCAAGGAGTACAAGTG
CAAGGTCTCCAACAAAGGCCTCCCAGCCCCCATCGAGAAAACCATCTCCA
AAACCAAAGGGCAGCCCCGAGAACCACAGGTGTACACCCTGCCCCCATCC
CGGGAGGAGATGACCAAGAACCAGGTCAGCCTGACCTGCCTGGTCAAAGG
CTTCTACCCCAGCGACATCGCCGTGGAGTGGGAGAGCAATGGGCAGCCGG
AGAACAACTACAAGACCACACCTCCCATGCTGGACTCCGACGGCTCCTTC
TTCCTCTACAGCAAGCTCACCGTGGACAAGAGCAGGTGGCAGCAGGGGAA
CGTCTTCTCATGCTCCGTGATGCATGAGGCTCTGCACAACCACTACACGC
AGAAGAGCCTCTCCCTGTCTCCGGGTAAATGA

Protein Sequence Defining the Full Length Heavy Chain Sequence of 5.27.A11 (SEQ ID NO: 137)

EVQLVESGGGLVQPGGSLRLSCAASGFAFSSHEMHWVRQGIGHSLEWVSA
IGLAGDTYYSDSVKGRFTISRENAKNTLYLEMNNLRVGDTAVYYCVKEGA
HWEFDSWGQGTLVTVSSASTKGPSVFPLAPCSRSTSESTAALGCLVKDYF
PEPVTVSWNSGALTSGVHTFPAVLQSSGLYSLSSVVTVPSSNFGTQTYTC
NVDHKPSNTKVDKTVERKCCVECPPCPAPPVAGPSVFLFPPKPKDTLMIS
RTPEVTCVVVDVSHEDPEVQPNWYVDGVEVHNAKTKPREEQFNSTFRVVS
VLTVVHQDWLNGKEYKCKVSNKGLPAPIEKTISKTKGQPREPQVYTLPPS
REEMTKNQVSLTCLVKGFYPSDIAVEWESNGQPENNYKTTPPMLDSDGSF
FLYSKLTVDKSRWQQGNVFSCSVMHEALHNHYTQKSLSLSPGK

Nucleic Acid Sequence Encoding the Full Length Lambda Light Chain Sequence of 5.27.A11 (SEQ ID NO: 138)

CAGTCTGTGCTGACGCAGCCGCCCTCAGTGTCTGGGGCCCCTGGACAGAG
GGTCATCATCTCCTGCACTGGGAGCAGCTCCAACCTCGGGGCAAGTCTTG
ATGTACACTGGTACCAGCGCCTTCCAGGGACAGGCCCCCGACTCCTCATC
TATGCTAACAACAATCGGCCCTCAGGGGTCCCTGACCGATTCTCTGGCTC
CAAGTCTGGCACCTCAGCCTCCCTGGCCATCACCGGGCTCCAGGCTGAAG
ATGAGGGTGATTATTACTGCCAGTCCTATGACGGCAGCCTGAGGGTGTTC
GGCGGAGGGACCAGGCTGACCGTCCTAGGTCAGCCCAAGGCTGCCCCCTC
GGTCACTCTGTTCCCGCCCTCCTCTGAGGAGCTTCAAGCCAACAAGGCCA
CACTGGTGTGTCTCATAAGTGACTTCTACCCGGGAGCCGTGACAGTGGCC
TGGAAGGCAGATAGCAGCCCCGTCAAGGCGGGAGTGGAGACCACCACACC
CTCCAAACAAAGCAACAACAAGTACGCGGCCAGCAGCTACCTGAGCCTGA
CGCCTGAGCAGTGGAAGTCCCACAAAAGCTACAGCTGCCAGGTCACGCAT
GAAGGGAGCACCGTGGAGAAGACAGTGGCCCCTACAGAATGTTCA

Protein Sequence Defining the Full Length Lambda Light Chain Sequence of 5.27.A11 (SEQ ID NO: 139)

QSVLTQPPSVSGAPGQRVIISCTGSSSNLGASLDVHWYQRLPGTGPRLLI
YANNNRPSGVPDRFSGSKSGTSASLAITGLQAEDEGDYYCQSYDGSLRVF
GGGTRLTVLGQPKAAPSVTLFPPSSEELQANKATLVCLISDFYPGAVTVA
WKADSSPVKAGVETTTPSKQSNNKYAASSYLSLTPEQWKSHKSYSCQVTH
EGSTVEKTVAPTECS

Nucleic Acid Sequence Encoding the Full Length Heavy Chain Sequence of 5.6.H2 (SEQ ID NO: 140)

CAGGTGCAGCTGGTGGAGTCTGGGGGAGGCTTGTTCAAGCCTGGAGGGTC
CCTGAGACTGTCCTGTGCAGCCTCTGGATTCACCTTCAGTGACTACTACA
TGAGCTGGATCCGGCAGGCTCCAGGGAAGGGGCTGGAGTGGATTTCATAC
ATTAGTAGTAGTGGTAGGACCATAAAGTACGCAGACTCTGTGAAGGGCCG
ATTCACCATCTCCAGGGACAACGCCAAGAACTCACTTTATCTGCAAATGA
ACAGCCTGAGAGCCGAGGACACGGCCGTGTATTATTGTGCGAGAGATGGA
GGATATTGTGGAAGTGCAAACTGCCTTCATGACGCTTTTGATATCTGGGG
CCAAGGGACAATGGTCACCGTCTCGAGTGCCTCCACCAAGGGCCCATCGG
TCTTCCCCCTGGCGCCCTGCTCCAGGAGCACCTCCGAGAGCACAGCCGCC
CTGGGCTGCCTGGTCAAGGACTACTTCCCCGAACCGGTGACGGTGTCGTG
GAACTCAGGCGCTCTGACCAGCGGCGTGCACACCTTCCCAGCTGTCCTAC
AGTCCTCAGGACTCTACTCCCTCAGCAGCGTGGTGACCGTGCCCTCCAGC
AACTTCGGCACCCAGACCTACACCTGCAACGTAGATCACAAGCCCAGCAA
CACCAAGGTGGACAAGACAGTTGAGCGCAAATGTTGTGTCGAGTGCCCAC
CGTGCCCAGCACCACCTGTGGCAGGACCGTCAGTCTTCCTCTTCCCCCCA
AAACCCAAGGACACCCTCATGATCTCCCGGACCCCTGAGGTCACGTGCGT
GGTGGTGGACGTGAGCCACGAAGACCCCGAGGTCCAGTTCAACTGGTACG
TGGACGGCGTGGAGGTGCATAATGCCAAGACAAAGCCACGGGAGGAGCAG
TTCAACAGCACGTTCCGTGTGGTCAGCGTCCTCACCGTTGTGCACCAGGA
CTGGCTGAACGGCAAGGAGTACAAGTGCAAGGTCTCCAACAAAGGCCTCC
CAGCCCCCATCGAGAAAACCATCTCCAAAACCAAAGGGCAGCCCCGAGAA
CCACAGGTGTACACCCTGCCCCCATCCCGGGAGGAGATGACCAAGAACCA
GGTCAGCCTGACCTGCCTGGTCAAAGGCTTCTACCCCAGCGACATCGCCG
TGGAGTGGGAGAGCAATGGGCAGCCGGAGAACAACTACAAGACCACACCT
CCCATGCTGGACTCCGACGGCTCCTTCTTCCTCTACAGCAAGCTCACCGT
GGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCG
GGTAAATGA

Protein Sequence Defining the Full Length Heavy Chain Sequence of 5.6.H2 (SEQ ID NO: 141)

QVQLVESGGGLFKPGGSLRLSCAASGFTFSDYYMSWIRQAPGKGLEWISY
ISSSGRTIKYADSVKGRFTISRDNAKNSLYLQMNSLRAEDTAVYYCARDG
GYCGSANCLHDAFDIWGQGTMVTVSSASTKGPSVFPLAPCSRSTSESTAA
LGCLVKDYFPEPVTVSWNSGALTSGVHTFPAVLQSSGLYSLSSVVTVPSS
NFGTQTYTCNVDHKPSNTKVDKTVERKCCVECPPCPAPPVAGPSVFLFPP
KPKDTLMISRTPEVTCVVVDVSHEDPEVQFNWYVDGVEVHNAKTKPREEQ
FNSTFRVVSVLTVVHQDWLNGKEYKCKVSNKGLPAPIEKTISKTKGQPRE
PQVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLSP
GK

Nucleic Acid Sequence Encoding the Full Length Kappa Light Chain Sequence of 5.6.H2 (SEQ ID NO: 142)

GACATCCAGATGACCCAGTCTCCTTCCACCCTGTCTGCATCTGTAGGAGA
CAGAGTCACCATCACTTGCCGGGCCAGTCAGAGTATTAGTAGCTGGTTGG
CCTGGTATCAGCAGAAACCAGGGAAAGCCCCTAAGCTCCTGATCTATAAG
GCGTCTAGTTTAGAAAGTGGGGTCCCATCAAGGTTCAGCGGCAGTGGATC
TGGGACAGAATTCACTCTCACCATCAGCAGCCTGCAGCCTGATGATTTTG
CAACTTATTACTGCCAACAGTATAATAGTTATTCGACGTTCGGCCAAGGG
ACCAAGGTGGAAATCAAACGAACTGTGGCTGCACCATCTGTCTTCATCTT
CCCGCCATCTGATGAGCAGTTGAAATCTGGAACTGCCTCTGTTGTGTGCC
TGCTGAATAACTTCTATCCCAGAGAGGCCAAAGTACAGTGGAAGGTGGAT
AACGCCCTCCAATCGGGTAACTCCCAGGAGAGTGTCACAGAGCAGGACAG
CAAGGACAGCACCTACAGCCTCAGCAGCACCCTGACGCTGAGCAAAGCAG
ACTACGAGAAACACAAAGTCTACGCCTGCGAAGTCACCCATCAGGGCCTG
AGCTCGCCCGTCACAAAGAGCTTCAACAGGGGAGAGTGT

Protein Sequence Defining the Full Length Kappa Light Chain Sequence of 5.6.H2 (SEQ ID NO: 143)

DIQMTQSPSTLSASVGDRVTITCRASQSISSWLAWYQQKPGKAPKLLIYK
ASSLESGVPSRFSGSGSGTEFTLTISSLQPDDFATYYCQQYNSYSTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

Nucleic Acid Sequence Encoding the Full Length Heavy Chain Sequence of 5.6.H9 (SEQ ID NO: 144)

CAGGTCACCTTGAAGGAGTCTGGTCCCACGCTGGTGAAACCCACACAGAC
CCTCACGCTGACGTGCACCTTCTCTGGGTTTTCGCTCAGCACTAGTGGAG
AGGGTGTGGCCTGGATCCGTCAGGCCCCAGGAAAGGCCCTGGAGTGGCTT
GCACTCATTTATTGGGATGATGATAAGCGCTACAACCCATCTCTGAAGAG
CAGGCTCACCATCACCAAGGACAACTCCAGAAACCAAGTGGTCCTTACAA
TGACCAACATGGACCCTGTGGACACAGCCACATATTACTGTGTACACAGG
GGGATGAATAGGGTTTTTGGGGTAGTTTATAATTCGGGCTGGTTCGACCC
CTGGGGCCAGGGAACCCTGGTCACCGTCTCGAGTGCCTCCACCAAGGGCC
CATCGGTCTTCCCCCTGGCGCCCTGCTCCAGGAGCACCTCCGAGAGCACA
GCCGCCCTGGGCTGCCTGGTCAAGGACTACTTCCCCGAACCGGTGACGGT
GTCGTGGAACTCAGGCGCTCTGACCAGCGGCGTGCACACCTTCCCAGCTG
TCCTACAGTCCTCAGGACTCTACTCCCTCAGCAGCGTGGTGACCGTGCCC
TCCAGCAACTTCGGCACCCAGACCTACACCTGCAACGTAGATCACAAGCC
CAGCAACACCAAGGTGGACAAGACAGTTGAGCGCAAATGTTGTGTCGAGT
GCCCACCGTGCCCAGCACCACCTGTGGCAGGACCGTCAGTCTTCCTCTTC
CCCCCAAAACCCAAGGACACCCTCATGATCTCCCGGACCCCTGAGGTCAC
GTGCGTGGTGGTGGACGTGAGCCACGAAGACCCCGAGGTCCAGTTCAACT
GGTACGTGGACGGCGTGGAGGTGCATAATGCCAAGACAAAGCCACGGGAG
GAGCAGTTCAACAGCACGTTCCGTGTGGTCAGCGTCCTCACCGTTGTGCA
CCAGGACTGGCTGAACGGCAAGGAGTACAAGTGCAAGGTCTCCAACAAAG
GCCTCCCAGCCCCCATCGAGAAAACCATCTCCAAAACCAAAGGGCAGCCC
CGAGAACCACAGGTGTACACCCTGCCCCCATCCCGGGAGGAGATGACCAA
GAACCAGGTCAGCCTGACCTGCCTGGTCAAAGGCTTCTACCCCAGCGACA
TCGCCGTGGAGTGGGAGAGCAATGGGCAGCCGGAGAACAACTACAAGACC
ACACCTCCCATGCTGGACTCCGACGGCTCCTTCTTCCTCTACAGCAAGCT
CACCGTGGACAAGAGCAGGTGGCAGCAGGGGAACGTCTTCTCATGCTCCG
TGATGCATGAGGCTCTGCACAACCACTACACGCAGAAGAGCCTCTCCCTG
TCTCCGGGTAAATGA

Protein Sequence Defining the Full Length Heavy Chain Sequence of 5.6.H9 (SEQ ID NO: 145)

QVTLKESGPTLVKPTQTLTLTCTFSGFSLSTSGEGVAWIRQAPGKALEW
LALIYWDDDKRYNPSLKSRLTITKDNSRNQVVLTMTNMDPVDTATYYCV
HRGMNRVFGVVYNSGWFDPWGQGTLVTVSSASTKGPSVFPLAPCSRSTS
ESTAALGCLVKDYFPEPVTVSWNSGALTSGVHTFPAVLQSSGLYSLSSV
VTVPSSNFGTQTYTCNVDHKPSNTKVDKTVERKCCVECPPCPAPPVAGP
SVFLFPPKPKDTLMISRTPEVTCVVVDVSHEDPEVQPNWYVDGVEVHNA
KTKPREEQFNSTFRVVSVLTVVHQDWLNGKEYKCKVSNKGLPAPIEKTI
SKTKGQPREPQVYTLPPSREEMTKNQVSLTCLVKGFYPSDIAVEWESNG
QPENNYKTTPPMLDSDGSFFLYSKLTVDKSRWQQGNVFSCSVMHEALHN
HYTQKSLSLSPGK

Nucleic Acid Sequence Encoding the Full Length Kappa Light Chain Sequence of 5.6.H9 (SEQ ID NO: 146)

GAAATTGTGTTGACGCAGTCTCCAGGCACCCTGTCTTTGTCTCCAGGGG
AAAGAGCCACCCTCTCCTGTAGGGCCAGTCAGAGCGTTAGAAGCAGCCA
GCTAGCCTGGTACCAGCAGAAGCCTGGCCAGGCTCCCAGCCTCCTCATG
TATGACGCATCTAAGAGGGCCACTGGCATCCCAGACAGGTTCAGTGGCA
GTGGGTCTGGGACAGACTTCACTCTCACCATCAGCAGACTGGAGCCTGA
AGATTTTGTAGTGTATTTCTGTCAGCAGTATGATACATCACCGTGGACG
TTCGGCCAAGGGACCAAGGTGGAAATCAAACGAACTGTGGCTGCACCAT
CTGTCTTCATCTTCCCGCCATCTGATGAGCAGTTGAAATCTGGAACTGC
CTCTGTTGTGTGCCTGCTGAATAACTTCTATCCCAGAGAGGCCAAAGTA
CAGTGGAAGGTGGATAACGCCCTCCAATCGGGTAACTCCCAGGAGAGTG
TCACAGAGCAGGACAGCAAGGACAGCACCTACAGCCTCAGCAGCACCCT
GACGCTGAGCAAAGCAGACTACGAGAAACACAAAGTCTACGCCTGCGAA
GTCACCCATCAGGGCCTGAGCTCGCCCGTCACAAAGAGCTTCAACAGGG
GAGAGTGT

Protein Sequence Defining the Full Length Kappa Light Chain Sequence of 5.6.H9

(SEQ ID NO: 147)

EIVLTQSPGTLSLSPGERATLSCRASQSVRSSQLAWYQQKPGQAPSLLM
YDASKRATGIPDRFSGSGSGTDFTLTISRLEPEDFVVYFCQQYDTSPWT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

Nucleic Acid Sequence Encoding the Full Length Heavy Chain Sequence of 8.51.G10 (SEQ ID NO: 310)

CAGATCACCTTGAAGGAGTCTGGTCCTACGCTGGTGAACCCCACACAGA
CCCTCACGCTGACCTGCACCTTTTCTGGGTTCTCATTCACCTCTAGTGG
AGAGGCTGTGGGCTGGATCCGTCAGCCCCCAGGAAAGGCCCTGGAATGG
CTTGCTCACATTTATTGGGATGATGATCCGCGTTATAGTACATCTCTGA
AGAGCAGACTCACCATCACCAAGGACACCTCCAAAAATCAGGTGGTCCT
CTCTATGACCGACATTGACCCTGCGGACACAGGCACATATTACTGTGCA
CACCTGAGACGCCCATCCCTTTGGAGTCCTTATTATCTTTTTGACTGCT
GGGGCCAGGGAACCCTGGTCACCGTCTCGAGTGCCTCCACCAAGGGCCC
ATCGGTCTTCCCCCTGGCGCCCTGCTCCAGGAGCACCTCCGAGAGCACA
GCCGCCCTGGGCTGCCTGGTCAAGGACTACTTCCCCGAACCGGTGACGG
TGTCGTGGAACTCAGGCGCTCTGACCAGCGGCGTGCACACCTTCCCAGC
TGTCCTACAGTCCTCAGGACTCTACTCCCTCAGCAGCGTGGTGACCGTG
CCCTCCAGCAACTTCGGCACCCAGACCTACACCTGCAACGTAGATCACA
AGCCCAGCAACACCAAGGTGGACAAGACAGTTGAGCGCAAATGTTGTGT
CGAGTGCCCACCGTGCCCAGCACCACCTGTGGCAGGACCGTCAGTCTTC
CTCTTCCCCCCAAAACCCAAGGACACCCTCATGATCTCCCGGACCCCTG
AGGTCACGTGCGTGGTGGTGGACGTGAGCCACGAAGACCCCGAGGTCCA
GTTCAACTGGTACGTGGACGGCGTGGAGGTGCATAATGCCAAGACAAAG
CCACGGGAGGAGCAGTTCAACAGCACGTTCCGTGTGGTCAGCGTCCTCA
CCGTTGTGCACCAGGACTGGCTGAACGGCAAGGAGTACAAGTGCAAGGT
CTCCAACAAAGGCCTCCCAGCCCCCATCGAGAAAACCATCTCCAAAACC
AAAGGGCAGCCCCGAGAACCACAGGTGTACACCCTGCCCCCATCCCGGG
AGGAGATGACCAAGAACCAGGTCAGCCTGACCTGCCTGGTCAAAGGCTT
CTACCCCAGCGACATCGCCGTGGAGTGGGAGAGCAATGGGCAGCCGGAG
AACAACTACAAGACCACACCTCCCATGCTGGACTCCGACGGCTCCTTCT
TCCTCTACAGCAAGCTCACCGTGGACAAGAGCAGGTGGCAGCAGGGGAA
CGTCTTCTCATGCTCCGTGATGCATGAGGCTCTGCACAACCACTACACG
CAGAAGAGCCTCTCCCTGTCTCCGGGTAAA

Protein Sequence Defining the Full Length Heavy Chain Sequence of 8.51.G10 (SEQ ID NO: 311)

QITLKESGPTLVNPTQTLTLTCTFSGFSFTSSGEAVGWIRQPPGKALEW
LAHIYWDDDPRYSTSLKSRLTITKDTSKNQVVLSMTDIDPADTGTYYCA
HLRRPSLWSPYYLFDCWGQGTLVTVSSSTKGPSVFPLAPCSRSTSESTA
ALGCLVKDYFPEPVTVSWNSGALTSGVHTFPAVLQSSGLYSLSSVVTVP
SSNFGTQTYTCNVDHKPSNTKVDKTVERKCCVECPPCPAPPVAGPSVFL
FPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTK
GQPREPQVYTLPPSREEMTKNQVSLTCLVKGFYPSDIAVEWESNGQPEN
NYKTTPPMLDSDGSFFLYSKLTVDKSRWQQGNVFSCSVMHEALHNHYTQ
KSLSLSPGK

Nucleic Acid Sequence Encoding the Full Length Kappa Light Chain Sequence of 8.51.G10 (SEQ ID NO: 312)

GACATCCAGATGACCCAGTCTCCATCCTCCCTGTCTGCATCTGTTGGAG
ACAGAGTCACCATCACTTGCCGGGCAAGTCAGAGTATTAGTAGCTATTT
AAATTGGTATCAACTGAAACCAGGACAAGCCCCTAAGCTCCTGATCTAT
GGTGCATCCAGTTTGGAAAATGGGGTCCCTTCACGGTTCAGTGGCAGTG
GCTCTGGGCCAGATTTCACTCTCACCATTAGCAGTCTGCAACCTGAAGA
TTTTGCAACTTACTACTGTCAACAGAGTTACAAAATGTGGTCATTCGGC
CAAGGGACCAAGGTGGAAATCCAACGAACTGTGGCTGCACCATCTGTCT
TCATCTTCCCGCCATCTGATGAGCAGTTGAAATCTGGAACTGCCTCTGT
TGTGTGCCTGCTGAATAACTTCTATCCCAGAGAGGCCAAAGTACAGTGG
AAGGTGGATAACGCCCTCCAATCGGGTAACTCCCAGGAGAGTGTCACAG
AGCAGGACAGCAAGGACAGCACCTACAGCCTCAGCAGCACCCTGACGCT
GAGCAAAGCAGACTACGAGAAACACAAAGTCTACGCCTGCGAAGTCACC
CATCAGGGCCTGAGCTCGCCCGTCACAAAGAGCTTCAACAGGGGAGAGT
GT

Protein Sequence Defining the Full Length Kappa Light Chain Sequence of 8.51.G10 (SEQ ID NO: 313)

DIQMTQSPSSLSASVGDRVTITCRASQSISSYLNWYQLKPGQAPKLLIY
GASSLENGVPSRFSGSGSGPDFTLTISSLQPEDFATYYCQQSYKMWSFG
QGTKVEIQRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC

Nucleic Acid Sequence Encoding the Full Length Heavy Chain Sequence of 5.24A.A7 (SEQ ID NO: 314)

CAGGTTCAGCTGGTGCAGTCTGGAGTTGAGGTGAAGAAGCCTGGGGCCT
CAGTGAAGGTCTCCTGCAAGGCTTCTGGCTACACCTTTACCAACTTTGG
CTTCAGTTGGGTGCGGCAGGCCCCAGGACAAGGGCTTGAGTGGCTGGGA
TGGATCAACACTCATAACGGTAACACAAACTATGCACCGAGATTCCAAG
ACAGAGTCACCCTGACCACAGACACATCCACGACCACAGCCTACATGGA
GCTGAGGAGCCTGAGATCTGAGGACACGGCCGTGTATTATTGTGCGAGA
GATCTTGGGGGAAGTAGTTCCGCCTACTGGGGACAGGGAACCCTGGTCA
CCGTCTCGAGTGCCTCCACCAAGGGCCCATCGGTCTTCCCCCTGGCGCC
CTGCTCCAGGAGCACCTCCGAGAGCACAGCCGCCCTGGGCTGCCTGGTC
AAGGACTACTTCCCCGAACCGGTGACGGTGTCGTGGAACTCAGGCGCTC
TGACCAGCGGCGTGCACACCTTCCCAGCTGTCCTACAGTCCTCAGGACT
CTACTCCCTCAGCAGCGTGGTGACCGTGCCCTCCAGCAACTTCGGCACC
CAGACCTACACCTGCAACGTAGATCACAAGCCCAGCAACACCAAGGTGG
ACAAGACAGTTGAGCGCAAATGTTGTGTCGAGTGCCCACCGTGCCCAGC
ACCACCTGTGGCAGGACCGTCAGTCTTCCTCTTCCCCCCAAAACCCAAG
GACACCCTCATGATCTCCCGGACCCCTGAGGTCACGTGCGTGGTGGTGG
ACGTGAGCCACGAAGACCCCGAGGTCCAGTTCAACTGGTACGTGGACGG
CGTGGAGGTGCATAATGCCAAGACAAAGCCACGGGAGGAGCAGTTCAAC
AGCACGTTCCGTGTGGTCAGCGTCCTCACCGTTGTGCACCAGGACTGGC
TGAACGGCAAGGAGTACAAGTGCAAGGTCTCCAACAAAGGCCTCCCAGC
CCCCATCGAGAAAACCATCTCCAAAACCAAAGGGCAGCCCCGAGAACCA
CAGGTGTACACCCTGCCCCCATCCCGGGAGGAGATGACCAAGAACCAGG
TCAGCCTGACCTGCCTGGTCAAAGGCTTCTACCCCAGCGACATCGCCGT
GGAGTGGGAGAGCAATGGGCAGCCGGAGAACAACTACAAGACCACACCT
CCCATGCTGGACTCCGACGGCTCCTTCTTCCTCTACAGCAAGCTCACCG
TGGACAAGAGCAGGTGGCAGCAGGGGAACGTCTTCTCATGCTCCGTGAT
GCATGAGGCTCTGCACAACCACTACACGCAGAAGAGCCTCTCCCTGTCT
CCGGGTAAA

Protein Sequence Defining the Full Length Heavy Chain Sequence of 5.24A.A7 (SEQ ID NO: 315)

QVQLVQSGVEVKKPGASVKVSCKASGYTFTNFGFSWVRQAPGQGLEWLG
WINTHNGNTNYAPRFQDRVTLTTDTSTTTAYMELRSLRSEDTAVYYCAR
DLGGSSSAYWGQGTLVTVSSSTKGPSVFPLAPCSRSTSESTAALGCLVK
DYFPEPVTVSWNSGALTSGVHTFPAVLQSSGLYSLSSVVTVPSSNFGTQ
TYTCNVDHKPSNTKVDKTVERKCCVECPPCPAPPVAGPSVFLFPPKPKD
TLMISRTPEVTCVVVDVSHEDPEVQPNWYVDGVEVHNAKTKPREEQFNS
TFRVVSVLTVVHQDWLNGKEYKCKVSNKGLPAPIEKTISKTKGQPREPQ
VYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSP
GK

Nucleic Acid Sequence Encoding the Full Length Kappa Light Chain Sequence of 5.24A.A7 (SEQ ID NO: 316)

GAAATTGTGTTGACGCAGTCTCCAGGCACCCTGTCTTTGTCTCCAGGGG
AGAGAGCCACCCTCTCTTGCAGGGCCAGTCACAGTGGTACCAACAACTA
CATCGCCTGGTACCAGCAGAAACCTGGCCAGCCTCCCAGGCTCCTCATC
TATGGTGCATCCAGCAGGGCCCCTGGCATCTCAGAGAGGTTCAGCGGCA
GTGGGTCTGGGACAGACTTCACTCTCATCATCAGCAGACTGGAGCCTGA
AGACTTTGGAGTATATTACTGTGAGCAGTATGGGAACTCACCTCTGACG
TTCGGCCAAGGGACCAAGGTGGAAATCAAACGAACTGTGGCTGCACCAT
CTGTCTTCATCTTCCCGCCATCTGATGAGCAGTTGAAATCTGGAACTGC
CTCTGTTGTGTGCCTGCTGAATAACTTCTATCCCAGAGAGGCCAAAGTA
CAGTGGAAGGTGGATAACGCCCTCCAATCGGGTAACTCCCAGGAGAGTG
TCACAGAGCAGGACAGCAAGGACAGCACCTACAGCCTCAGCAGCACCCT
GACGCTGAGCAAAGCAGACTACGAGAAACACAAAGTCTACGCCTGCGAA
GTCACCCATCAGGGCCTGAGCTCGCCCGTCACAAAGAGCTTCAACAGGG
GAGAGTGT

Protein Sequence Defining the Full Length Kappa Light Chain Sequence of 5.24A.A7 (SEQ ID NO: 317)

EIVLTQSPGTLSLSPGERATLSCRASHSGTNNYIAWYQQKPGQPPRLLI
YGASSRAPGISERFSGSGSGTDFTLIISRLEPEDFGVYYCEQYGNSPLT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

Nucleic Acid Sequence Encoding the Full Length Heavy Chain Sequence of 5.24A.F3 (SEQ ID NO: 318)

CAGGTGCAGCTGCAGGAGTCGGGCCCAGGACTGGTGGAGTCTTCGGACG
TCCTGTCCCTCACCTGCACTGTCTCTGGTGGCTCCATCAGTAATGACTA
TTGGGCCTGGATCCGGCAGACCCCAGGGAAGGGACTGGAGTGGATTGGG
TCTGTCTATTACAGTGGCAGAACCAGTTTCAGCCCCTCCCTCAAGAGTC
GAGTCACTATGTCAGTGGACACGACCAAGAACCAATTCTTCCTGAACCT
GGGCTCTCTGACCGTTGCGGACACGGCCGTGTATTACTGTGCGAGAACT
ACAAGGTTGTCCTTTGACTGGTTAGTACACTACTATGGAATGGACGTCT
GGGGCCAGGGGACCACGGTCACCGTCTCGAGTGCCTCCACCAAGGGCCC
ATCGGTCTTCCCCCTGGCGCCCTGCTCCAGGAGCACCTCCGAGAGCACA
GCCGCCCTGGGCTGCCTGGTCAAGGACTACTTCCCCGAACCGGTGACGG
TGTCGTGGAACTCAGGCGCTCTGACCAGCGGCGTGCACACCTTCCCAGC
TGTCCTACAGTCCTCAGGACTCTACTCCCTCAGCAGCGTGGTGACCGTG
CCCTCCAGCAACTTCGGCACCCAGACCTACACCTGCAACGTAGATCACA
AGCCCAGCAACACCAAGGTGGACAAGACAGTTGAGCGCAAATGTTGTGT
CGAGTGCCCACCGTGCCCAGCACCACCTGTGGCAGGACCGTCAGTCTTC
CTCTTCCCCCCAAAACCCAAGGACACCCTCATGATCTCCCGGACCCCTG
AGGTCACGTGCGTGGTGGTGGACGTGAGCCACGAAGACCCCGAGGTCCA
GTTCAACTGGTACGTGGACGGCGTGGAGGTGCATAATGCCAAGACAAAG
CCACGGGAGGAGCAGTTCAACAGCACGTTCCGTGTGGTCAGCGTCCTCA
CCGTTGTGCACCAGGACTGGCTGAACGGCAAGGAGTACAAGTGCAAGGT
CTCCAACAAAGGCCTCCCAGCCCCCATCGAGAAAACCATCTCCAAAACC
AAAGGGCAGCCCCGAGAACCACAGGTGTACACCCTGCCCCCATCCCGGG
AGGAGATGACCAAGAACCAGGTCAGCCTGACCTGCCTGGTCAAAGGCTT
CTACCCCAGCGACATCGCCGTGGAGTGGGAGAGCAATGGGCAGCCGGAG
AACAACTACAAGACCACACCTCCCATGCTGGACTCCGACGGCTCCTTCT
TCCTCTACAGCAAGCTCACCGTGGACAAGAGCAGGTGGCAGCAGGGGAA
CGTCTTCTCATGCTCCGTGATGCATGAGGCTCTGCACAACCACTACACG
CAGAAGAGCCTCTCCCTGTCTCCGGGTAAA

Protein Sequence Defining the Full Length Heavy Chain Sequence of 5.24A.F3 (SEQ ID NO: 319)

QVQLQESGPGLVESSDVLSLTCTVSGGSISNDYWAWIRQTPGKGLEWIG
SVYYSGRTSFSPSLKSRVTMSVDTTKNQFFLNLGSLTVADTAVYYCART
TRLSFDWLVHYYGMDVWGQGTTVTVSSSTKGPSVFPLAPCSRSTSESTA
ALGCLVKDYFPEPVTVSWNSGALTSGVHTFPAVLQSSGLYSLSSVVTVP
SSNFGTQTYTCNVDHKPSNTKVDKTVERKCCVECPPCPAPPVAGPSVFL
FPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTK
GQPREPQVYTLPPSREEMTKNQVSLTCLVKGFYPSDIAVEWESNGQPEN
NYKTTPPMLDSDGSFFLYSKLTVDKSRWQQGNVFSCSVMHEALHNHYTQ
KSLSLSPGK

Nucleic Acid Sequence Encoding the Full Length Kappa Light Chain Sequence of 5.24A.F3 (SEQ ID NO: 320)

GACATCCAGATGACCCAGTCTCCATCGTCCCTGACTGCATCTGTAGGAG
ACAGAGTCACCATTATTTGTCGGGCAAGTCAGCCCATTGGCAGCTATTT
GAATTGGTATCAGCAGAGTCCAGGGAAAGCCCCTAAACTCCTCATCTAT
GCTGCATCCACTTTGCAGAGTGGGGTCCCATCAAGGTTCAGTGGCAGTG
GATCTGGGACAGATTTCACTCTCAACATCAGTGGTCTGCAACCTGAAGA
TTTTGCAACTTACTACTGTCAACAGAGTTACAATAACCCTTTGTTCACC
TTCGGCCCTGGGACCAAAGTGGATATCAAACGAACTGTGGCTGCACCAT
CTGTCTTCATCTTCCCGCCATCTGATGAGCAGTTGAAATCTGGAACTGC
CTCTGTTGTGTGCCTGCTGAATAACTTCTATCCCAGAGAGGCCAAAGTA
CAGTGGAAGGTGGATAACGCCCTCCAATCGGGTAACTCCCAGGAGAGTG
TCACAGAGCAGGACAGCAAGGACAGCACCTACAGCCTCAGCAGCACCCT
GACGCTGAGCAAAGCAGACTACGAGAAACACAAAGTCTACGCCTGCGAA
GTCACCCATCAGGGCCTGAGCTCGCCCGTCACAAAGAGCTTCAACAGGG
GAGAGTGT

Protein Sequence Defining the Full Length Kappa Light Chain Sequence of 5.24A.F3 (SEQ ID NO: 321)

DIQMTQSPSSLTASVGDRVTIICRASQPIGSYLNWYQQSPGKAPKLLIY
AASTLQSGVPSRFSGSGSGTDFTLNISGLQPEDFATYYCQQSYNNPLFT
FGPGTKVDIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

Nucleic Acid Sequence Encoding the Full Length Heavy Chain Sequence of 5.8B.H4 (SEQ ID NO: 322)

GAGGTGCAGCTGGTGGAGTCTGGGGGAAGTCTGGTACGGCCGGGGGGGT
CCCTGCGACTCTCCTGTGCAGCCTCTGGATTCATCTTTGATGATTATGG
CATGAGTTGGGTCCGCCAAGTTCCAGGGGAGGGCCTGGAGTGGGTCGCC
GGCATTACTTGGGATGGCGTGAACACAGATTATGCAGACTCCGTGAAGG
GCCGCTTCACCATCTCCAGAGACAACGCCAAGAACTCCCTGTTTCTCCT
AATGAACAGTCTCAGAGCCGAGGACACGGCCCTGTATCACTGTGCGAGA
GCCAGATACTACCAAGGCACTGGCCTCCGGTCCTACCTCGACGAGTGGG
GCCAGGGAACCCTGGTCACCGTCTCGAGTGCCTCCACCAAGGGCCCATC
GGTCTTCCCCCTGGCGCCCTGCTCCAGGAGCACCTCCGAGAGCACAGCC
GCCCTGGGCTGCCTGGTCAAGGACTACTTCCCCGAACCGGTGACGGTGT
CGTGGAACTCAGGCGCTCTGACCAGCGGCGTGCACACCTTCCCAGCTGT
CCTACAGTCCTCAGGACTCTACTCCCTCAGCAGCGTGGTGACCGTGCCC
TCCAGCAACTTCGGCACCCAGACCTACACCTGCAACGTAGATCACAAGC
CCAGCAACACCAAGGTGGACAAGACAGTTGAGCGCAAATGTTGTGTCGA
GTGCCCACCGTGCCCAGCACCACCTGTGGCAGGACCGTCAGTCTTCCTC
TTCCCCCCAAAACCCAAGGACACCCTCATGATCTCCCGGACCCCTGAGG
TCACGTGCGTGGTGGTGGACGTGAGCCACGAAGACCCCGAGGTCCAGTT
CAACTGGTACGTGGACGGCGTGGAGGTGCATAATGCCAAGACAAAGCCA
CGGGAGGAGCAGTTCAACAGCACGTTCCGTGTGGTCAGCGTCCTCACCG
TTGTGCACCAGGACTGGCTGAACGGCAAGGAGTACAAGTGCAAGGTCTC
CAACAAAGGCCTCCCAGCCCCCATCGAGAAAACCATCTCCAAAACCAAA
GGGCAGCCCCGAGAACCACAGGTGTACACCCTGCCCCCATCCCGGGAGG
AGATGACCAAGAACCAGGTCAGCCTGACCTGCCTGGTCAAAGGCTTCTA
CCCCAGCGACATCGCCGTGGAGTGGGAGAGCAATGGGCAGCCGGAGAAC
AACTACAAGACCACACCTCCCATGCTGGACTCCGACGGCTCCTTCTTCC
TCTACAGCAAGCTCACCGTGGACAAGAGCAGGTGGCAGCAGGGGAACGT
CTTCTCATGCTCCGTGATGCATGAGGCTCTGCACAACCACTACACGCAG
AAGAGCCTCTCCCTGTCTCCGGGTAAA

Protein Sequence Defining the Full Length Heavy Chain Sequence of 5.8B.H4 (SEQ ID NO: 323)

EVQLVESGGSLVRPGGSLRLSCAASGFIFDDYGMSWVRQVPGEGLEWVA
GITWDGVNTDYADSVKGRFTISRDNAKNSLFLLMNSLRAEDTALYHCAR
ARYYQGTGLRSYLDEWGQGTLVTVSSSTKGPSVFPLAPCSRSTSESTAA
LGCLVKDYFPEPVTVSWNSGALTSGVHTFPAVLQSSGLYSLSSVVTVPS
SNFGTQTYTCNVDHKPSNTKVDKTVERKCCVECPPCPAPPVAGPSVFLF
PPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENN
YKTTPPMLDSDGSFFLYSKLTVDKSRWQQGNVFSCSVMHEALHNHYTQK
SLSLSPGK

Nucleic Acid Sequence Encoding the Full Length Kappa Light Chain Sequence of 5.8B.H4 (SEQ ID NO: 324)

GACATCCAGATGACCCAGTCTCCATCCTCCGTGTCTGCATCTGTAGGAG
AAAGAGTCACTATCACTTGTCGGGCGACTCAGGGTATTGACAACTGGTT
GGCCTGGTATCAGCATAAACCAGGGAAAGCCCCGAAGCTCCTGATTTAT
GCTGCATCCACTTTGCAAAGTGGGGTCCCGTCCAGGTTCAGCGGCAGTG
GATCTGGGACAGATTTCACTCTCACCATCAGCAGCGTGCAGCCTGAAGA
TGTTGCAACTTACTTTTGTCAACAGGCTAAGAATTTCCCTCGGGGAGGA
TTCACTTTCGGCCCTGGGACCAAAGTCGCTCTCAAACGAACTGTGGCTG
CACCATCTGTCTTCATCTTCCCGCCATCTGATGAGCAGTTGAAATCTGG
AACTGCCTCTGTTGTGTGCCTGCTGAATAACTTCTATCCCAGAGAGGCC
AAAGTACAGTGGAAGGTGGATAACGCCCTCCAATCGGGTAACTCCCAGG
AGAGTGTCACAGAGCAGGACAGCAAGGACAGCACCTACAGCCTCAGCAG
CACCCTGACGCTGAGCAAAGCAGACTACGAGAAACACAAAGTCTACGCC
TGCGAAGTCACCCATCAGGGCCTGAGCTCGCCCGTCACAAAGAGCTTCA
ACAGGGGAGAGTGT

Protein Sequence Defining the Full Length Kappa Light Chain Sequence of 5.8B.H4 (SEQ ID NO: 325)

DIQMTQSPSSVSASVGERVTITCRATQGIDNWLAWYQHKPGKAPKLLIY
AASTLQSGVPSRFSGSGSGTDFTLTISSVQPEDVATYFCQQAKNFPRGG
FTFGPGTKVALKRTVAAPSVFIFPPSDEQLKSGTASVVCLLNNFYPREA
KVQWKVDNALQSGNSQESVTEQDSKDSTYSLSSTLTLSKADYEKHKVYA
CEVTHQGLSSPVTKSFNRGEC

Nucleic Acid Sequence Encoding the Full Length Heavy Chain Sequence of 26.51.E1 (SEQ ID NO: 326)

CAGGTGCAGCTGGTGGAGTCTGGGGGAGGCGTGGTCCAGCCTGGGAGGT
CCCTGAGACTCTCCTGTGCAGCCTCTGGATTCACCTTCAGTAAATTTGC
CATGCACTGGGTCCGCCAGGCTCCAGGCAAGGGGCTGGAGTGGGTGGCA
GCTATAGCGTCTGATGGATATAATAAATACTATGCAGACTCCGTGAAGG
GCCAATTCACCGTCTCCAGAGACACTTCCAGGAACACGCTGTATCTCCA
AATGGACAGCCTGAGACCTGATGACACGGCTATTTATTACTGTGCGTAC
GGTCACTTGTCTTTTTGGTTCGACCCCTGGGGCCAGGGAACCCTGGTCA
CCGTCTCGAGTGCCTCCACCAAGGGCCCATCGGTCTTCCCCCTGGCGCC
CTGCTCCAGGAGCACCTCCGAGAGCACAGCCGCCCTGGGCTGCCTGGTC
AAGGACTACTTCCCCGAACCGGTGACGGTGTCGTGGAACTCAGGCGCTC
TGACCAGCGGCGTGCACACCTTCCCAGCTGTCCTACAGTCCTCAGGACT
CTACTCCCTCAGCAGCGTGGTGACCGTGCCCTCCAGCAACTTCGGCACC
CAGACCTACACCTGCAACGTAGATCACAAGCCCAGCAACACCAAGGTGG
ACAAGACAGTTGAGCGCAAATGTTGTGTCGAGTGCCCACCGTGCCCAGC
ACCACCTGTGGCAGGACCGTCAGTCTTCCTCTTCCCCCCAAAACCCAAG
GACACCCTCATGATCTCCCGGACCCCTGAGGTCACGTGCGTGGTGGTGG
ACGTGAGCCACGAAGACCCCGAGGTCCAGTTCAACTGGTACGTGGACGG
CGTGGAGGTGCATAATGCCAAGACAAAGCCACGGGAGGAGCAGTTCAAC
AGCACGTTCCGTGTGGTCAGCGTCCTCACCGTTGTGCACCAGGACTGGC
TGAACGGCAAGGAGTACAAGTGCAAGGTCTCCAACAAAGGCCTCCCAGC
CCCCATCGAGAAAACCATCTCCAAAACCAAAGGGCAGCCCCGAGAACCA
CAGGTGTACACCCTGCCCCCATCCCGGGAGGAGATGACCAAGAACCAGG
TCAGCCTGACCTGCCTGGTCAAAGGCTTCTACCCCAGCGACATCGCCGT
GGAGTGGGAGAGCAATGGGCAGCCGGAGAACAACTACAAGACCACACCT
CCCATGCTGGACTCCGACGGCTCCTTCTTCCTCTACAGCAAGCTCACCG
TGGACAAGAGCAGGTGGCAGCAGGGGAACGTCTTCTCATGCTCCGTGAT
GCATGAGGCTCTGCACAACCACTACACGCAGAAGAGCCTCTCCCTGTCT
CCGGGTAAA

Protein Sequence Defining the Full Length Heavy Chain Sequence of 26.51.E1 (SEQ ID NO: 327)

QVQLVESGGGVVQPGRSLRLSCAASGFTFSKFAMHWVRQAPGKGLEWVA
AIASDGYNKYYADSVKGQFTVSRDTSRNTLYLQMDSLRPDDTAIYYCAY
GHLSFWFDPWGQGTLVTVSSSTKGPSVFPLAPCSRSTSESTAALGCLVK
DYFPEPVTVSWNSGALTSGVHTFPAVLQSSGLYSLSSVVTVPSSNFGTQ
TYTCNVDHKPSNTKVDKTVERKCCVECPPCPAPPVAGPSVFLFPPKPKD
TLMISRTPEVTCVVVDVSHEDPEVQPNWYVDGVEVHNAKTKPREEQFNS
TFRVVSVLTVVHQDWLNGKEYKCKVSNKGLPAPIEKTISKTKGQPREPQ
VYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSP
GK

Nucleic Acid Sequence Encoding the Full Length Kappa Light Chain Sequence of 26.51.E1 (SEQ ID NO: 328)

GACATCCAGATGACCCAGTCTCCATCCTCCCTGTCCGCATCTGTAGGAG
ACAGAGTCACCATCACTTGCCGGGCAAGTCAGGACATTGGAAATGGCTT
AGGCTGGTATCAACAGATACCAGGGAAAGCCCCTAAGCGCCTGATCTAT
GCTGCATCCTCTTTGCAAAGTGGGGTCCCATCAAGGTTCAGCGGCAGTG
GTTCTGGGACAGAGTTCACTCTCACAATCCACAGCCTGCAGCCTGAAGA
TTTTGCAACTTATTACTGTCTACAGCATAATTTTTACCCTCGCACTTTT
GGCCAGGGGACCAAGCTGGAGATCAAGCGAACTGTGGCTGCACCATCTG
TCTTCATCTTCCCGCCATCTGATGAGCAGTTGAAATCTGGAACTGCCTC
TGTTGTGTGCCTGCTGAATAACTTCTATCCCAGAGAGGCCAAAGTACAG
TGGAAGGTGGATAACGCCCTCCAATCGGGTAACTCCCAGGAGAGTGTCA
CAGAGCAGGACAGCAAGGACAGCACCTACAGCCTCAGCAGCACCCTGAC
GCTGAGCAAAGCAGACTACGAGAAACACAAAGTCTACGCCTGCGAAGTC
ACCCATCAGGGCCTGAGCTCGCCCGTCACAAAGAGCTTCAACAGGGGAG
AGTGT

Protein Sequence Defining the Full Length Kappa Light Chain Sequence of 26.51.E1 (SEQ ID NO: 329)

DIQMTQSPSSLSASVGDRVTITCRASQDIGNGLGWYQQIPGKAPKRLIY
AASSLQSGVPSRFSGSGSGTEFTLTIHSLQPEDFATYYCLQHNFYPRTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Nucleic Acid Sequence Encoding the Full Length Heavy Chain Sequence of 22.21.A7 (SEQ ID NO: 330)

CAGGTGCAGCTGGTGCAGTCTGGGGCTGAGGTGAAGAAGCCTGGGGCCT
CAGTGAAGGTCTCCTGCAAGGCTTCTGGATACACCTTCACCGACTACTA
TATACACTGGGTGCGACAGGCCCCTGGACAAGGGCTTGAGTGGATGGGA
TGGATCAACCCTTATAGTGGTGGCACAAACTATGCACCGAAGTTTCAGG
GCAGGTTCACCATGGCCAGGGACACGTCCATCACCACAACCTACTTGGA
CCTGGGCAGGCTGACGTCTAGCGACACGGCCATTTATTTCTGTGCGAGA
GATTTCTTCAGAGCAGCAGCTGGCACGGCCGGAGACTACTGGGGCCAGG
GAACCCTGGTCACCGTCTCGAGTGCCTCCACCAAGGGCCCATCGGTCTT
CCCCCTGGCGCCCTGCTCCAGGAGCACCTCCGAGAGCACAGCCGCCCTG
GGCTGCCTGGTCAAGGACTACTTCCCCGAACCGGTGACGGTGTCGTGGA
ACTCAGGCGCTCTGACCAGCGGCGTGCACACCTTCCCAGCTGTCCTACA
GTCCTCAGGACTCTACTCCCTCAGCAGCGTGGTGACCGTGCCCTCCAGC
AACTTCGGCACCCAGACCTACACCTGCAACGTAGATCACAAGCCCAGCA
ACACCAAGGTGGACAAGACAGTTGAGCGCAAATGTTGTGTCGAGTGCCC
ACCGTGCCCAGCACCACCTGTGGCAGGACCGTCAGTCTTCCTCTTCCCC
CCAAAACCCAAGGACACCCTCATGATCTCCCGGACCCCTGAGGTCACGT
GCGTGGTGGTGGACGTGAGCCACGAAGACCCCGAGGTCCAGTTCAACTG
GTACGTGGACGGCGTGGAGGTGCATAATGCCAAGACAAAGCCACGGGAG
GAGCAGTTCAACAGCACGTTCCGTGTGGTCAGCGTCCTCACCGTTGTGC
ACCAGGACTGGCTGAACGGCAAGGAGTACAAGTGCAAGGTCTCCAACAA
AGGCCTCCCAGCCCCCATCGAGAAAACCATCTCCAAAACCAAAGGGCAG
CCCCGAGAACCACAGGTGTACACCCTGCCCCCATCCCGGGAGGAGATGA
CCAAGAACCAGGTCAGCCTGACCTGCCTGGTCAAAGGCTTCTACCCCAG
CGACATCGCCGTGGAGTGGGAGAGCAATGGGCAGCCGGAGAACAACTAC
AAGACCACACCTCCCATGCTGGACTCCGACGGCTCCTTCTTCCTCTACA
GCAAGCTCACCGTGGACAAGAGCAGGTGGCAGCAGGGGAACGTCTTCTC
ATGCTCCGTGATGCATGAGGCTCTGCACAACCACTACACGCAGAAGAGC
CTCTCCCTGTCTCCGGGTAAA

Protein Sequence Defining the Full Length Heavy Chain Sequence of 22.21.A7 (SEQ ID NO: 331)

QVQLVQSGAEVKKPGASVKVSCKASGYTFTDYYIHWVRQAPGQGLEWMG
WINPYSGGTNYAPKFQGRFTMARDTSITTTYLDLGRLTSSDTAIYFCAR
DFFRAAAGTAGDYWGQGTLVTVSSSTKGPSVFPLAPCSRSTSESTAALG
CLVKDYFPEPVTVSWNSGALTSGVHTFPAVLQSSGLYSLSSVVTVPSSN
FGTQTYTCNVDHKPSNTKVDKTVERKCCVECPPCPAPPVAGPSVFLFPP
KPKDTLMISRTPEVTCVVVDVSHEDPEVQFNWYVDGVEVHNAKTKPREE
QFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYK
TTPPMLDSDGSFFLYSKLTVDKSRWQQGNVFSCSVMHEALHNHYTQKSL
SLSPGK

Nucleic Acid Sequence Encoding the Full Length Kappa Light Chain Sequence of 22.21.A7 (SEQ ID NO: 332)

GACATCGTGATGACCCAGTCTCCAGAAGACTCCCTGGCTGTGTCTCTGG
GCGAGAGGGCCACCATCAACTGCTCCAGCCAGAGTGCTTTATACAGCCC
CAGCAGTAAGACCTACTTAGCTTGGTACCAGCAGAGACCGGGACACCCT
CCTAAGCTTCTCATTTACTGGGCATCTACCCGGGTATCCGGGGTCCCTG
ACCGATTCAGTGGCAGCGGGTCTGGGACAGATTTCACTCTCACCATCGG
CAGCCTGCAGGCTGAAGATGTGGCAGTTTATTACTGTCAGTACTATTAT
GGTACTGTCACTTTCGGCGGAGGGACCAAGGTGGAGATCAAACGAACTG
TGGCTGCACCATCTGTCTTCATCTTCCCGCCATCTGATGAGCAGTTGAA
ATCTGGAACTGCCTCTGTTGTGTGCCTGCTGAATAACTTCTATCCCAGA
GAGGCCAAAGTACAGTGGAAGGTGGATAACGCCCTCCAATCGGGTAACT
CCCAGGAGAGTGTCACAGAGCAGGACAGCAAGGACAGCACCTACAGCCT
CAGCAGCACCCTGACGCTGAGCAAAGCAGACTACGAGAAACACAAAGTC
TACGCCTGCGAAGTCACCCATCAGGGCCTGAGCTCGCCCGTCACAAAGA
GCTTCAACAGGGGAGAGTGT

Protein Sequence Defining the Full Length Kappa Light Chain Sequence of 22.21.A7 (SEQ ID NO: 333)

ADIVMTQSPDSLAVSLGERATINCSSQSALYSPSSKTYLAWYQQRPGHP
PKLLIYWASTRVSGVPDRFSGSGSGTDFTLTIGSLQAEDVAVYYCQYYY
GTVTFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

Nucleic Acid Sequence Encoding the Full Length Heavy Chain Sequence of 22.18A.E9 (SEQ ID NO: 334)

CAGGTGCAGCTGCAGGAGTCGGGCCCAAGACTGGTGAAGCCTTCGGAGA
CCCTGTCCCTCACCTGCACTGTCTCTGGTGGGTCCATCAATAGTTATTA
TTGGAGTTGGATCCGGCAGCCCGTCGGGAAGGGACTGGAGTGGATTGGG
CGTATGTATCCCGGTGGTACCTCCAGTTACAATTCATCCCTCAAGAGTC
GAGTCATCATGTCGGTAGACACGTCGAAGAACCAGTTTTCTCTGAACCT
CACTTCTGTGACCGCCGCAGACACGGCCGTATATTTCTGTGCGAGAGAT
CCAACATATCACCATGGCACTAGTGGTTATGTGGGGACTTTTGATCTCT
GGGGCCAAGGGACAATGGTCACCGTCTCGAGTGCCTCCACCAAGGGCCC
ATCGGTCTTCCCCCTGGCGCCCTGCTCCAGGAGCACCTCCGAGAGCACA
GCCGCCCTGGGCTGCCTGGTCAAGGACTACTTCCCCGAACCGGTGACGG
TGTCGTGGAACTCAGGCGCTCTGACCAGCGGCGTGCACACCTTCCCAGC
TGTCCTACAGTCCTCAGGACTCTACTCCCTCAGCAGCGTGGTGACCGTG
CCCTCCAGCAACTTCGGCACCCAGACCTACACCTGCAACGTAGATCACA
AGCCCAGCAACACCAAGGTGGACAAGACAGTTGAGCGCAAATGTTGTGT
CGAGTGCCCACCGTGCCCAGCACCACCTGTGGCAGGACCGTCAGTCTTC
CTCTTCCCCCCAAAACCCAAGGACACCCTCATGATCTCCCGGACCCCTG
AGGTCACGTGCGTGGTGGTGGACGTGAGCCACGAAGACCCCGAGGTCCA
GTTCAACTGGTACGTGGACGGCGTGGAGGTGCATAATGCCAAGACAAAG
CCACGGGAGGAGCAGTTCAACAGCACGTTCCGTGTGGTCAGCGTCCTCA
CCGTTGTGCACCAGGACTGGCTGAACGGCAAGGAGTACAAGTGCAAGGT
CTCCAACAAAGGCCTCCCAGCCCCCATCGAGAAAACCATCTCCAAAACC
AAAGGGCAGCCCCGAGAACCACAGGTGTACACCCTGCCCCCATCCCGGG
AGGAGATGACCAAGAACCAGGTCAGCCTGACCTGCCTGGTCAAAGGCTT
CTACCCCAGCGACATCGCCGTGGAGTGGGAGAGCAATGGGCAGCCGGAG
AACAACTACAAGACCACACCTCCCATGCTGGACTCCGACGGCTCCTTCT
TCCTCTACAGCAAGCTCACCGTGGACAAGAGCAGGTGGCAGCAGGGGAA
CGTCTTCTCATGCTCCGTGATGCATGAGGCTCTGCACAACCACTACACG
CAGAAGAGCCTCTCCCTGTCTCCGGGTAAA

Protein Sequence Defining the Full Length Heavy Chain Sequence of 22.18A.E9 (SEQ ID NO: 335)

QVQLQESGPRLVKPSETLSLTCTVSGGSINSYYWSWIRQPVGKGLEWIG
RMYPGGTSSYNSSLKSRVIMSVDTSKNQFSLNLTSVTAADTAVYFCARD
PTYHHGTSGYVGTFDLWGQGTMVTVSSSTKGPSVFPLAPCSRSTSESTA
ALGCLVKDYFPEPVTVSWNSGALTSGVHTFPAVLQSSGLYSLSSVVTVP
SSNFGTQTYTCNVDHKPSNTKVDKTVERKCCVECPPCPAPPVAGPSVFL
FPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTK
GQPREPQVYTLPPSREEMTKNQVSLTCLVKGFYPSDIAVEWESNGQPEN
NYKTTPPMLDSDGSFFLYSKLTVDKSRWQQGNVFSCSVMHEALHNHYTQ
KSLSLSPGK

Nucleic Acid Sequence Encoding the Full Length Kappa Light Chain Sequence of 22.18A.E9 (SEQ ID NO: 336)

GAAATAGTGATGACGCAGTCTCCAGCCACCCTGTCTGCGTCTCCAGGGG
AAAGAGTCACCCTCTCCTGCAGGGCCAGTCCGAGTGTTCGCAACAATTT
AGCCTGGTACCAGCAGAAACCTGGCCAGCCTCCTAGGCTCCTCATCTAT
GGTGCATCCACCAAGGTCATGGGTACTCCAACCAGGTTCAGTGGCAGTG
GGTCTGGGACAGAGTTCACTCTCACCATCAGCAGCCTGCAGTCTGAAGA
TTTTGCAGTTTATTACTGTCAGCAGTATAATAGCTGGCCTCCGTACACT
TTTAGCCAGGGGACCAAACTGGAGATCAAACGAACTGTGGCTGCACCAT
CTGTCTTCATCTTCCCGCCATCTGATGAGCAGTTGAAATCTGGAACTGC
CTCTGTTGTGTGCCTGCTGAATAACTTCTATCCCAGAGAGGCCAAAGTA
CAGTGGAAGGTGGATAACGCCCTCCAATCGGGTAACTCCCAGGAGAGTG
TCACAGAGCAGGACAGCAAGGACAGCACCTACAGCCTCAGCAGCACCCT
GACGCTGAGCAAAGCAGACTACGAGAAACACAAAGTCTACGCCTGCGAA
GTCACCCATCAGGGCCTGAGCTCGCCCGTCACAAAGAGCTTCAACAGGG
GAGAGTGT

Protein Sequence Defining the Full Length Kappa Light Chain Sequence of 22.18A.E9 (SEQ ID NO: 337)

DIVMTQSPATLSASPGERVTLSCRASPSVRNNLAWYQQKPGQPPRLLIY
GASTKVMGTPTRFSGSGSGTEFTLTISSLQSEDFAVYYCQQYNSWPPY
TFSQGTKLEIKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC

Nucleic Acid Sequence Encoding the Full Length Heavy Chain Sequence of 5.52.H10 (SEQ ID NO: 338)

GAGGTGCAGCTGGTGGAGTCGGGGGGAGGCCTGGTCAGGCCTGGGGAGT
CCCTGAGACTCTCCTGTGCAGGCTCTGGATTCACCTTCAGCATGTTTACC
CTGAACTGGGTCCGCCAGGCTCCAGGGAAGGGACTGGAGTGGGTCGCAT
CTATTAGTGGTAGTAGCAGTTTAGTGTTTCATGAAGACTCAGTGAAG
GGCCGATTCACCATCTCCAGAGACAACGCCAAGAACTCAGTATATCTGC
AAATGAACAGCCTGAGAGCCGAAGACACGGCTATTTATTACTGTGTGA
GAGGAGATAGTCGCGACTATTGGGGCCAGGGAACCCTGGTCACCGTCT
CGAGTGCCTCCACCAAGGGCCCATCGGTCTTCCCCCTGGCGCCCTGCT
CCAGGAGCACCTCCGAGAGCACAGCCGCCCTGGGCTGCCTGGTCAAGGA
CTACTTCCCCGAACCGGTGACGGTGTCGTGGAACTCAGGCGCTCTGACC
AGCGGCGTGCACACCTTCCCAGCTGTCCTACAGTCCTCAGGACTCTACT
CCCTCAGCAGCGTGGTGACCGTGCCCTCCAGCAACTTCGGCACCCAGAC
CTACACCTGCAACGTAGATCACAAGCCCAGCAACACCAAGGTGGACAAG
ACAGTTGAGCGCAAATGTTGTGTCGAGTGCCCACCGTGCCCAGCACCAC
CTGTGGCAGGACCGTCAGTCTTCCTCTTCCCCCCAAAACCCAAGGACAC
CCTCATGATCTCCCGGACCCCTGAGGTCACGTGCGTGGTGGTGGACGTG
AGCCACGAAGACCCCGAGGTCCAGTTCAACTGGTACGTGGACGGCGTGG
AGGTGCATAATGCCAAGACAAAGCCACGGGAGGAGCAGTTCAACAGCAC
GTTCCGTGTGGTCAGCGTCCTCACCGTTGTGCACCAGGACTGGCTGAAC
GGCAAGGAGTACAAGTGCAAGGTCTCCAACAAAGGCCTCCCAGCCCCCA
TCGAGAAAACCATCTCCAAAACCAAAGGGCAGCCCCGAGAACCACAGGT
GTACACCCTGCCCCCATCCCGGGAGGAGATGACCAAGAACCAGGTCAGC
CTGACCTGCCTGGTCAAAGGCTTCTACCCCAGCGACATCGCCGTGGAGT
GGGAGAGCAATGGGCAGCCGGAGAACAACTACAAGACCACACCTCCCAT
GCTGGACTCCGACGGCTCCTTCTTCCTCTACAGCAAGCTCACCGTGGAC
AAGAGCAGGTGGCAGCAGGGGAACGTCTTCTCATGCTCCGTGATGCATG
AGGCTCTGCACAACCACTACACGCAGAAGAGCCTCTCCCTGTCTCCGGG
TAAA

Protein Sequence Defining the Full Length Heavy Chain Sequence of 5.52.H10 (SEQ ID NO: 339)

EVQLVESGGGLVRPGESLRLSCAGSGFTFSMFTLNWVRQAPGKGLEWVA
SISGSSSLVFHEDSVKGRFTISRDNAKNSVYLQMNSLRAEDTAIYYCV
RGDSRDYWGQGTLVTVSSSTKGPSVFPLAPCSRSTSESTAALGCLVKD
YFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDT
LMISRTPEVTCVVVDVSHEDPEVQFNWYVDGVEVHNAKTKPREEQFNST
FRVVSVLTVVHQDWLNGKEYKCKVSNKGLPAPIEKTISKTKGQPREPQV
YTLPPSREEMTKNQVSLTCLVKGFYPSDIAVEWESNGQPENNYKTTPPM
LDSDGSFFLYSKLTVDKSRWQQGNVFSCSVMHEALHNHYTQKSLSLSPGK

Nucleic Acid Sequence Encoding the Full Length Kappa Light Chain Sequence of 5.52.H10 (SEQ ID NO: 340)

CAGTCTGTGTTGACGCAGCCGCCCTCAGTGTCTGCGGCCCCAGGACAGA
AGGTCACCATCTCCTGCTCTGGAAGGACCTCCAATATTGGAAATAACCA
TGTATCCTGGTACCAGCAATTCCCAGGAACAGCCCCCAAAGTCCTCATT
TATCGCAATGATAGGCGACCCTCAGGGATTCCTGATCGATTCTCTGGCT
CCAAGTCTGACACGTCAGCCACCCTGGGCATCACCGGGCTCCAGACTGG
GGACGAGGCCGATTATTACTGCGGAACATGGGACAACAGCCTGAGTGGT
TGGGTGTTCGGCGGAGGGACCAAGCTGACCGTCCTAGGTCAGCCCAAGG
CTGCCCCCTCGGTCACTCTGTTCCCGCCCTCCTCTGAGGAGCTTCAAGC
CAACAAGGCCACACTGGTGTGTCTCATAAGTGACTTCTACCCGGGAGTC
GTGACAGTGGCCTGGAAGGCAGATAGCAGCCCCGTCAAGGCGGGAGTGG
AGACCACCACACCCTCCAAACAAAGCAACAACAAGTACGCGGCCAGCAG
CTACCTGAGCCTGACGCCTGAGCAGTGGAAGTCCCACAAAAGCTACAGC
TGCCAGGTCACGCATGAAGGGAGCACCGTGGAGAAGACAGTGGCCCCTG
CAGAATGCTCA

Protein Sequence Defining the Full Length Kappa Light Chain Sequence of 5.52.H10 (SEQ ID NO: 341)

QSVLTQPPSVSAAPGQKVTISCSGRTSNIGNNHVSWYQQFPGTAPKVLI
YRNDRRPSGIPDRFSGSKSDTSATLGITGLQTGDEADYYCGTWDNSLSG
WVFGGGTKLTVLGQPKAAPSVTLFPPSSEELQANKATLVCLISDFYPGV
VTVAWKADSSPVKAGVETTTPSKQSNNKYAASSYLSLTPEQWKSHKSYS
CQVTHEGSTVEKTVAPAECS

Nucleic Acid Sequence Encoding the Full Length Heavy Chain Sequence of 5.15.C1 with an IgG1 Constant Region (SEQ ID NO: 342)

CAGGTGCAGCTGGTGCAGTCTGGGGCTGAGGTGAAGAGGCCTGGGGCCTC
AGTGAAGGTCTCCTGTGAGACTTCTGGATATAGCTTCACCACTCATGAT
TTGAACTGGGTGCGACAGGCCGCTGGACAGGGGCTTGAGTGGATGGGAT
GGATGAACCCTAACACTGGTGACACATCCTACGCACAGAGGTTCCAGGG
CAGAGTCACCATGATCAGGGACGTCTCCATAAGTACAGCCTACTTGGAA
TTGACAGGCCTGACATTTGATGACACGGCCGTCTACTTCTGTGCGAGGT
CGAATTCTGCACCTGTTTCAACTCTTTTACCCGCTGATGCTTTTGATGT
CTGGGGCCAAGGGACAATGGTCACCGTCTCGAGTGCCTCCACCAAGGGC
CCATCGGTCTTCCCCCTGGCGCCCTGCTCCAGGAGCACCTCCGAGAGCA
CAGCCGCCCTGGGCTGCCTGGTCAAGGACTACTTCCCCGAACCGGTGAC
GGTGTCGTGGAACTCAGGCGCTCTGACCAGCGGCGTGCACACCTTCCCA
GCTGTCCTACAGTCCTCAGGACTCTACTCCCTCAGCAGCGTGGTGACCG
TGCCCTCCAGCAACTTCGGCACCCAGACCTACACCTGCAACGTAGATCA
CAAGCCCAGCAACACCAAGGTGGACAAGACAGTTGAGCGCAAATGTTGT
GTCGAGTGCCCACCGTGCCCAGCACCACCTGTGGCAGGACCGTCAGTCT
TCCTCTTCCCCCCAAAACCCAAGGACACCCTCATGATCTCCCGGACCCC
TGAGGTCACGTGCGTGGTGGTGGACGTGAGCCACGAAGACCCCGAGGTC
CAGTTCAACTGGTACGTGGACGGCGTGGAGGTGCATAATGCCAAGACAA
AGCCACGGGAGGAGCAGTTCAACAGCACGTTCCGTGTGGTCAGCGTCCT
CACCGTTGTGCACCAGGACTGGCTGAACGGCAAGGAGTACAAGTGCAAG
GTCTCCAACAAAGGCCTCCCAGCCCCCATCGAGAAAACCATCTCCAAAA
CCAAAGGGCAGCCCCGAGAACCACAGGTGTACACCCTGCCCCCATCCCG
GGAGGAGATGACCAAGAACCAGGTCAGCCTGACCTGCCTGGTCAAAGGC
TTCTACCCCAGCGACATCGCCGTGGAGTGGGAGAGCAATGGGCAGCCGG
AGAACAACTACAAGACCACACCTCCCATGCTGGACTCCGACGGCTCCTT
CTTCCTCTACAGCAAGCTCACCGTGGACAAGAGCAGGTGGCAGCAGGGG
AACGTCTTCTCATGCTCCGTGATGCATGAGGCTCTGCACAACCACTACA
CGCAGAAGAGCCTCTCCCTGTCTCCGGGTAAA

Protein Sequence Defining the Full Length Heavy Chain Sequence of 5.15.C1 with an IgG1 Constant Region (SEQ ID NO: 343)

QVQLVQSGAEVKRPGASVKVSCETSGYSFTTDLNWVRQAAGQGLEWMGW
MNPNTGDTSYAQRFQGRVTMIRDVSISTAYLELTGLTFDDTAVYFCARS
NSAPVSTLLPADAFDVWGQGTMVTVSSASTKGPSVFPLAPSSKSTSGGT
AALGCLVKDYFPEPVTVSWNSGALTSGVTFPAVLQSSGLYSLSSVVTVP
SSSLGTQTYICNVNKPSNTKVDKKVEPKSCDKTTCPPCPAPELLGGPSV
FLFPPKPKDTLMISRTPEVTCVVVDVSEDPEVKFNWYVDGVEVNAKTKP
REEQYNSTYRVVSVLTVLQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMEALNYTQKSLS
LSPGK

Nucleic Acid Sequence Encoding the Full Length Kappa Chain Sequence of 5.15.C1 (SEQ ID NO: 344)

GACATCCAGATGACCCAGTCTCCATCCTCCCTGTCTGCATCTATAGGAG
ACACAGTCACCATCACTTGCCGGGCAAGTCAGATCATTAACAGCTATTT
AAATTGGTATCAGCAAAAACCAGGGGAAGCCCCTAAACTCCTGATCTAT
GCTGTATCCAATTTGCACCGTGGGGTCCCCTCAAGGTTCAGTGGCAG
TGGGTCTGCGGCAGATTTCACTCTCACTATCAGTGGTCTGCAACCTGAA
GATTTTGCAACTTACTACTGTCAACAGACTTACACTACCGTTGCGATCA
CCTTCGGCCAAGGGACACGACTGGACATTAAACGAACTGTGGCTGCACC
ATCTGTCTTCATCTTCCCGCCATCTGATGAGCAGTTGAAATCTGGAACT
GCCTCTGTTGTGTGCCTGCTGAATAACTTCTATCCCAGAGAGGCCAAAG
TACAGTGGAAGGTGGATAACGCCCTCCAATCGGGTAACTCCCAGGAGAG
TGTCACAGAGCAGGACAGCAAGGACAGCACCTACAGCCTCAGCAGCACC
CTGACGCTGAGCAAAGCAGACTACGAGAAACACAAAGTCTACGCCTGCG
AAGTCACCCATCAGGGCCTGAGCTCGCCCGTCACAAAGAGCTTCAACAG
GGGAGAGTGT

Protein Sequence Defining the Full Length Kappa Chain Sequence of 5.15.C1 (SEQ ID NO: 345)

DIQMTQSPSSLSASIGDTVTITCRASQIINSYLNWYQQKPGEAPKLLIY
AVSNLHRGVPSRFSGSGSAADFTLTISGLQPEDFATYYCQQTYTTVAIT
FGQGTRLDIKRTVAAPSVFIFPPSDEQLKSGTASVVCLLNNFYPREAKV
QWKVDNALQSGNSQESVTEQDSKDSTYSLSSTLTLSKADYEKHKVYACEVT
HQGLSSPVTKSFNRGEC

Nucleic Acid Sequence Encoding the Full Length Heavy Chain Sequence of 5.54.E6 with an IgG1 Constant Region (SEQ ID NO: 346)

CAGGTGCAGCTGGTGCAGTCTGGGGCGGAGGTGAAGAAGCCTGGGTCCT
CGGTGAACGTCTCCTGTAAGGCCTCCGCAGGGACCCTCAGCGGTTATACC
ATCAACTGGTTGCGCCAGGCCCCTGGACGAGGGTTTGAGTGGCTGGGAA
GGATCATCCCTCACCTTGGAATAGGAAACAACGCACATAACTGGGAGGC
CAGAGTCACCCTTACCGCCGACAAGTCCACGGACACTACCTACCTGGAG
TTGCGCGACCTGAGATCTGACGACGTGGCCATTTATTACTGCGCGGTG
GAGTTTTACCGGCTGGCAACGGTCACAACCCCAACCCTTGACTTCTGG
GGCCAGGGAACCCTGGTCACCGTCTCGAGTGCCTCCACCAAGGGCCCAT
CGGTCTTCCCCCTGGCGCCCTGCTCCAGGAGCACCTCCGAGAGCACAGC
CGCCCTGGGCTGCCTGGTCAAGGACTACTTCCCCGAACCGGTGACGGTG
TCGTGGAACTCAGGCGCTCTGACCAGCGGCGTGCACACCTTCCCAGCTG
TCCTACAGTCCTCAGGACTCTACTCCCTCAGCAGCGTGGTGACCGTGCC
CTCCAGCAACTTCGGCACCCAGACCTACACCTGCAACGTAGATCACAAG
CCCAGCAACACCAAGGTGGACAAGACAGTTGAGCGCAAATGTTGTGTCG
AGTGCCCACCGTGCCCAGCACCACCTGTGGCAGGACCGTCAGTCTTCCT
CTTCCCCCCAAAACCCAAGGACACCCTCATGATCTCCCGGACCCCTGAG
GTCACGTGCGTGGTGGTGGACGTGAGCCACGAAGACCCCGAGGTCCAGT
TCAACTGGTACGTGGACGGCGTGGAGGTGCATAATGCCAAGACAAAGCC
ACGGGAGGAGCAGTTCAACAGCACGTTCCGTGTGGTCAGCGTCCTCACC
GTTGTGCACCAGGACTGGCTGAACGGCAAGGAGTACAAGTGCAAGGTCT
CCAACAAAGGCCTCCCAGCCCCCATCGAGAAAACCATCTCCAAAACCAA
AGGGCAGCCCCGAGAACCACAGGTGTACACCCTGCCCCCATCCCGGGAG
GAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAA
CAACTACAAGACCACACCTCCCATGCTGGACTCCGACGGCTCCTTCTTC
CTCTAAGCAAGCTCACCGTGGACAAGAGCAGGTGGCAGCAGGGGAACGT
CTTCTCATGCTCCGTGATGCATGAGGCTCTGCACAACCACTACACGCAG
AAGAGCCTCTCCCTGTCTCCGGGTAAA

Protein Sequence Defining the Full Length Heavy Chain Sequence of 5.54.E6 with an IgG1 Constant Region (SEQ ID NO: 347)

QVQLVQSGAEVKKPGSSVNVSCKASAGTLSGYTINWLRQAPGRGFEWLG
RIIPLGIGNNANWEARVTLTADKSTDTTYLELRDLRSDDVAIYYCAVEF
YRLATVTTPTLDFWGQGTLVTVSSASTKGPSVFPLAPSSKSTSGGTAAL
GCLVKDYFPEPVTVSWNSGALTSGVTFPAVLQSSGLYSLSSVVTVPSSSLG
TQTYICNVNKPSNTKVDKKVEPKSCDKTTCPPCPAPELLGGPSVFLFPPKP
KDTLMISRTPEVTCVVVDVSEDPEVKFNWYVDGVEVNAKTKPREEQYNSTY
RVVSVLTVLQDWLNGKEYKCKVSNKALPAPIEKTISKAKGQPREPQVYTLP
PSRDELTKNQVSLTCLVKGFYPSDIAVEWESNGQPENNYKTTPPVLDSDGS
FFLYSKLTVDKSRWQQGNVFSCSVMEALNYTQKSLSLSPGK

Nucleic Acid Sequence Encoding the Full Length Lambda Chain Sequence of 5.54.E6 (SEQ ID NO: 348)

TCCTATGTGCTGACTCAGCCACCCTCGGTGTCCGTGGCCCCAGGAACGA
CGGCCAGGATTACCTGTGAGGGGGACCACATTGGAGCTAAAAGTGTGCA
CTGGTACCAGAGGAAGCCAGGCCAGGCCCCTGTGTTGGTCGTCTATATGA
ATAGTGACCGGCCCTCAGGGATCCCTGAGCGATTGTCTGGCTCCAACTCT
GGGAACACGGCCACCCTGAGTATCAGCAGGGTCGAGGCCGAGGATGAGG
CCGACTATTATTGTCAGGTTTGGGATAGTTTTCGCGATCATCAGGTAT
TCGGCGGAGGGACCAAGTTGACCGTCCTGAGTCAGCCCAAGGCTGCCCC
CTCGGTCACTCTGTTCCCGCCCTCCTCTGAGGAGCTTCAAGCCAACAAG
GCCACACTGGTGTGTCTCATAAGTGACTTCTACCCGGGAGCCGTGACAG
TGGCCTGGAAGGCAGATAGCAGCCCCGTCAAGGCGGGAGTGGAGACCAC
CACACCCTCCAAACAAGGCAACAACAAGTACGCGGCCAGCAGCTATCTG
AGCCTGACGCCTGAGCAGTGGAAGTCCCACAGAAGCTACAGCTGCCAGG
TCACGCATGAAGGGAGCACCGTGGAGAAGACAGTGGCCCCTACAGAATG
TTCA

Protein Sequence Defining the Full Length Lambda Chain Sequence of 5.54.E6 (SEQ ID NO: 349)

SYVLTQPPSVSVAPGTTARITCEGDHIGAKSVHWYQRKPGQAPVLVVYM
NSDRPSGIPERLSGSNSGNTATLSISRVEAEDEADYYCQVWDSFRDHQV
FGGGTKLTVLSQPKAAPSVTLFPPSSEELQANKATLVCLISDFYPGAVT
VAWKADSSPVKAGVETTTPSKQGNNKYAASSYLSLTPEQWKSHRSYSCQ
VTHEGSTVEKTVAPTECS

Nucleic Acid Sequence Encoding the Full Length Heavy Chain Sequence of 5.55.D2 with an IgG1 Constant Region (SEQ ID NO: 350)

CAGGTCACCTTGAAGGAGTCTGGTCCTGCTATAGTGAAACCCACACAGA
CCCTCACGCTGACCTGCACCTTCTCTGGGTTCTCACTCCGAACTAGTG
GAGTGGGTGTGGGCTGGATCCGTCAGCCCCCAGGAAAGGCCCTTGAGTG
GCTTGCACTCATTTACTGGGATGATGATAAGCGCTACAACCCATCTCTG
AAGACCAGACTCACCATCACCAAGGACTCCTCCAGAAACCAGGTGTTCC
TCACAATGACCAACATGGACCCTGTGGACACAGCCACATACTTCTGTGC
ACACCGACAACATATTACAAACAGTTTTGGATTGGTTATTGCGAATGAT
GCTTTTGACATTTGGGGCCAAGGGACAATGGTCACCGTCTCGAGTGCCT
CCACCAAGGGCCCATCGGTCTTCCCCCTGGCGCCCTGCTCCAGGAGCAC
CTCCGAGAGCACAGCCGCCCTGGGCTGCCTGGTCAAGGACTACTTCCCC
GAACCGGTGACGGTGTCGTGGAACTCAGGCGCTCTGACCAGCGGCGTGC
ACACCTTCCCAGCTGTCCTACAGTCCTCAGGACTCTACTCCCTCAGCAG
CGTGGTGACCGTGCCCTCCAGCAACTTCGGCACCCAGACCTACACCTGC
AACGTAGATCACAAGCCCAGCAACACCAAGGTGGACAAGACAGTTGAGC
GCAAATGTTGTGTCGAGTGCCCACCGTGCCCAGCACCACCTGTGGCAGG
ACCGTCAGTCTTCCTCTTCCCCCCAAAACCCAAGGACACCCTCATGATC
TCCCGGACCCCTGAGGTCACGTGCGTGGTGGTGGACGTGAGCCACGAAG
ACCCCGAGGTCCAGTTCAACTGGTACGTGGACGGCGTGGAGGTGCATAA
TGCCAAGACAAAGCCACGGGAGGAGCAGTTCAACAGCACGTTCCGTGTG
GTCAGCGTCCTCACCGTTGTGCACCAGGACTGGCTGAACGGCAAGGAGT
ACAAGTGCAAGGTCTCCAACAAAGGCCTCCCAGCCCCCATCGAGAAAAC
CATCTCCAAAACCAAAGGGCAGCCCCGAGAACCACAGGTGTACACCCTG
CCCCCATCCCGGGAGGAGATGACCAAGAACCAGGTCAGCCTGACCTGCC
TGGTCAAAGGCTTCTACCCCAGCGACATCGCCGTGGAGTGGGAGAGCAA
TGGGCAGCCGGAGAACAACTACAAGACCACACCTCCCATGCTGGACTCC
GACGGCTCCTTCTTCCTCTACAGCAAGCTCACCGTGGACAAGAGCAGGT
GGCAGCAGGGGAACGTCTTCTCATGCTCCGTGATGCATGAGGCTCTGCA
CAACCACTACACGCAGAAGAGCCTCTCCCTGTCTCCGGGTAAA

Protein Sequence Defining the Full Length Heavy Chain Sequence of 5.55.D2 with an IgG1 Constant Region (SEQ ID NO: 351)

QVTLKESGPAIVKPTQTLTLTCTFSGFSLRTSGVGVGWIRQPPGKALEW
LALIYWDDDKRYNPSLKTRLTITKDSSRNQVFLTMTNMDPVDTATYFCA
RQITNSFGLVIANDAFDIWGQGTMVTVSSASTKGPSVFPLAPSSKSTSG
GTAALGCLVKDYFPEPVTVSWNSGALTSGVTFPAVLQSSGLYSLSSVVT
VPSSSLGTQTYICNVNKPSNTKVDKKVEPKSCDKTTCPPCPAPELLGGP
SVFLFPPKPKDTLMISRTPEVTCVVVDVSEDPEVKFNWYVDGVEVNAKT
KPREEQYNSTYRVVSVLTVLQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMEALNYTQKS
LSLSPGK

Nucleic Acid Sequence Encoding the Full Length Lambda Chain Sequence of 5.55.D2 (SEQ ID NO: 352)

CAGTCTGCCCTGACTCAGCCTCCGTCCGCGTCCGGGTCTCCTGGACAGT
CAGTCACCATCTCCTGCACTGGAACCAGCAGTGACGTTGGTCGTTATA
ATTATGTCGCCTGGTATCAACAACACCCAGGCAGAGCCCCCAGACTCA
TAATTTATGGGGTCACTAAGCGGCCCTCAGGGGTCCCTGATCGCTTC
TCTGGCTCCAAGTCTGGCAACACGGCCTCCCTGACCGTCTCTGGGCTCC
AGGCTGAAGATGAGGCTGATTATTATTGCAGTTCATATGCAGGCAGCA
ACTTTTTGGAGGTGTTCGGCGGAGGGACCAAGCTGACCGTCCTAGGT
CAGCCCAAGGCTGCCCCCTCGGTCACTCTGTTCCCGCCCTCCTCTGAGG
AGCTTCAAGCCAACAAGGCCACACTGGTGTGTCTCATAAGTGACTTCTA
CCCGGGAGCCGTGACAGTGGCCTGGAAGGCAGATAGCAGCCCCGTCAAG
GCGGGAGTGGAGACCACCACACCCTCCAAACAAAGCAACAACAAGTACG
CGGCCAGCAGCTACCTGAGCCTGACGCCTGAGCAGTGGAAGTCCCACAG
AAGCTACAGCTGCCAGGTCACGCATGAAGGGAGCACCGTGGAGAAGACA
GTTGCCCCTACGGAATGTTCA

Protein Sequence Defining the Full Length Lambda Chain Sequence of 5.55.D2 (SEQ ID NO: 353)

QSALTQPPSASGSPGQSVTISCTGTSSDVGRYNYVAWYQQHPGRAPR
LIIYGVTKRPSGVPDRFSGSKSGNTASLTVSGLQAEDEADYYCS
SYAGSNFLEVFGGGTKLTVLGQPKAAPSVTLFPPSSEELQANKATLVC
LISDFYPGAVTVAWKADSSPVKAGVETTTPSKQSNNKYAASSYLSLTPE
QWKSHRSYSCQVTHEGSTVEKTVAPTECS

Nucleic Acid Sequence Encoding the Full Length Heavy Chain Sequence of 22.14.A1 with an IgG1 Constant Region (SEQ ID NO: 354)

CAGGTGCAGCTGGTGCAGTCTGGGGCTGAGGTGAAGAAGCCTGGGTCCT
CGGTGAAGGTCTCCTGCAAGGCTTCTGGAGGCACCTTCAGCAGCTATG
CAATCAACTGGGTGCGACAGGCCCCTGGACAAGGTCTTGAGTGGATGG
GAGGGATCATCCCTATGTTTGATACAGCAATCTACGCACAGATGTTC
CAGGGCAGAGTCACCATCACCGCGGACGAATCCACGAGCACAGCCTACAT
GGAGCTGAGCAGCCTGAGATCTGACGACACGGCCGTATATTACTGTGC
GAGAGATCGAAACGATTATTATGACAGTAGTGGTTATTCAGGCGCCTT
TGACCAGTGGGGCCAGGGAACCCTGGTCACCGTCTCGAGTGCCTCCA
CCAAGGGCCCATCGGTCTTCCCCCTGGCGCCCTGCTCCAGGAGCACCTC
CGAGAGCACAGCCGCCCTGGGCTGCCTGGTCAAGGACTACTTCCCCGAA
CCGGTGACGGTGTCGTGGAACTCAGGCGCTCTGACCAGCGGCGTGCACA
CCTTCCCAGCTGTCCTACAGTCCTCAGGACTCTACTCCCTCAGCAGCGT
GGTGACCGTGCCCTCCAGCAACTTCGGCACCCAGACCTACACCTGCAAC
GTAGATCACAAGCCCAGCAACACCAAGGTGGACAAGACAGTTGAGCGCA
AATGTTGTGTCGAGTGCCCACCGTGCCCAGCACCACCTGTGGCAGGACC
GTCAGTCTTCCTCTTCCCCCCAAAACCCAAGGACACCCTCATGATCTCC
CGGACCCCTGAGGTCACGTGCGTGGTGGTGGACGTGAGCCACGAAGACC
CCGAGGTCCAGTTCAACTGGTACGTGGACGGCGTGGAGGTGCATAATGC
CAAGACAAAGCCACGGGAGGAGCAGTTCAACAGCACGTTCCGTGTGGTC
AGCGTCCTCACCGTTGTGCACCAGGACTGGCTGAACGGCAAGGAGTACA
AGTGCAAGGTCTCCAACAAAGGCCTCCCAGCCCCCATCGAGAAAACCAT
CTCCAAAACCAAAGGGCAGCCCCGAGAACCACAGGTGTACACCCTGCCC
CCATCCCGGGAGGAGATGACCAAGAACCAGGTCAGCCTGACCTGCCTGG
TCAAAGGCTTCTACCCCAGCGACATCGCCGTGGAGTGGGAGAGCAATGG
GCAGCCGGAGAACAACTACAAGACCACACCTCCCATGCTGGACTCCGAC
GGCTCCTTCTTCCTCTACAGCAAGCTCACCGTGGACAAGAGCAGGTGGC
AGCAGGGGAACGTCTTCTCATGCTCCGTGATGCATGAGGCTCTGCACAA
CCACTACACGCAGAAGAGCCTCTCCCTGTCTCCGGGTAAA

Protein Sequence Defining the Full Length Heavy Chain Sequence of 22.14.A1 with an IgG1 Constant Region (SEQ ID NO: 355)

QVQLVQSGAEVKKPGSSVKVSCKASGGTFSSYAINWVRQAPGQGLEW
MGGIIPMFDTAIYAQMFQGRVTITADESTSTAYMELSSLRSDDTAVY
YCARDRNDYYDSSGYSGAFDQWGQGTLVTVSSASTKGPSVFPLAPS
SKSTSGGTAALGCLVKDYFPEPVTVSWNSGALTSGVTFPAVLQSSGLYS
LSSVVTVPSSSLGTQTYICNVNKPSNTKVDKKVEPKSCDKTTCPPCPAP
ELLGGPSVFLFPPKPKDTLMISRTPEVTCVVVDVSEDPEVKFNWYVDGV
EVNAKTKPREEQYNSTYRVVSVLTVLQDWLNGKEYKCKVSNKALPAPIE
KTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMEAL
NYTQKSLSLSPGK

Nucleic Acid Sequence Encoding the Full Length Kappa Chain Sequence of 22.14.A1 (SEQ ID NO: 356)

GAAATTGTGTTGACGCAGTCTCCAGGCGCCCTGTCTTTGTCTCCAGGGG
AAAGAGCCACCCTCTCCTGCAGGGCCAGTCAGAGTGTTAGCAGCAGCT
ACTTAGCCTGGTACCAGCAGAAACCTGGCCAGGCTCCCAGGCTCCTCA
TCTATGGTGCATCCAGCAGGGCCACTGGCATCCCAGACAGGTTCAGT
GGCAGTGGGTCTGGGACAGACTTCACTCTCACAATCAGCAGACTGGAGC
CTGAGGATTTTGCAGTGTATTACTGTCAGCAGTATGGTAGCTCACCTC
GCACTTTTGGCCAGGGGACCAAGCTGGAGATCAAACGAACTGTGGCT
GCACCATCTGTCTTCATCTTCCCGCCATCTGATGAGCAGTTGAAATCTG
GAACTGCCTCTGTTGTGTGCCTGCTGAATAACTTCTATCCCAGAGAGGC
CAAAGTACAGTGGAAGGTGGATAACGCCCTCCAATCGGGTAACTCCCAG
GAGAGTGTCACAGAGCAGGACAGCAAGGACAGCACCTACAGCCTCAGCA
GCACCCTGACGCTGAGCAAAGCAGACTACGAGAAACACAAAGTCTACGC
CTGCGAAGTCACCCATCAGGGCCTGAGCTCGCCCGTCACAAAGAGCTTC
AACAGGGGAGAGTGTTAGAACGCGTG

Protein Sequence Defining the Full Length Kappa Chain Sequence of 22.14.A1 (SEQ ID NO: 357)

EIVLTQSPGALSLSPGERATLSCRASQSVSSSYLAWYQQKPGQAPRL
LIYGASSRATGIPDRFSGSGSGTDFTLTISRLEPEDFAVYYCQQYG
SSPRTFGQGTKLEIKRTVAAPSVFIFPPSDEQLKSGTASVVCLLNNF
YPREAKVQWKVDNALQSGNSQESVTEQDSKDSTYSLSSTLTLSKADYEK
HKVYACEVTHQGLSSPVTKSFNRGEC

Nucleic Acid Sequence Encoding the Full Length Heavy Chain Sequence of 26.53.B4 with an IgG1 Constant Region (SEQ ID NO: 358)

GAGGTGCAGCTGGTGGAGTCCGGGGGAGGCTTGGTCCAGCCTGGGGGGT
CCCTGAAACTCTCCTGTGCAGCCTCTGGGTTCAGCTTCAGTGGCTCTG
CTGTGGAATGGGTCCGCCAGGCTTCCGGGAGAGGGCTGGAGTGGATTG
GCCATATAAGAACCAAGACCAAAAATTACGCGACAGCGTATGCTGCG
TCGGTGCAAGGCAGGTTCACCATCTTCAGAGATGATTCAAGGAACACGG
CGTATCTGCAGATGAACAGTCTTAAAACCGAGGACACGGCCGTGTATTA
TTGTTCCAGACACATCGAATTTAGCAGTTCGTCCGGGCCCCAGATGG
GCCAGGGAACCCTGGTCACCGTCTCGAGTGCCTCCACCAAGGGCCCAT
CGGTCTTCCCCCTGGCGCCCTGCTCCAGGAGCACCTCCGAGAGCACAGC
CGCCCTGGGCTGCCTGGTCAAGGACTACTTCCCCGAACCGGTGACGGTG
TCGTGGAACTCAGGCGCTCTGACCAGCGGCGTGCACACCTTCCCAGCTG
TCCTACAGTCCTCAGGACTCTACTCCCTCAGCAGCGTGGTGACCGTGCC
CTCCAGCAACTTCGGCACCCAGACCTACACCTGCAACGTAGATCACAAG
CCCAGCAACACCAAGGTGGACAAGACAGTTGAGCGCAAATGTTGTGTCG
AGTGCCCACCGTGCCCAGCACCACCTGTGGCAGGACCGTCAGTCTTCCT
CTTCCCCCCAAAACCCAAGGACACCCTCATGATCTCCCGGACCCCTGAG
GTCACGTGCGTGGTGGTGGACGTGAGCCACGAAGACCCCGAGGTCCAGT
TCAACTGGTACGTGGACGGCGTGGAGGTGCATAATGCCAAGACAAAGCC
ACGGGAGGAGCAGTTCAACAGCACGTTCCGTGTGGTCAGCGTCCTCACC
GTTGTGCACCAGGACTGGCTGAACGGCAAGGAGTACAAGTGCAAGGTCT
CCAACAAAGGCCTCCCAGCCCCCATCGAGAAAACCATCTCCAAAACCAA
AGGGCAGCCCCGAGAACCACAGGTGTACACCCTGCCCCCATCCCGGGAG
GAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAA
CAACTACAAGACCACACCTCCCATGCTGGACTCCGACGGCTCCTTCTTC
CTCTACAGCAAGCTCACCGTGGACAAGAGCAGGTGGCAGCAGGGGAACG
TCTTCTCATGCTCCGTGATGCATGAGGCTCTGCACAACCACTACACGCA
GAAGAGCCTCTCCCTGTCTCCGGGTAAA

Protein Sequence Defining the Full Length Heavy Chain Sequence of 26.53.B4 with an IgG1 Constant Region (SEQ ID NO: 359)

EVQLVESGGGLVQPGGSLKLSCAASGFSFSGSAVEWVRQASGRGLEW
IGIRTKTKNYATAYAASVQGRFTIFRDDSRNTAYLQMNSLKTEDTAV
YYCSRIEFSSSSGPQMGQGTLVTVSSASTKGPSVFPLAPSSKSTSG
GTAALGCLVKDYFPEPVTVSWNSGALTSGVTFPAVLQSSGLYSLSSVVT
VPSSSLGTQTYICNVNKPSNTKVDKKVEPKSCDKTTCPPCPAPELLGGP
SVFLFPPKPKDTLMISRTPEVTCVVVDVSEDPEVKFNWYVDGVEVNAKT
KPREEQYNSTYRVVSVLTVLQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMEALNYTQKS
LSLSPGK

Nucleic Acid Sequence Encoding the Full Length Kappa Chain Sequence of 26.53.B4 (SEQ ID NO: 360)

GACATCCAGATGACCCAGTCTCCTTCCACCCTGTCTGCATCTGTAGGCG
ACACAGTCACCATCACTTGCCAGGCCAGTCAGAATATTATTACTTGG
TTGGCCTGGTATCAGCAGAAACCAGGGAAAGCCCCTAACATCCTCATCT
ATAAGGCGTCTGGTTTAGAGAGTGGGGTCCCACCGAGGTTCAGCGGC
AGTGGGTCGGGGACAGAATTCACTCTCACCATCAGCAGCCTGCAGCCTG
ATGATGTTGCAACTTATTATTGCCTACAATATAAAACTGAGCCGTGGA
CGTTCGGCCTAGGGACCAAGGTGGAGATCAGACGAACTGTGGCTGCA
CCATCTGTCTTCATCTTCCCGCCATCTGATGAGCAGTTGAAATCTGGAA
CTGCCTCTGTTGTGTGCCTGCTGAATAACTTCTATCCCAGAGAGGCCAA
AGTACAGTGGAAGGTGGATAACGCCCTCCAATCGGGTAACTCCCAGGAG
AGTGTCACAGAGCAGGACAGCAAGGACAGCACCTACAGCCTCAGCAGCA
CCCTGACGCTGAGCAAAGCAGACTACGAGAAACACAAAGTCTACGCCTG
CGAAGTCACCCATCAGGGCCTGAGCTCGCCCGTCACAAAGAGCTTCAAC
AGGGGAGAGTGT

Protein Sequence Defining the Full Length Kappa Chain Sequence of 26.53.B4 (SEQ ID NO: 361)

DIQMTQSPSTLSASVGDTVTITCQASQNIITWLAWYQQKPGKAPNIL
IYKASGLESGVPPRFSGSGSGTEFTLTISSLQPDDVATYYCLQYKT
EPWTFGLGTKVEIRRTVAAPSVFIFPPSDEQLKSGTASVVCLLNNFY
PREAKVQWKVDNALQSGNSQESVTEQDSKDSTYSLSSTLTLSKADYEKH
KVYACEVTHQGLSSPVTKSFNRGEC

Nucleic Acid Sequence Encoding the Full Length Heavy Chain Sequence of 5.63.E2 (SEQ ID NO: 362)

GAGGTGCAGCTGGTGGAGTCTGGGGGAGGCTTGGCCCAGCCGGGGGAGT
CCCTGAGACTCTCCTGTGCAGCCTCCGGATTCACCTTTAGTGCCTATT
GGATGAGCTGGGTCCGCCAGACTCCAGGGAAGGGGCTGGAGTGGGTGG
CCAACATTAACCAGGGTGGAGATAAGACAGACTATTTGGACTCTGTG
AAGGGCCGATTCACCGTCTCCAGAGACAATGCCAAGAACTTAGTGTTTC
TGCAAATGGCCAGCCTGAGAGCCGACGACACGGCCGTGTATTACTGTG
GGAGAGGTACAAACCAAGACTACTGGGGCCAGGGAACCCTGGTCACCG
TCTCGAGTGCCTCCACCAAGGGCCCATCGGTCTTCCCCCTGGCACCCT
CCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTG
ACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCA
GACCTACATCTGCAACGTGAATCACAAGCCCAGCAACACCAAGGTGGAC
AAGAAAGTTGAGCCCAAATCTTGTGACAAAACTCACACATGCCCACCGT
GCCCAGCACCTGAACTCCTGGGGGGACCGTCAGTCTTCCTCTTCCCCCC
AAAACCCAAGGACACCCTCATGATCTCCCGGACCCCTGAGGTCACATGC
GTGGTGGTGGACGTGAGCCACGAAGACCCTGAGGTCAAGTTCAACTGGT
ACGTGGACGGCGTGGAGGTGCATAATGCCAAGACAAAGCCGCGGGAGGA
GCAGTACAACAGCACGTACCGTGTGGTCAGCGTCCTCACCGTCCTGCAC
CAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCC
CCGAGAACCACAGGTGTACACCCTGCCCCCATCCCGGGATGAGCTGACC
AAGAACCAGGTCAGCCTGACCTGCCTGGTCAAAGGCTTCTATCCCAGCG
ACATCGCCGTGGAGTGGGAGAGCAATGGGCAGCCGGAGAACAACTACAA
GACCACGCCTCCCGTGCTGGACTCCGACGGCTCCTTCTTCCTCTACAGC
AAGCTCACCGTGGACAAGAGCAGGTGGCAGCAGGGGAACGTCTTCTCAT
GCTCCGTGATGCATGAGGCTCTGCACAACCACTACACGCAGAAGAGCCT
CTCCCTGTCTCCGGGTAAA

Protein Sequence Defining the Full Length Heavy Chain Sequence of 5.63.E2 (SEQ ID NO: 363)

EVQLVESGGGLAQPGESLRLSCAASGFTFSAYWMSWVRQTPGKGLEW
VANINQGGDKTDYLDSVKGRFTVSRDNAKNLVFLQMASLRADDTAVY
YCGRGTNQDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHY
TQKSLSLSPGK

Nucleic Acid Sequence Encoding the Full Length Lambda Light Chain Sequence of 5.63.E2 (SEQ ID NO: 364)

CAGTCTGTGCTGACTCAGCCACCCTCAGCGTCTGGGACCCCCGGGCAGA
GGGTCACCATCTCTTGTTCTGGAAGCAGCTCCAATATCGGAAGTAATA
ATGTATACTGGTACCAACAGGTCCCAGGAACGGCCCCCAAATTTCTCA
TCTATCCGAATAGTGAGCGGCCCTCAGGGGTCCCTGACCGATTCTCT
GCCTCCAAGTCTGGCACCTCAGCCTCCCTGGCCATCAGTGGACTCCGGT
CCGAGGATGAGGCTACTTATTACTGTGCAGCATGGGATGACAGCCTAA
GAACTTATGTCTTCGCATCTGGGACCAAGGTCACCGTCCTAGGTCAG
CCCAAGGCCAACCCCACTGTCACTCTGTTCCCGCCCTCCTCTGAGGAGC
TCCAAGCCAACAAGGCCACACTAGTGTGTCTGATCAGTGACTTCTACCC
GGGAGCTGTGACAGTGGCCTGGAAGGCAGATGGCAGCCCCGTCAAGGCG
GGAGTGGAGACCACCAAACCCTCCAAACAGAGCAACAACAAGTACGCGG
CCAGCAGCTACCTGAGCCTGACGCCCGAGCAGTGGAAGTCCCACAGAAG
CTACAGCTGCCAGGTCACGCATGAAGGGAGCACCGTGGAGAAGACAGTG
GCCCCTACAGAATGTTCA

Protein Sequence Defining the Full Length Lambda Light Chain Sequence of 5.63.E2 (SEQ ID NO: 365)

QSVLTQPPSASGTPGQRVTISCSGSSSNIGSNNVYWYQQVPGTAPKF
LIYPNSERPSGVPDRFSASKSGTSASLAISGLRSEDEATYYCAAWD
DSLRTYVFASGTKVTVLGQPKANPTVTLFPPSSEELQANKATLVCLI
SDFYPGAVTVAWKADGSPVKAGVETTKPSKQSNNKYAASSYLSLTPEQW
KSHRSYSCQVTHEGSTVEKTVAPTECS

Nucleic Acid Sequence Encoding the Full Length Heavy Chain Sequence of 5.64.G4 (SEQ ID NO: 366)

CAGGTGCAGCTGGTGGAGTCTGGGGGAGGCGTGGTCCAGCCTGGGAGGT
CCCTGAGACTCTCCTGTGCAGCCTCCGGATTCACCCTCAGGAGTTATG
GCATGCACTGGGTCCGCCAGGCTCCAGGCAAGGGCCTGGAGTGGGTGG
CAGTTATATCATATGATGGAAGTAAGACATATTATAGAGACTCCGTG
AAGGGCCGATTCACCATCTCCAGAGACAATTCTAAGAACACACTGTATT
TGCAAATGAACAGCCTGCGAGTTGAGGACACGGCTGTATATTATTGTG
CGAAAGAGGATAATTTGGGATCTGATGTTTTTGATAGATGGGGCCAAG
GGACAATGGTCACCGTCTCGAGTGCCTCCACCAAGGGCCCATCGGTCT
TCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGG
AACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAG
CAGCTTGGGCACCCAGACCTACATCTGCAACGTGAATCACAAGCCCAGC
AACACCAAGGTGGACAAGAAAGTTGAGCCCAAATCTTGTGACAAAACTC
ACACATGCCCACCGTGCCCAGCACCTGAACTCCTGGGGGGACCGTCAGT
CTTCCTCTTCCCCCCAAAACCCAAGGACACCCTCATGATCTCCCGGACC
CCTGAGGTCACATGCGTGGTGGTGGACGTGAGCCACGAAGACCCTGAGG
TCAAGTTCAACTGGTACGTGGACGGCGTGGAGGTGCATAATGCCAAGAC
AAAGCCGCGGGAGGAGCAGTACAACAGCACGTACCGTGTGGTCAGCGTC
CTCACCGTCCTGCACCAGGACTGGCTGAATGGCAAGGAGTACAAGTGCA
AGGTCTCCAACAAAGCCCTCCCAGCCCCCATCGAGAAAACCATCTCCAA
AGCCAAAGGGCAGCCCCGAGAACCACAGGTGTACACCCTGCCCCCATCC
CGGGATGAGCTGACCAAGAACCAGGTCAGCCTGACCTGCCTGGTCAAAG
GCTTCTATCCCAGCGACATCGCCGTGGAGTGGGAGAGCAATGGGCAGCC
GGAGAACAACTACAAGACCACGCCTCCCGTGCTGGACTCCGACGGCTCC
TTCTTCCTCTACAGCAAGCTCACCGTGGACAAGAGCAGGTGGCAGCAGG
GGAACGTCTTCTCATGCTCCGTGATGCATGAGGCTCTGCACAACCACTA
CACGCAGAAGAGCCTCTCCCTGTCTCCGGGTAAA

Protein Sequence Defining the Full Length Heavy Chain Sequence of 5.64.G4 (SEQ ID NO: 367)

QVQLVESGGGVVQPGRSLRLSCAASGFTLRSYGMHWVRQAPGKGLEWVA
VISYDGSKTYYRDSVKGRFTISRDNSKNTLYLQMNSLRVEDTAVYYCAK
EDNLGSDVFDRWGQGTMVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK

Nucleic Acid Sequence Encoding the Full Length Lambda Light Chain Sequence of 5.64.G4 (SEQ ID NO: 368)

GACATCGTGATGACCCAGTCTCCTTCCACCCTGTCTGCATCTGTAGGAG
ACAGAGTTACCATCACTTGTCGGGCCAGTCAGAGTGCCAGTCAGTGGTT
GGCCTGGTATCAGCAAAAACCAGGGAAAGCCCCTAATCTCCTAATCTAT
AAGGCGTCTAATTTACATAGTGGGGTCCCATCAAGGTTCAGCGGCAGTG
GATCTGGGACCGAATTCACTCTCACCATCAGCAGCCTACAGCCTGATGA
TTTTGCAACTTATTACTGCCAACAATACAACATTTATCCGTGGACGTTC
GGCCAAGGGACCAAGGTGGAAGTGAGACGAACTGTGGCTGCACCATCTG
TCTTCATCTTCCCGCCATCTGATGAGCAGTTGAAATCTGGAACTGCCTC
TGTTGTGTGCCTGCTGAATAACTTCTATCCCAGAGAGGCCAAAGTACAG
TGGAAGGTGGATAACGCCCTCCAATCGGGTAACTCCCAGGAGAGTGTCA
CAGAGCAGGACAGCAAGGACAGCACCTACAGCCTCAGCAGCACCCTGAC
GCTGAGCAAAGCAGACTACGAGAAACACAAAGTCTACGCCTGCGAAGTC
ACCCATCAGGGCCTGAGCTCGCCCGTCACAAAGAGCTTCAACAGGGGAG
AGTGTT

Protein Sequence Defining the Full Length Lambda Light Chain Sequence of 5.64.G4 (SEQ ID NO: 369)

DIVMTQSPSTLSASVGDRVTITCRASQSASQWLAWYQQKPGKAPNLLIY
KASNLHSGVPSRFSGSGSGTEFTLTISSLQPDDFATYYCQQYNIYPWTF
GQGTKVEVRRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Nucleic Acid Sequence Encoding the Full Length Heavy Chain Sequence of 43.52.A11 (SEQ ID NO: 370)

CAGGTCCAGCTGGTGCAGTCTGGGGCTGAGGTGAAGGAGCCAGGGTCCT
CGGTGAGGGTCTCCTGCAAGGCTTCTGGACGCATCTTCCGGAATTATGC
TGTCAGCTGGGTGCGACAGGCCCCTGGACAAGGGCTTGAGTGGGTGGGC
CGGTTCATCCCTTCGGAAGGAATAACAAACTACATTCCGACATTCCGGG
ACAGACTCACCTTTACTGCGGACAAGTCCACGAACACAGCCTTCATTGA
CCTGACTAACCTGAAATATGAGGACACGGCCATATATTACTGTGTGGGT
GGCAGGCCAGGCGCTTTCGTCCCCTTTGACTCCTGGGGCCAGGGAACCC
TGGTCACCGTCTCGAGTGCCTCCACCAAGGGCCCATCGGTCTTCCCCCT
GGCACCCTCCTCCAAGAGCACCTCTGGGGGCACAGCGGCCCTGGGCTGC
CTGGTCAAGGACTACTTCCCCGAACCGGTGACGGTGTCGTGGAACTCAG
GCGCCCTGACCAGCGGCGTGCACACCTTCCCGGCTGTCCTACAGTCCTC
AGGACTCTACTCCCTCAGCAGCGTGGTGACCGTGCCCTCCAGCAGCTTG
GGCACCCAGACCTACATCTGCAACGTGAATCACAAGCCCAGCAACACCA
AGGTGGACAAGAAAGTTGAGCCCAAATCTTGTGACAAAACTCACACATG
CCCACCGTGCCCAGCACCTGAACTCCTGGGGGGACCGTCAGTCTTCCTC
TTCCCCCCAAAACCCAAGGACACCCTCATGATCTCCCGGACCCCTGAGG
TCACATGCGTGGTGGTGGACGTGAGCCACGAAGACCCTGAGGTCAAGTT
CAACTGGTACGTGGACGGCGTGGAGGTGCATAATGCCAAGACAAAGCCG
CGGGAGGAGCAGTACAACAGCACGTACCGTGTGGTCAGCGTCCTCACCG
TCCTGCACCAGGACTGGCTGAATGGCAAGGAGTACAAGTGCAAGGTCTC
CAACAAAGCCCTCCCAGCCCCCATCGAGAAAACCATCTCCAAAGCCAAA
GGGCAGCCCCGAGAACCACAGGTGTACACCCTGCCCCCATCCCGGGATG
AGCTGACCAAGAACCAGGTCAGCCTGACCTGCCTGGTCAAAGGCTTCTA
TCCCAGCGACATCGCCGTGGAGTGGGAGAGCAATGGGCAGCCGGAGAAC
AACTACAAGACCACGCCTCCCGTGCTGGACTCCGACGGCTCCTTCTTCC
TCTACAGCAAGCTCACCGTGGACAAGAGCAGGTGGCAGCAGGGGAACGT
CTTCTCATGCTCCGTGATGCATGAGGCTCTGCACAACCACTACACGCAG
AAGAGCCTCTCCCTGTCTCCGGGTAAA

Protein Sequence Defining the Full Length Heavy Chain Sequence of 43.52.A11 (SEQ ID NO: 371)

QVQLVQSGAEVKEPGSSVRVSCKASGRIFRNYAVSWVRQAPGQGLEWVG
RFIPSEGITNYIPTFRDRLTFTADKSTNTAFIDLTNLKYEDTAIYYCVG
GRPGAFVPFDSWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK

Nucleic Acid Sequence Encoding the Full Length Kappa Light Chain Sequence of 43.52.A11 (SEQ ID NO: 372)

GAAATAGTGATGACGCAGTCTCCTGCCACCCTGTCTGTGTCTCCAGGGG
AAAGAGCCACCCTCTCCTGCAGGGCCAGTCAGAGTGTCAGGGGCAACTT
AGCCTGGTACCAGCAAAAACCTGGCCAGGCTCCCAGGCTTCTCATGTAT
GATGCTTCTACCAGGGCCACTAGTTTCCCAGCCCGGTTCAGTGGCAGTG
GGTCTGGGACAGAGTTCACTCTCACCATCAGCAGTGTGCAGTCTGAAGA
TTTTGCACTTTATTACTGTCAGCAGTATAATGACTGGCCCCGGACTTTT
GGCCAGGGGACCAAGGTGGAGATCAAACGAACTGTGGCTGCACCATCTG
TCTTCATCTTCCCGCCATCTGATGAGCAGTTGAAATCTGGAACTGCCTC
TGTTGTGTGCCTGCTGAATAACTTCTATCCCAGAGAGGCCAAAGTACAG
TGGAAGGTGGATAACGCCCTCCAATCGGGTAACTCCCAGGAGAGTGTCA
CAGAGCAGGACAGCAAGGACAGCACCTACAGCCTCAGCAGCACCCTGAC
GCTGAGCAAAGCAGACTACGAGAAACACAAAGTCTACGCCTGCGAAGTC
ACCCATCAGGGCCTGAGCTCGCCCGTCACAAAGAGCTTCAACAGGGGAG
AGTGTT

Protein Sequence Defining the Full Length Kappa Light Chain Sequence of 43.52.A11 (SEQ ID NO: 373)

EIVMTQSPATLSVSPGERATLSCRASQSVRGNLAWYQQKPGQAPRLLMY
DASTRATSFPARFSGSGSGTEFTLTISSVQSEDFALYYCQQYNDWPRTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Nucleic Acid Sequence Encoding the Full Length Heavy Chain Sequence of 43.52.E12 (SEQ ID NO: 374)

CAGGTGCAGCTGGTGCAGTCTGGGGCTGAGGTGAAGAAGCCTGGGTCCT
CGGTGAGGGTCTCCTGCAGGGTTTCTGGAGGCAGTTTCTCCAGTTATGA
CGTACACTGGGTGCGACAGGCCCCTGGACAAGGGCTTGAGTGGGTGGGA
AGGAGCGTCTCACGTGCTGACAGCTCAAGTCACACACGGGAGTTCGGGG
ACAGAGTCACGATTATCGCGGACAAATCCACGAGTACAGTTTATTTGAC
ACTGACCAGTCTGACCTCTGACGACACGGCCATCTATTACTGTGCGGCA
GGGGATTGTAGTGATGGCGGGTGCTTCCGCACACCCCTGCACATCTGGG
GCCAGGGCACCCTGGTCACCGTCTCGAGTGCCTCCACCAAGGGCCCATC
GGTCTTCCCCCTGGCACCCTCCTCCAAGAGCACCTCTGGGGGCACAGCG
GCCCTGGGCTGCCTGGTCAAGGACTACTTCCCCGAACCGGTGACGGTGT
CGTGGAACTCAGGCGCCCTGACCAGCGGCGTGCACACCTTCCCGGCTGT
CCTACAGTCCTCAGGACTCTACTCCCTCAGCAGCGTGGTGACCGTGCCC
TCCAGCAGCTTGGGCACCCAGACCTACATCTGCAACGTGAATCACAAGC
CCAGCAACACCAAGGTGGACAAGAAAGTTGAGCCCAAATCTTGTGACAA
AACTCACACATGCCCACCGTGCCCAGCACCTGAACTCCTGGGGGGACCG
TCAGTCTTCCTCTTCCCCCCAAAACCCAAGGACACCCTCATGATCTCCC
GGACCCCTGAGGTCACATGCGTGGTGGTGGACGTGAGCCACGAAGACCC
TGAGGTCAAGTTCAACTGGTACGTGGACGGCGTGGAGGTGCATAATGCC
AAGACAAAGCCGCGGGAGGAGCAGTACAACAGCACGTACCGTGTGGTCA
GCGTCCTCACCGTCCTGCACCAGGACTGGCTGAATGGCAAGGAGTACAA
GTGCAAGGTCTCCAACAAAGCCCTCCCAGCCCCCATCGAGAAAACCATC
TCCAAAGCCAAAGGGCAGCCCCGAGAACCACAGGTGTACACCCTGCCCC
CATCCCGGGATGAGCTGACCAAGAACCAGGTCAGCCTGACCTGCCTGGT
CAAAGGCTTCTATCCCAGCGACATCGCCGTGGAGTGGGAGAGCAATGGG
CAGCCGGAGAACAACTACAAGACCACGCCTCCCGTGCTGGACTCCGACG
GCTCCTTCTTCCTCTACAGCAAGCTCACCGTGGACAAGAGCAGGTGGCA
GCAGGGGAACGTCTTCTCATGCTCCGTGATGCATGAGGCTCTGCACAAC
CACTACACGCAGAAGAGCCTCTCCCTGTCTCCGGGTAAA

Protein Sequence Defining the Full Length Heavy Chain Sequence of 43.52.E12 (SEQ ID NO: 375)

QVQLVQSGAEVKKPGSSVRVSCRVSGGSFSSYDVHWVRQAPGQGLEWVG
RSVSRADSSSHTREFGDRVTIIADKSTSTVYLTLTSLTSDDTAIYYCAA
GDCSDGGCFRTPLHIWGQGTLVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHN
HYTQKSLSLSPGK

Nucleic Acid Sequence Encoding the Full Length Kappa Light Chain Sequence of 43.52.E12 (SEQ ID NO: 376)

GAAATAGTGATGACGCAGTCTCCAGCCACTCTGTCTCTGTCTCCAGGGG
AAAGAGCCACCCTCTCCTGCAGGGCCAGTCAGAGTCTGAGAACCAACTT
AGCCTGGTACCAGCAGCGACCTGGCCAGGCTCCCAGGCTCCTCATCTCT
GGGGCATCCACGAGGGCCACTGGTATCCCAGCCAGGTTCAGTGGCAGTG
GGTCTGGGACAGAATTCACTCTCACCATTAGGAGCCTGCAGCCTGAAGA
TTTTGCAGTTTATTACTGTCAACAGTATCATGACTGGCCTCGGACGTTC
GGCCAAGGGACCAAGGTGGATTTCAAACGAACTGTGGCTGCACCATCTG
TCTTCATCTTCCCGCCATCTGATGAGCAGTTGAAATCTGGAACTGCCTC
TGTTGTGTGCCTGCTGAATAACTTCTATCCCAGAGAGGCCAAAGTACAG
TGGAAGGTGGATAACGCCCTCCAATCGGGTAACTCCCAGGAGAGTGTCA
CAGAGCAGGACAGCAAGGACAGCACCTACAGCCTCAGCAGCACCCTGAC
GCTGAGCAAAGCAGACTACGAGAAACACAAACTCTACGCCTGCGAAGTC
ACCCATCAGGGCCTGAGCTCGCCCGTCACAAAGAGCTTCAACAGGGGAG
AGTGTTAG

Protein Sequence Defining the Full Length Kappa Light Chain Sequence of 43.52.E12 (SEQ ID NO: 377)

EIVMTQSPATLSLSPGERATLSCRASQSLRTNLAWYQQRPGQAPRLLIS
GASTRATGIPARFSGSGSGTEFTLTIRSLQPEDFAVYYCQQYHDWPRTF
GQGTKVDFKRTVAAPSVFIFPPSDEQLKSGTASVVCLLNNFYPREAKVQ
WKVDNALQSGNSQESVTEQDSKDSTYSLSSTLTLSKADYEKHKLYACEV
THQGLSSPVTKSFNRGEC

Nucleic Acid Sequence Encoding the Full Length Heavy Chain Sequence of 43.62.E2 (SEQ ID NO: 378)

GAGGTGCAGCTGGTGCAGTCTGGGGCAGAAGTGAAAAAGGCCGGGGAGT
CTCTGAGGATCTCCTGTAAGGGTTCTGGATACAACTTTAATAGCTATTG
GATTGGCTGGGTGCGCCAGATGCCCGGGAAAGGCCTGGAGTGGATGGCA
ATCATCTATCCTGCTGACTCTGACAGGAGATATGGCCCGTCCTTCCAAG
GCCAGGTCACCATCTCAGTCGACAAGTCTATTAACACCGCGTACTTGCA
GTGGAGCAGCCTGAAGGCCTCGGACACCGCCATGTATTATTGTGCCAGA
CTCCTCAGAGTTAGTACTGGTTGGGAAGATGCTTTTGATCTGTGGGGCC
AAGGGACAATGGTCACCGTCTCGAGTGCCTCCACCAAGGGCCCATCGGT
CTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGT
GGAACTCAGGCGCCCTGACCAGCGGCGTGCACACCTTCCCGGCTGTCCT
ACAGTCCTCAGGACTCTACTCCCTCAGCAGCGTGGTGACCGTGCCCTCC
AGCAGCTTGGGCACCCAGACCTACATCTGCAACGTGAATCACAAGCCCA
GCAACACCAAGGTGGACAAGAAAGTTGAGCCCAAATCTTGTGACAAAAC
TCACACATGCCCACCGTGCCCAGCACCTGAACTCCTGGGGGGACCGTCA
GTCTTCCTCTTCCCCCCAAAACCCAAGGACACCCTCATGATCTCCCGGA
CCCCTGAGGTCACATGCGTGGTGGTGGACGTGAGCCACGAAGACCCTGA
GGTCAAGTTCAACTGGTACGTGGACGGCGTGGAGGTGCATAATGCCAAG
ACAAAGCCGCGGGAGGAGCAGTACAACAGCACGTACCGTGTGGTCAGCG
TCCTCACCGTCCTGCACCAGGACTGGCTGAATGGCAAGGAGTACAAGTG
CAAGGTCTCCAACAAAGCCCTCCCAGCCCCCATCGAGAAAACCATCTCC
AAAGCCAAAGGGCAGCCCCGAGAACCACAGGTGTACACCCTGCCCCCAT
CCCGGGATGAGCTGACCAAGAACCAGGTCAGCCTGACCTGCCTGGTCAA
AGGCTTCTATCCCAGCGACATCGCCGTGGAGTGGGAGAGCAATGGGCAG
CCGGAGAACAACTACAAGACCACGCCTCCCGTGCTGGACTCCGACGGCT
CCTTCTTCCTCTACAGCAAGCTCACCGTGGACAAGAGCAGGTGGCAGCA
GGGGAACGTCTTCTCATGCTCCGTGATGCATGAGGCTCTGCACAACCAC
TACACGCAGAAGAGCCTCTCCCTGTCTCCGGGTAAA

Protein Sequence Defining the Full Length Heavy Chain Sequence of 43.62.E2 (SEQ ID NO: 379)

EVQLVQSGAEVKKAGESLRISCKGSGYNFNSYWIGWVRQMPGKGLEWMA
IIYPADSDRRYGPSFQGQVTISVDKSINTAYLQWSSLKASDTAMYYCAR
LLRVSTGWEDAFDLWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK

Nucleic Acid Sequence Encoding the Full Length Kappa Light Chain Sequence of 43.62.E2 (SEQ ID NO: 380)

GACATCGTGATGACCCAGTCTCCAGACTCCCTGGTTGTGTCTCTGGGCG
AGAGGGCCACCATCAAGTGCCAGTCCAGCCAGAGTGTTTTGTACAGCTC
CACCAATAAGAACTATTTAGCCTGGTACCAGCAGAAATCGGGACAGCCA
CCTAAGCTACTCATTTACTGGGCCTCCACCCGGGAATCCGGGGTCCCTG
ACCGATTCATTGGTAGCGGCTCTAGGACAGATTTCACTCTCACCATCAG
CGACCTGCAGCCTGAGGATGCGGCAGTTTATTACTGTCAGCAGTATTAT
AGTGATTATCTCACTTTCGGCGGAGGGACCAAGGTGGAGATCAAACGAA
CTGTGGCTGCACCATCTGTCTTCATCTTCCCGCCATCTGATGAGCAGTT
GAAATCTGGAACTGCCTCTGTTGTGTGCCTGCTGAATAACTTCTATCCC
AGAGAGGCCAAAGTACAGTGGAAGGTGGATAACGCCCTCCAATCGGGTA
ACTCCCAGGAGAGTGTCACAGAGCAGGACAGCAAGGACAGCACCTACAG
CCTCAGCAGCACCCTGACGCTGAGCAAAGCAGACTACGAGAAACACAAA
GTCTACGCCTGCGAAGTCACCCATCAGGGCCTGAGCTCGCCCGTCACAA
AGAGCTTCAACAGGGGAGAGTGTTAG

Protein Sequence Defining the Full Length Kappa Light Chain Sequence of 43.62.E2 (SEQ ID NO: 381)

DIVMTQSPDSLVVSLGERATIKCQSSQSVLYSSTNKNYLAWYQQKSGQP
PKLLIYWASTRESGVPDRFIGSGSRTDFTLTISDLQPEDAAVYYCQQYY
SDYLTFGGGTKVEIKRTVAAPSVFIFPPSDEQLKSGTASVVCLLNNFYP
REAKVQWKVDNALQSGNSQESVTEQDSKDSTYSLSSTLTLSKADYEKHK
VYACEVTHQGLSSPVTKSFNRGEC

### Example 2

### Identification of Human S. aureus Antibodies

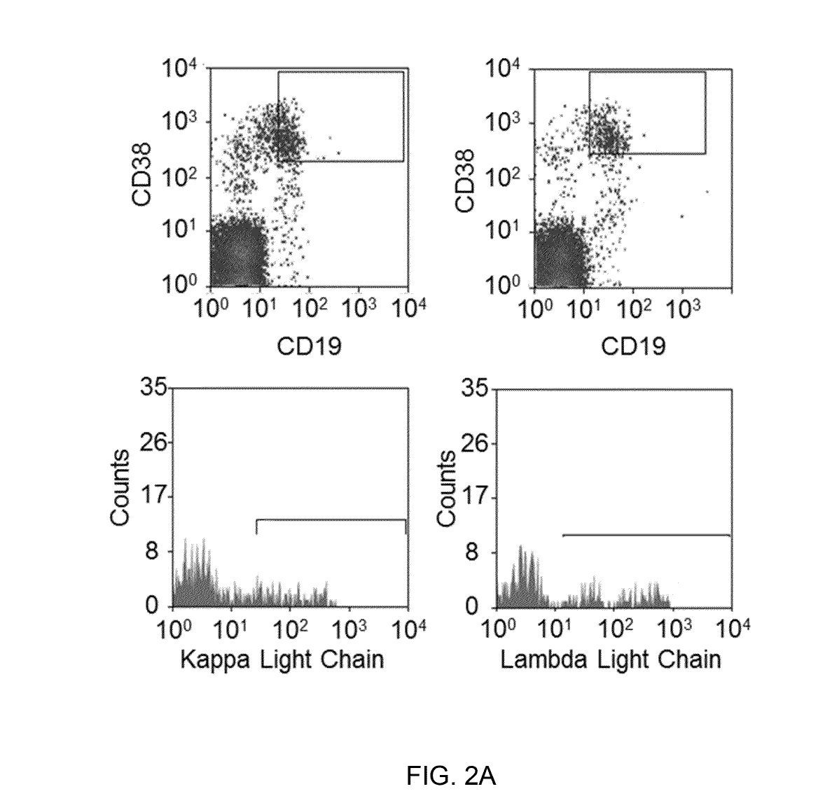

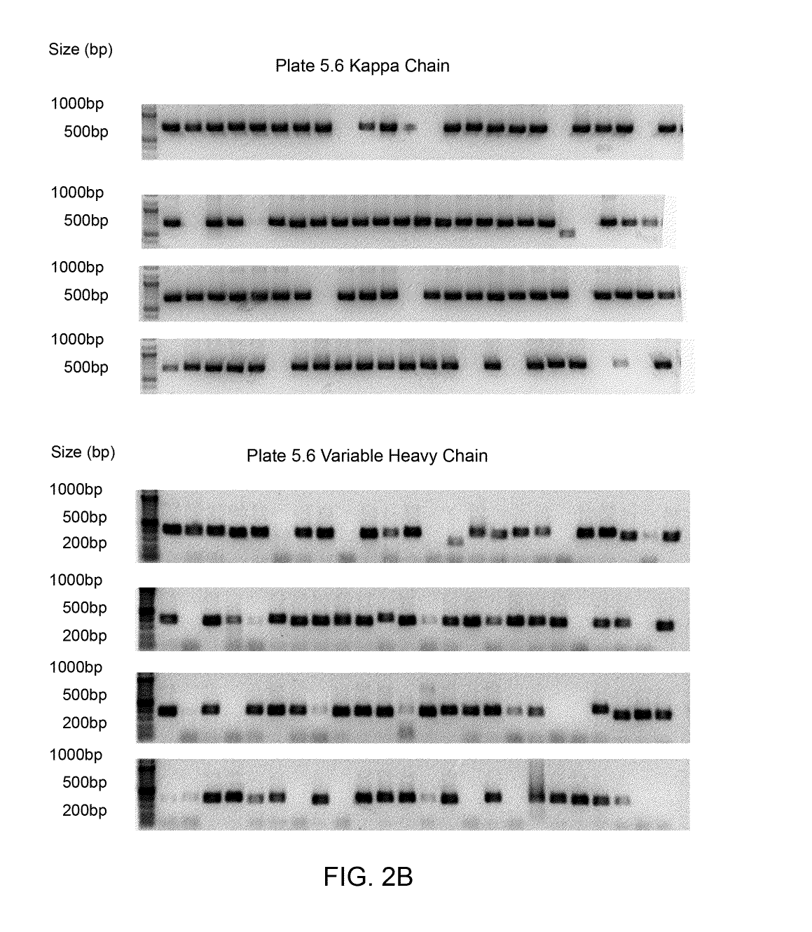

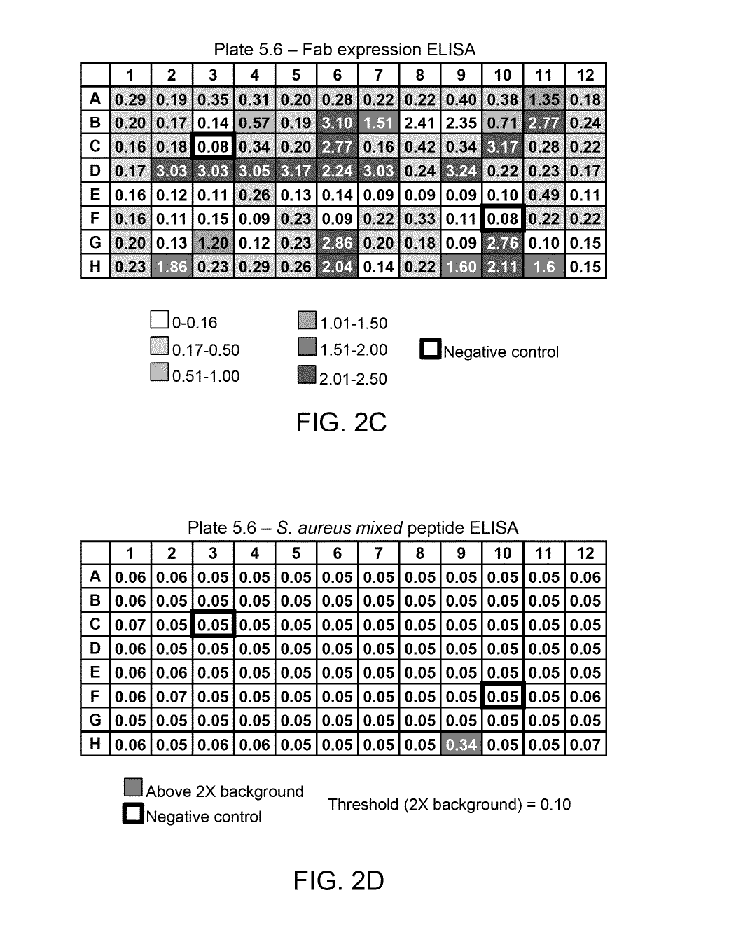

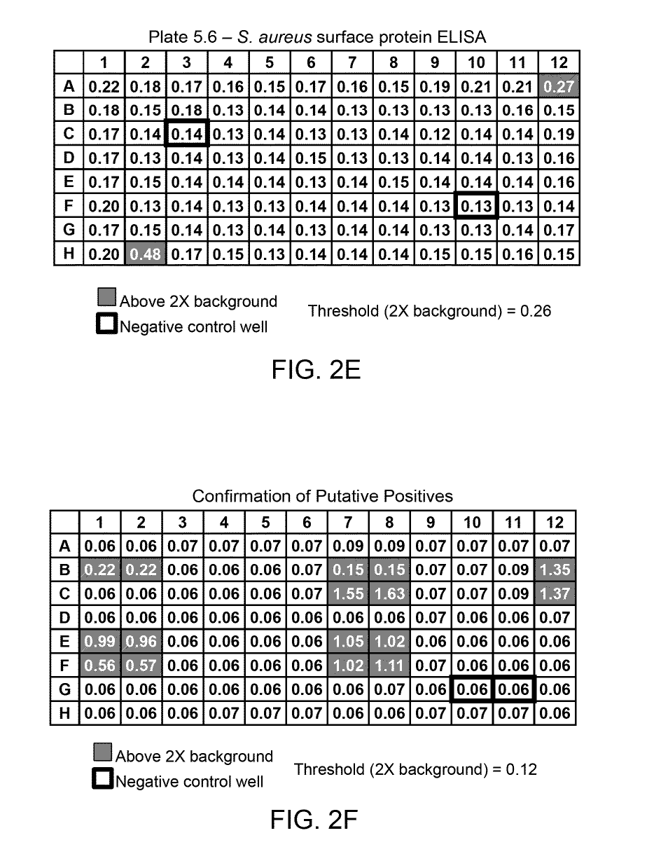

Healthy human subjects were recruited by posters located throughout the Roger Williams Medical Center campus. Blood from qualified subjects was processed through a Ficoll gradient to isolate PBMCs. Antibody-secreting plasma cells, identified by cell surface markers, were single-cell sorted into 96-well plates and subjected to RT-PCR to amplify antibody genes (FIG. 2A). As shown in FIG. 2A, the upper two panels show plasma cells with a clear CD38high/CD19+ population (R3 gate). The lower two panels show the lambda positive and kappa positive plasma cell populations (R4). Cells from the lambda and kappa positive populations were sorted into individual wells of 96-well PCR plates and heavy and light chains were recovered from single cells by RT-PCR with a cognate pair recovery rate of 73% (FIG. 2B). The variation in size between individual amplicons in the heavy chain gel suggests that a single heavy chain is being amplified in each well. Sequence data from cloned antibodies supports this conclusion. Amplified products (FIG. 2B) were cloned and expressed in mammalian cells (CHO-K1 cells) in 96-well format for screening.

Expressed antibodies were screened by ELISA to identify S. aureus-specific binders (FIG. 2C-E). Wells in which an antibody has been detected (values greater than two times background) are shaded, with color indicating relative expression levels with respect to background. Background is calculated as the average value of negative control wells (purple), left empty during sorting. FIG. 2D is a table showing data from plate 5.6 from a screening ELISA against S. aureus peptide antigens. Wells displaying 2× background (average value of purple wells) were scored as positive (e.g., well H9 was scored as positive). FIG. 2E is a table showing data from plate 5.6 from a screening ELISA against S. aureus surface proteins. Wells displaying 2× background (average value of purple wells) were scored as positive (e.g., wells H2 and A12 were scored as positive). Positives were reconfirmed through further ELISA testing (FIG. 2F), sequenced and expressed in a larger scale batch culture for in vitro testing and animal studies. Wells displaying 2× background (average value of purple wells) were scored as positive. Wells B12 and C12 were positive controls. Clones 5.6.H2 (wells B1, B2, B7 and B8) and 5.6.H9 (wells C7, C8) were confirmed as positives in this experiment, as well as several clones from other screening plates. Clone 5.6.A12 did not confirm and was discarded.

### Example 3

### Human Recombinant Polyclonal Anti-S. aureus Antibodies Protect Mice from Infection with Live S. aureus

A human recombinant polyclonal S. aureus antibody was generated by mixing individual antibodies (1.62.B9, 5.11.H10, 5.27.A11, 5.6.H2, and 5.6.H9; referred to herein as five antibody cocktail #1) equally to a total concentration of 1 mg/ml.

Mice were infected with a community associated methicillin-resistant (MRSA) strain, USA300, at a dose of 3.5×10⁸ colony forming units (CFU) or 5×10⁸ CFU, by intra-peritoneal injection. Immediately following infection, groups of 10 mice each were treated by intra-peritoneal injection with either PBS (control population), 110 mg/kg vancomycin, or 1.0 mg/kg of five antibody cocktail #1. Animals were observed at 2, 6, 18, 24, 48 and 72 hours post-treatment and live/dead status was noted.

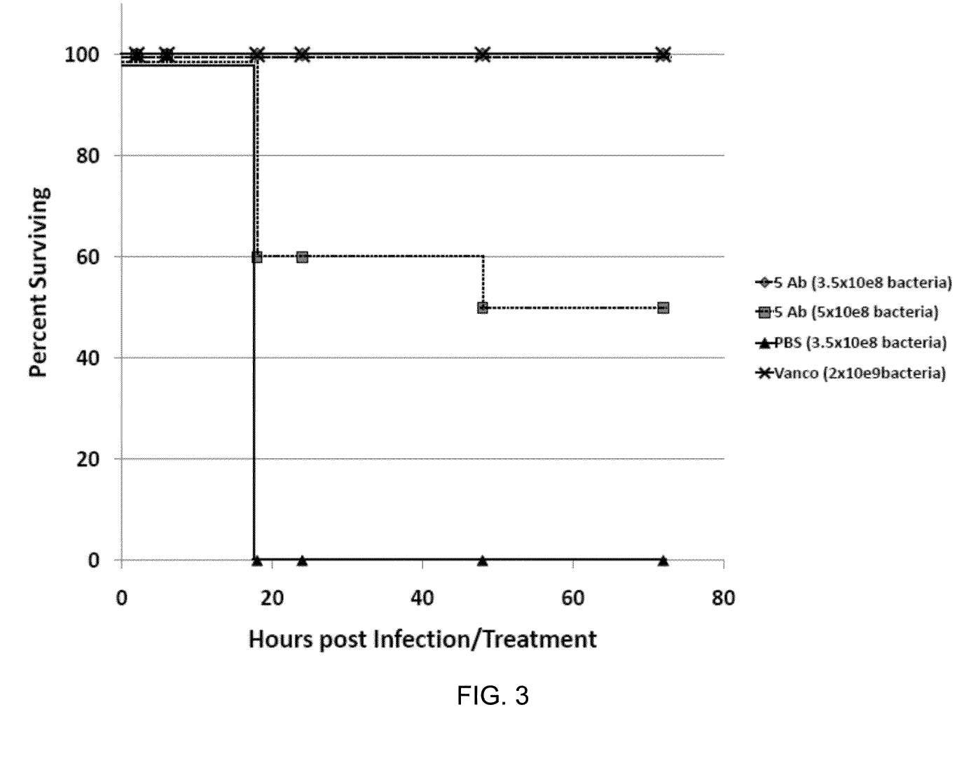

At 3.5×10⁸ CFU, all of the mice treated with five antibody cocktail #1 survived the full 72 hour study. At 5×10⁸ CFU, 50% of the mice treated with five antibody cocktail #1 survived the full 72 hour study (FIG. 3). None of the PBS control mice survived to 72 hours at either dose of S. aureus. Vancomycin, the positive control, rescued all of the mice.

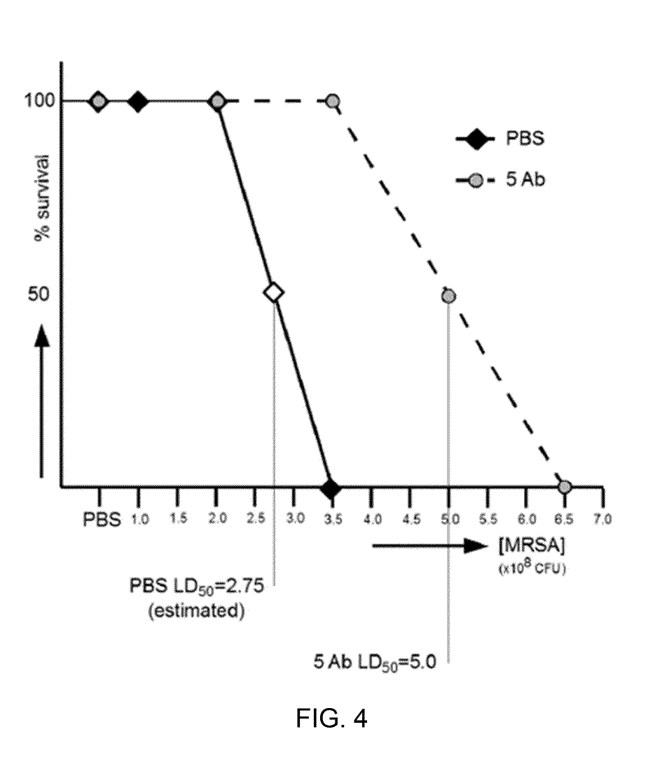

As shown in FIG. 4, the results of this experiment demonstrated that animals treated with PBS alone succumbed rapidly to infection with an LD₅₀ of 2.75×10⁸ CFU. Mice treated with five antibody cocktail #1 at a 1.0 mg/kg dose were fully protected from a dose of 3.5×10⁸ CFU and were more resistant to infection with an LD₅₀ of 5.0×10⁸ CFU, which was approximately twice the LD₅₀ dose for PBS treated animals. This represents a shift in the LD₅₀ curve from 2.75×10⁸ CFU to 5×10⁸ CFU with the five antibody cocktail, which is a significantly stronger effect than has been previously demonstrated with passive immunotherapy approaches. Therefore, the results of this experiment suggest that mice treated with five antibody cocktail #1 are more resistant to S. aureus infection and that fully human recombinant polyclonal antibody therapy is capable of protecting mice from lethal infections of MRSA.

A second human recombinant polyclonal S. aureus antibody was generated by mixing individual antibodies (1.62.B9, 5.11.H10, 5.27.A11, 5.6.H2, 5.7.D3, 5.27.G2, and 8.11.G6; referred to herein as the seven antibody cocktail) equally to a total concentration of 1 mg/ml.

Mice were infected with a methicillin-sensitive (MSSA) strain, Wood46, at a dose of 2×10⁸ colony forming units (CFU) or 4×10⁸ CFU, by intra-peritoneal injection. Immediately following infection, groups of 5 mice each were treated by intra-peritoneal injection with PBS (control population), 110 mg/kg vancomycin, or 1.0 mg/kg of the seven antibody cocktail. Animals were observed at 2, 6, 18, 24, 48 and 72 hours post-treatment and live/dead status was noted.

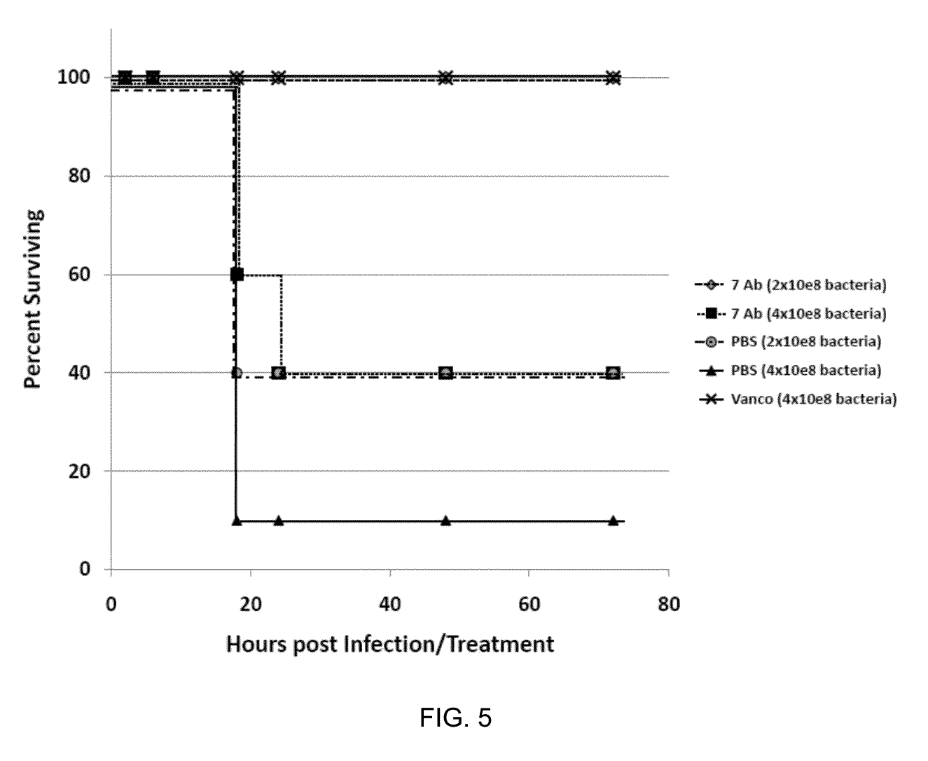

At 2×10⁸ CFU, all of the mice treated with the seven antibody cocktail survived the full 72 hour study, compared with 40% of the PBS control mice. At 4×10⁸ CFU, 40% of the mice treated with the seven antibody cocktail survived the full 72 hour study (FIG. 5), compared with 10% of the PBS control mice. Vancomycin, the positive control, rescued all of the mice.

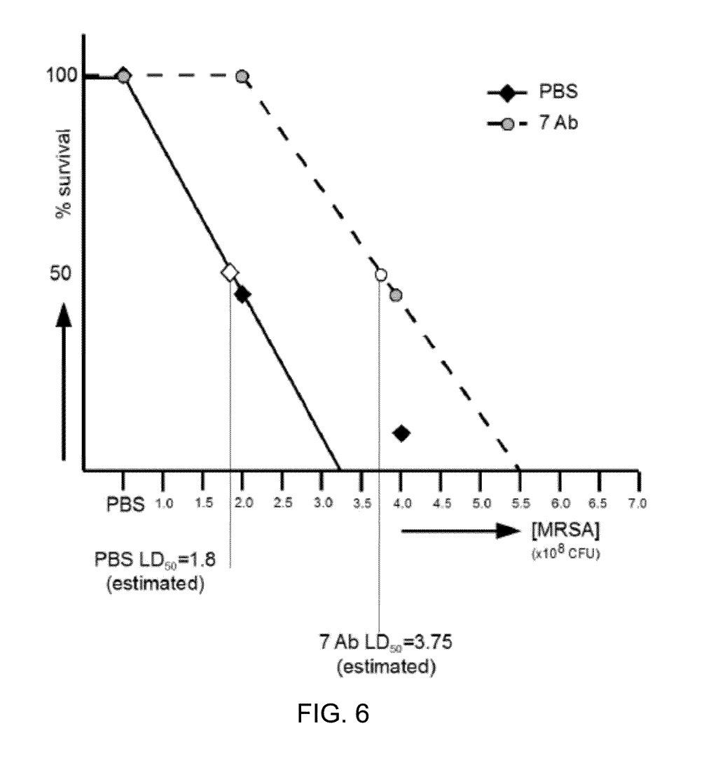

As shown in FIG. 6, the results of this experiment demonstrated that compared with animals treated with PBS alone, mice treated with the seven antibody cocktail at a 1.0 mg/kg dose were fully protected from a dose of 2×10⁸ CFU and were more resistant to infection with a dose of 4.0×10⁸ CFU. This represents a shift in the LD₅₀ curve from 1.8×10⁸ CFU to 3.75×10⁸ CFU with the seven antibody cocktail, which is a significantly stronger effect than has been previously demonstrated with passive immunotherapy approaches. Therefore, the results of this experiment suggest that mice treated with the seven antibody cocktail are more resistant to S. aureus infection and that fully human recombinant polyclonal antibody therapy is capable of protecting mice from lethal infections of MSSA.

In a third study, a comparison was made between two five antibody cocktails, a ten antibody cocktail and a nineteen antibody cocktail. Five antibody cocktail #2 comprised antibodies 5.11.H10, 18.43.D8, 5.23.C9, 5.27.A11 and 5.6.H9. Five antibody cocktail #3 comprised antibodies 22.18.E9, 5.52.H10, 9.51.H9, 8.51.G10 and 5.23.C12. The ten antibody cocktail comprised antibodies 5.11.H10, 18.43.D8, 5.23.C9, 5.27.A11, 5.6.H9, 22.18.E9, 5.52.H10, 9.51.H9, 8.51.G10 and 5.23.C12. The nineteen antibody cocktail comprised antibodies 5.11.H10, 18.43.D8, 5.23.C9, 5.27.A11, 5.6.H9, 22.18.E9, 5.52.H10, 9.51.H9, 8.51.G10, 5.23.C12, 5.24.F3, 5.24.A7, 5.8.H4, 26.51.E1, 5.19.F12, 8.51.G11, 22.22.E7, 22.21.A7, and 5.17.F8. In each cocktail, component antibodies were mixed at equal ratios to a total concentration of 1 mg/ml.

Mice were infected with a community associated methicillin-resistant (MRSA) strain, USA300, at a dose of 1×10⁹ colony forming units (CFU) by intra-peritoneal injection. Immediately following infection, groups of 8 mice each were treated by intra-peritoneal injection with PBS (control population), 10 mg/kg vancomycin, or 1.0 mg/kg of antibody cocktail. Animals were observed at 8, 16, 24, 32, 48, 56 and 72 hours post-treatment and live/dead status was noted.

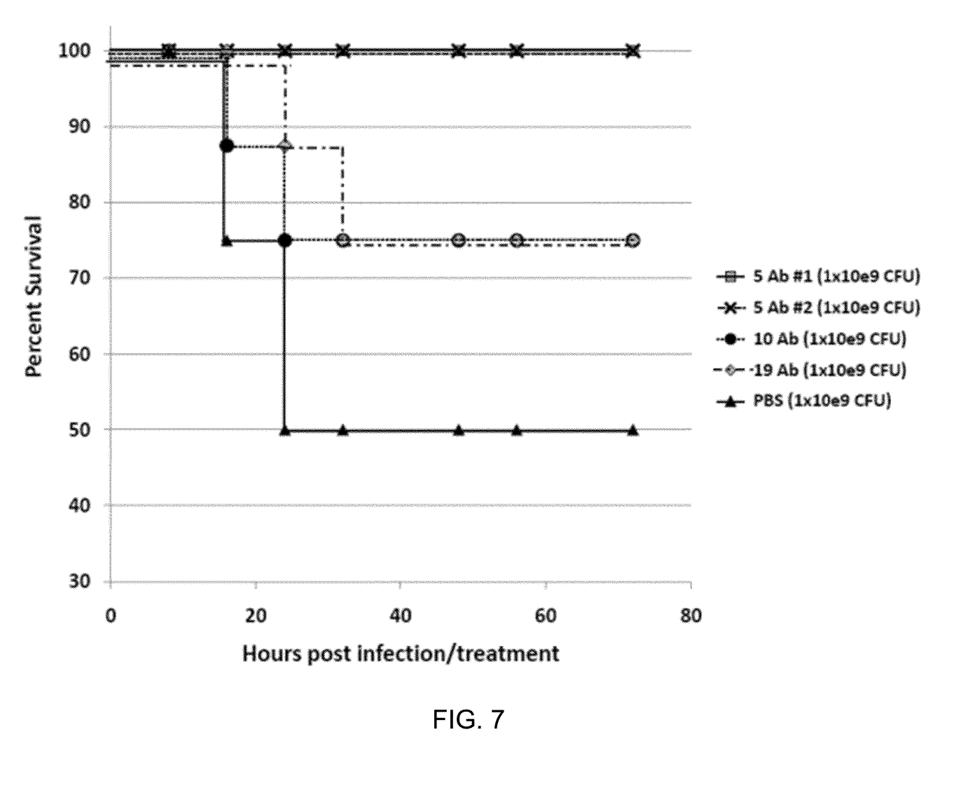

At 1×10⁹ CFU, all of the mice treated with either five antibody cocktail #2 or five antibody cocktail #3 survived the full 72 hour study, compared with 50% of the PBS control mice (FIG. 7). 75% of the mice treated with either the ten antibody cocktail or the nineteen antibody cocktail survived the full 72 hour study. Vancomycin, the positive control, rescued all of the mice.

### Example 4

### Antibody Reactivity Against Various S. aureus Strains

In this example, the antibodies disclosed herein were tested for reactivity against twelve different S. aureus strains grown on Tryptic Soy Agar (TSA) medium. The skilled person would understand that TSA is one or many conditions on which S. aureus may be grown and each conditions may result in the expression of a different proteome.

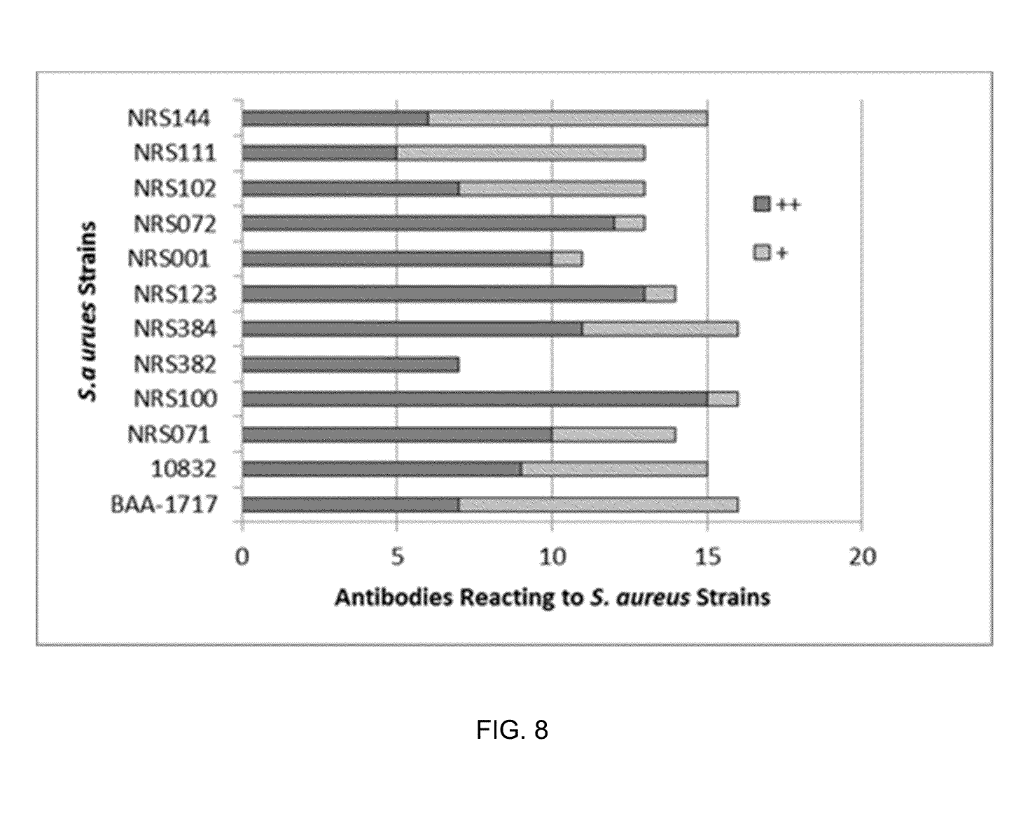

Each bacterial strain was spread on TSA and a single colony from each strain was used to inoculate a 5 ml trypticase soy broth (TSB) culture. The cultures were grown at 37° C. and then diluted before spread plating on TSA plates. The plates were grown overnight before being harvested. The bacteria were washed with PBS. The supernatants were filtered and stored at −20° C. before use. ELISA plates were coated with the supernatants, placed in an azide blocking buffer. The ELISA was run against the 26 different antibody clones as 1:10 diluted Fab fragments both in the presence of a protein A block. Any Fab that was reactive as determined by being sufficiently above background was marked with a (+), any Fab which was highly reactive was designated (++). Any FAb which was not reactive was designated with a (−), as is reflected in Table 4. The number of anti-S. aureus antibodies reacting with various S. aureus strains is shown in FIG. 8

The results suggest that not all antibodies bind each strain of S. aureus. A shown in Table 4, each different strain has a subset of different antibodies which bind. It is contemplated herein that these results reflect the different genomes and proteomes expressed by each strains. Under TSA conditions, antibodies 5.6.H9, 5.55.D2, 22.18A.E9, 9.51.H9, 5.11.H10, 5.23.C12, 5.52.H10, 18.43.D8, 8.51.G10 and 8.51.G11 each reacted with 8 or more S. aureus strains tested.

### Example 5

### In Vitro Opsonization of S. aureus in Human Blood is Augmented by Specific Antibody Cocktails

In this example, the capacity of specific antibody cocktails to enhance the opsonization of S. aureus bacteria by human blood cells was tested.

The following antibody cocktails were tested: antibody cocktail #1 was a five antibody cocktail including antibodies 5.11.H10, 5.23.C9, 5.52.H10, 26.51.E1 and 22.21.A7 at a total concentration of 1.0 mg/ml; antibody cocktail #2 was a five antibody cocktail including antibodies 5.11.H10, 5.27.A11, 5.6.H9, 18.43.D8 and 5.23.C9 at a total concentration of 1.0 mg/ml; and antibody cocktail #3 was a three antibody cocktail including antibodies 5.11.H10, 5.23.C9 and 5.52.H10 at a total concentration of 1.0 mg/ml.

100 μl of antibody (100 μg total) of each of antibody cocktails 1, 2, and 100 μl of S. aureus strain Wood-46 bacteria (1×10⁶ cells) were mixed and incubated for 30 minutes at 37° C. 100 μl of heparinized human blood was added to the antibody/bacteria suspension. The bacteria were counted at time=0 and after a one hour incubation by taking samples from the 300 μl volume and plating serial dilutions on TSA. The percent killing is calculated as [(Bacteria, Time=0 h)−Bacteria, Time=1 h)]/(Bacteria, Time=0)×100%.

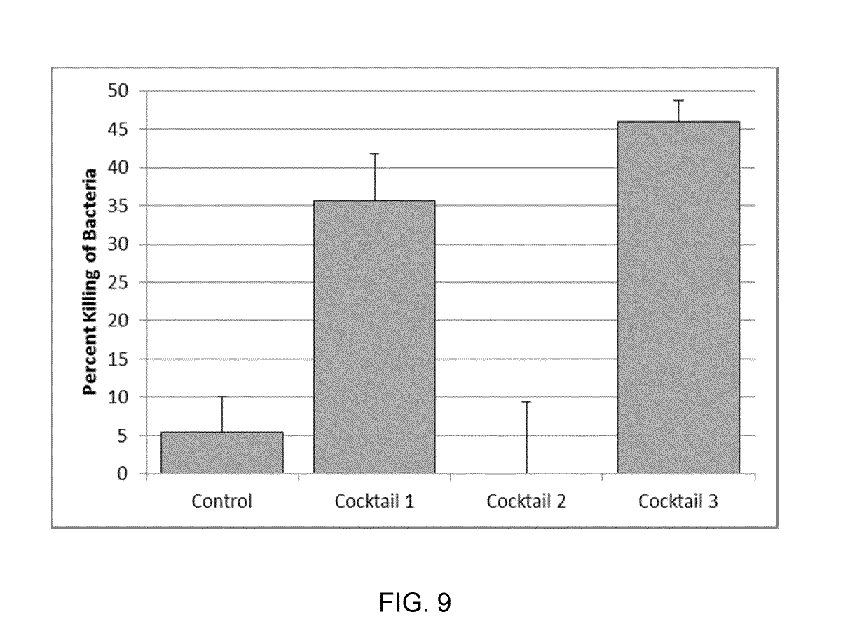

Antibody cocktails #1 and #3 each enhanced the ability of white blood cells to opsonize the S. aureus strain (FIG. 9). Antibody cocktail #2, which had previously been shown to provide protection in mice infected with a dose of 1×10⁹ CFUs of USA300 strain (see Example 3) and contains an antibody against the cell surface (e.g., 5.11.H10), did not enhance opsonization suggesting that opsonization is one part of the criteria needed to provide protection in vivo. The data also suggests that opsonization of S. aureus in human blood is enhanced by polyclonal antibodies, e.g., polyclonal antibodies that contain a mixture of cell surface binding antibodies with other antibodies targeting other cellular mechanisms.

TABLE 4

| | Common | | Anti-S. Aureus Antibodies | | | | | | |
|---|---|---|---|---|---|---|---|---|---|
| Strain | Name | Phenotype | 5.6.H9 | 5.11.H10 | 5.27.A11 | 5.17.F8** | 18.43.D8 | 5.19.F12 | 8.51.G11 |
| BAA-1717* | USA300 | MRSA | ++ | + | + | + | ++ | ++ | ++ |
| 10832* | Wood-46 | MSSA | ++ | ++ | − | − | − | − | − |
| NRS071 | Sanger 252 | HA-MRSA | ++ | ++ | ++ | − | + | − | ++ |
| NRS100 | COL | HA-MRSA | ++ | ++ | ++ | − | ++ | ++ | ++ |
| NRS382 | 626 | HR-MRSA | ++ | − | − | − | − | − | ++ |
| NRS384 | LAC | CA-MRSA | ++ | + | + | − | ++ | ++ | ++ |
| NRS123 | MW2 | CA-MRSA | ++ | ++ | ++ | − | ++ | − | ++ |
| NRS001 | Mu50 | VISA | ++ | ++ | ++ | ++ | ++ | − | − |
| NRS072 | Sanger 476 | CA-MSSA | ++ | ++ | ++ | − | ++ | − | − |
| NRS102 | Reynolds | | ++ | + | − | − | − | − | ++ |
| NRS111 | FRI913 | | ++ | − | − | − | + | − | ++ |
| NRS144 | RN4220 | MSSA | + | ++ | + | − | ++ | + | − |
| | | (Lab Strain) | | | | | | | |

| | Anti-S. Aureus Antibodies | | | | | | | | |
|---|---|---|---|---|---|---|---|---|---|
| Strain | 8.51.G10 | 22.22.E7 | 5.23.C9 | 5.23.C12 | 9.51.H9** | 5.24A.F3 | 5.24A.A7 | 5.8B.H4 | 5.52.H10 |
| BAA-1717* | ++ | − | + | + | ++ | + | − | − | + |
| 10832* | − | + | ++ | ++ | + | − | ++ | ++ | ++ |
| NRS071 | ++ | − | − | + | ++ | ++ | − | − | ++ |
| NRS100 | ++ | − | + | + | ++ | ++ | − | − | ++ |
| NRS382 | ++ | − | − | − | ++ | ++ | − | − | − |
| NRS384 | ++ | − | + | + | ++ | ++ | − | − | ++ |
| NRS123 | ++ | − | − | ++ | ++ | ++ | − | − | ++ |
| NRS001 | − | + | − | ++ | ++ | − | − | − | ++ |
| NRS072 | − | − | − | ++ | ++ | ++ | − | − | ++ |
| NRS102 | ++ | − | ++ | + | + | + | − | − | + |
| NRS111 | ++ | − | − | − | ++ | + | − | − | − |
| NRS144 | − | − | − | ++ | ++ | − | − | − | + |

| | Anti-S. Aureus Antibodies | | | | | | | |
|---|---|---|---|---|---|---|---|---|
| Strain | 22.18A.E9 | 22.21.A7** | 5.15.C1 | 5.54.E6** | 5.55.D2 | 26.51.E1 | 22.14.A1 | 26.53.B4 |
| BAA-1717* | − | − | + | − | + | − | − | − |
| 10832* | − | ++ | + | − | + | − | ++ | + |
| NRS071 | ++ | − | + | − | ++ | − | − | + |
| NRS100 | ++ | − | ++ | − | ++ | − | − | ++ |
| NRS382 | ++ | − | − | − | ++ | − | − | − |
| NRS384 | ++ | − | ++ | − | ++ | − | − | + |
| NRS123 | ++ | − | ++ | − | ++ | − | − | + |
| NRS001 | − | − | ++ | − | − | − | − | ++ |
| NRS072 | ++ | − | ++ | − | ++ | − | − | ++ |
| NRS102 | ++ | − | − | − | ++ | − | ++ | + |
| NRS111 | ++ | − | − | − | − | − | − | − |
| NRS144 | − | − | − | − | − | − | − | ++ |

Strains were obtained from either the America Tissue Culture Collection (ATCC) or from National Repository for Staphylococcus aureus. Strains obtained from the ATCC are designated with an *.
**Antibody expression may be underrepresented in this experiment.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

1.-44. (canceled)

45. A method for treating or preventing Staphylococcus aureus (S. aureus) infection in a mammal, the method comprising administering an effective amount of a recombinant monoclonal antibody mixture, wherein the antibody mixture comprises at least two human antibodies, each of which binds to and neutralizes a S. aureus toxin or antigen conserved among at least three different strains of S. aureus;
and wherein each antibody in the antibody mixture binds an epitope that is not bound by another member of the antibody mixture.

46. The method of claim 45, wherein the mammal is a human.

47. The method of claim 45, wherein the Staphylococcus aureus infection is a methicillin-resistant Staphylococcus aureus (MRSA) infection.

48. The method of claim 45, wherein the Staphylococcus aureus infection is a methicillin-sensitive Staphylococcus aureus (MSSA) infection.

49. The method of claim 45, wherein the Staphylococcus aureus infection is a vancomycin-resistant Staphylococcus aureus (VRSA) infection.

50. The method of claim 45, wherein the Staphylococcus aureus infection is a vancomycin-intermediate Staphylococcus aureus (VISA) infection.

51. (canceled)

52. The method of claim 45, wherein the antibody mixture comprises at least one of the antibodies identified in Table 2.

53. The method of claim 52, wherein the antibody mixture comprises at least 3 of the antibodies identified in Table 2.

54. The method of claim 45, wherein the at least three different strains of S. aureus includes at least one of the strains identified in Table 4.

55. The method of claim 54, wherein the at least three different strains of S. aureus includes at least five of the strains identified in Table 4.

56. The method of claim 45, wherein the antibody mixture comprises a combination of any two antibodies as set forth in Table 1.

57. The method of claim 45, wherein at least one of the antibodies in the antibody mixture binds delta-toxin.

58. The method of claim 45, wherein at least one of the antibodies in the antibody mixture binds toxin phenol soluble modulin beta-1.

59. The method of claim 45, wherein at least one of the antibodies in the antibody mixture is selected from the group consisting of:
(a) an antibody comprising CDR1, CDR2 and CDR3 of heavy chain amino acid sequence SEQ ID NO: 38 and CDR1, CDR2, CDR3 of light chain amino acid sequence SEQ ID NO: 40 of 5.6.H9;
(b) an antibody comprising CDR1, CDR2 and CDR3 of heavy chain amino acid sequence SEQ ID NO: 173 and CDR1, CDR2, CDR3 of light chain amino acid sequence SEQ ID NO: 175 of 22.18A.E9;
(c) an antibody comprising CDR1, CDR2 and CDR3 of heavy chain amino acid sequence SEQ ID NO: 11 and CDR1, CDR2, CDR3 of light chain amino acid sequence SEQ ID NO: 13 of 5.11.H10;
(d) an antibody comprising CDR1, CDR2 and CDR3 of heavy chain amino acid sequence SEQ ID NO: 20 and CDR1, CDR2, CDR3 of light chain amino acid sequence SEQ ID NO: 22 of 5.27.A11;
(e) an antibody comprising CDR1, CDR2 and CDR3 of heavy chain amino acid sequence SEQ ID NO: 47 and CDR1, CDR2, CDR3 of light chain amino acid sequence SEQ ID NO: 49 of 5.17.F8;
(f) an antibody comprising CDR1, CDR2 and CDR3 of heavy chain amino acid sequence SEQ ID NO: 56 and CDR1, CDR2, CDR3 of light chain amino acid sequence SEQ ID NO: 58 of 5.19.F12;
(g) an antibody comprising CDR1, CDR2 and CDR3 of heavy chain amino acid sequence SEQ ID NO: 65 and CDR1, CDR2, CDR3 of light chain amino acid sequence SEQ ID NO: 67 of 5.23.C9;
(h) an antibody comprising CDR1, CDR2 and CDR3 of heavy chain amino acid sequence SEQ ID NO: 74 and CDR1, CDR2, CDR3 of light chain amino acid sequence SEQ ID NO: 76 of 5.23.C12;
(i) an antibody comprising CDR1, CDR2 and CDR3 of heavy chain amino acid sequence SEQ ID NO: 83 and CDR1, CDR2, CDR3 of light chain amino acid sequence SEQ ID NO: 85 of 8.51.G11;
(j) an antibody comprising CDR1, CDR2 and CDR3 of heavy chain amino acid sequence SEQ ID NO: 92 and CDR1, CDR2, CDR3 of light chain amino acid sequence SEQ ID NO: 94 of 9.51.H9;
(k) an antibody comprising CDR1, CDR2 and CDR3 of heavy chain amino acid sequence SEQ ID NO: 101 and CDR1, CDR2, CDR3 of light chain amino acid sequence SEQ ID NO: 103 of 18.43.D8;
(l) an antibody comprising CDR1, CDR2 and CDR3 of heavy chain amino acid sequence SEQ ID NO: 110 and CDR1, CDR2, CDR3 of light chain amino acid sequence SEQ ID NO: 112 of 22.22.E7;
(m) an antibody comprising CDR1, CDR2 and CDR3 of heavy chain amino acid sequence SEQ ID NO: 149 and CDR1, CDR2, CDR3 of light chain amino acid sequence SEQ ID NO: 151 of 8.51.G10;
(n) an antibody comprising CDR1, CDR2 and CDR3 of heavy chain amino acid sequence SEQ ID NO: 153 and CDR1, CDR2, CDR3 of light chain amino acid sequence SEQ ID NO: 155 of 5.24A.A7;
(o) an antibody comprising CDR1, CDR2 and CDR3 of heavy chain amino acid sequence SEQ ID NO: 157 and CDR1, CDR2, CDR3 of light chain amino acid sequence SEQ ID NO: 159 of 5.24A.F3;
(p) an antibody comprising CDR1, CDR2 and CDR3 of heavy chain amino acid sequence SEQ ID NO: 161 and CDR1, CDR2, CDR3 of light chain amino acid sequence SEQ ID NO: 163 of 5.8B.H4;
(q) an antibody comprising CDR1, CDR2 and CDR3 of heavy chain amino acid sequence SEQ ID NO: 165 and CDR1, CDR2, CDR3 of light chain amino acid sequence SEQ ID NO: 167 of 26.51.E1;
(r) an antibody comprising CDR1, CDR2 and CDR3 of heavy chain amino acid sequence SEQ ID NO: 169 and CDR1, CDR2, CDR3 of light chain amino acid sequence SEQ ID NO: 171 of 22.21.A7;
(s) an antibody comprising CDR1, CDR2 and CDR3 of heavy chain amino acid sequence SEQ ID NO: 177 and CDR1, CDR2, CDR3 of light chain amino acid sequence SEQ ID NO: 179 of 5.52.H10;
(t) an antibody comprising CDR1, CDR2 and CDR3 of heavy chain amino acid sequence SEQ ID NO: 181 and CDR1, CDR2, CDR3 of light chain amino acid sequence SEQ ID NO: 183 of 5.15.C1;
(u) an antibody comprising CDR1, CDR2 and CDR3 of heavy chain amino acid sequence SEQ ID NO: 185 and CDR1, CDR2, CDR3 of light chain amino acid sequence SEQ ID NO: 187 of 5.54.E6;
(v) an antibody comprising CDR1, CDR2 and CDR3 of heavy chain amino acid sequence SEQ ID NO: 189 and CDR1, CDR2, CDR3 of light chain amino acid sequence SEQ ID NO: 191 of 5.55.D2;
(w) an antibody comprising CDR1, CDR2 and CDR3 of heavy chain amino acid sequence SEQ ID NO: 193 and CDR1, CDR2, CDR3 of light chain amino acid sequence SEQ ID NO: 195 of 22.14.A1;
(x) an antibody comprising CDR1, CDR2 and CDR3 of heavy chain amino acid sequence SEQ ID NO: 197 and CDR1, CDR2, CDR3 of light chain amino acid sequence SEQ ID NO: 199 of 26.53.B4;
(y) an antibody comprising CDR1, CDR2 and CDR3 of heavy chain amino acid sequence SEQ ID NO: 201 and CDR1, CDR2, CDR3 of light chain amino acid sequence SEQ ID NO: 203 of 5.63.E2;
(z) an antibody comprising CDR1, CDR2 and CDR3 of heavy chain amino acid sequence SEQ ID NO: 205 and CDR1, CDR2, CDR3 of light chain amino acid sequence SEQ ID NO: 207 of 5.64.G4;
(aa) an antibody comprising CDR1, CDR2 and CDR3 of heavy chain amino acid sequence SEQ ID NO: 209 and CDR1, CDR2, CDR3 of light chain amino acid sequence SEQ ID NO: 211 of 43.52.A11;
(ab) an antibody comprising CDR1, CDR2 and CDR3 of heavy chain amino acid sequence SEQ ID NO: 213 and CDR1, CDR2, CDR3 of light chain amino acid sequence SEQ ID NO: 215 of 43.52.E12; and
(ac) an antibody comprising CDR1, CDR2 and CDR3 of heavy chain amino acid sequence SEQ ID NO: 217 and CDR1, CDR2, CDR3 of light chain amino acid sequence SEQ ID NO: 219 of 43.62.E2.

60. The method of claim 45, wherein at least one of the antibodies in the antibody mixture is selected from the group consisting of:
(a) an immunoglobulin heavy chain variable region comprising the amino acid sequence of SEQ ID NO: 11 (5.11.H10), and an immunoglobulin light chain variable region comprising the amino acid sequence of SEQ ID NO: 13 (5.11.H10);
(b) an immunoglobulin heavy chain variable region comprising the amino acid sequence of SEQ ID NO: 20 (5.27.A11), and an immunoglobulin light chain variable region comprising the amino acid sequence of SEQ ID NO: 22 (5.27.A11);
(c) an immunoglobulin heavy chain variable region comprising the amino acid sequence of SEQ ID NO: 38 (5.6.H9), and an immunoglobulin light chain variable region comprising the amino acid sequence of SEQ ID NO: 40 (5.6.H9);
(d) an immunoglobulin heavy chain variable region comprising the amino acid sequence of SEQ ID NO: 47 (5.17.F8), and an immunoglobulin light chain variable region comprising the amino acid sequence of SEQ ID NO: 49 (5.17.F8);
(e) an immunoglobulin heavy chain variable region comprising the amino acid sequence of SEQ ID NO: 56 (5.19.F12), and an immunoglobulin light chain variable region comprising the amino acid sequence of SEQ ID NO: 58 (5.19.F12);
(f) an immunoglobulin heavy chain variable region comprising the amino acid sequence of SEQ ID NO: 65 (5.23.C9), and an immunoglobulin light chain variable region comprising the amino acid sequence of SEQ ID NO: 67 (5.23.C9);
(g) an immunoglobulin heavy chain variable region comprising the amino acid sequence of SEQ ID NO: 74 (5.23.C12), and an immunoglobulin light chain variable region comprising the amino acid sequence of SEQ ID NO: 76 (5.23.C12);
(h) an immunoglobulin heavy chain variable region comprising the amino acid sequence of SEQ ID NO: 83 (8.51.G11), and an immunoglobulin light chain variable region comprising the amino acid sequence of SEQ ID NO: 85 (8.51.G11);
(i) an immunoglobulin heavy chain variable region comprising the amino acid sequence of SEQ ID NO: 92 (9.51.H9), and an immunoglobulin light chain variable region comprising the amino acid sequence of SEQ ID NO: 94 (9.51.H9);
(j) an immunoglobulin heavy chain variable region comprising the amino acid sequence of SEQ ID NO: 101 (18.43.D8), and an immunoglobulin light chain variable region comprising the amino acid sequence of SEQ ID NO: 103 (18.43.D8);
(k) an immunoglobulin heavy chain variable region comprising the amino acid sequence of SEQ ID NO: 110 (22.22.E7), and an immunoglobulin light chain variable region comprising the amino acid sequence of SEQ ID NO: 112 (22.22.E7);
(l) an immunoglobulin heavy chain variable region comprising the amino acid sequence of SEQ ID NO: 149(8.51.G10), and an immunoglobulin light chain variable region comprising the amino acid sequence of SEQ ID NO: 151 (8.51.G10);
(m) an immunoglobulin heavy chain variable region comprising the amino acid sequence of SEQ ID NO: 153 (5.24A.A7), and an immunoglobulin light chain variable region comprising the amino acid sequence of SEQ ID NO: 155 (5.24A.A7);
(n) an immunoglobulin heavy chain variable region comprising the amino acid sequence of SEQ ID NO: 157 (5.24A.F3), and an immunoglobulin light chain variable region comprising the amino acid sequence of SEQ ID NO: 159 (5.24A.F3);
(o) an immunoglobulin heavy chain variable region comprising the amino acid sequence of SEQ ID NO: 161 (5.8B.H4), and an immunoglobulin light chain variable region comprising the amino acid sequence of SEQ ID NO: 163 (5.8B.H4);
(p) an immunoglobulin heavy chain variable region comprising the amino acid sequence of SEQ ID NO: 165 (26.51.E1), and an immunoglobulin light chain variable region comprising the amino acid sequence of SEQ ID NO: 167 (26.51.E1);
(q) an immunoglobulin heavy chain variable region comprising the amino acid sequence of SEQ ID NO: 169 (22.21.A7), and an immunoglobulin light chain variable region comprising the amino acid sequence of SEQ ID NO: 171 (22.21.A7);
(r) an immunoglobulin heavy chain variable region comprising the amino acid sequence of SEQ ID NO: 173 (22.18A.E9), and an immunoglobulin light chain variable region comprising the amino acid sequence of SEQ ID NO: 175 (22.18A.E9);
(s) an immunoglobulin heavy chain variable region comprising the amino acid sequence of SEQ ID NO: 177 (5.52.H10), and an immunoglobulin light chain variable region comprising the amino acid sequence of SEQ ID NO: 179 (5.52.H10);
(t) an immunoglobulin heavy chain variable region comprising the amino acid sequence of SEQ ID NO: 181 (5.15.C1), and an immunoglobulin light chain variable region comprising the amino acid sequence of SEQ ID NO: 191 (5.15.C1);
(u) an immunoglobulin heavy chain variable region comprising the amino acid sequence of SEQ ID NO: 185 (5.54.E6), and an immunoglobulin light chain variable region comprising the amino acid sequence of SEQ ID NO: 187 (5.54.E6);
(v) an immunoglobulin heavy chain variable region comprising the amino acid sequence of SEQ ID NO: 189 (5.55.D2), and an immunoglobulin light chain variable region comprising the amino acid sequence of SEQ ID NO: 191 (5.55.D2);
(w) an immunoglobulin heavy chain variable region comprising the amino acid sequence of SEQ ID NO: 193 (22.14.A1), and an immunoglobulin light chain variable region comprising the amino acid sequence of SEQ ID NO: 195 (22.14.A1); and
(x) an immunoglobulin heavy chain variable region comprising the amino acid sequence of SEQ ID NO: 197 (26.53.B4), and an immunoglobulin light chain variable region comprising the amino acid sequence of SEQ ID NO: 199 (26.53.B4);
(y) an immunoglobulin heavy chain variable region comprising the amino acid sequence of SEQ ID NO: 201 (5.63.E2), and an immunoglobulin light chain variable region comprising the amino acid sequence of SEQ ID NO: 203 (5.63.E2);
(z) an immunoglobulin heavy chain variable region comprising the amino acid sequence of SEQ ID NO: 205 (5.64.G4), and an immunoglobulin light chain variable region comprising the amino acid sequence of SEQ ID NO: 207 (5.64.G4);
(aa) an immunoglobulin heavy chain variable region comprising the amino acid sequence of SEQ ID NO: 209 (43.52.A11), and an immunoglobulin light chain variable region comprising the amino acid sequence of SEQ ID NO: 211 (43.52.A11);
(ab) an immunoglobulin heavy chain variable region comprising the amino acid sequence of SEQ ID NO: 213 (43.52.E12), and an immunoglobulin light chain variable region comprising the amino acid sequence of SEQ ID NO: 215 (43.52.E12); and
(ac) an immunoglobulin heavy chain variable region comprising the amino acid sequence of SEQ ID NO: 217 (43.62.E2), and an immunoglobulin light chain variable region comprising the amino acid sequence of SEQ ID NO: 219 (43.62.E2).

61. An anti-Staphylococcus aureus (S. aureus) antibody or antigen-binding fragment thereof selected from the group consisting of:
(a) an antibody comprising CDR1, CDR2 and CDR3 of heavy chain amino acid sequence SEQ ID NO: 38 and CDR1, CDR2, CDR3 of light chain amino acid sequence SEQ ID NO: 40 of 5.6.H9;
(b) an antibody comprising CDR1, CDR2 and CDR3 of heavy chain amino acid sequence SEQ ID NO: 173 and CDR1, CDR2, CDR3 of light chain amino acid sequence SEQ ID NO: 175 of 22.18A.E9;
(c) an antibody comprising CDR1, CDR2 and CDR3 of heavy chain amino acid sequence SEQ ID NO: 11 and CDR1, CDR2, CDR3 of light chain amino acid sequence SEQ ID NO: 13 of 5.11.H10;
(d) an antibody comprising CDR1, CDR2 and CDR3 of heavy chain amino acid sequence SEQ ID NO: 20 and CDR1, CDR2, CDR3 of light chain amino acid sequence SEQ ID NO: 22 of 5.27.A11;
(e) an antibody comprising CDR1, CDR2 and CDR3 of heavy chain amino acid sequence SEQ ID NO: 47 and CDR1, CDR2, CDR3 of light chain amino acid sequence SEQ ID NO: 49 of 5.17.F8;
(f) an antibody comprising CDR1, CDR2 and CDR3 of heavy chain amino acid sequence SEQ ID NO: 56 and CDR1, CDR2, CDR3 of light chain amino acid sequence SEQ ID NO: 58 of 5.19.F12;
(g) an antibody comprising CDR1, CDR2 and CDR3 of heavy chain amino acid sequence SEQ ID NO: 65 and CDR1, CDR2, CDR3 of light chain amino acid sequence SEQ ID NO: 67 of 5.23.C9;
(h) an antibody comprising CDR1, CDR2 and CDR3 of heavy chain amino acid sequence SEQ ID NO: 74 and CDR1, CDR2, CDR3 of light chain amino acid sequence SEQ ID NO: 76 of 5.23.C12;
(i) an antibody comprising CDR1, CDR2 and CDR3 of heavy chain amino acid sequence SEQ ID NO: 83 and CDR1, CDR2, CDR3 of light chain amino acid sequence SEQ ID NO: 85 of 8.51.G11;
(j) an antibody comprising CDR1, CDR2 and CDR3 of heavy chain amino acid sequence SEQ ID NO: 92 and CDR1, CDR2, CDR3 of light chain amino acid sequence SEQ ID NO: 94 of 9.51.H9;
(k) an antibody comprising CDR1, CDR2 and CDR3 of heavy chain amino acid sequence SEQ ID NO: 101 and CDR1, CDR2, CDR3 of light chain amino acid sequence SEQ ID NO: 103 of 18.43.D8;
(l) an antibody comprising CDR1, CDR2 and CDR3 of heavy chain amino acid sequence SEQ ID NO: 110 and CDR1, CDR2, CDR3 of light chain amino acid sequence SEQ ID NO: 112 of 22.22.E7;
(m) an antibody comprising CDR1, CDR2 and CDR3 of heavy chain amino acid sequence SEQ ID NO: 149 and CDR1, CDR2, CDR3 of light chain amino acid sequence SEQ ID NO: 151 of 8.51.G10;
(n) an antibody comprising CDR1, CDR2 and CDR3 of heavy chain amino acid sequence SEQ ID NO: 153 and CDR1, CDR2, CDR3 of light chain amino acid sequence SEQ ID NO: 155 of 5.24A.A7;
(o) an antibody comprising CDR1, CDR2 and CDR3 of heavy chain amino acid sequence SEQ ID NO: 157 and CDR1, CDR2, CDR3 of light chain amino acid sequence SEQ ID NO: 159 of 5.24A.F3;
(p) an antibody comprising CDR1, CDR2 and CDR3 of heavy chain amino acid sequence SEQ ID NO: 161 and CDR1, CDR2, CDR3 of light chain amino acid sequence SEQ ID NO: 163 of 5.8B.H4;
(q) an antibody comprising CDR1, CDR2 and CDR3 of heavy chain amino acid sequence SEQ ID NO: 165 and CDR1, CDR2, CDR3 of light chain amino acid sequence SEQ ID NO: 167 of 26.51.E1;
(r) an antibody comprising CDR1, CDR2 and CDR3 of heavy chain amino acid sequence SEQ ID NO: 169 and CDR1, CDR2, CDR3 of light chain amino acid sequence SEQ ID NO: 171 of 22.21.A7;
(s) an antibody comprising CDR1, CDR2 and CDR3 of heavy chain amino acid sequence SEQ ID NO: 177 and CDR1, CDR2, CDR3 of light chain amino acid sequence SEQ ID NO: 179 of 5.52.H10;
(t) an antibody comprising CDR1, CDR2 and CDR3 of heavy chain amino acid sequence SEQ ID NO: 181 and CDR1, CDR2, CDR3 of light chain amino acid sequence SEQ ID NO: 183 of 5.15.C1;
(u) an antibody comprising CDR1, CDR2 and CDR3 of heavy chain amino acid sequence SEQ ID NO: 185 and CDR1, CDR2, CDR3 of light chain amino acid sequence SEQ ID NO: 187 of 5.54.E6;
(v) an antibody comprising CDR1, CDR2 and CDR3 of heavy chain amino acid sequence SEQ ID NO: 189 and CDR1, CDR2, CDR3 of light chain amino acid sequence SEQ ID NO: 191 of 5.55.D2;
(w) an antibody comprising CDR1, CDR2 and CDR3 of heavy chain amino acid sequence SEQ ID NO: 193 and CDR1, CDR2, CDR3 of light chain amino acid sequence SEQ ID NO: 195 of 22.14.A1;
(x) an antibody comprising CDR1, CDR2 and CDR3 of heavy chain amino acid sequence SEQ ID NO: 197 and CDR1, CDR2, CDR3 of light chain amino acid sequence SEQ ID NO: 199 of 26.53.B4;
(y) an antibody comprising CDR1, CDR2 and CDR3 of heavy chain amino acid sequence SEQ ID NO: 201 and CDR1, CDR2, CDR3 of light chain amino acid sequence SEQ ID NO: 203 of 5.63.E2;
(z) an antibody comprising CDR1, CDR2 and CDR3 of heavy chain amino acid sequence SEQ ID NO: 205 and CDR1, CDR2, CDR3 of light chain amino acid sequence SEQ ID NO: 207 of 5.64.G4;
(aa) an antibody comprising CDR1, CDR2 and CDR3 of heavy chain amino acid sequence SEQ ID NO: 209 and CDR1, CDR2, CDR3 of light chain amino acid sequence SEQ ID NO: 211 of 43.52.A11;
(ab) an antibody comprising CDR1, CDR2 and CDR3 of heavy chain amino acid sequence SEQ ID NO: 213 and CDR1, CDR2, CDR3 of light chain amino acid sequence SEQ ID NO: 215 of 43.52.E12; and
(ac) an antibody comprising CDR1, CDR2 and CDR3 of heavy chain amino acid sequence SEQ ID NO: 217 and CDR1, CDR2, CDR3 of light chain amino acid sequence SEQ ID NO: 219 of 43.62.E2.

62. An anti-Staphylococcus aureus (S. aureus) human monoclonal antibody selected from the antibodies listed in Table 2.

63. A monoclonal antibody mixture that neutralizes Staphylococcus aureus (S. aureus) or a toxin, cell surface antigen or immunomodulating agent thereof, the mixture comprising at least two antibodies as set forth in Table 1 and combinations thereof.

64. The Method of claim 45, wherein the Staphylococcus aureus infection is an infection resulting from one or more strains of antibiotic-resistant Staphylococcus aureus.


SEQUENCE LISTING (381 sequences)

SEQ ID NO: 1 - DNA, 343 bp, Homo sapiens
gaggtgcagctggtggagtctgggggaggcttggtaaagcctggggggtcccttagactctcctgtgcagcctctggattcactttcagtaacgcctggatgacctgggtccgccaggctccagggaaggggctggagtgggttggccgtattaagagcaaaactgatggtgggacaacagactacgctgcacccgtgaaaggcagattcaccatctcaagagatgattcaataaacacactgtatctgcaaatgaacagcctgaaaaccgaggacacagccgtgtattattgtaggacggggggctactggggccagggaaccctggtcaccgtctcgagtg

SEQ ID NO: 2 - protein, 114 aa, Homo sapiens
EVQLVESGGGLVKPGGSLRLSCAASGFTFSNAWMTWVRQAPGKGLEWVGRIKSKTDGGTTDYAAPVKGRFTISRDDSINTLYLQMNSLKTEDTAVYYCRTGGYWGQGTLVTVSS

SEQ ID NO: 3 - DNA, 328 bp, Homo sapiens
caggctgtggtgactcaggagccctcactgactgtgtccccaggagggacagtcactctcacctgtggctccagcactggagctgtcaccagtggtcattatccctactggttccagcagaagcctggccaagcccccaggacactgatttatgatacaagcaagaaacattcctggacacctgcccggttctcaggctccctccttgggggcaaagctgccctgaccctttcgggtgcgcagcctgaggatgaggctgagtattactgcttggtctccgatagtggtgctcggatcttcggacctgggaccaaggtcaccgtcctag

SEQ ID NO: 4 - protein, 109 aa, Homo sapiens
QAVVTQEPSLTVSPGGTVTLTCGSSTGAVTSGHYPYWFQQKPGQAPRTLIYDTSKKHSWTPARFSGSLLGGKAALTLSGAQPEDEAEYYCLVSDSGARIFGPGTKVTVL

SEQ ID NO: 5 - protein, 8 aa, Homo sapiens
GFTFSNAW

SEQ ID NO: 6 - protein, 10 aa, Homo sapiens
IKSKTDGGTT

SEQ ID NO: 7 - protein, 7 aa, Homo sapiens
CRTGGYW

SEQ ID NO: 8 - protein, 9 aa, Homo sapiens
TGAVTSGHY

SEQ ID NO: 9 - protein, 11 aa, Homo sapiens
CLVSDSGARIF

SEQ ID NO: 10 - DNA, 364 bp, Homo sapiens
caggttcagctggtgcagtctggacctgaaatgaagaagcctggggcctcagtgaaggtctcctgtcaggcctctggttacacctttaccattaatggaattagttgggtgcgacaggcccctggacaacggcctgagtggatggggtggatcagcactagtaatggtcagacaaactttgcacagaatttccagggcagactcaccttgaccacagacacatccacgaccacggccttcatggagttgatgagcctcacatctgccgacacggccgtatactactgtgcgagaaatccatacggcaattcgcgcagattctttgactactggggccagggcaccctggtcaccgtctcgagtg

SEQ ID NO: 11 - protein, 121 aa, Homo sapiens
QVQLVQSGPEMKKPGASVKVSCQASGYTFTINGISWVRQAPGQRPEWMGWISTSNGQTNFAQNFQGRLTLTTDTSTTTAFMELMSLTSADTAVYYCARNPYGNSRRFFDYWGQGTLVTVSS

SEQ ID NO: 12 - DNA, 322 bp, Homo sapiens
gacatccagatgacccagtctccatcctccctgtctgcctctgttggcgacagagtcatcatcacttgccggacaagtcagaccatcaccaattacataaattggtatcagcaaaaaccagggaaagccccgcagctcctgatctatggtgtatccagtttgcaaagtggggtcccatcaaggttcagtggcggtggatctgggacagaattcactctcaccatcagcagtctgcaacctgaagactttgcaacttattactgtcaacagagttatagggcccctcaaacattcggccaagggaccaagctggaaatcaaac

SEQ ID NO: 13 - protein, 107 aa, Homo sapiens
DIQMTQSPSSLSASVGDRVIITCRTSQTITNYINWYQQKPGKAPQLLIYGVSSLQSGVPSRFSGGGSGTEFTLTISSLQPEDFATYYCQQSYRAPQTFGQGTKLEIK

SEQ ID NO: 14 - protein, 8 aa, Homo sapiens
GYTFTING

SEQ ID NO: 15 - protein, 8 aa, Homo sapiens
ISTSNGQT

SEQ ID NO: 16 - protein, 16 aa, Homo sapiens
CARNPYGNSRRFFDYW

SEQ ID NO: 17 - protein, 6 aa, Homo sapiens
QTITNY

SEQ ID NO: 18 - protein, 11 aa, Homo sapiens
CQQSYRAPQTF

SEQ ID NO: 19 - DNA, 352 bp, Homo sapiens
gaggtgcagctggtggagtctgggggaggcttggtgcagccgggggggtctctgagactctcttgtgcagcctctggtttcgccttcagcagccacgagatgcactgggttcgtcaagggataggacacagtttggagtgggtctcagctattggccttgctggtgacacatattattcagactccgtgaagggccgcttcaccatctccagagaaaacgccaagaacactttgtatcttgaaatgaacaatttgagagtcggcgacacggctgtctattactgtgtcaaagaaggggctcattgggaatttgactcctggggccagggaaccctggtcaccgtctcgagtg

SEQ ID NO: 20 - protein, 117 aa, Homo sapiens
EVQLVESGGGLVQPGGSLRLSCAASGFAFSSHEMHWVRQGIGHSLEWVSAIGLAGDTYYSDSVKGRFTISRENAKNTLYLEMNNLRVGDTAVYYCVKEGAHWEFDSWGQGTLVTVSS

SEQ ID NO: 21 - DNA, 328 bp, Homo sapiens
cagtctgtgctgacgcagccgccctcagtgtctggggcccctggacagagggtcatcatctcctgcactgggagcagctccaacctcggggcaagtcttgatgtacactggtaccagcgccttccagggacaggcccccgactcctcatctatgctaacaacaatcggccctcaggggtccctgaccgattctctggctccaagtctggcacctcagcctccctggccatcaccgggctccaggctgaagatgagggtgattattactgccagtcctatgacggcagcctgagggtgttcggcggagggaccaggctgaccgtcctag

SEQ ID NO: 22 - protein, 109 aa, Homo sapiens
QSVLTQPPSVSGAPGQRVIISCTGSSSNLGASLDVHWYQRLPGTGPRLLIYANNNRPSGVPDRFSGSKSGTSASLAITGLQAEDEGDYYCQSYDGSLRVFGGGTRLTVL

SEQ ID NO: 23 - protein, 8 aa, Homo sapiens
GFAFSSHE

SEQ ID NO: 24 - protein, 7 aa, Homo sapiens
IGLAGDT

SEQ ID NO: 25 - protein, 13 aa, Homo sapiens
CVKEGAHWEFDSW

SEQ ID NO: 26 - protein, 9 aa, Homo sapiens
SSNLGASLD

SEQ ID NO: 27 - protein, 11 aa, Homo sapiens [sequence omitted]

SEQ ID NO: 28 - DNA, 379 bp, Homo sapiens
caggtgcagctggtggagtctgggggaggcttgttcaagcctggagggtccctgagactgtcctgtgcagcctctggattcaccttcagtgactactacatgagctggatccggcaggctccagggaaggggctggagtggatttcatacattagtagtagtggtaggaccataaagtacgcagactctgtgaagggccgattcaccatctccagggacaacgccaagaactcactttatctgcaaatgaacagcctgagagccgaggacacggccgtgtattattgtgcgagagatggaggatattgtggaagtgcaaactgccttcatgacgcttttgatatctggggccaagggacaatggtcaccgtctcgagtg

SEQ ID NO: 29 - protein, 126 aa, Homo sapiens
QVQLVESGGGLFKPGGSLRLSCAASGFTFSDYYMSWIRQAPGKGLEWISYISSSGRTIKYADSVKGRFTISRDNAKNSLYLQMNSLRAEDTAVYYCARDGGYCGSANCLHDAFDIWGQGTMVTVSS

SEQ ID NO: 30 - DNA, 319 bp, Homo sapiens
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattcgacgttcggccaagggaccaaggtggaaatcaaac

SEQ ID NO: 31 - protein, 106 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNSYSTFGQGTKVEIK

SEQ ID NO: 32 - protein, 8 aa, Homo sapiens
GFTFSDYY

SEQ ID NO: 33 - protein, 11 aa, Homo sapiens
ISYISSSGRTI

SEQ ID NO: 34 - protein, 21 aa, Homo sapiens
CARDGGYCGSANCLHDAFDIW

SEQ ID NO: 35 - protein, 6 aa, Homo sapiens
QSISSW

SEQ ID NO: 36 - protein, 10 aa, Homo sapiens [sequence omitted]

SEQ ID NO: 37 - DNA, 385 bp, Homo sapiens
caggtcaccttgaaggagtctggtcccacgctggtgaaacccacacagaccctcacgctgacgtgcaccttctctgggttttcgctcagcactagtggagagggtgtggcctggatccgtcaggccccaggaaaggccctggagtggcttgcactcatttattgggatgatgataagcgctacaacccatctctgaagagcaggctcaccatcaccaaggacaactccagaaaccaagtggtccttacaatgaccaacatggaccctgtggacacagccacatattactgtgtacacagggggatgaatagggtttttggggtagtttataattcgggctggttcgacccctggggccagggaaccctggtcaccgtctcgagtg

SEQ ID NO: 38 - protein, 128 aa, Homo sapiens
QVTLKESGPTLVKPTQTLTLTCTFSGFSLSTSGEGVAWIRQAPGKALEWLALIYWDDDKRYNPSLKSRLTITKDNSRNQVVLTMTNMDPVDTATYYCVHRGMNRVFGVVYNSGWFDPWGQGTLVTVSS

SEQ ID NO: 39 - DNA, 325 bp, Homo sapiens
gaaattgtgttgacgcagtctccaggcaccctgtctttgtctccaggggaaagagccaccctctcctgtagggccagtcagagcgttagaagcagccagctagcctggtaccagcagaagcctggccaggctcccagcctcctcatgtatgacgcatctaagagggccactggcatcccagacaggttcagtggcagtgggtctgggacagacttcactctcaccatcagcagactggagcctgaagattttgtagtgtatttctgtcagcagtatgatacatcaccgtggacgttcggccaagggaccaaggtggaaatcaaac

SEQ ID NO: 40 - protein, 108 aa, Homo sapiens
EIVLTQSPGTLSLSPGERATLSCRASQSVRSSQLAWYQQKPGQAPSLLMYDASKRATGIPDRFSGSGSGTDFTLTISRLEPEDFVVYFCQQYDTSPWTFGQGTKVEIK

SEQ ID NO: 41 - protein, 10 aa, Homo sapiens
GFSLSTSGEG

SEQ ID NO: 42 - protein, 7 aa, Homo sapiens
IYWDDDK

SEQ ID NO: 43 - protein, 22 aa, Homo sapiens
CVHRGMNRVFGVVYNSGWFDPW

SEQ ID NO: 44 - protein, 7 aa, Homo sapiens
QSVRSSQ

SEQ ID NO: 45 - protein, 11 aa, Homo sapiens [sequence omitted]

SEQ ID NO: 46 - DNA, 358 bp, Homo sapiens
caggtgcagctacagcagtggggcgcaggactgttgaagccttcggagaccctgtccctcagttgcgctgtctccggtacgtccttcagtgattactactgggcctggatccgtcagcccccagggaaggggctggagtggattggggaaatcaatcagagtggacacactaacgacaacccgtccttcaagagtcgagtcaccatgttagtagacaagtccaagaaccagttctccctgaggatgacttctgtgaccgccgcggacacggctgtatattactgtgcgagatttccttgggtgggacccgagggttttgaccactggggccagggaaccctggtcaccgtctcgagtg

SEQ ID NO: 47 - protein, 119 aa, Homo sapiens
QVQLQQWGAGLLKPSETLSLSCAVSGTSFSDYYWAWIRQPPGKGLEWIGEINQSGHTNDNPSFKSRVTMLVDKSKNQFSLRMTSVTAADTAVYYCARFPWVGPEGFDHWGQGTLVTVSS

SEQ ID NO: 48 - DNA, 382 bp, Homo sapiens
gaaattgtgctgactcagtctccagactttcagtctgggactccaaaggagatggtcaccatcacctgccgggccagtcagaccattggtaacagcttacactggtaccaacagaaaccaggtcagtctccaaagctcctcatcaaatatggttcccagtccatgtcaggggtcccctcgcggttcagtggcagtggatttgggacagatttcaccctgaccatcaacagcctggaagctgaagatgctgcagtgtattactgtcttcagagtggtggttttccgtggacgttcggccaagggaccaaggtggagatcagacgaactgtggctgcaccatctgtcttcatcttcccgccatctgatgagcagttgaaatctg

SEQ ID NO: 49 - protein, 107 aa, Homo sapiens
EIVLTQSPDFQSGTPKEMVTITCRASQTIGNSLHWYQQKPGQSPKLLIKYGSQSMSGVPSRFSGSGFGTDFTLTINSLEAEDAAVYYCLQSGGFPWTFGQGTKVEIR

SEQ ID NO: 50 - protein, 8 aa, Homo sapiens
GTSFSDYY

SEQ ID NO: 51 - protein, 7 aa, Homo sapiens
INQSGHT

SEQ ID NO: 52 - protein, 15 aa, Homo sapiens
CARFPWVGPEGFDHW

SEQ ID NO: 53 - protein, 6 aa, Homo sapiens
QTIGNS

SEQ ID NO: 54 - protein, 11 aa, Homo sapiens
CLQSGGFPWTF

SEQ ID NO: 55 - DNA, 367 bp, Homo sapiens
caggtccagctggtacagtctggggctgagatgaagaagcctggggcctcagtgaaggtctcctgcaagggttccggatccagtctcccttatctttcgatacaatgggtgcgacaggcccctggaaaaggccttgagtggatgggaggctttgatcctgaagacggtgaaacaatctacaacgagaaattcgagggcagaatcaccatggccgaggacacatcgacagacacagcctacatggaactcagcagcctcacatctgacgacacggccgtctattattgcgcaacaagcctcatccgtaacggtctttatgaagcgttcgaacactggggccagggaaccctggtcaccgtctcgagtg

SEQ ID NO: 56 - protein, 122 aa, Homo sapiens
QVQLVQSGAEMKKPGASVKVSCKGSGSSLPYLSIQWVRQAPGKGLEWMGGFDPEDGETIYNEKFEGRITMAEDTSTDTAYMELSSLTSDDTAVYYCATSLIRNGLYEAFEHWGQGTLVTVSS

SEQ ID NO: 57 - DNA, 322 bp, Homo sapiens
gacatccagatgacccagtctccatcctccctgtctgcatctgtaggagacagagtcaccatcacttgccaggcgagtgatgacattaaaaagaatttaaattggtatcaacaaaaaacagggacagcccctaagctcctggtctacgacgcatccaatttggagacaggggtcccatcgaggttccgtggacgtgcatctgggacacaatatactttcaccatcacgaatctgcagcctcaagattctggaatatattactgtcaacaatatgacaatgtcccgctcagttttggcggagggaccaaggtggaggtcagac

SEQ ID NO: 58 - protein, 107 aa, Homo sapiens
DIQMTQSPSSLSASVGDRVTITCQASDDIKKNLNWYQQKTGTAPKLLVYDASNLETGVPSRFRGRASGTQYTFTITNLQPQDSGIYYCQQYDNVPLSFGGGTKVEVR

SEQ ID NO: 59 - protein, 8 aa, Homo sapiens
GSSLPYLS

SEQ ID NO: 60 - protein, 8 aa, Homo sapiens
FDPEDGET

SEQ ID NO: 61 - protein, 17 aa, Homo sapiens
CATSLIRNGLYEAFEHW

SEQ ID NO: 62 - protein, 6 aa, Homo sapiens
DDIKKN

SEQ ID NO: 63 - protein, 11 aa, Homo sapiens [sequence omitted]

SEQ ID NO: 64 - DNA, 382 bp, Homo sapiens
caggtgcagctggtgcagtctggggctgaggtgaagatgcctgagtcctcggtgagggtctcctgcaggatttctggaggcaacttcaaaaattatatttttaactgggtgcgacaggcccccggacaagggcttgagtgggttggaaggatcaacccaaaagttggaaatgaaaatcacatacagagatatgggaacagactcaccctcaccgcggacgattccaggagaacagtttacatggagttgagcagcctgacaacagacgacacggccgtatattattgtgcgacattacgtgttattggtaaccggagactacgaccggtgggcggaatttttgatatctggggccaagggacaatggtcaccgtctcgagtg

SEQ ID NO: 65 - protein, 127 aa, Homo sapiens
QVQLVQSGAEVKMPESSVRVSCRISGGNFKNYIFNWVRQAPGQGLEWVGRINPKVGNENHIQRYGNRLTLTADDSRRTVYMELSSLTTDDTAVYYCATLRVIGNRRLRPVGGIFDIWGQGTMVTVSS

SEQ ID NO: 66 - DNA, 322 bp, Homo sapiens
gacatccagatgacccagtctccatcctccctgtctgcgtctgtgggagacagagtcaccatcacttgccaggcgagtcaggacgtgagtcactatgtaaattggtatcaacaaaaaccagggaaagccccaaaagccctggtctacgaggcatccaatttggaaacaggggtcccatcaagattcagtggaagtggatctgggacacatttcaccttcaccatcaacagcctgcagcctgaagatatcgcgacttatttctgtcaacatgatgcagagctgcctccctctttcggaggagggaccaaggtggagatcaaac

SEQ ID NO: 67 - protein, 107 aa, Homo sapiens
DIQMTQSPSSLSASVGDRVTITCQASQDVSHYVNWYQQKPGKAPKALVYEASNLETGVPSRFSGSGSGTHFTFTINSLQPEDIATYFCQHDAELPPSFGGGTKVEIK

SEQ ID NO: 68 - protein, 8 aa, Homo sapiens
GGNFKNYI

SEQ ID NO: 69 - protein, 8 aa, Homo sapiens
INPKVGNE

SEQ ID NO: 70 - protein, 22 aa, Homo sapiens
CATLRVIGNRRLRPVGGIFDIW

SEQ ID NO: 71 - protein, 6 aa, Homo sapiens
QDVSHY

SEQ ID NO: 72 - protein, 11 aa, Homo sapiens
CQHDAELPPSF

SEQ ID NO: 73 - DNA, 364 bp, Homo sapiens
caggttcagctggtgcagtctggagctgaagtgaagaagcctggggcctcagtgaaggtctcctgcaagtcttctggtcactcctttaccgcctatggcatcagctgggtgcgacaggcccctggacaagggcttgagtggatggggtggatcagcactaacaatggcaacacaaactatagagagaagttccaggacagactctccttgaccacggacacatccacgagcacagtctacatggaactgaggagcctgacatctgacgacacggccgtgtattactgtgcgagaaatccttatggcaattcgcggagattcttagactactggggccagggaaccctggtcaccgtctcgagtg

SEQ ID NO: 74 - protein, 121 aa, Homo sapiens
QVQLVQSGAEVKKPGASVKVSCKSSGHSFTAYGISWVRQAPGQGLEWMGWISTNNGNTNYREKFQDRLSLTTDTSTSTVYMELRSLTSDDTAVYYCARNPYGNSRRFLDYWGQGTLVTVSS

SEQ ID NO: 75 - DNA, 322 bp, Homo sapiens
gacatccagatgacccagtctccatcctccctgtctgcatctgtcggagacagagtcatcatcacttgccgggcaagtcagaacattagcccctatttaaattggtatcagcagaagccagggagagcccctgagctcctgatctatggtacgaccactgtacaaactggggtcccgtcacggttcagtggcattggatctgggacagatttcactctcaccatcagcagtctgcaacctgaagattttgcaacttattactgtcaacagagctacagggcccctcaaacgttcggccaagggaccaaggtggaagtcaagc

SEQ ID NO: 76 - protein, 107 aa, Homo sapiens
DIQMTQSPSSLSASVGDRVIITCRASQNISPYLNWYQQKPGRAPELLIYGTTTVQTGVPSRFSGIGSGTDFTLTISSLQPEDFATYYCQQSYRAPQTFGQGTKVEVK

SEQ ID NO: 77 - protein, 8 aa, Homo sapiens
GHSFTAYG

SEQ ID NO: 78 - protein, 8 aa, Homo sapiens
ISTNNGNT

SEQ ID NO: 79 - protein, 16 aa, Homo sapiens
CARNPYGNSRRFLDYW

SEQ ID NO: 80 - protein, 6 aa, Homo sapiens
QNISPY

SEQ ID NO: 81 - protein, 11 aa, Homo sapiens
CQQSYRAPQTF

SEQ ID NO: 82 - DNA, 376 bp, Homo sapiens
caggtcaccttgaaggagtctggtcctccgctggtgaatcccacagagaccctcacggtgacctgcaatttccctggcttctcattcacctctagtggagagggtgtgggctggatccgtcagcccccaggaaaggccctggaatggcttgctcacatttattgggatggcgataagcggttcaacacatctctgaaaaacagactcaacatcaccaaggacacctctaagaatcaggtggtcctcactttgaccaacgtggcccctgtggacacagccacgtatttctgtgtccacctgacacgcccatccttttggagtccctattacctttttgacctctggggccagggaaccctggtcaccgtctcgagtg

SEQ ID NO: 83 - protein, 125 aa, Homo sapiens
QVTLKESGPPLVNPTETLTVTCNFPGFSFTSSGEGVGWIRQPPGKALEWLAHIYWDGDKRFNTSLKNRLNITKDTSKNQVVLTLTNVAPVDTATYFCVHLTRPSFWSPYYLFDLWGQGTLVTVSS

SEQ ID NO: 84 - DNA, 319 bp, Homo sapiens
gacatccagttgacccagtctccgccctccctgtctgcatctgtcggagacagtgtcaccataacttgccgggcaagtcaatatattagtacgtatttaaattggtatcagcagaaaccggggaaagcccccagactcttgatctatggtgcatccaatttgcacagtggggtctcatcaaggttcagtggcagtagatctgggacagatttcactctcaccataaccagtctacagcctgaagattttgcgacttactactgccaacagagttccactttgtggtcgttcggccaagggaccaaggtggagatgcacc

SEQ ID NO: 85 - protein, 106 aa, Homo sapiens
DIQLTQSPPSLSASVGDSVTITCRASQYISTYLNWYQQKPGKAPRLLIYGASNLHSGVSSRFSGSRSGTDFTLTITSLQPEDFATYYCQQSSTLWSFGQGTKVEMH

SEQ ID NO: 86 - protein, 10 aa, Homo sapiens
GFSFTSSGEG

SEQ ID NO: 87 - protein, 7 aa, Homo sapiens
IYWDGDK

SEQ ID NO: 88 - protein, 19 aa, Homo sapiens
CVHLTRPSFWSPYYLFDLW

SEQ ID NO: 89 - protein, 7 aa, Homo sapiens
QYISTYL

SEQ ID NO: 90 - protein, 10 aa, Homo sapiens
CQQSSTLWSF

SEQ ID NO: 91 - DNA, 358 bp, Homo sapiens
caggttcagctggtgcagtctggaggtgaaatgaggaaacctggggcctcattgaaggtctcctgcaaggcctctaattacaccttcaccagttttggtctgacctggctgcgacaggcccctgggcaaggccctgagtggctgggctggatcagtccttataatggaaacaccaactatgcacagaacttccgcgacagactcaccatgaccacagacacgtccacgagcacagcctacatggagctgaagagcctgacatctgacgacacggccgtctattactgtgccagaactaactgggggaattggtacctcgatctctggggccctggcaccctggtcactgtctcgagtg

SEQ ID NO: 92 - protein, 119 aa, Homo sapiens
QVQLVQSGGEMRKPGASLKVSCKASNYTFTSFGLTWLRQAPGQGPEWLGWISPYNGNTNYAQNFRDRLTMTTDTSTSTAYMELKSLTSDDTAVYYCARTNWGNWYLDLWGPGTLVTVSS

SEQ ID NO: 93 - DNA, 340 bp, Homo sapiens
gacatcgtgatgacccagtctccagactccctggctgtggctctgggcgagcgggccaccatcagctgcaagtccagccagactatcttgttcggctccaacaacaagaactacttacagtggtttcagcagaaacccggacagcctcctaagttactcatttactgggcatctacccgggaatccggggtccctgaccgattcagtggcgacgggtctgggacagacttcactctctccatcgacaacctacaggctgaagatgtggcactttattattgccagcaatattttagtaatccgtatactttcggccaggggaccaaattggagatcaaac

SEQ ID NO: 94 - protein, 113 aa, Homo sapiens
DIVMTQSPDSLAVALGERATISCKSSQTILFGSNNKNYLQWFQQKPGQPPKLLIYWASTRESGVPDRFSGDGSGTDFTLSIDNLQAEDVALYYCQQYFSNPYTFGQGTKLEIK

SEQ ID NO: 95 - protein, 8 aa, Homo sapiens
NYTFTSFG

SEQ ID NO: 96 - protein, 8 aa, Homo sapiens
ISPYNGNT

SEQ ID NO: 97 - protein, 14 aa, Homo sapiens
CARTNWGNWYLDLW

SEQ ID NO: 98 - protein, 12 aa, Homo sapiens
QTILFGSNNKNY

SEQ ID NO: 99 - protein, 11 aa, Homo sapiens
CQQYFSNPYTF

SEQ ID NO: 100 - DNA, 361 bp, Homo sapiens
caggtccagcttgtgcagtctggggctgaggtgaagaagcctggggcctcagtgaaaattgcctgcaagacctctggatacatcttcgttgactatgctgtacaatgggtgcgccaggcccccggacagaggcttgagtggatggggtggctccagtcagccagtggcaagacgagctcgtcacatagtctccaagacagagtctccattacccgggacacatccgcgaacacagcctacttggaactgaggagtctgacttctgaagatacggctgtctactactgtgcgagagggagtataagtcattacgccccctttgactactggggccagggaaccctggtcaccgtctcgagtg

SEQ ID NO: 101 - protein, 120 aa, Homo sapiens
QVQLVQSGAEVKKPGASVKIACKTSGYIFVDYAVQWVRQAPGQRLEWMGWLQSASGKTSSSHSLQDRVSITRDTSANTAYLELRSLTSEDTAVYYCARGSISHYAPFDYWGQGTLVTVSS

SEQ ID NO: 102 - DNA, 340 bp, Homo sapiens
gacatcgtgatgacccagtctccagactccctggctgtgtctctgggcgagagggccaccatcgactgcaagtccagtcagaatattttatatacttccaacaataagagtttcttagcttggtaccaacagaaaccaggacagcctcctaaattgctcatttactgggcgtctacccgagagttcggggtccctgcgcgattcagtggcagcgggtctgggacagatttcactctcaccatcaacaacctgcagggtgaagatgtggccatttattactgtcaacagtatttttctactccatacacttttggccaggggaccaagctggagatcaaac

SEQ ID NO: 103 - protein, 113 aa, Homo sapiens
DIVMTQSPDSLAVSLGERATIDCKSSQNILYTSNNKSFLAWYQQKPGQPPKLLIYWASTREFGVPARFSGSGSGTDFTLTINNLQGEDVAIYYCQQYFSTPYTFGQGTKLEIK

SEQ ID NO: 104 - protein, 8 aa, Homo sapiens
GYIFVDYA

SEQ ID NO: 105 - protein, 8 aa, Homo sapiens
LQSASGKT

SEQ ID NO: 106 - protein, 15 aa, Homo sapiens
CARGSISHYAPFDYW

SEQ ID NO: 107 - protein, 12 aa, Homo sapiens
QNILYTSNNKSF

SEQ ID NO: 108 - protein, 11 aa, Homo sapiens [sequence omitted]

SEQ ID NO: 109 - DNA, 361 bp, Homo sapiens
caggtgcagctgcaggagtcgggcccaggactggtgaagccctcggagaccctgtccctcacgtgcactgtctctggtgcctacatcactaatgactactggagctggatccggcagcccgccgggaagggactggcgtggattgggcgtctctataccagtgggagcaccaactacaacccctccctcaagagtcgagtcaccatgtcagtagacacgtccaaaaaccagttctccctgaggctcacctctgtgaccgccgcggacacggccgtttattattgtgcgagagatgagtgtagtggtggtgggtgcttccatgagaactggggccagggaaccctggtcaccgtctcgagtg

SEQ ID NO: 110 - protein, 120 aa, Homo sapiens
QVQLQESGPGLVKPSETLSLTCTVSGAYITNDYWSWIRQPAGKGLAWIGRLYTSGSTNYNPSLKSRVTMSVDTSKNQFSLRLTSVTAADTAVYYCARDECSGGGCFHENWGQGTLVTVSS

SEQ ID NO: 111 - DNA, 325 bp, Homo sapiens
gaaattgtgttgacgcagtctccaggcaccctgtctttgtctccaggggaaagagccaccctctcctgcagggccagccagagtattagcagcagctacttggcctggtaccagcagaagcgtggccaggctcccaggctcctcatctatggtgcatccagtagggccactggcatcccagacaggttcagtggcagtgggactgggacagacttcactctcaccatcagcagactggagcctgacgattttgcactttattactgtcagcagtatgataggtcaccgctcactttcggcggagggaccaaggtggagatcaaac

SEQ ID NO: 112 - protein, 108 aa, Homo sapiens
EIVLTQSPGTLSLSPGERATLSCRASQSISSSYLAWYQQKRGQAPRLLIYGASSRATGIPDRFSGSGTGTDFTLTISRLEPDDFALYYCQQYDRSPLTFGGGTKVEIK

SEQ ID NO: 113 - protein, 8 aa, Homo sapiens
GAYITNDY

SEQ ID NO: 114 - protein, 7 aa, Homo sapiens
LYTSGST

SEQ ID NO: 115 - protein, 16 aa, Homo sapiens
CARDECSGGGCFHENW

SEQ ID NO: 116 - protein, 7 aa, Homo sapiens
QSISSSY

SEQ ID NO: 117 - protein, 11 aa, Homo sapiens [sequence omitted]

SEQ ID NO: 118 - DNA, 1591 bp, Homo sapiens
cctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagcaggctcagcgctcctgcctggacgcatcccggctatgcagccccagtccagggcagcaaggcaggccccgtctgcctcttcacccggagcctctgcccgccccactcatgctcagggagagggtcttctggctttttcccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtcctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 119 - DNA, 989 bp, Homo sapiens
cctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 120 - protein, 329 aa, Homo sapiens
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - DNA, 1584 bp, Homo sapiens
cctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaaggtgggacccgcggggtatgagggccacatggacagaggccggctcggcccaccctctgccctgggagtgaccgctgtgccaacctctgtccctacagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 122 - DNA, 977 bp, Homo sapiens
cctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 123 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 124 - DNA, 320 bp, Homo sapiens
gaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 125 - protein, 107 aa, Homo sapiens [sequence omitted]

SEQ ID NO: 126 - DNA, 317 bp, Homo sapiens
gtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 127 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 128 - DNA, 1323 bp, Homo sapiens
gaggtgcagctggtggagtctgggggaggcttggtaaagcctggggggtcccttagactctcctgtgcagcctctggattcactttcagtaacgcctggatgacctgggtccgccaggctccagggaaggggctggagtgggttggccgtattaagagcaaaactgatggtgggacaacagactacgctgcacccgtgaaaggcagattcaccatctcaagagatgattcaataaacacactgtatctgcaaatgaacagcctgaaaaccgaggacacagccgtgtattattgtaggacggggggctactggggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 129 - protein, 440 aa, Homo sapiens
EVQLVESGGGLVKPGGSLRLSCAASGFTFSNAWMTWVRQAPGKGLEWVGRIKSKTDGGTTDYAAPVKGRFTISRDDSINTLYLQMNSLKTEDTAVYYCRTGGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 130 - DNA, 645 bp, Homo sapiens
caggctgtggtgactcaggagccctcactgactgtgtccccaggagggacagtcactctcacctgtggctccagcactggagctgtcaccagtggtcattatccctactggttccagcagaagcctggccaagcccccaggacactgatttatgatacaagcaagaaacattcctggacacctgcccggttctcaggctccctccttgggggcaaagctgccctgaccctttcgggtgcgcagcctgaggatgaggctgagtattactgcttggtctccgatagtggtgctcggatcttcggacctgggaccaaggtcaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 131 - protein, 215 aa, Homo sapiens
QAVVTQEPSLTVSPGGTVTLTCGSSTGAVTSGHYPYWFQQKPGQAPRTLIYDTSKKHSWTPARFSGSLLGGKAALTLSGAQPEDEAEYYCLVSDSGARIFGPGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 132 - DNA, 1344 bp, Homo sapiens
caggttcagctggtgcagtctggacctgaaatgaagaagcctggggcctcagtgaaggtctcctgtcaggcctctggttacacctttaccattaatggaattagttgggtgcgacaggcccctggacaacggcctgagtggatggggtggatcagcactagtaatggtcagacaaactttgcacagaatttccagggcagactcaccttgaccacagacacatccacgaccacggccttcatggagttgatgagcctcacatctgccgacacggccgtatactactgtgcgagaaatccatacggcaattcgcgcagattctttgactactggggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 133 - protein, 447 aa, Homo sapiens [sequence omitted]

SEQ ID NO: 134 - DNA, 642 bp, Homo sapiens
gacatccagatgacccagtctccatcctccctgtctgcctctgttggcgacagagtcatcatcacttgccggacaagtcagaccatcaccaattacataaattggtatcagcaaaaaccagggaaagccccgcagctcctgatctatggtgtatccagtttgcaaagtggggtcccatcaaggttcagtggcggtggatctgggacagaattcactctcaccatcagcagtctgcaacctgaagactttgcaacttattactgtcaacagagttatagggcccctcaaacattcggccaagggaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 135 - protein, 214 aa, Homo sapiens
DIQMTQSPSSLSASVGDRVIITCRTSQTITNYINWYQQKPGKAPQLLIYGVSSLQSGVPSRFSGGGSGTEFTLTISSLQPEDFATYYCQQSYRAPQTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - DNA, 1332 bp, Homo sapiens
gaggtgcagctggtggagtctgggggaggcttggtgcagccgggggggtctctgagactctcttgtgcagcctctggtttcgccttcagcagccacgagatgcactgggttcgtcaagggataggacacagtttggagtgggtctcagctattggccttgctggtgacacatattattcagactccgtgaagggccgcttcaccatctccagagaaaacgccaagaacactttgtatcttgaaatgaacaatttgagagtcggcgacacggctgtctattactgtgtcaaagaaggggctcattgggaatttgactcctggggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 137 - protein, 443 aa, Homo sapiens
EVQLVESGGGLVQPGGSLRLSCAASGFAFSSHEMHWVRQGIGHSLEWVSAIGLAGDTYYSDSVKGRFTISRENAKNTLYLEMNNLRVGDTAVYYCVKEGAHWEFDSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 138 - DNA, 645 bp, Homo sapiens
cagtctgtgctgacgcagccgccctcagtgtctggggcccctggacagagggtcatcatctcctgcactgggagcagctccaacctcggggcaagtcttgatgtacactggtaccagcgccttccagggacaggcccccgactcctcatctatgctaacaacaatcggccctcaggggtccctgaccgattctctggctccaagtctggcacctcagcctccctggccatcaccgggctccaggctgaagatgagggtgattattactgccagtcctatgacggcagcctgagggtgttcggcggagggaccaggctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 139 - protein, 215 aa, Homo sapiens
QSVLTQPPSVSGAPGQRVIISCTGSSSNLGASLDVHWYQRLPGTGPRLLIYANNNRPSGVPDRFSGSKSGTSASLAITGLQAEDEGDYYCQSYDGSLRVFGGGTRLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 140 - DNA, 1359 bp, Homo sapiens
caggtgcagctggtggagtctgggggaggcttgttcaagcctggagggtccctgagactgtcctgtgcagcctctggattcaccttcagtgactactacatgagctggatccggcaggctccagggaaggggctggagtggatttcatacattagtagtagtggtaggaccataaagtacgcagactctgtgaagggccgattcaccatctccagggacaacgccaagaactcactttatctgcaaatgaacagcctgagagccgaggacacggccgtgtattattgtgcgagagatggaggatattgtggaagtgcaaactgccttcatgacgcttttgatatctggggccaagggacaatggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 141 - protein, 452 aa, Homo sapiens
QVQLVESGGGLFKPGGSLRLSCAASGFTFSDYYMSWIRQAPGKGLEWISYISSSGRTIKYADSVKGRFTISRDNAKNSLYLQMNSLRAEDTAVYYCARDGGYCGSANCLHDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 142 - DNA, 639 bp, Homo sapiens
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattcgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 143 - protein, 213 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNSYSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - DNA, 1365 bp, Homo sapiens
caggtcaccttgaaggagtctggtcccacgctggtgaaacccacacagaccctcacgctgacgtgcaccttctctgggttttcgctcagcactagtggagagggtgtggcctggatccgtcaggccccaggaaaggccctggagtggcttgcactcatttattgggatgatgataagcgctacaacccatctctgaagagcaggctcaccatcaccaaggacaactccagaaaccaagtggtccttacaatgaccaacatggaccctgtggacacagccacatattactgtgtacacagggggatgaatagggtttttggggtagtttataattcgggctggttcgacccctggggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 145 - protein, 454 aa, Homo sapiens
QVTLKESGPTLVKPTQTLTLTCTFSGFSLSTSGEGVAWIRQAPGKALEWLALIYWDDDKRYNPSLKSRLTITKDNSRNQVVLTMTNMDPVDTATYYCVHRGMNRVFGVVYNSGWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 146 - DNA, 645 bp, Homo sapiens
gaaattgtgttgacgcagtctccaggcaccctgtctttgtctccaggggaaagagccaccctctcctgtagggccagtcagagcgttagaagcagccagctagcctggtaccagcagaagcctggccaggctcccagcctcctcatgtatgacgcatctaagagggccactggcatcccagacaggttcagtggcagtgggtctgggacagacttcactctcaccatcagcagactggagcctgaagattttgtagtgtatttctgtcagcagtatgatacatcaccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 147 - protein, 215 aa, Homo sapiens
EIVLTQSPGTLSLSPGERATLSCRASQSVRSSQLAWYQQKPGQAPSLLMYDASKRATGIPDRFSGSGSGTDFTLTISRLEPEDFVVYFCQQYDTSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - DNA, 376 bp, Homo sapiens
cagatcaccttgaaggagtctggtcctacgctggtgaaccccacacagaccctcacgctgacctgcaccttttctgggttctcattcacctctagtggagaggctgtgggctggatccgtcagcccccaggaaaggccctggaatggcttgctcacatttattgggatgatgatccgcgttatagtacatctctgaagagcagactcaccatcaccaaggacacctccaaaaatcaggtggtcctctctatgaccgacattgaccctgcggacacaggcacatattactgtgcacacctgagacgcccatccctttggagtccttattatctttttgactgctggggccagggaaccctggtcaccgtctcgagtg

SEQ ID NO: 149 - protein, 125 aa, Homo sapiens
QITLKESGPTLVNPTQTLTLTCTFSGFSFTSSGEAVGWIRQPPGKALEWLAHIYWDDDPRYSTSLKSRLTITKDTSKNQVVLSMTDIDPADTGTYYCAHLRRPSLWSPYYLFDCWGQGTLVTVSS

SEQ ID NO: 150 - DNA, 319 bp, Homo sapiens
gacatccagatgacccagtctccatcctccctgtctgcatctgttggagacagagtcaccatcacttgccgggcaagtcagagtattagtagctatttaaattggtatcaactgaaaccaggacaagcccctaagctcctgatctatggtgcatccagtttggaaaatggggtcccttcacggttcagtggcagtggctctgggccagatttcactctcaccattagcagtctgcaacctgaagattttgcaacttactactgtcaacagagttacaaaatgtggtcattcggccaagggaccaaggtggaaatccaac

SEQ ID NO: 151 - protein, 106 aa, Homo sapiens
DIQMTQSPSSLSASVGDRVTITCRASQSISSYLNWYQLKPGQAPKLLIYGASSLENGVPSRFSGSGSGPDFTLTISSLQPEDFATYYCQQSYKMWSFGQGTKVEIQ

SEQ ID NO: 152 - DNA, 355 bp, Homo sapiens
caggttcagctggtgcagtctggagttgaggtgaagaagcctggggcctcagtgaaggtctcctgcaaggcttctggctacacctttaccaactttggcttcagttgggtgcggcaggccccaggacaagggcttgagtggctgggatggatcaacactcataacggtaacacaaactatgcaccgagattccaagacagagtcaccctgaccacagacacatccacgaccacagcctacatggagctgaggagcctgagatctgaggacacggccgtgtattattgtgcgagagatcttgggggaagtagttccgcctactggggacagggaaccctggtcaccgtctcgagtg

SEQ ID NO: 153 - protein, 118 aa, Homo sapiens
QVQLVQSGVEVKKPGASVKVSCKASGYTFTNFGFSWVRQAPGQGLEWLGWINTHNGNTNYAPRFQDRVTLTTDTSTTTAYMELRSLRSEDTAVYYCARDLGGSSSAYWGQGTLVTVSS

SEQ ID NO: 154 - DNA, 325 bp, Homo sapiens
gaaattgtgttgacgcagtctccaggcaccctgtctttgtctccaggggagagagccaccctctcttgcagggccagtcacagtggtaccaacaactacatcgcctggtaccagcagaaacctggccagcctcccaggctcctcatctatggtgcatccagcagggcccctggcatctcagagaggttcagcggcagtgggtctgggacagacttcactctcatcatcagcagactggagcctgaagactttggagtatattactgtgagcagtatgggaactcacctctgacgttcggccaagggaccaaggtggaaatcaaac

SEQ ID NO: 155 - protein, 108 aa, Homo sapiens
EIVLTQSPGTLSLSPGERATLSCRASHSGTNNYIAWYQQKPGQPPRLLIYGASSRAPGISERFSGSGSGTDFTLIISRLEPEDFGVYYCEQYGNSPLTFGQGTKVEIK

SEQ ID NO: 156 - DNA, 376 bp, Homo sapiens
caggtgcagctgcaggagtcgggcccaggactggtggagtcttcggacgtcctgtccctcacctgcactgtctctggtggctccatcagtaatgactattgggcctggatccggcagaccccagggaagggactggagtggattgggtctgtctattacagtggcagaaccagtttcagcccctccctcaagagtcgagtcactatgtcagtggacacgaccaagaaccaattcttcctgaacctgggctctctgaccgttgcggacacggccgtgtattactgtgcgagaactacaaggttgtcctttgactggttagtacactactatggaatggacgtctggggccaggggaccacggtcaccgtctcgagtg

SEQ ID NO: 157 - protein, 125 aa, Homo sapiens
QVQLQESGPGLVESSDVLSLTCTVSGGSISNDYWAWIRQTPGKGLEWIGSVYYSGRTSFSPSLKSRVTMSVDTTKNQFFLNLGSLTVADTAVYYCARTTRLSFDWLVHYYGMDVWGQGTTVTVSS

SEQ ID NO: 158 - DNA, 385 bp, Homo sapiens
gacatccagatgacccagtctccatcgtccctgactgcatctgtaggagacagagtcaccattatttgtcgggcaagtcagcccattggcagctatttgaattggtatcagcagagtccagggaaagcccctaaactcctcatctatgctgcatccactttgcagagtggggtcccatcaaggttcagtggcagtggatctgggacagatttcactctcaacatcagtggtctgcaacctgaagattttgcaacttactactgtcaacagagttacaataaccctttgttcaccttcggccctgggaccaaagtggatatcaaacgaactgtggctgcaccatctgtcttcatcttcccgccatctgatgagcagttgaaatctg

SEQ ID NO: 159 - protein, 108 aa, Homo sapiens
DIQMTQSPSSLTASVGDRVTIICRASQPIGSYLNWYQQSPGKAPKLLIYAASTLQSGVPSRFSGSGSGTDFTLNISGLQPEDFATYYCQQSYNNPLFTFGPGTKVDIK

SEQ ID NO: 160 - DNA, 373 bp, Homo sapiens
gaggtgcagctggtggagtctgggggaagtctggtacggccgggggggtccctgcgactctcctgtgcagcctctggattcatctttgatgattatggcatgagttgggtccgccaagttccaggggagggcctggagtgggtcgccggcattacttgggatggcgtgaacacagattatgcagactccgtgaagggccgcttcaccatctccagagacaacgccaagaactccctgtttctcctaatgaacagtctcagagccgaggacacggccctgtatcactgtgcgagagccagatactaccaaggcactggcctccggtcctacctcgacgagtggggccagggaaccctggtcaccgtctcgagtg

SEQ ID NO: 161 - protein, 124 aa, Homo sapiens
EVQLVESGGSLVRPGGSLRLSCAASGFIFDDYGMSWVRQVPGEGLEWVAGITWDGVNTDYADSVKGRFTISRDNAKNSLFLLMNSLRAEDTALYHCARARYYQGTGLRSYLDEWGQGTLVTVSS

SEQ ID NO: 162 - DNA, 383 bp, Homo sapiens
gacatccagatgacccagtctccatcctccgtgtctgcatctgtaggagaaagagtcactatcacttgtcgggcgactcagggtattgacaactggttggcctggtatcagcataaaccagggaaagccccgaagctcctgatttatgctgcatccactttgcaaagtggggtcccgtccaggttcagcggcagtggatctgggacagatttcactctcaccatcagcagcgtgcagcctgaagatgttgcaacttacttttgtcaacaggctaagaatttccctcggggaggattcactttcggccctgggaccaaagtcgctctcaaacgaactgtggctgcaccatctgtcttcatcttcccgccatctgatgagcagtt

SEQ ID NO: 163 - protein, 110 aa, Homo sapiens
DIQMTQSPSSVSASVGERVTITCRATQGIDNWLAWYQHKPGKAPKLLIYAASTLQSGVPSRFSGSGSGTDFTLTISSVQPEDVATYFCQQAKNFPRGGFTFGPGTKVALK

SEQ ID NO: 164 - DNA, 355 bp, Homo sapiens
caggtgcagctggtggagtctgggggaggcgtggtccagcctgggaggtccctgagactctcctgtgcagcctctggattcaccttcagtaaatttgccatgcactgggtccgccaggctccaggcaaggggctggagtgggtggcagctatagcgtctgatggatataataaatactatgcagactccgtgaagggccaattcaccgtctccagagacacttccaggaacacgctgtatctccaaatggacagcctgagacctgatgacacggctatttattactgtgcgtacggtcacttgtctttttggttcgacccctggggccagggaaccctggtcaccgtctcgagtg

SEQ ID NO: 165 - protein, 118 aa, Homo sapiens
QVQLVESGGGVVQPGRSLRLSCAASGFTFSKFAMHWVRQAPGKGLEWVAAIASDGYNKYYADSVKGQFTVSRDTSRNTLYLQMDSLRPDDTAIYYCAYGHLSFWFDPWGQGTLVTVSS

SEQ ID NO: 166 - DNA, 382 bp, Homo sapiens
gacatccagatgacccagtctccatcctccctgtccgcatctgtaggagacagagtcaccatcacttgccgggcaagtcaggacattggaaatggcttaggctggtatcaacagataccagggaaagcccctaagcgcctgatctatgctgcatcctctttgcaaagtggggtcccatcaaggttcagcggcagtggttctgggacagagttcactctcacaatccacagcctgcagcctgaagattttgcaacttattactgtctacagcataatttttaccctcgcacttttggccaggggaccaagctggagatcaagcgaactgtggctgcaccatctgtcttcatcttcccgccatctgatgagcagttgaaatctg

SEQ ID NO: 167 - protein, 107 aa, Homo sapiens
DIQMTQSPSSLSASVGDRVTITCRASQDIGNGLGWYQQIPGKAPKRLIYAASSLQSGVPSRFSGSGSGTEFTLTIHSLQPEDFATYYCLQHNFYPRTFGQGTKLEIK

SEQ ID NO: 168 - DNA, 367 bp, Homo sapiens
caggtgcagctggtgcagtctggggctgaggtgaagaagcctggggcctcagtgaaggtctcctgcaaggcttctggatacaccttcaccgactactatatacactgggtgcgacaggcccctggacaagggcttgagtggatgggatggatcaacccttatagtggtggcacaaactatgcaccgaagtttcagggcaggttcaccatggccagggacacgtccatcaccacaacctacttggacctgggcaggctgacgtctagcgacacggccatttatttctgtgcgagagatttcttcagagcagcagctggcacggccggagactactggggccagggaaccctggtcaccgtctcgagtg

SEQ ID NO: 169 - protein, 122 aa, Homo sapiens
QVQLVQSGAEVKKPGASVKVSCKASGYTFTDYYIHWVRQAPGQGLEWMGWINPYSGGTNYAPKFQGRFTMARDTSITTTYLDLGRLTSSDTAIYFCARDFFRAAAGTAGDYWGQGTLVTVSS

SEQ ID NO: 170 - DNA, 397 bp, Homo sapiens
gacatcgtgatgacccagtctccagaagactccctggctgtgtctctgggcgagagggccaccatcaactgctccagccagagtgctttatacagccccagcagtaagacctacttagcttggtaccagcagagaccgggacaccctcctaagcttctcatttactgggcatctacccgggtatccggggtccctgaccgattcagtggcagcgggtctgggacagatttcactctcaccatcggcagcctgcaggctgaagatgtggcagtttattactgtcagtactattatggtactgtcactttcggcggagggaccaaggtggagatcaaacgaactgtggctgcaccatctgtcttcatcttcccgccatctgatgagcagttgaaatctg

SEQ ID NO: 171 - protein, 112 aa, Homo sapiens
ADIVMTQSPDSLAVSLGERATINCSSQSALYSPSSKTYLAWYQQRPGHPPKLLIYWASTRVSGVPDRFSGSGSGTDFTLTIGSLQAEDVAVYYCQYYYGTVTFGGGTKVEIK

SEQ ID NO: 172 - DNA, 376 bp, Homo sapiens
caggtgcagctgcaggagtcgggcccaagactggtgaagccttcggagaccctgtccctcacctgcactgtctctggtgggtccatcaatagttattattggagttggatccggcagcccgtcgggaagggactggagtggattgggcgtatgtatcccggtggtacctccagttacaattcatccctcaagagtcgagtcatcatgtcggtagacacgtcgaagaaccagttttctctgaacctcacttctgtgaccgccgcagacacggccgtatatttctgtgcgagagatccaacatatcaccatggcactagtggttatgtggggacttttgatctctggggccaagggacaatggtcaccgtctcgagtg

SEQ ID NO: 173 - protein, 125 aa, Homo sapiens
QVQLQESGPRLVKPSETLSLTCTVSGGSINSYYWSWIRQPVGKGLEWIGRMYPGGTSSYNSSLKSRVIMSVDTSKNQFSLNLTSVTAADTAVYFCARDPTYHHGTSGYVGTFDLWGQGTMVTVSS

SEQ ID NO: 174 - DNA, 385 bp, Homo sapiens
gaaatagtgatgacgcagtctccagccaccctgtctgcgtctccaggggaaagagtcaccctctcctgcagggccagtccgagtgttcgcaacaatttagcctggtaccagcagaaacctggccagcctcctaggctcctcatctatggtgcatccaccaaggtcatgggtactccaaccaggttcagtggcagtgggtctgggacagagttcactctcaccatcagcagcctgcagtctgaagattttgcagtttattactgtcagcagtataatagctggcctccgtacacttttagccaggggaccaaactggagatcaaacgaactgtggctgcaccatctgtcttcatcttcccgccatctgatgagcagttgaaatctg

SEQ ID NO: 175 - protein, 108 aa, Homo sapiens
DIVMTQSPATLSASPGERVTLSCRASPSVRNNLAWYQQKPGQPPRLLIYGASTKVMGTPTRFSGSGSGTEFTLTISSLQSEDFAVYYCQQYNSWPPYTFSQGTKLEIK

SEQ ID NO: 176 - DNA, 346 bp, Homo sapiens
gaggtgcagctggtggagtcggggggaggcctggtcaggcctggggagtccctgagactctcctgtgcaggctctggattcaccttcagcatgtttaccctgaactgggtccgccaggctccagggaagggactggagtgggtcgcatctattagtggtagtagcagtttagtgtttcatgaagactcagtgaagggccgattcaccatctccagagacaacgccaagaactcagtatatctgcaaatgaacagcctgagagccgaagacacggctatttattactgtgtgagaggagatagtcgcgactattggggccagggaaccctggtcaccgtctcgagtg

SEQ ID NO: 177 - protein, 115 aa, Homo sapiens
EVQLVESGGGLVRPGESLRLSCAGSGFTFSMFTLNWVRQAPGKGLEWVASISGSSSLVFHEDSVKGRFTISRDNAKNSVYLQMNSLRAEDTAIYYCVRGDSRDYWGQGTLVTVSS

SEQ ID NO: 178 - DNA, 331 bp, Homo sapiens
cagtctgtgttgacgcagccgccctcagtgtctgcggccccaggacagaaggtcaccatctcctgctctggaaggacctccaatattggaaataaccatgtatcctggtaccagcaattcccaggaacagcccccaaagtcctcatttatcgcaatgataggcgaccctcagggattcctgatcgattctctggctccaagtctgacacgtcagccaccctgggcatcaccgggctccagactggggacgaggccgattattactgcggaacatgggacaacagcctgagtggttgggtgttcggcggagggaccaagctgaccgtcctag

SEQ ID NO: 179 - protein, 110 aa, Homo sapiens
QSVLTQPPSVSAAPGQKVTISCSGRTSNIGNNHVSWYQQFPGTAPKVLIYRNDRRPSGIPDRFSGSKSDTSATLGITGLQTGDEADYYCGTWDNSLSGWVFGGGTKLTVL

SEQ ID NO: 180 - DNA, 379 bp, Homo sapiens
caggtgcagctggtgcagtctggggctgaggtgaagaggcctggggcctcagtgaaggtctcctgtgagacttctggatatagcttcaccactcatgatttgaactgggtgcgacaggccgctggacaggggcttgagtggatgggatggatgaaccctaacactggtgacacatcctacgcacagaggttccagggcagagtcaccatgatcagggacgtctccataagtacagcctacttggaattgacaggcctgacatttgatgacacggccgtctacttctgtgcgaggtcgaattctgcacctgtttcaactcttttacccgctgatgcttttgatgtctggggccaagggacaatggtcaccgtctcgagtg

SEQ ID NO: 181 - protein, 125 aa, Homo sapiens
QVQLVQSGAEVKRPGASVKVSCETSGYSFTTDLNWVRQAAGQGLEWMGWMNPNTGDTSYAQRFQGRVTMIRDVSISTAYLELTGLTFDDTAVYFCARSNSAPVSTLLPADAFDVWGQGTMVTVSS

SEQ ID NO: 182 - DNA, 325 bp, Homo sapiens
gacatccagatgacccagtctccatcctccctgtctgcatctataggagacacagtcaccatcacttgccgggcaagtcagatcattaacagctatttaaattggtatcagcaaaaaccaggggaagcccctaaactcctgatctatgctgtatccaatttgcaccgtggggtcccctcaaggttcagtggcagtgggtctgcggcagatttcactctcactatcagtggtctgcaacctgaagattttgcaacttactactgtcaacagacttacactaccgttgcgatcaccttcggccaagggacacgactggacattaaac

SEQ ID NO: 183 - protein, 108 aa, Homo sapiens
DIQMTQSPSSLSASIGDTVTITCRASQIINSYLNWYQQKPGEAPKLLIYAVSNLHRGVPSRFSGSGSAADFTLTISGLQPEDFATYYCQQTYTTVAITFGQGTRLDIK

SEQ ID NO: 184 - DNA, 373 bp, Homo sapiens
caggtgcagctggtgcagtctggggcggaggtgaagaagcctgggtcctcggtgaacgtctcctgtaaggcctccgcagggaccctcagcggttataccatcaactggttgcgccaggcccctggacgagggtttgagtggctgggaaggatcatccctcaccttggaataggaaacaacgcacataactgggaggccagagtcacccttaccgccgacaagtccacggacactacctacctggagttgcgcgacctgagatctgacgacgtggccatttattactgcgcggtggagttttaccggctggcaacggtcacaaccccaacccttgacttctggggccagggaaccctggtcaccgtctcgagtg

SEQ ID NO: 185 - protein, 122 aa, Homo sapiens
QVQLVQSGAEVKKPGSSVNVSCKASAGTLSGYTINWLRQAPGRGFEWLGRIIPLGIGNNANWEARVTLTADKSTDTTYLELRDLRSDDVAIYYCAVEFYRLATVTTPTLDFWGQGTLVTVSS

SEQ ID NO: 186 - DNA, 325 bp, Homo sapiens
tcctatgtgctgactcagccaccctcggtgtccgtggccccaggaacgacggccaggattacctgtgagggggaccacattggagctaaaagtgtgcactggtaccagaggaagccaggccaggcccctgtgttggtcgtctatatgaatagtgaccggccctcagggatccctgagcgattgtctggctccaactctgggaacacggccaccctgagtatcagcagggtcgaggccgaggatgaggccgactattattgtcaggtttgggatagttttcgcgatcatcaggtattcggcggagggaccaagttgaccgtcctga

SEQ ID NO: 187 - protein, 108 aa, Homo sapiens
SYVLTQPPSVSVAPGTTARITCEGDHIGAKSVHWYQRKPGQAPVLVVYMNSDRPSGIPERLSGSNSGNTATLSISRVEAEDEADYYCQVWDSFRDHQVFGGGTKLTVL

SEQ ID NO: 188 - DNA, 388 bp, Homo sapiens
caggtcaccttgaaggagtctggtcctgctatagtgaaacccacacagaccctcacgctgacctgcaccttctctgggttctcactccgaactagtggagtgggtgtgggctggatccgtcagcccccaggaaaggcccttgagtggcttgcactcatttactgggatgatgataagcgctacaacccatctctgaagaccagactcaccatcaccaaggactcctccagaaaccaggtgttcctcacaatgaccaacatggaccctgtggacacagccacatacttctgtgcacaccgacaacatattacaaacagttttggattggttattgcgaatgatgcttttgacatttggggccaagggacaatggtcaccgtctcgagtg

SEQ ID NO: 189 - protein, 127 aa, Homo sapiens
QVTLKESGPAIVKPTQTLTLTCTFSGFSLRTSGVGVGWIRQPPGKALEWLALIYWDDDKRYNPSLKTRLTITKDSSRNQVFLTMTNMDPVDTATYFCARQITNSFGLVIANDAFDIWGQGTMVTVSS

SEQ ID NO: 190 - DNA, 334 bp, Homo sapiens
cagtctgccctgactcagcctccgtccgcgtccgggtctcctggacagtcagtcaccatctcctgcactggaaccagcagtgacgttggtcgttataattatgtcgcctggtatcaacaacacccaggcagagcccccagactcataatttatggggtcactaagcggccctcaggggtccctgatcgcttctctggctccaagtctggcaacacggcctccctgaccgtctctgggctccaggctgaagatgaggctgattattattgcagttcatatgcaggcagcaactttttggaggtgttcggcggagggaccaagctgaccgtcctag

SEQ ID NO: 191 - protein, 111 aa, Homo sapiens
QSALTQPPSASGSPGQSVTISCTGTSSDVGRYNYVAWYQQHPGRAPRLIIYGVTKRPSGVPDRFSGSKSGNTASLTVSGLQAEDEADYYCSSYAGSNFLEVFGGGTKLTVL

SEQ ID NO: 192 - DNA, 379 bp, Homo sapiens
caggtgcagctggtgcagtctggggctgaggtgaagaagcctgggtcctcggtgaaggtctcctgcaaggcttctggaggcaccttcagcagctatgcaatcaactgggtgcgacaggcccctggacaaggtcttgagtggatgggagggatcatccctatgtttgatacagcaatctacgcacagatgttccagggcagagtcaccatcaccgcggacgaatccacgagcacagcctacatggagctgagcagcctgagatctgacgacacggccgtatattactgtgcgagagatcgaaacgattattatgacagtagtggttattcaggcgcctttgaccagtggggccagggaaccctggtcaccgtctcgagtg

SEQ ID NO: 193 - protein, 126 aa, Homo sapiens
QVQLVQSGAEVKKPGSSVKVSCKASGGTFSSYAINWVRQAPGQGLEWMGGIIPMFDTAIYAQMFQGRVTITADESTSTAYMELSSLRSDDTAVYYCARDRNDYYDSSGYSGAFDQWGQGTLVTVSS

SEQ ID NO: 194 - DNA, 325 bp, Homo sapiens
gaaattgtgttgacgcagtctccaggcgccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcacaatcagcagactggagcctgaggattttgcagtgtattactgtcagcagtatggtagctcacctcgcacttttggccaggggaccaagctggagatcaaac

SEQ ID NO: 195 - protein, 108 aa, Homo sapiens
EIVLTQSPGALSLSPGERATLSCRASQSVSSSYLAWYQQKPGQAPRLLIYGASSRATGIPDRFSGSGSGTDFTLTISRLEPEDFAVYYCQQYGSSPRTFGQGTKLEIK

SEQ ID NO: 196 - DNA, 367 bp, Homo sapiens
gaggtgcagctggtggagtccgggggaggcttggtccagcctggggggtccctgaaactctcctgtgcagcctctgggttcagcttcagtggctctgctgtggaatgggtccgccaggcttccgggagagggctggagtggattggccatataagaaccaagaccaaaaattacgcgacagcgtatgctgcgtcggtgcaaggcaggttcaccatcttcagagatgattcaaggaacacggcgtatctgcagatgaacagtcttaaaaccgaggacacggccgtgtattattgttccagacacatcgaatttagcagttcgtccgggccccagatgggccagggaaccctggtcaccgtctcgagtg

SEQ ID NO: 197 - protein, 120 aa, Homo sapiens
EVQLVESGGGLVQPGGSLKLSCAASGFSFSGSAVEWVRQASGRGLEWIGIRTKTKNYATAYAASVQGRFTIFRDDSRNTAYLQMNSLKTEDTAVYYCSRIEFSSSSGPQMGQGTLVTVSS

SEQ ID NO: 198 - DNA, 322 bp, Homo sapiens
gacatccagatgacccagtctccttccaccctgtctgcatctgtaggcgacacagtcaccatcacttgccaggccagtcagaatattattacttggttggcctggtatcagcagaaaccagggaaagcccctaacatcctcatctataaggcgtctggtttagagagtggggtcccaccgaggttcagcggcagtgggtcggggacagaattcactctcaccatcagcagcctgcagcctgatgatgttgcaacttattattgcctacaatataaaactgagccgtggacgttcggcctagggaccaaggtggagatcagac

SEQ ID NO: 199 - protein, 107 aa, Homo sapiens
DIQMTQSPSTLSASVGDTVTITCQASQNIITWLAWYQQKPGKAPNILIYKASGLESGVPPRFSGSGSGTEFTLTISSLQPDDVATYYCLQYKTEPWTFGLGTKVEIR

SEQ ID NO: 200 - DNA, 346 bp, Homo sapiens
gaggtgcagctggtggagtctgggggaggcttggcccagccgggggagtccctgagactctcctgtgcagcctccggattcacctttagtgcctattggatgagctgggtccgccagactccagggaaggggctggagtgggtggccaacattaaccagggtggagataagacagactatttggactctgtgaagggccgattcaccgtctccagagacaatgccaagaacttagtgtttctgcaaatggccagcctgagagccgacgacacggccgtgtattactgtgggagaggtacaaaccaagactactggggccagggaaccctggtcaccgtctcgagtg

SEQ ID NO: 201 - protein, 115 aa, Homo sapiens
EVQLVESGGGLAQPGESLRLSCAASGFTFSAYWMSWVRQTPGKGLEWVANINQGGDKTDYLDSVKGRFTVSRDNAKNLVFLQMASLRADDTAVYYCGRGTNQDYWGQGTLVTVSS

SEQ ID NO: 202 - DNA, 323 bp, Homo sapiens
cagtctgtgctgactcagccaccctcagcgtctgggacccccgggcagagggtcaccatctcttgttctggaagcagctccaatatcggaagtaataatgtatactggtaccaacaggtcccaggaacggcccccaaatttctcatctatccgaatagtgagcggccctcaggggtccctgaccgattctctgcctccaagtctggcacctcagcctccctggccatcagtggactccggtccgaggatgaggctacttattactgtgcagcatgggatgacagcctaagaacttatgtcttcgcatctgggaccaaggtcac

SEQ ID NO: 203 - protein, 110 aa, Homo sapiens
QSVLTQPPSASGTPGQRVTISCSGSSSNIGSNNVYWYQQVPGTAPKFLIYPNSERPSGVPDRFSASKSGTSASLAISGLRSEDEATYYCAAWDDSLRTYVFASGTKVTVL

SEQ ID NO: 204 - DNA, 361 bp, Homo sapiens
caggtgcagctggtggagtctgggggaggcgtggtccagcctgggaggtccctgagactctcctgtgcagcctccggattcaccctcaggagttatggcatgcactgggtccgccaggctccaggcaagggcctggagtgggtggcagttatatcatatgatggaagtaagacatattatagagactccgtgaagggccgattcaccatctccagagacaattctaagaacacactgtatttgcaaatgaacagcctgcgagttgaggacacggctgtatattattgtgcgaaagaggataatttgggatctgatgtttttgatagatggggccaagggacaatggtcaccgtctcgagtg

SEQ ID NO: 205 - protein, 120 aa, Homo sapiens
QVQLVESGGGVVQPGRSLRLSCAASGFTLRSYGMHWVRQAPGKGLEWVAVISYDGSKTYYRDSVKGRFTISRDNSKNTLYLQMNSLRVEDTAVYYCAKEDNLGSDVFDRWGQGTMVTVSS

SEQ ID NO: 206 - DNA, 322 bp, Homo sapiens
gacatcgtgatgacccagtctccttccaccctgtctgcatctgtaggagacagagttaccatcacttgtcgggccagtcagagtgccagtcagtggttggcctggtatcagcaaaaaccagggaaagcccctaatctcctaatctataaggcgtctaatttacatagtggggtcccatcaaggttcagcggcagtggatctgggaccgaattcactctcaccatcagcagcctacagcctgatgattttgcaacttattactgccaacaatacaacatttatccgtggacgttcggccaagggaccaaggtggaagtgagac

SEQ ID NO: 207 - protein, 107 aa, Homo sapiens
DIVMTQSPSTLSASVGDRVTITCRASQSASQWLAWYQQKPGKAPNLLIYKASNLHSGVPSRFSGSGSGTEFTLTISSLQPDDFATYYCQQYNIYPWTFGQGTKVEVR

SEQ ID NO: 208 - DNA, 361 bp, Homo sapiens
caggtccagctggtgcagtctggggctgaggtgaaggagccagggtcctcggtgagggtctcctgcaaggcttctggacgcatcttccggaattatgctgtcagctgggtgcgacaggcccctggacaagggcttgagtgggtgggccggttcatcccttcggaaggaataacaaactacattccgacattccgggacagactcacctttactgcggacaagtccacgaacacagccttcattgacctgactaacctgaaatatgaggacacggccatatattactgtgtgggtggcaggccaggcgctttcgtcccctttgactcctggggccagggaaccctggtcaccgtctcgagtg

SEQ ID NO: 209 - protein, 120 aa, Homo sapiens
QVQLVQSGAEVKEPGSSVRVSCKASGRIFRNYAVSWVRQAPGQGLEWVGRFIPSEGITNYIPTFRDRLTFTADKSTNTAFIDLTNLKYEDTAIYYCVGGRPGAFVPFDSWGQGTLVTVSS

SEQ ID NO: 210 - DNA, 321 bp, Homo sapiens
gaaatagtgatgacgcagtctcctgccaccctgtctgtgtctccaggggaaagagccaccctctcctgcagggccagtcagagtgtcaggggcaacttagcctggtaccagcaaaaacctggccaggctcccaggcttctcatgtatgatgcttctaccagggccactagtttcccagcccggttcagtggcagtgggtctgggacagagttcatctcaccatcagcagtgtgcagtctgaagattttgcactttattactgtcagcagtataatgactggccccggacttttggccaggggaccaaggtggagatcaaac

SEQ ID NO: 211 - protein, 107 aa, Homo sapiens
EIVMTQSPATLSVSPGERATLSCRASQSVRGNLAWYQQKPGQAPRLLMYDASTRATSFPARFSGSGSGTEFTLTISSVQSEDFALYYCQQYNDWPRTFGQGTKVEIK

SEQ ID NO: 212 - DNA, 373 bp, Homo sapiens
caggtgcagctggtgcagtctggggctgaggtgaagaagcctgggtcctcggtgagggtctcctgcagggtttctggaggcagtttctccagttatgacgtacactgggtgcgacaggcccctggacaagggcttgagtgggtgggaaggagcgtctcacgtgctgacagctcaagtcacacacgggagttcggggacagagtcacgattatcgcggacaaatccacgagtacagtttatttgacactgaccagtctgacctctgacgacacggccatctattactgtgcggcaggggattgtagtgatggcgggtgcttccgcacacccctgcacatctggggccagggcaccctggtcaccgtctcgagtg

SEQ ID NO: 213 - protein, 124 aa, Homo sapiens
QVQLVQSGAEVKKPGSSVRVSCRVSGGSFSSYDVHWVRQAPGQGLEWVGRSVSRADSSSHTREFGDRVTIIADKSTSTVYLTLTSLTSDDTAIYYCAAGDCSDGGCFRTPLHIWGQGTLVTVSS

SEQ ID NO: 214 - DNA, 322 bp, Homo sapiens
gaaatagtgatgacgcagtctccagccactctgtctctgtctccaggggaaagagccaccctctcctgcagggccagtcagagtctgagaaccaacttagcctggtaccagcagcgacctggccaggctcccaggctcctcatctctggggcatccacgagggccactggtatcccagccaggttcagtggcagtgggtctgggacagaattcactctcaccattaggagcctgcagcctgaagattttgcagtttattactgtcaacagtatcatgactggcctcggacgttcggccaagggaccaaggtggatttcaaac

SEQ ID NO: 215 - protein, 107 aa, Homo sapiens
EIVMTQSPATLSLSPGERATLSCRASQSLRTNLAWYQQRPGQAPRLLISGASTRATGIPARFSGSGSGTEFTLTIRSLQPEDFAVYYCQQYHDWPRTFGQGTKVDFK

SEQ ID NO: 216 - DNA, 370 bp, Homo sapiens
gaggtgcagctggtgcagtctggggcagaagtgaaaaaggccggggagtctctgaggatctcctgtaagggttctggatacaactttaatagctattggattggctgggtgcgccagatgcccgggaaaggcctggagtggatggcaatcatctatcctgctgactctgacaggagatatggcccgtccttccaaggccaggtcaccatctcagtcgacaagtctattaacaccgcgtacttgcagtggagcagcctgaaggcctcggacaccgccatgtattattgtgccagactcctcagagttagtactggttgggaagatgcttttgatctgtggggccaagggacaatggtcaccgtctcgagtg

SEQ ID NO: 217 - protein, 123 aa, Homo sapiens
EVQLVQSGAEVKKAGESLRISCKGSGYNFNSYWIGWVRQMPGKGLEWMAIIYPADSDRRYGPSFQGQVTISVDKSINTAYLQWSSLKASDTAMYYCARLLRVSTGWEDAFDLWGQGTMVTVSS

SEQ ID NO: 218 - DNA, 340 bp, Homo sapiens
gacatcgtgatgacccagtctccagactccctggttgtgtctctgggcgagagggccaccatcaagtgccagtccagccagagtgttttgtacagctccaccaataagaactatttagcctggtaccagcagaaatcgggacagccacctaagctactcatttactgggcctccacccgggaatccggggtccctgaccgattcattggtagcggctctaggacagatttcactctcaccatcagcgacctgcagcctgaggatgcggcagtttattactgtcagcagtattatagtgattatctcactttcggcggagggaccaaggtggagatcaaac

SEQ ID NO: 219 - protein, 113 aa, Homo sapiens
DIVMTQSPDSLVVSLGERATIKCQSSQSVLYSSTNKNYLAWYQQKSGQPPKLLIYWASTRESGVPDRFIGSGSRTDFTLTISDLQPEDAAVYYCQQYYSDYLTFGGGTKVEIK

SEQ ID NO: 220 - protein, 10 aa, Homo sapiens
GFSFTSSGEA

SEQ ID NO: 221 - protein, 7 aa, Homo sapiens
IYWDDDP

SEQ ID NO: 222 - protein, 19 aa, Homo sapiens
CAHLRRPSLWSPYYLFDCW

SEQ ID NO: 223 - protein, 5 aa, Homo sapiens
QSISS

SEQ ID NO: 224 - protein, 10 aa, Homo sapiens
CQQSYKMWSF

SEQ ID NO: 225 - protein, 8 aa, Homo sapiens
GYTFTNFG

SEQ ID NO: 226 - protein, 8 aa, Homo sapiens
INTHNGNT

SEQ ID NO: 227 - protein, 17 aa, Homo sapiens
CARDFFRAAAGTAGDYW

SEQ ID NO: 228 - protein, 7 aa, Homo sapiens
HSGTNNY

SEQ ID NO: 229 - protein, 10 aa, Homo sapiens
CQQSYKMWSF

SEQ ID NO: 230 - protein, 8 aa, Homo sapiens
GGSISNDY

SEQ ID NO: 231 - protein, 7 aa, Homo sapiens
VYYSGRT

SEQ ID NO: 232 - protein, 21 aa, Homo sapiens
CARTTRLSFDWLVHYYGMDVW

SEQ ID NO: 233 - protein, 6 aa, Homo sapiens
QPIGSY

SEQ ID NO: 234 - protein, 12 aa, Homo sapiens
CQQSYNNPLFTF

SEQ ID NO: 235 - protein, 8 aa, Homo sapiens
GFIFDDYG

SEQ ID NO: 236 - protein, 8 aa, Homo sapiens
ITWDGVNT

SEQ ID NO: 237 - protein, 19 aa, Homo sapiens
CARARYYQGTGLRSYLDEW

SEQ ID NO: 238 - protein, 6 aa, Homo sapiens
QGIDNW

SEQ ID NO: 239 - protein, 14 aa, Homo sapiens
CQQAKNFPRGGFTF

SEQ ID NO: 240 - protein, 8 aa, Homo sapiens
GFTFSKFA

SEQ ID NO: 241 - protein, 8 aa, Homo sapiens
IASDGYNK

SEQ ID NO: 242 - protein, 13 aa, Homo sapiens
CAYGHLSFWFDPW

SEQ ID NO: 243 - protein, 6 aa, Homo sapiens
QDIGNG

SEQ ID NO: 244 - protein, 11 aa, Homo sapiens
CLQHNFYPRTF

SEQ ID NO: 245 - protein, 8 aa, Homo sapiens
GYTFTDYY

SEQ ID NO: 246 - protein, 8 aa, Homo sapiens
INPYSGGT

SEQ ID NO: 247 - protein, 17 aa, Homo sapiens
CARDFFRAAAGTAGDYW

SEQ ID NO: 248 - protein, 12 aa, Homo sapiens
QSALYSPSSKTY

SEQ ID NO: 249 - protein, 10 aa, Homo sapiens
CQYYYGTVTF

SEQ ID NO: 250 - protein, 8 aa, Homo sapiens
GGSINSYY

SEQ ID NO: 251 - protein, 7 aa, Homo sapiens
MYPGGTS

SEQ ID NO: 252 - protein, 21 aa, Homo sapiens
CARDPTYHHGTSGYVGTFDLW

SEQ ID NO: 253 - protein, 6 aa, Homo sapiens
PSVRNN

SEQ ID NO: 254 - protein, 12 aa, Homo sapiens [sequence omitted]

SEQ ID NO: 255 - protein, 8 aa, Homo sapiens
GFTFSMFT

SEQ ID NO: 256 - protein, 8 aa, Homo sapiens
ISGSSSLV

SEQ ID NO: 257 - protein, 10 aa, Homo sapiens [sequence omitted]

SEQ ID NO: 258 - protein, 8 aa, Homo sapiens
TSNIGNNH

SEQ ID NO: 259 - protein, 13 aa, Homo sapiens [sequence omitted]

SEQ ID NO: 260 - protein, 7 aa, Homo sapiens
GYSFTTD

SEQ ID NO: 261 - protein, 8 aa, Homo sapiens
MNPNTGDT

SEQ ID NO: 262 - protein, 21 aa, Homo sapiens
CARSNSAPVSTLLPADAFDVW

SEQ ID NO: 263 - protein, 6 aa, Homo sapiens
QIINSY

SEQ ID NO: 264 - protein, 12 aa, Homo sapiens
CQQTYTTVAITF

SEQ ID NO: 265 - protein, 8 aa, Homo sapiens
AGTLSGYT

SEQ ID NO: 266 - protein, 7 aa, Homo sapiens
IIPLGIG

SEQ ID NO: 267 - protein, 19 aa, Homo sapiens
CAVEFYRLATVTTPTLDFW

SEQ ID NO: 268 - protein, 6 aa, Homo sapiens
HIGAKS

SEQ ID NO: 269 - protein, 13 aa, Homo sapiens
CQVWDSFRDHQVF

SEQ ID NO: 270 - protein, 10 aa, Homo sapiens
GFSLRTSGVG

SEQ ID NO: 271 - protein, 7 aa, Homo sapiens
IYWDDDK

SEQ ID NO: 272 - protein, 21 aa, Homo sapiens
CARQITNSFGLVIANDAFDIW

SEQ ID NO: 273 - protein, 9 aa, Homo sapiens
SSDVGRYNY

SEQ ID NO: 274 - protein, 13 aa, Homo sapiens
CSSYAGSNFLEVF

SEQ ID NO: 275 - protein, 8 aa, Homo sapiens
GGTFSSYA

SEQ ID NO: 276 - protein, 8 aa, Homo sapiens
IIPMFDTA

SEQ ID NO: 277 - protein, 21 aa, Homo sapiens
CARDRNDYYDSSGYSGAFDQW

SEQ ID NO: 278 - protein, 7 aa, Homo sapiens
QSVSSSY

SEQ ID NO: 279 - protein, 11 aa, Homo sapiens [sequence omitted]

SEQ ID NO: 280 - protein, 8 aa, Homo sapiens
GFSFSGSA

SEQ ID NO: 281 - protein, 10 aa, Homo sapiens
IRTKTKNYAT

SEQ ID NO: 282 - protein, 14 aa, Homo sapiens
CSRIEFSSSSGPQM

SEQ ID NO: 283 - protein, 6 aa, Homo sapiens
QNIITW

SEQ ID NO: 284 - protein, 11 aa, Homo sapiens
CLQYKTEPWTF

SEQ ID NO: 285 - protein, 8 aa, Homo sapiens
GFTFSAYW

SEQ ID NO: 286 - protein, 8 aa, Homo sapiens
INQGGDKT

SEQ ID NO: 287 - protein, 10 aa, Homo sapiens
CGRGTNQDYW

SEQ ID NO: 288 - protein, 8 aa, Homo sapiens
SSNIGSNN

SEQ ID NO: 289 - protein, 13 aa, Homo sapiens
CAAWDDSLRTYVF

SEQ ID NO: 290 - protein, 8 aa, Homo sapiens
GFTLRSYG

SEQ ID NO: 291 - protein, 8 aa, Homo sapiens
ISYDGSKT

SEQ ID NO: 292 - protein, 15 aa, Homo sapiens
CAKEDNLGSDVFDRW

SEQ ID NO: 293 - protein, 6 aa, Homo sapiens
QSASQW

SEQ ID NO: 294 - protein, 11 aa, Homo sapiens [sequence omitted]

SEQ ID NO: 295 - protein, 8 aa, Homo sapiens
GRIFRNYA

SEQ ID NO: 296 - protein, 8 aa, Homo sapiens
FIPSEGIT

SEQ ID NO: 297 - protein, 15 aa, Homo sapiens
CVGGRPGAFVPFDSW

SEQ ID NO: 298 - protein, 6 aa, Homo sapiens
QSVRGN

SEQ ID NO: 299 - protein, 11 aa, Homo sapiens [sequence omitted]

SEQ ID NO: 300 - protein, 8 aa, Homo sapiens
GGSFSSYD

SEQ ID NO: 301 - protein, 8 aa, Homo sapiens
SVSRADSS

SEQ ID NO: 302 - protein, 19 aa, Homo sapiens
CAAGDCSDGGCFRTPLHIW

SEQ ID NO: 303 - protein, 6 aa, Homo sapiens
QSLRTN

SEQ ID NO: 304 - protein, 11 aa, Homo sapiens [sequence omitted]

SEQ ID NO: 305 - protein, 8 aa, Homo sapiens
GYNFNSYW

SEQ ID NO: 306 - protein, 8 aa, Homo sapiens
IYPADSDR

SEQ ID NO: 307 - protein, 18 aa, Homo sapiens
CARLLRVSTGWEDAFDLW

SEQ ID NO: 308 - protein, 12 aa, Homo sapiens
QSVLYSSTNKNY

SEQ ID NO: 309 - protein, 11 aa, Homo sapiens
CQQYYSDYLTF

SEQ ID NO: 310 - DNA, 1353 bp, Homo sapiens
cagatcaccttgaaggagtctggtcctacgctggtgaaccccacacagaccctcacgctgacctgcaccttttctgggttctcattcacctctagtggagaggctgtgggctggatccgtcagcccccaggaaaggccctggaatggcttgctcacatttattgggatgatgatccgcgttatagtacatctctgaagagcagactcaccatcaccaaggacacctccaaaaatcaggtggtcctctctatgaccgacattgaccctgcggacacaggcacatattactgtgcacacctgagacgcccatccctttggagtccttattatctttttgactgctggggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 311 - protein, 450 aa, Homo sapiens
QITLKESGPTLVNPTQTLTLTCTFSGFSFTSSGEAVGWIRQPPGKALEWLAHIYWDDDPRYSTSLKSRLTITKDTSKNQVVLSMTDIDPADTGTYYCAHLRRPSLWSPYYLFDCWGQGTLVTVSSSTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 312 - DNA, 639 bp, Homo sapiens
gacatccagatgacccagtctccatcctccctgtctgcatctgttggagacagagtcaccatcacttgccgggcaagtcagagtattagtagctatttaaattggtatcaactgaaaccaggacaagcccctaagctcctgatctatggtgcatccagtttggaaaatggggtcccttcacggttcagtggcagtggctctgggccagatttcactctcaccattagcagtctgcaacctgaagattttgcaacttactactgtcaacagagttacaaaatgtggtcattcggccaagggaccaaggtggaaatccaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 313 - protein, 213 aa, Homo sapiens
DIQMTQSPSSLSASVGDRVTITCRASQSISSYLNWYQLKPGQAPKLLIYGASSLENGVPSRFSGSGSGPDFTLTISSLQPEDFATYYCQQSYKMWSFGQGTKVEIQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 314 - DNA, 1332 bp, Homo sapiens
caggttcagctggtgcagtctggagttgaggtgaagaagcctggggcctcagtgaaggtctcctgcaaggcttctggctacacctttaccaactttggcttcagttgggtgcggcaggccccaggacaagggcttgagtggctgggatggatcaacactcataacggtaacacaaactatgcaccgagattccaagacagagtcaccctgaccacagacacatccacgaccacagcctacatggagctgaggagcctgagatctgaggacacggccgtgtattattgtgcgagagatcttgggggaagtagttccgcctactggggacagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 315 - protein, 443 aa, Homo sapiens
QVQLVQSGVEVKKPGASVKVSCKASGYTFTNFGFSWVRQAPGQGLEWLGWINTHNGNTNYAPRFQDRVTLTTDTSTTTAYMELRSLRSEDTAVYYCARDLGGSSSAYWGQGTLVTVSSSTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 316 - DNA, 645 bp, Homo sapiens
gaaattgtgttgacgcagtctccaggcaccctgtctttgtctccaggggagagagccaccctctcttgcagggccagtcacagtggtaccaacaactacatcgcctggtaccagcagaaacctggccagcctcccaggctcctcatctatggtgcatccagcagggcccctggcatctcagagaggttcagcggcagtgggtctgggacagacttcactctcatcatcagcagactggagcctgaagactttggagtatattactgtgagcagtatgggaactcacctctgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 317 - protein, 215 aa, Homo sapiens
EIVLTQSPGTLSLSPGERATLSCRASHSGTNNYIAWYQQKPGQPPRLLIYGASSRAPGISERFSGSGSGTDFTLIISRLEPEDFGVYYCEQYGNSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 318 - DNA, 1353 bp, Homo sapiens
caggtgcagctgcaggagtcgggcccaggactggtggagtcttcggacgtcctgtccctcacctgcactgtctctggtggctccatcagtaatgactattgggcctggatccggcagaccccagggaagggactggagtggattgggtctgtctattacagtggcagaaccagtttcagcccctccctcaagagtcgagtcactatgtcagtggacacgaccaagaaccaattcttcctgaacctgggctctctgaccgttgcggacacggccgtgtattactgtgcgagaactacaaggttgtcctttgactggttagtacactactatggaatggacgtctggggccaggggaccacggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 319 - protein, 450 aa, Homo sapiens
QVQLQESGPGLVESSDVLSLTCTVSGGSISNDYWAWIRQTPGKGLEWIGSVYYSGRTSFSPSLKSRVTMSVDTTKNQFFLNLGSLTVADTAVYYCARTTRLSFDWLVHYYGMDVWGQGTTVTVSSSTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 320 - DNA, 645 bp, Homo sapiens
gacatccagatgacccagtctccatcgtccctgactgcatctgtaggagacagagtcaccattatttgtcgggcaagtcagcccattggcagctatttgaattggtatcagcagagtccagggaaagcccctaaactcctcatctatgctgcatccactttgcagagtggggtcccatcaaggttcagtggcagtggatctgggacagatttcactctcaacatcagtggtctgcaacctgaagattttgcaacttactactgtcaacagagttacaataaccctttgttcaccttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 321 - protein, 215 aa, Homo sapiens
DIQMTQSPSSLTASVGDRVTIICRASQPIGSYLNWYQQSPGKAPKLLIYAASTLQSGVPSRFSGSGSGTDFTLNISGLQPEDFATYYCQQSYNNPLFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 322 - DNA, 1350 bp, Homo sapiens
gaggtgcagctggtggagtctgggggaagtctggtacggccgggggggtccctgcgactctcctgtgcagcctctggattcatctttgatgattatggcatgagttgggtccgccaagttccaggggagggcctggagtgggtcgccggcattacttgggatggcgtgaacacagattatgcagactccgtgaagggccgcttcaccatctccagagacaacgccaagaactccctgtttctcctaatgaacagtctcagagccgaggacacggccctgtatcactgtgcgagagccagatactaccaaggcactggcctccggtcctacctcgacgagtggggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 323 - protein, 449 aa, Homo sapiens
EVQLVESGGSLVRPGGSLRLSCAASGFIFDDYGMSWVRQVPGEGLEWVAGITWDGVNTDYADSVKGRFTISRDNAKNSLFLLMNSLRAEDTALYHCARARYYQGTGLRSYLDEWGQGTLVTVSSSTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 324 - DNA, 651 bp, Homo sapiens
gacatccagatgacccagtctccatcctccgtgtctgcatctgtaggagaaagagtcactatcacttgtcgggcgactcagggtattgacaactggttggcctggtatcagcataaaccagggaaagccccgaagctcctgatttatgctgcatccactttgcaaagtggggtcccgtccaggttcagcggcagtggatctgggacagatttcactctcaccatcagcagcgtgcagcctgaagatgttgcaacttacttttgtcaacaggctaagaatttccctcggggaggattcactttcggccctgggaccaaagtcgctctcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 325 - protein, 217 aa, Homo sapiens
DIQMTQSPSSVSASVGERVTITCRATQGIDNWLAWYQHKPGKAPKLLIYAASTLQSGVPSRFSGSGSGTDFTLTISSVQPEDVATYFCQQAKNFPRGGFTFGPGTKVALKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 326 - DNA, 1332 bp, Homo sapiens
caggtgcagctggtggagtctgggggaggcgtggtccagcctgggaggtccctgagactctcctgtgcagcctctggattcaccttcagtaaatttgccatgcactgggtccgccaggctccaggcaaggggctggagtgggtggcagctatagcgtctgatggatataataaatactatgcagactccgtgaagggccaattcaccgtctccagagacacttccaggaacacgctgtatctccaaatggacagcctgagacctgatgacacggctatttattactgtgcgtacggtcacttgtctttttggttcgacccctggggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 327 - protein, 443 aa, Homo sapiens
QVQLVESGGGVVQPGRSLRLSCAASGFTFSKFAMHWVRQAPGKGLEWVAAIASDGYNKYYADSVKGQFTVSRDTSRNTLYLQMDSLRPDDTAIYYCAYGHLSFWFDPWGQGTLVTVSSSTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 328 - DNA, 642 bp, Homo sapiens
gacatccagatgacccagtctccatcctccctgtccgcatctgtaggagacagagtcaccatcacttgccgggcaagtcaggacattggaaatggcttaggctggtatcaacagataccagggaaagcccctaagcgcctgatctatgctgcatcctctttgcaaagtggggtcccatcaaggttcagcggcagtggttctgggacagagttcactctcacaatccacagcctgcagcctgaagattttgcaacttattactgtctacagcataatttttaccctcgcacttttggccaggggaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 329 - protein, 214 aa, Homo sapiens
DIQMTQSPSSLSASVGDRVTITCRASQDIGNGLGWYQQIPGKAPKRLIYAASSLQSGVPSRFSGSGSGTEFTLTIHSLQPEDFATYYCLQHNFYPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 330 - DNA, 1344 bp, Homo sapiens
caggtgcagctggtgcagtctggggctgaggtgaagaagcctggggcctcagtgaaggtctcctgcaaggcttctggatacaccttcaccgactactatatacactgggtgcgacaggcccctggacaagggcttgagtggatgggatggatcaacccttatagtggtggcacaaactatgcaccgaagtttcagggcaggttcaccatggccagggacacgtccatcaccacaacctacttggacctgggcaggctgacgtctagcgacacggccatttatttctgtgcgagagatttcttcagagcagcagctggcacggccggagactactggggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 331 - protein, 447 aa, Homo sapiens
QVQLVQSGAEVKKPGASVKVSCKASGYTFTDYYIHWVRQAPGQGLEWMGWINPYSGGTNYAPKFQGRFTMARDTSITTTYLDLGRLTSSDTAIYFCARDFFRAAAGTAGDYWGQGTLVTVSSSTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 332 - DNA, 657 bp, Homo sapiens
gacatcgtgatgacccagtctccagaagactccctggctgtgtctctgggcgagagggccaccatcaactgctccagccagagtgctttatacagccccagcagtaagacctacttagcttggtaccagcagagaccgggacaccctcctaagcttctcatttactgggcatctacccgggtatccggggtccctgaccgattcagtggcagcgggtctgggacagatttcactctcaccatcggcagcctgcaggctgaagatgtggcagtttattactgtcagtactattatggtactgtcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 333 - protein, 219 aa, Homo sapiens
ADIVMTQSPDSLAVSLGERATINCSSQSALYSPSSKTYLAWYQQRPGHPPKLLIYWASTRVSGVPDRFSGSGSGTDFTLTIGSLQAEDVAVYYCQYYYGTVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - DNA, 1353 bp, Homo sapiens
caggtgcagctgcaggagtcgggcccaagactggtgaagccttcggagaccctgtccctcacctgcactgtctctggtgggtccatcaatagttattattggagttggatccggcagcccgtcgggaagggactggagtggattgggcgtatgtatcccggtggtacctccagttacaattcatccctcaagagtcgagtcatcatgtcggtagacacgtcgaagaaccagttttctctgaacctcacttctgtgaccgccgcagacacggccgtatatttctgtgcgagagatccaacatatcaccatggcactagtggttatgtggggacttttgatctctggggccaagggacaatggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 335 - protein, 450 aa, Homo sapiens
QVQLQESGPRLVKPSETLSLTCTVSGGSINSYYWSWIRQPVGKGLEWIGRMYPGGTSSYNSSLKSRVIMSVDTSKNQFSLNLTSVTAADTAVYFCARDPTYHHGTSGYVGTFDLWGQGTMVTVSSSTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 336 - DNA, 645 bp, Homo sapiens
gaaatagtgatgacgcagtctccagccaccctgtctgcgtctccaggggaaagagtcaccctctcctgcagggccagtccgagtgttcgcaacaatttagcctggtaccagcagaaacctggccagcctcctaggctcctcatctatggtgcatccaccaaggtcatgggtactccaaccaggttcagtggcagtgggtctgggacagagttcactctcaccatcagcagcctgcagtctgaagattttgcagtttattactgtcagcagtataatagctggcctccgtacacttttagccaggggaccaaactggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 337 - protein, 215 aa, Homo sapiens
DIVMTQSPATLSASPGERVTLSCRASPSVRNNLAWYQQKPGQPPRLLIYGASTKVMGTPTRFSGSGSGTEFTLTISSLQSEDFAVYYCQQYNSWPPYTFSQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 338 - DNA, 1323 bp, Homo sapiens
gaggtgcagctggtggagtcggggggaggcctggtcaggcctggggagtccctgagactctcctgtgcaggctctggattcaccttcagcatgtttaccctgaactgggtccgccaggctccagggaagggactggagtgggtcgcatctattagtggtagtagcagtttagtgtttcatgaagactcagtgaagggccgattcaccatctccagagacaacgccaagaactcagtatatctgcaaatgaacagcctgagagccgaagacacggctatttattactgtgtgagaggagatagtcgcgactattggggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 339 - protein, 440 aa, Homo sapiens
EVQLVESGGGLVRPGESLRLSCAGSGFTFSMFTLNWVRQAPGKGLEWVASISGSSSLVFHEDSVKGRFTISRDNAKNSVYLQMNSLRAEDTAIYYCVRGDSRDYWGQGTLVTVSSSTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 340 - DNA, 648 bp, Homo sapiens
cagtctgtgttgacgcagccgccctcagtgtctgcggccccaggacagaaggtcaccatctcctgctctggaaggacctccaatattggaaataaccatgtatcctggtaccagcaattcccaggaacagcccccaaagtcctcatttatcgcaatgataggcgaccctcagggattcctgatcgattctctggctccaagtctgacacgtcagccaccctgggcatcaccgggctccagactggggacgaggccgattattactgcggaacatgggacaacagcctgagtggttgggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagtcgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctgcagaatgctca

SEQ ID NO: 341 - protein, 216 aa, Homo sapiens
QSVLTQPPSVSAAPGQKVTISCSGRTSNIGNNHVSWYQQFPGTAPKVLIYRNDRRPSGIPDRFSGSKSDTSATLGITGLQTGDEADYYCGTWDNSLSGWVFGGGTKLTVLGQPKAAPSVTLFPPSSEELQANKATLVCLISDFYPGVVTVAWKADSSPVKAGVETTTPSKQSNNKYAASSYLSLTPEQWKSHKSYSCQVTHEGSTVEKTVAPAECS

SEQ ID NO: 342 - DNA, 1356 bp, Homo sapiens
caggtgcagctggtgcagtctggggctgaggtgaagaggcctggggcctcagtgaaggtctcctgtgagacttctggatatagcttcaccactcatgatttgaactgggtgcgacaggccgctggacaggggcttgagtggatgggatggatgaaccctaacactggtgacacatcctacgcacagaggttccagggcagagtcaccatgatcagggacgtctccataagtacagcctacttggaattgacaggcctgacatttgatgacacggccgtctacttctgtgcgaggtcgaattctgcacctgtttcaactcttttacccgctgatgcttttgatgtctggggccaagggacaatggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 343 - protein, 446 aa, Homo sapiens
QVQLVQSGAEVKRPGASVKVSCETSGYSFTTDLNWVRQAAGQGLEWMGWMNPNTGDTSYAQRFQGRVTMIRDVSISTAYLELTGLTFDDTAVYFCARSNSAPVSTLLPADAFDVWGQGTMVTVSSASTKGPSVFPLAPSSKSTSGGTAALGCLVKDYFPEPVTVSWNSGALTSGVTFPAVLQSSGLYSLSSVVTVPSSSLGTQTYICNVNKPSNTKVDKKVEPKSCDKTTCPPCPAPELLGGPSVFLFPPKPKDTLMISRTPEVTCVVVDVSEDPEVKFNWYVDGVEVNAKTKPREEQYNSTYRVVSVLTVLQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMEALNYTQKSLSLSPGK

SEQ ID NO: 344 - DNA, 645 bp, Homo sapiens
gacatccagatgacccagtctccatcctccctgtctgcatctataggagacacagtcaccatcacttgccgggcaagtcagatcattaacagctatttaaattggtatcagcaaaaaccaggggaagcccctaaactcctgatctatgctgtatccaatttgcaccgtggggtcccctcaaggttcagtggcagtgggtctgcggcagatttcactctcactatcagtggtctgcaacctgaagattttgcaacttactactgtcaacagacttacactaccgttgcgatcaccttcggccaagggacacgactggacattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 345 - protein, 215 aa, Homo sapiens
DIQMTQSPSSLSASIGDTVTITCRASQIINSYLNWYQQKPGEAPKLLIYAVSNLHRGVPSRFSGSGSAADFTLTISGLQPEDFATYYCQQTYTTVAITFGQGTRLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 346 - DNA, 1349 bp, Homo sapiens
caggtgcagctggtgcagtctggggcggaggtgaagaagcctgggtcctcggtgaacgtctcctgtaaggcctccgcagggaccctcagcggttataccatcaactggttgcgccaggcccctggacgagggtttgagtggctgggaaggatcatccctcaccttggaataggaaacaacgcacataactgggaggccagagtcacccttaccgccgacaagtccacggacactacctacctggagttgcgcgacctgagatctgacgacgtggccatttattactgcgcggtggagttttaccggctggcaacggtcacaaccccaacccttgacttctggggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctaagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 347 - protein, 443 aa, Homo sapiens
QVQLVQSGAEVKKPGSSVNVSCKASAGTLSGYTINWLRQAPGRGFEWLGRIIPLGIGNNANWEARVTLTADKSTDTTYLELRDLRSDDVAIYYCAVEFYRLATVTTPTLDFWGQGTLVTVSSASTKGPSVFPLAPSSKSTSGGTAALGCLVKDYFPEPVTVSWNSGALTSGVTFPAVLQSSGLYSLSSVVTVPSSSLGTQTYICNVNKPSNTKVDKKVEPKSCDKTTCPPCPAPELLGGPSVFLFPPKPKDTLMISRTPEVTCVVVDVSEDPEVKFNWYVDGVEVNAKTKPREEQYNSTYRVVSVLTVLQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMEALNYTQKSLSLSPGK

SEQ ID NO: 348 - DNA, 642 bp, Homo sapiens
tcctatgtgctgactcagccaccctcggtgtccgtggccccaggaacgacggccaggattacctgtgagggggaccacattggagctaaaagtgtgcactggtaccagaggaagccaggccaggcccctgtgttggtcgtctatatgaatagtgaccggccctcagggatccctgagcgattgtctggctccaactctgggaacacggccaccctgagtatcagcagggtcgaggccgaggatgaggccgactattattgtcaggtttgggatagttttcgcgatcatcaggtattcggcggagggaccaagttgaccgtcctgagtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaggcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 349 - protein, 214 aa, Homo sapiens
SYVLTQPPSVSVAPGTTARITCEGDHIGAKSVHWYQRKPGQAPVLVVYMNSDRPSGIPERLSGSNSGNTATLSISRVEAEDEADYYCQVWDSFRDHQVFGGGTKLTVLSQPKAAPSVTLFPPSSEELQANKATLVCLISDFYPGAVTVAWKADSSPVKAGVETTTPSKQGNNKYAASSYLSLTPEQWKSHRSYSCQVTHEGSTVEKTVAPTECS

SEQ ID NO: 350 - DNA, 1365 bp, Homo sapiens
caggtcaccttgaaggagtctggtcctgctatagtgaaacccacacagaccctcacgctgacctgcaccttctctgggttctcactccgaactagtggagtgggtgtgggctggatccgtcagcccccaggaaaggcccttgagtggcttgcactcatttactgggatgatgataagcgctacaacccatctctgaagaccagactcaccatcaccaaggactcctccagaaaccaggtgttcctcacaatgaccaacatggaccctgtggacacagccacatacttctgtgcacaccgacaacatattacaaacagttttggattggttattgcgaatgatgcttttgacatttggggccaagggacaatggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 351 - protein, 448 aa, Homo sapiens
QVTLKESGPAIVKPTQTLTLTCTFSGFSLRTSGVGVGWIRQPPGKALEWLALIYWDDDKRYNPSLKTRLTITKDSSRNQVFLTMTNMDPVDTATYFCARQITNSFGLVIANDAFDIWGQGTMVTVSSASTKGPSVFPLAPSSKSTSGGTAALGCLVKDYFPEPVTVSWNSGALTSGVTFPAVLQSSGLYSLSSVVTVPSSSLGTQTYICNVNKPSNTKVDKKVEPKSCDKTTCPPCPAPELLGGPSVFLFPPKPKDTLMISRTPEVTCVVVDVSEDPEVKFNWYVDGVEVNAKTKPREEQYNSTYRVVSVLTVLQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMEALNYTQKSLSLSPGK

SEQ ID NO: 352 - DNA, 651 bp, Homo sapiens
cagtctgccctgactcagcctccgtccgcgtccgggtctcctggacagtcagtcaccatctcctgcactggaaccagcagtgacgttggtcgttataattatgtcgcctggtatcaacaacacccaggcagagcccccagactcataatttatggggtcactaagcggccctcaggggtccctgatcgcttctctggctccaagtctggcaacacggcctccctgaccgtctctgggctccaggctgaagatgaggctgattattattgcagttcatatgcaggcagcaactttttggaggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagttgcccctacggaatgttca

SEQ ID NO: 353 - protein, 217 aa, Homo sapiens
QSALTQPPSASGSPGQSVTISCTGTSSDVGRYNYVAWYQQHPGRAPRLIIYGVTKRPSGVPDRFSGSKSGNTASLTVSGLQAEDEADYYCSSYAGSNFLEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 354 - DNA, 1356 bp, Homo sapiens
caggtgcagctggtgcagtctggggctgaggtgaagaagcctgggtcctcggtgaaggtctcctgcaaggcttctggaggcaccttcagcagctatgcaatcaactgggtgcgacaggcccctggacaaggtcttgagtggatgggagggatcatccctatgtttgatacagcaatctacgcacagatgttccagggcagagtcaccatcaccgcggacgaatccacgagcacagcctacatggagctgagcagcctgagatctgacgacacggccgtatattactgtgcgagagatcgaaacgattattatgacagtagtggttattcaggcgcctttgaccagtggggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 355 - protein, 447 aa, Homo sapiens
QVQLVQSGAEVKKPGSSVKVSCKASGGTFSSYAINWVRQAPGQGLEWMGGIIPMFDTAIYAQMFQGRVTITADESTSTAYMELSSLRSDDTAVYYCARDRNDYYDSSGYSGAFDQWGQGTLVTVSSASTKGPSVFPLAPSSKSTSGGTAALGCLVKDYFPEPVTVSWNSGALTSGVTFPAVLQSSGLYSLSSVVTVPSSSLGTQTYICNVNKPSNTKVDKKVEPKSCDKTTCPPCPAPELLGGPSVFLFPPKPKDTLMISRTPEVTCVVVDVSEDPEVKFNWYVDGVEVNAKTKPREEQYNSTYRVVSVLTVLQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMEALNYTQKSLSLSPGK

SEQ ID NO: 356 - DNA, 656 bp, Homo sapiens
gaaattgtgttgacgcagtctccaggcgccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcacaatcagcagactggagcctgaggattttgcagtgtattactgtcagcagtatggtagctcacctcgcacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaacgcgtg

SEQ ID NO: 357 - protein, 215 aa, Homo sapiens
EIVLTQSPGALSLSPGERATLSCRASQSVSSSYLAWYQQKPGQAPRLLIYGASSRATGIPDRFSGSGSGTDFTLTISRLEPEDFAVYYCQQYGSSPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 358 - DNA, 1344 bp, Homo sapiens
gaggtgcagctggtggagtccgggggaggcttggtccagcctggggggtccctgaaactctcctgtgcagcctctgggttcagcttcagtggctctgctgtggaatgggtccgccaggcttccgggagagggctggagtggattggccatataagaaccaagaccaaaaattacgcgacagcgtatgctgcgtcggtgcaaggcaggttcaccatcttcagagatgattcaaggaacacggcgtatctgcagatgaacagtcttaaaaccgaggacacggccgtgtattattgttccagacacatcgaatttagcagttcgtccgggccccagatgggccagggaaccctggtcaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 359 - protein, 441 aa, Homo sapiens
EVQLVESGGGLVQPGGSLKLSCAASGFSFSGSAVEWVRQASGRGLEWIGIRTKTKNYATAYAASVQGRFTIFRDDSRNTAYLQMNSLKTEDTAVYYCSRIEFSSSSGPQMGQGTLVTVSSASTKGPSVFPLAPSSKSTSGGTAALGCLVKDYFPEPVTVSWNSGALTSGVTFPAVLQSSGLYSLSSVVTVPSSSLGTQTYICNVNKPSNTKVDKKVEPKSCDKTTCPPCPAPELLGGPSVFLFPPKPKDTLMISRTPEVTCVVVDVSEDPEVKFNWYVDGVEVNAKTKPREEQYNSTYRVVSVLTVLQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMEALNYTQKSLSLSPGK

SEQ ID NO: 360 - DNA, 642 bp, Homo sapiens
gacatccagatgacccagtctccttccaccctgtctgcatctgtaggcgacacagtcaccatcacttgccaggccagtcagaatattattacttggttggcctggtatcagcagaaaccagggaaagcccctaacatcctcatctataaggcgtctggtttagagagtggggtcccaccgaggttcagcggcagtgggtcggggacagaattcactctcaccatcagcagcctgcagcctgatgatgttgcaacttattattgcctacaatataaaactgagccgtggacgttcggcctagggaccaaggtggagatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 361 - protein, 214 aa, Homo sapiens
DIQMTQSPSTLSASVGDTVTITCQASQNIITWLAWYQQKPGKAPNILIYKASGLESGVPPRFSGSGSGTEFTLTISSLQPDDVATYYCLQYKTEPWTFGLGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 362 - DNA, 1335 bp, Homo sapiens
gaggtgcagctggtggagtctgggggaggcttggcccagccgggggagtccctgagactctcctgtgcagcctccggattcacctttagtgcctattggatgagctgggtccgccagactccagggaaggggctggagtgggtggccaacattaaccagggtggagataagacagactatttggactctgtgaagggccgattcaccgtctccagagacaatgccaagaacttagtgtttctgcaaatggccagcctgagagccgacgacacggccgtgtattactgtgggagaggtacaaaccaagactactggggccagggaaccctggtcaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 363 - protein, 445 aa, Homo sapiens
EVQLVESGGGLAQPGESLRLSCAASGFTFSAYWMSWVRQTPGKGLEWVANINQGGDKTDYLDSVKGRFTVSRDNAKNLVFLQMASLRADDTAVYYCGRGTNQDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 364 - DNA, 648 bp, Homo sapiens
cagtctgtgctgactcagccaccctcagcgtctgggacccccgggcagagggtcaccatctcttgttctggaagcagctccaatatcggaagtaataatgtatactggtaccaacaggtcccaggaacggcccccaaatttctcatctatccgaatagtgagcggccctcaggggtccctgaccgattctctgcctccaagtctggcacctcagcctccctggccatcagtggactccggtccgaggatgaggctacttattactgtgcagcatgggatgacagcctaagaacttatgtcttcgcatctgggaccaaggtcaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 365 - protein, 216 aa, Homo sapiens
QSVLTQPPSASGTPGQRVTISCSGSSSNIGSNNVYWYQQVPGTAPKFLIYPNSERPSGVPDRFSASKSGTSASLAISGLRSEDEATYYCAAWDDSLRTYVFASGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 366 - DNA, 1350 bp, Homo sapiens
caggtgcagctggtggagtctgggggaggcgtggtccagcctgggaggtccctgagactctcctgtgcagcctccggattcaccctcaggagttatggcatgcactgggtccgccaggctccaggcaagggcctggagtgggtggcagttatatcatatgatggaagtaagacatattatagagactccgtgaagggccgattcaccatctccagagacaattctaagaacacactgtatttgcaaatgaacagcctgcgagttgaggacacggctgtatattattgtgcgaaagaggataatttgggatctgatgtttttgatagatggggccaagggacaatggtcaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 367 - protein, 450 aa, Homo sapiens
QVQLVESGGGVVQPGRSLRLSCAASGFTLRSYGMHWVRQAPGKGLEWVAVISYDGSKTYYRDSVKGRFTISRDNSKNTLYLQMNSLRVEDTAVYYCAKEDNLGSDVFDRWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - DNA, 643 bp, Homo sapiens
gacatcgtgatgacccagtctccttccaccctgtctgcatctgtaggagacagagttaccatcacttgtcgggccagtcagagtgccagtcagtggttggcctggtatcagcaaaaaccagggaaagcccctaatctcctaatctataaggcgtctaatttacatagtggggtcccatcaaggttcagcggcagtggatctgggaccgaattcactctcaccatcagcagcctacagcctgatgattttgcaacttattactgccaacaatacaacatttatccgtggacgttcggccaagggaccaaggtggaagtgagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtt

SEQ ID NO: 369 - protein, 214 aa, Homo sapiens
DIVMTQSPSTLSASVGDRVTITCRASQSASQWLAWYQQKPGKAPNLLIYKASNLHSGVPSRFSGSGSGTEFTLTISSLQPDDFATYYCQQYNIYPWTFGQGTKVEVRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 370 - DNA, 1350 bp, Homo sapiens
caggtccagctggtgcagtctggggctgaggtgaaggagccagggtcctcggtgagggtctcctgcaaggcttctggacgcatcttccggaattatgctgtcagctgggtgcgacaggcccctggacaagggcttgagtgggtgggccggttcatcccttcggaaggaataacaaactacattccgacattccgggacagactcacctttactgcggacaagtccacgaacacagccttcattgacctgactaacctgaaatatgaggacacggccatatattactgtgtgggtggcaggccaggcgctttcgtcccctttgactcctggggccagggaaccctggtcaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 371 - protein, 450 aa, Homo sapiens
QVQLVQSGAEVKEPGSSVRVSCKASGRIFRNYAVSWVRQAPGQGLEWVGRFIPSEGITNYIPTFRDRLTFTADKSTNTAFIDLTNLKYEDTAIYYCVGGRPGAFVPFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - DNA, 643 bp, Homo sapiens
gaaatagtgatgacgcagtctcctgccaccctgtctgtgtctccaggggaaagagccaccctctcctgcagggccagtcagagtgtcaggggcaacttagcctggtaccagcaaaaacctggccaggctcccaggcttctcatgtatgatgcttctaccagggccactagtttcccagcccggttcagtggcagtgggtctgggacagagttcactctcaccatcagcagtgtgcagtctgaagattttgcactttattactgtcagcagtataatgactggccccggacttttggccaggggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtt

SEQ ID NO: 373 - protein, 214 aa, Homo sapiens
EIVMTQSPATLSVSPGERATLSCRASQSVRGNLAWYQQKPGQAPRLLMYDASTRATSFPARFSGSGSGTEFTLTISSVQSEDFALYYCQQYNDWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 374 - DNA, 1362 bp, Homo sapiens
caggtgcagctggtgcagtctggggctgaggtgaagaagcctgggtcctcggtgagggtctcctgcagggtttctggaggcagtttctccagttatgacgtacactgggtgcgacaggcccctggacaagggcttgagtgggtgggaaggagcgtctcacgtgctgacagctcaagtcacacacgggagttcggggacagagtcacgattatcgcggacaaatccacgagtacagtttatttgacactgaccagtctgacctctgacgacacggccatctattactgtgcggcaggggattgtagtgatggcgggtgcttccgcacacccctgcacatctggggccagggcaccctggtcaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 375 - protein, 454 aa, Homo sapiens
QVQLVQSGAEVKKPGSSVRVSCRVSGGSFSSYDVHWVRQAPGQGLEWVGRSVSRADSSSHTREFGDRVTIIADKSTSTVYLTLTSLTSDDTAIYYCAAGDCSDGGCFRTPLHIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 376 - DNA, 645 bp, Homo sapiens
gaaatagtgatgacgcagtctccagccactctgtctctgtctccaggggaaagagccaccctctcctgcagggccagtcagagtctgagaaccaacttagcctggtaccagcagcgacctggccaggctcccaggctcctcatctctggggcatccacgagggccactggtatcccagccaggttcagtggcagtgggtctgggacagaattcactctcaccattaggagcctgcagcctgaagattttgcagtttattactgtcaacagtatcatgactggcctcggacgttcggccaagggaccaaggtggatttcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgttag

SEQ ID NO: 377 - protein, 214 aa, Homo sapiens
EIVMTQSPATLSLSPGERATLSCRASQSLRTNLAWYQQRPGQAPRLLISGASTRATGIPARFSGSGSGTEFTLTIRSLQPEDFAVYYCQQYHDWPRTFGQGTKVDFKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 378 - DNA, 1359 bp, Homo sapiens
gaggtgcagctggtgcagtctggggcagaagtgaaaaaggccggggagtctctgaggatctcctgtaagggttctggatacaactttaatagctattggattggctgggtgcgccagatgcccgggaaaggcctggagtggatggcaatcatctatcctgctgactctgacaggagatatggcccgtccttccaaggccaggtcaccatctcagtcgacaagtctattaacaccgcgtacttgcagtggagcagcctgaaggcctcggacaccgccatgtattattgtgccagactcctcagagttagtactggttgggaagatgcttttgatctgtggggccaagggacaatggtcaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 379 - protein, 453 aa, Homo sapiens
EVQLVQSGAEVKKAGESLRISCKGSGYNFNSYWIGWVRQMPGKGLEWMAIIYPADSDRRYGPSFQGQVTISVDKSINTAYLQWSSLKASDTAMYYCARLLRVSTGWEDAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 380 - DNA, 663 bp, Homo sapiens
gacatcgtgatgacccagtctccagactccctggttgtgtctctgggcgagagggccaccatcaagtgccagtccagccagagtgttttgtacagctccaccaataagaactatttagcctggtaccagcagaaatcgggacagccacctaagctactcatttactgggcctccacccgggaatccggggtccctgaccgattcattggtagcggctctaggacagatttcactctcaccatcagcgacctgcagcctgaggatgcggcagtttattactgtcagcagtattatagtgattatctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 381 - protein, 220 aa, Homo sapiens
DIVMTQSPDSLVVSLGERATIKCQSSQSVLYSSTNKNYLAWYQQKSGQPPKLLIYWASTRESGVPDRFIGSGSRTDFTLTISDLQPEDAAVYYCQQYYSDYLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC